(19) United States
(12) Patent Application Publication
(10) Pub. No.: US 2023/0200221 A1
(43) Pub. Date: Jun. 22, 2023

(54) LIGHT-EMITTING DEVICE INCLUDING CONDENSED CYCLIC COMPOUND, ELECTRONIC APPARATUS INCLUDING THE LIGHT-EMITTING DEVICE, AND THE CONDENSED CYCLIC COMPOUND

(71) Applicant: Samsung Display Co., Ltd., Yongin-si (KR)

(72) Inventors: Hankyu Pak, Yongin-si (KR); Dongjun Kim, Yongin-si (KR); Chaeyeong Kim, Yongin-si (KR); Byeongwook Yoo, Yongin-si (KR); Seowon Cho, Yongin-si (KR); Sohee Jo, Yongin-si (KR)

(21) Appl. No.: 18/063,031

(22) Filed: Dec. 7, 2022

(30) Foreign Application Priority Data
Dec. 20, 2021 (KR) 10-2021-0183127

(51) Int. Cl. H10K 85/60; C07C 211/54; C07D 209/82; C07D 307/91; C07D 333/76; H10K 50/15

(52) U.S. Cl. CPC H01L 51/006; C07C 211/54; H01L 51/0056; H01L 51/0072; C07D 209/82; H01L 51/0073; C07D 307/91; H01L 51/0074; C07D 333/76; H01L 51/5056

(57) ABSTRACT

A condensed cyclic compound represented by Formula 1, a light-emitting device including the condensed cyclic compound, and an electronic apparatus including the light-emitting device:

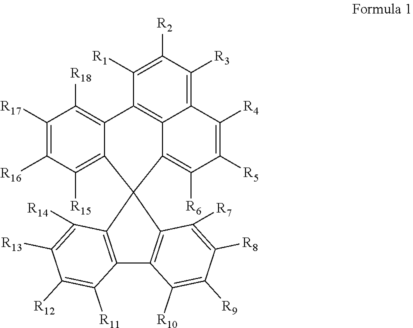

wherein substituents of Formula 1 are the same as described herein.

### CROSS-REFERENCE TO RELATED APPLICATION

This application claims priority to and the benefit of Korean Patent Application No. 10-2021-0183127, filed on Dec. 20, 2021, in the Korean Intellectual Property Office, the disclosure of which is incorporated herein by reference in its entirety.

### BACKGROUND

### 1. Field

One or more embodiments relate to a light-emitting device including a condensed cyclic compound, an electronic apparatus including the light-emitting device, and the condensed cyclic compound.

### 2. Description of the Related Art

Self-emissive devices from among light-emitting devices relatively have, as compared with other devices of the related art, wide viewing angles, high contrast ratios, short response times, and/or excellent or suitable characteristics in terms of luminance, driving voltage, and/or response speed, and produce full-color images.

In a light-emitting device, a first electrode is located on a substrate, and a hole transport region, an emission layer, an electron transport region, and a second electrode are sequentially formed on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. The excitons may transition from an excited state to a ground state, thereby generating light.

### SUMMARY

Aspects according to one or more embodiments of the present disclosure are directed toward a light-emitting device including a condensed cyclic compound, an electronic apparatus including the light-emitting device, and the condensed cyclic compound.

Additional aspects will be set forth in part in the description which follows and, in part, will be apparent from the description, or may be learned by practice of the presented embodiments of the disclosure.

According to one or more embodiments, a condensed cyclic compound is represented by Formula 1.

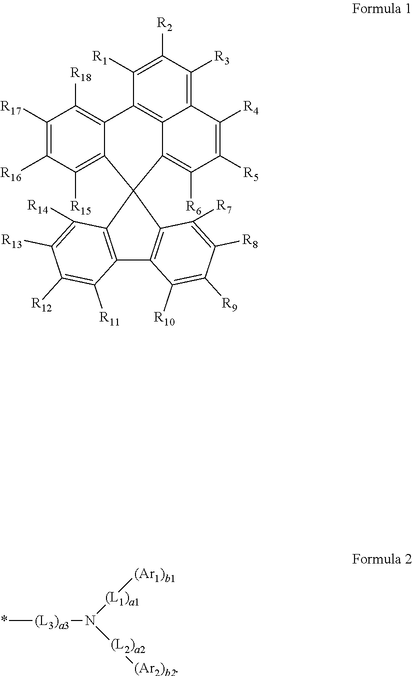

In Formulae 1 and 2,

at least one selected from the group consisting of R₇ to R₉, R₁₂ to R₁₄, and R₁₆ may be a group represented by Formula 2, and the others (remainder ones of these groups) may each independently be hydrogen or the same as in the description of R₁₀ₐ,

R₁ to R₆, R₁₀, R₁₁, R₁₅, R₁₇, and R₁₈ may each independently be hydrogen or the same as in the description of R₁₀ₐ, and at least one selected from the group consisting of R₁ to R₆, R₁₀, R₁₁, R₁₅, R₁₇, and R₁₈ may not be hydrogen,

L₁ to L₃ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,

a1 to a3 may each independently be an integer from 1 to 3,

Ar₁ and Ar₂ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

b1 and b2 may each independently be an integer from 1 to 10,

* indicates a binding site to a neighboring atom,

R₁₀ₐ may be

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroarylalkyl group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof,

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof, or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂),

Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group, a C₁-C₆₀ heterocyclic group, a C₇-C₆₀ aryl alkyl group, or a C₂-C₆₀ heteroaryl alkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, a light-emitting device includes a first electrode,

a second electrode facing the first electrode,

an interlayer between the first electrode and the second electrode and including an emission layer, and

at least one condensed cyclic compound represented by Formula 1.

According to one or more embodiments, an electronic apparatus includes the light-emitting device.

### BRIEF DESCRIPTION OF THE DRAWINGS

The above and other aspects, features, and enhancements of certain embodiments of the disclosure will be more apparent from the following description taken in conjunction with the accompanying drawings, in which:

FIG. 1 is a schematic cross-sectional view of a light-emitting device according to an embodiment; and

FIG. 2 is a schematic cross-sectional view of an electronic apparatus according to an embodiment of the present disclosure.

FIG. 3 is a schematic cross-sectional view of an electronic apparatus according to an embodiment of the present disclosure.

### DETAILED DESCRIPTION

Reference will now be made in more detail to embodiments, examples of which are illustrated in the accompanying drawings, wherein like reference numerals refer to like elements throughout, and duplicative descriptions thereof may not be provided. In this regard, the present embodiments may have different forms and should not be construed as being limited to the descriptions set forth herein. Accordingly, the embodiments are merely described below, by referring to the drawings, to explain aspects of the present description. As used herein, the term “and/or” includes any and all combinations of one or more of the associated listed items. Throughout the disclosure, the expression, such as “at least one of a, b or c”, “at least one selected from a, b, and c”, “at least one selected from the ground consisting of a, b, and c”, etc., indicates only a, only b, only c, both (e.g., simultaneously) a and b, both (e.g., simultaneously) a and c, both (e.g., simultaneously) b and c, all of a, b, and c, or variation(s) thereof.

According to an embodiment, a condensed cyclic compound may be represented by Formula 1:

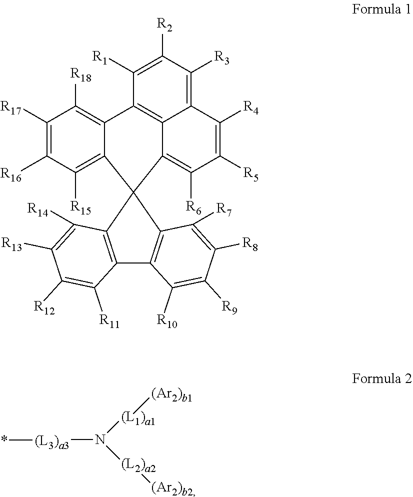

wherein, in Formulae 1 and 2,

at least one selected from the group consisting of R₇ to R₉, R₁₂ to R₁₄, and R₁₆ may be a group represented by Formula 2, and the others (remainder ones of these groups) may each independently be hydrogen or the same as in the description of R₁₀ₐ below,

The term “the others” as used herein in connection with R₇ to R₉, R₁₂ to R₁₄, and R₁₆ indicate R₇ to R₉, R₁₂ to R₁₄, and R₁₆ that are not a group represented by Formula 2 from among R₇ to R₉, R₁₂ to R₁₄, and R₁₆. That is, the term “the others” as used herein in connection with R₇ to R₉, R₁₂ to R₁₄, and R₁₆ refers to remainder ones of R₇ to R₉, R₁₂ to R₁₄, and R₁₆ after excluding those that are each a group represented by Formula 2. For example, when R₈ is a group represented by Formula 2, “the others” may indicate (refers to) R₇, R₉, R₁₂ to R₁₄, and R₁₆ (refer to Compound 306, etc.).

R₁ to R₆, R₁₀, R₁₁, R₁₅, R₁₇, and R₁₈ may each independently be hydrogen or the same as in the description of R₁₀ₐ below, and at least one selected from the group consisting of R₁ to R₆, R₁₀, R₁₁, R₁₅, R₁₇, and R₁₈ may not be hydrogen.

The phrase “at least one selected from the group consisting of R₁ to R₆, R₁₀, R₁₁, R₁₅, R₁₇, and R₁₈ is not hydrogen” as used herein refers to that at least one selected from the group consisting of R₆, R₁₀, R₁₁, R₁₅, R₁₇, and R₁₈ is the same as described in R₁₀ₐ below (one of functional groups described in R₁₀ₐ) and the others (e.g., remainder ones of these groups) are each hydrogen. For example, when R₆ is a functional group described in connection with R₁₀ₐ, R₁ to R₅, R₁₀, R₁₁, R₁₅, R₁₇, and R₁₈ may each be hydrogen (refer to Compound 1, etc.).

In an embodiment, two or more adjacent groups of R₁ to R₁₈ may not be bonded to each other to form a condensed ring.

In an embodiment, in Formula 1, two selected from the group consisting of R₇ to R₉, R₁₂ to R₁₄, and R₁₆ may each be a group represented by Formula 2 and the others (e.g., remainder ones of these groups) may each independently be hydrogen or the same as in the description of R₁₀ₐ.

For example, i) R₇ and R₁₂, R₇ and R₁₃, R₇ and R₁₄, R₈ and R₁₂, R₈ and R₁₃, R₈ and R₁₄, R₉ and R₁₂, R₉ and R₁₃, R₉ and R₁₄, R₇ and R₁₆, R₈ and R₁₆, R₉ and R₁₆, R₁₂ and R₁₆, R₁₃ and R₁₆, or R₁₄ and R₁₆ may each be a group represented by Formula 2, and ii) R₇ to R₉, R₁₂ to R₁₄, and R₁₆, which are not groups represented by Formula 2 from among R₇ to R₉, R₁₂ to R₁₄, and R₁₆ may each independently be hydrogen or the same as in the description of R₁₀ₐ.

In an embodiment, in Formula 1, two selected from the group consisting of R₁ to R₆, R₁₀, R₁₁, R₁₅, R₁₇, and R₁₈ may each independently be the same as in the description of R₁₀ₐ and the others (e.g., remainder ones of these groups) may each be hydrogen.

For example, in R₁ to R₆, R₁₀, R₁₁, R₁₅, R₁₇, and R₁₈, i) one selected from the group consisting of R₁ to R₃ and one selected from the group consisting of R₄ to R₆ may each independently be the same as in the description of R₁₀ₐ, and ii) the others (e.g., remainder ones of these groups) may each be hydrogen.

For example, in R₁ to R₆, R₁₀, R₁₁, R₁₅, R₁₇, and R₁₈, i) one selected from the group consisting of R₁ to R₃ and one selected from the group consisting of R₁₀ and R₁₁ may each independently be the same as in the description of R₁₀ₐ, and ii) the others (e.g., remainder ones of these groups) may each be hydrogen.

For example, in R₁ to R₆, R₁₀, R₁₁, R₁₅, R₁₇, and R₁₈, i) one selected from the group consisting of R₁ to R₃ and one selected from the group consisting of R₁₅ and R₁₇ may each independently be the same as in the description of R₁₀ₐ, and ii) the others (e.g., remainder ones of these groups) may each be hydrogen.

For example, in R₁ to R₆, R₁₀, R₁₁, R₁₅, R₁₇, and R₁₈, i) one selected from the group consisting of R₄ to R₆ and one selected from the group consisting of R₁₀ and R₁₁ may each independently be the same as in the description of R₁₀ₐ, and ii) the others (e.g., remainder ones of these groups) may each be hydrogen.

For example, in R₁ to R₆, R₁₀, R₁₁, R₁₅, R₁₇, and R₁₈, i) one selected from the group consisting of R₄ to R₆ and one selected from the group consisting of R₁₅ and R₁₇ may each independently be the same as in the description of R₁₀ₐ, and ii) the others (e.g., remainder ones of these groups) may each be hydrogen.

For example, in R₁ to R₆, R₁₀, R₁₁, R₁₅, R₁₇, and R₁₈, i) one selected from the group consisting of R₁₀ and R₁₁ and one selected from the group consisting of R₁₅ and R₁₇ may each independently be the same as in the description of R₁₀ₐ, and ii) the others (e.g., remainder ones of these groups) may each be hydrogen.

In an embodiment, in Formula 1, one selected from the group consisting of R₇ to R₉, R₁₂ to R₁₄, and R₁₆ may be a group represented by Formula 2, and ii) the others (e.g., remainder ones of these groups) may each independently be hydrogen or the same as in the description of R₁₀ₐ.

In an embodiment, in Formula 1, i) one selected from the group consisting of R₇ to R₉, R₁₂ to R₁₄, and R₁₆ may be a group represented by Formula 2, and ii) the others (e.g., remainder ones of these groups) may each independently be: hydrogen, deuterium, —F, —Cl, —Br, —I, a hydroxyl group, a cyano group, a nitro group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a C₁-C₂₀ alkyl phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indenyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzosilolyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzofluorenyl group, a benzocarbazolyl group, a naphthobenzofuranyl group, a naphthobenzothiophenyl group, a naphthobenzosilolyl group, a dibenzofluorenyl group, a dibenzocarbazolyl group, a dinaphthofuranyl group, a dinaphthothiophenyl group, a dinaphthosilolyl group, an indenocarbazolyl group, an indolocarbazolyl group, a benzofuranocarbazolyl group, a benzothienocarbazolyl group, a benzosilolocarbazolyl group, an imidazopyridinyl group, an imidazopyrimidinyl group, an azafluorenyl group, an azacarbazolyl group, an azadibenzofuranyl group, an azadibenzothiophenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a C₁-C₂₀ alkyl phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indenyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzosilolyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzofluorenyl group, a benzocarbazolyl group, a naphthobenzofuranyl group, a naphthobenzothiophenyl group, a naphthobenzosilolyl group, a dibenzofluorenyl group, a dibenzocarbazolyl group, a dinaphthofuranyl group, a dinaphthothiophenyl group, a dinaphthosilolyl group, an indenocarbazolyl group, an indolocarbazolyl group, a benzofuranocarbazolyl group, a benzothienocarbazolyl group, a benzosilolocarbazolyl group, an imidazopyridinyl group, an imidazopyrimidinyl group, —Si(Q₂₁)(Q₂₂)(Q₂₃), —B(Q₂₁)(Q₂₂), —P(Q₂₁)(Q₂₂), —C(═O)(Q₃₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁) or —P(═O)(Q₃₁)(Q₃₂),

Q₂₁ to Q₂₃ and Q₃₁ to Q₃₃ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with deuterium, a C₁-C₂₀ alkyl group, a phenyl group, a biphenyl group, a pyridinyl group, a pyrimidinyl group, a pyridazinyl group, a pyrazinyl group, a triazinyl group, or any combination thereof.

In an embodiment, in Formula 1, one selected from the group consisting of R₇ to R₉, R₁₂ to R₁₄, and R₁₆ may be a group represented by Formula 2, and the others (e.g., remainder ones of these groups) may each be hydrogen.

For example, one selected from the group consisting of R₈ and R₁₃ may be a group represented by Formula 2, and the others (e.g., remainder ones of these groups), R₇ to R₉, R₁₂ to R₁₄, and R₁₆, which are not groups represented by Formula 2, may each be hydrogen.

For example, R₈ may be a group represented by Formula 2, and R₇, R₉, R₁₂ to R₁₄, and R₁₆ may each be hydrogen.

For example, R₁₃ may be a group represented by Formula 2, and R₇ to R₉, R₁₂, R₁₄, and R₁₆ may each be hydrogen.

For example, R₁₂ may be a group represented by Formula 2, and R₇ to R₉, R₁₃, R₁₄, and R₁₆ may each be hydrogen.

For example, R₁₄ may be a group represented by Formula 2, and R₇ to R₉, R₁₂, R₁₃, and R₁₆ may each be hydrogen.

For example, R₁₆ may be a group represented by Formula 2, and R₇ to R₉, and R₁₂ to R₁₄ may each be hydrogen.

In an embodiment, in Formula 1, one selected from the group consisting of R₁ to R₆, R₁₀, R₁₁, R₁₅, R₁₇, and R₁₈ may not be hydrogen, and the others (e.g., remainder ones of these groups) may each be hydrogen. For example, in Formula 1, one selected from the group consisting of R₁ to R₆, R₁₀, R₁₁, R₁₅, R₁₇, and R₁₈ may be the same as in the description of R₁₀ₐ, and the others (e.g., remainder ones of these groups) may each be hydrogen.

In one or more embodiments, in R₁ to R₆, R₁₀, R₁₁, R₁₅, R₁₇, and R₁₈, i) one selected from the group consisting of R₁ to R₃, one selected from the group consisting of R₄ to R₆, one selected from the group consisting of R₁₀ and R₁₁, or one selected from the group consisting of R₁₅ and R₁₇ may be the same as in the description of R₁₀ₐ, and ii) the others (e.g., remainder ones of these groups) may each be hydrogen.

For example, in R₁ to R₆, R₁₀, R₁₁, R₁₅, R₁₇, and R₁₈, i) any one selected from the group consisting of R₃, R₆, R₁₀, R₁₁, and R₁₇ may not be hydrogen, and ii) the others (e.g., remainder ones of these groups) may each be hydrogen.

In an embodiment, in Formula 1, i) one selected from the group consisting of R₁ to R₆, R₁₀, R₁₁, R₁₅, R₁₇, and R₁₈ may be: deuterium, —F, —Cl, —Br, —I, a hydroxyl group, a cyano group, a nitro group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a pyridinyl group, a pyrimidinyl group, or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a C₁-C₂₀ alkyl phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indenyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzosilolyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzofluorenyl group, a benzocarbazolyl group, a naphthobenzofuranyl group, a naphthobenzothiophenyl group, a naphthobenzosilolyl group, a dibenzofluorenyl group, a dibenzocarbazolyl group, a dinaphthofuranyl group, a dinaphthothiophenyl group, a dinaphthosilolyl group, an indenocarbazolyl group, an indolocarbazolyl group, a benzofuranocarbazolyl group, a benzothienocarbazolyl group, a benzosilolocarbazolyl group, an imidazopyridinyl group, an imidazopyrimidinyl group, an azafluorenyl group, an azacarbazolyl group, an azadibenzofuranyl group, an azadibenzothiophenyl group, or an azadibenzosilolyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a C₁-C₂₀ alkyl phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indenyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzosilolyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzofluorenyl group, a benzocarbazolyl group, a naphthobenzofuranyl group, a naphthobenzothiophenyl group, a naphthobenzosilolyl group, a dibenzofluorenyl group, a dibenzocarbazolyl group, a dinaphthofuranyl group, a dinaphthothiophenyl group, a dinaphthosilolyl group, an indenocarbazolyl group, an indolocarbazolyl group, a benzofuranocarbazolyl group, a benzothienocarbazolyl group, a benzosilolocarbazolyl group, an imidazopyridinyl group, an imidazopyrimidinyl group, —Si(Q₂₁)(Q₂₂)(Q₂₃), —B(Q₂₁)(Q₂₂), —P(Q₂₁)(Q₂₂), —C(═O)(Q₃₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and

ii) the others (e.g., remainder ones of these groups) may each be hydrogen,

Q₂₁ to Q₂₃ and Q₃₁ to Q₃₃ may each independently be:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or

an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with deuterium, a C₁-C₂₀ alkyl group, a phenyl group, a biphenyl group, a pyridinyl group, a pyrimidinyl group, a pyridazinyl group, a pyrazinyl group, a triazinyl group, or any combination thereof.

In an embodiment, in Formula 1, one selected from the group consisting of R₇ to R₉, R₁₂ to R₁₄, and R₁₆ may be a group represented by Formula 2, and the others (e.g., remainder ones of these groups) may each independently be hydrogen, deuterium, —F, —Cl, —Br, —I, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, a dibenzosilolyl group, —Si(Q₃₁)(Q₃₂)(Q₃₃), or —B(Q₃₁)(Q₃₂),

in Formula 1, one selected from the group consisting of R₁ to R₆, R₁₀, R₁₁, R₁₅, R₁₇, and R₁₈ may be a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, a dibenzosilolyl group, —Si(Q₃₁)(Q₃₂)(Q₃₃), or —B(Q₃₁)(Q₃₂), and the others (e.g., remainder ones of these groups) may each be hydrogen.

For example, in R₁ to R₆, R₁₀, R₁₁, R₁₅, R₁₇, and R₁₈, i) any one selected from the group consisting of R₃, R₆, R₁₀, R₁₁, and R₁₇ may be a methyl group, an ethyl group, an n-propyl group, an isopropyl group, an n-butyl group, a sec-butyl group, an isobutyl group, a tert-butyl group, an n-pentyl group, a tert-pentyl group, neopentyl group, an isopentyl group, a sec-pentyl group, 3-pentyl group, a sec-isopentyl group, an n-hexyl group, an isohexyl group, a sec-hexyl group, a tert-hexyl group, a C₁-C₂₀ alkoxy group, a cyclopentyl group, a cyclohexyl group, a cycloheptyl group, a cyclopentenyl group, a cyclohexenyl group, a phenyl group, a biphenyl group (for example, refer to the group represented by Formulae 7-4 to 7-6, etc.), a naphthyl group (for example, refer to the group represented by Formula 7-2 or 7-3, etc.), a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, or a triphenylenyl group, and ii) the others (e.g., remainder ones of these groups) may each be hydrogen.

For example, R₃ may be a C₁-C₂₀ alkyl group, a C₃-C₁ cycloalkyl group, a phenyl group, or a naphthyl group, and R₁, R₂, R₄ to R₆, R₁₀, R₁₁, R₁₅, R₁₇, and R₁₈ may each be hydrogen.

For example, Re may be a C₁-C₂₀ alkyl group, a C₃-C₁₀ cycloalkyl group, a phenyl group, or a naphthyl group, and R₁ to R₅, R₁₀, R₁₁, R₁₅, R₁₇, and R₁₈ may each be hydrogen.

For example, R₁₀ may be a C₁-C₂₀ alkyl group, a C₃-C₁ cycloalkyl group, a phenyl group, or a naphthyl group, and R₁ to R₆, R₁₁, R₁₅, R₁₇, and R₁₈ may each be hydrogen.

For example, R₁₁ may be a C₁-C₂₀ alkyl group, a C₃-C₁ cycloalkyl group, a phenyl group, or a naphthyl group, and R₁ to R₆, R₁₀, R₁₅, R₁₇, and R₁₈ may each be hydrogen.

For example, R₁₇ may be a C₁-C₂₀ alkyl group, a C₃-C₁ cycloalkyl group, a phenyl group, or a naphthyl group, and R₁ to R₆, R₁₀, R₁₁, R₁₅, and R₁₈ may each be hydrogen.

When one selected from the group consisting of R₁ to R₆, R₁₀, R₁₁, R₁₅, R₁₇, and R₁₈ is an aryl group such as a phenyl group or a naphthyl group, through a π-conjugation expansion of the condensed cyclic compound, the condensed cyclic compound may have excellent or suitable molecular structure stabilization effect in a polaron state. For example, when Re is an aryl group such as a phenyl group or a naphthyl group, the aryl group may be positioned in parallel to a fluorene moiety of the condensed cyclic compound, and thus, the condensed cyclic compound may have excellent or suitable molecular structure stabilization effect in the polaron state.

When one selected from the group consisting of R₁ to R₆, R₁₀, R₁₁, R₁₅, R₁₇, and R₁₈ is an alkyl group such as an ethyl group, the condensed cyclic compound may have excellent or suitable effect of reducing an intermolecular interaction of the condensed cyclic compounds and reducing a packing density of the molecules.

In Formula 2, L₁ to L₃ may each independently be a single bond, a C₅-C₃₀ carbocyclic group unsubstituted or substituted with at least one R₁₀ₐ, or a C₁-C₆₀ heterocyclic group unsubstituted or substituted with at least one R₁₀ₐ, and

a1 to a3 may each independently be an integer from 1 to 3.

In an embodiment, L₁ to L₃ may each independently be: a single bond; or

a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-a fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ, and

R₁₀ₐ is the same as described herein.

In an embodiment, L₁ to L₃ may each independently be: a single bond; or

a group represented by one of Formulae 3-1 to 3-40:

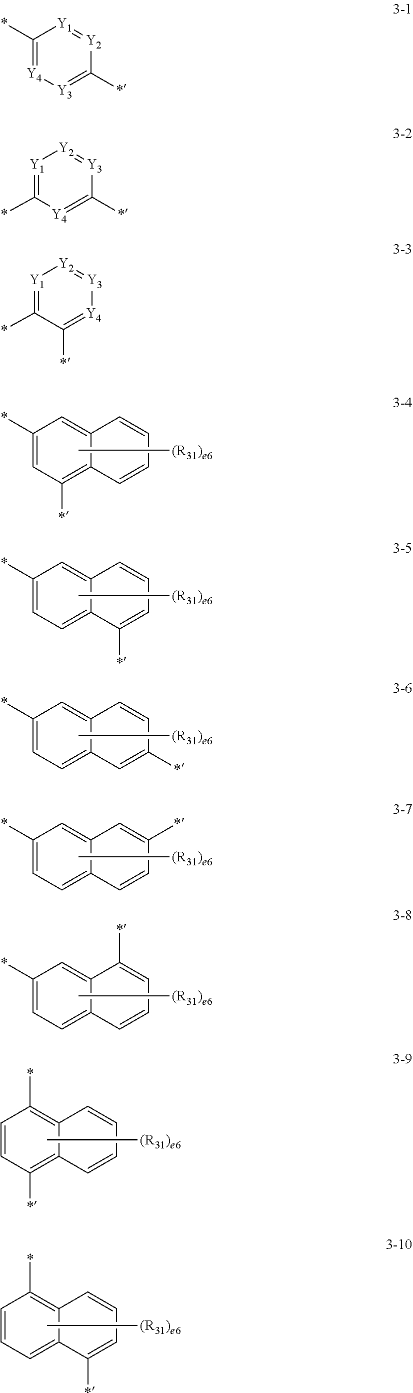

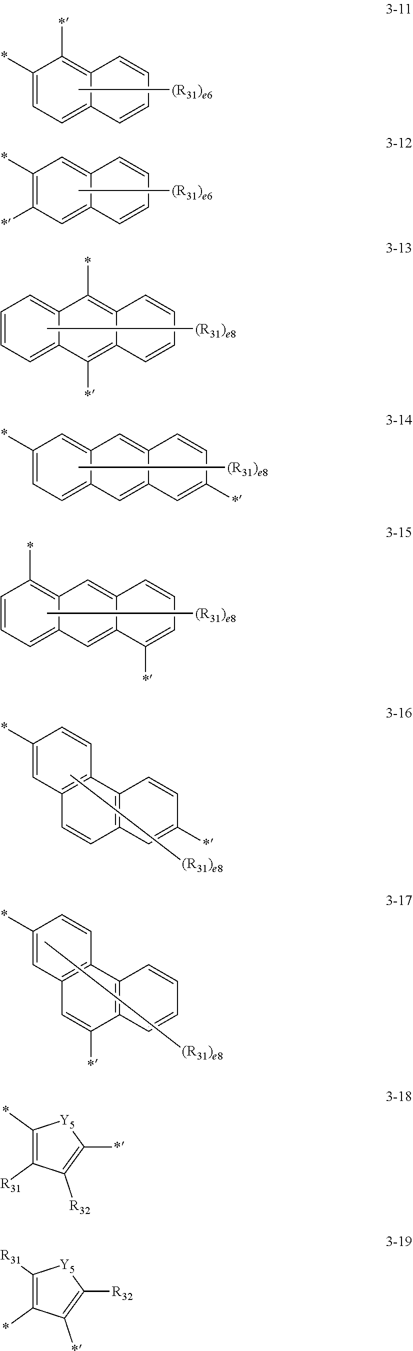

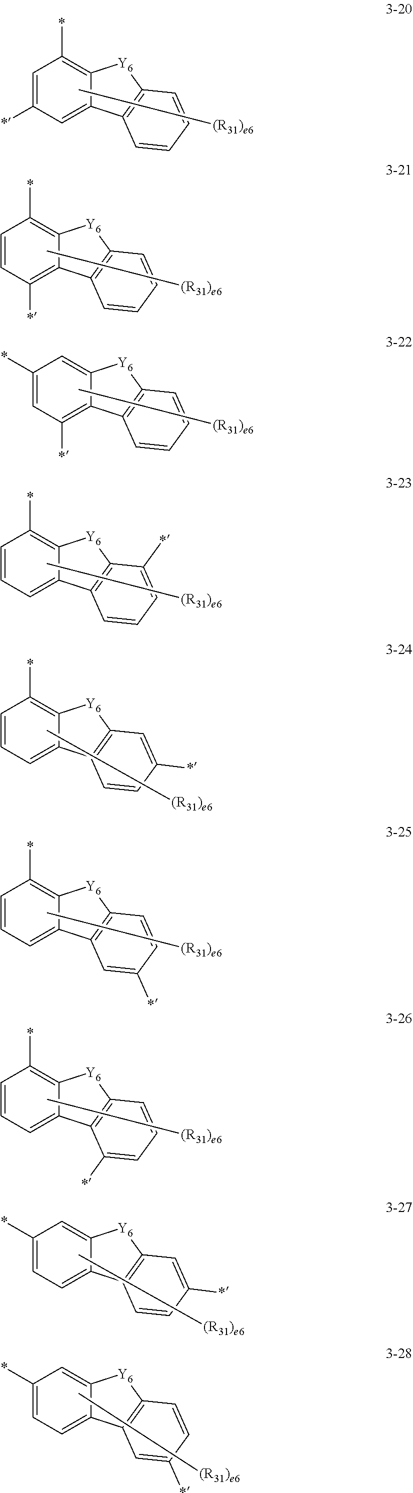

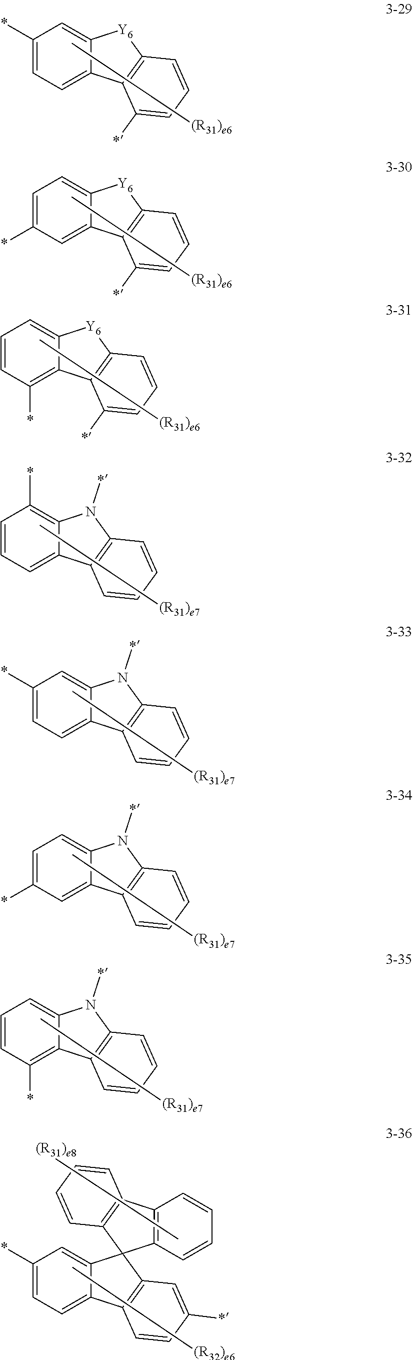

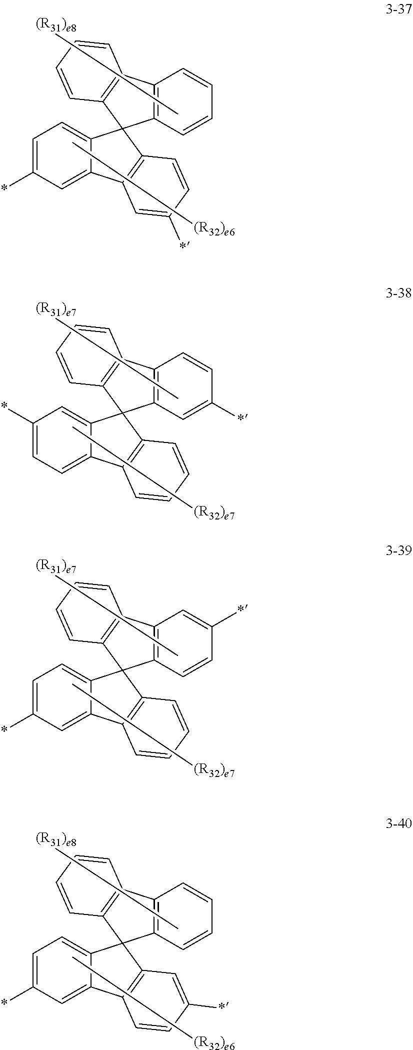

wherein, in Formulae 3-1 to 3-40,

Y₁ may be N or C(R₃₃),

Y₂ may be N or C(R₃₄),

Y₃ may be N or C(R₃₅),

Y₄ may be N or C(R₃₆),

Y₅ may be O, S, or Se,

Y₆ may be O, S, Se, N(R₃₇), or C(R₃₇)(R₃₈),

R₃₁ to R₃₈ may each independently be hydrogen or the same as in the description of R₁₀ₐ,

e4 may be an integer from 0 to 4,

e6 may be an integer from 0 to 6,

e7 may be an integer from 0 to 7,

e8 may be an integer from 0 to 9, and

* and *′ each indicate a binding site to an adjacent atom.

In one or more embodiments, L₁ to L₃ may each independently be: a single bond; or

a group represented by one of Formulae 4-1 to 4-3:

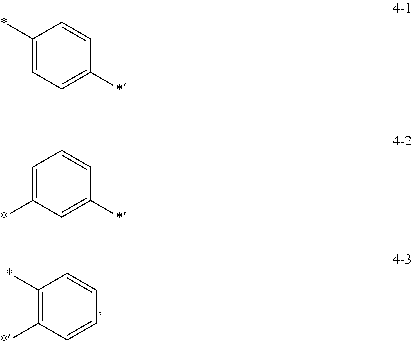

wherein, * and *′ in Formulae 4-1 to 4-3 each indicate a binding site to an adjacent atom.

In an embodiment, a1 to a3 may each independently be 1 or 2.

For example, a1 to a3 may each be 1.

In Formula 2, Ar₁ and Ar₂ may each independently be a C₅-C₆₀ carbocyclic group unsubstituted or substituted with at least one R₁₀ₐ or a C₁-C₆₀ heterocyclic group unsubstituted or substituted with at least one R₁₀ₐ,

b1 and b2 may each independently be an integer from 1 to 10, and

* in Formula 2 is a binding site to an adjacent atom.

In this regard, two or more adjacent groups of Ar₁, Ar₂, and L₁ to L₃ may not be bonded to each other to form a condensed ring.

In an embodiment, Ar₁ and Ar₂ may each independently be:

a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a benzoxazolyl group, a dibenzofuranyl group, a dibenzothiophenyl group, a benzocarbazolyl group, a dibenzocarbazolyl group, or a dibenzosilolyl group, each unsubstituted or substituted with deuterium, —F, —Cl, —Br, —I, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a bicyclo[2.2.2]octyl group, a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, an indenyl group, a fluorenyl group, a spiro-bifluorenyl group, a benzofluorenyl group, a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, —Si(Q₃₁)(Q₃₂)(Q₃₃), —B(Q₃₁)(Q₃₂), or any combination thereof, wherein

Q₃₁ to Q₃₃ are respectively the same as described herein.

In an embodiment, Ar₁ and Ar₂ may each independently be a group represented by one of Formulae 5-1 to 5-80:

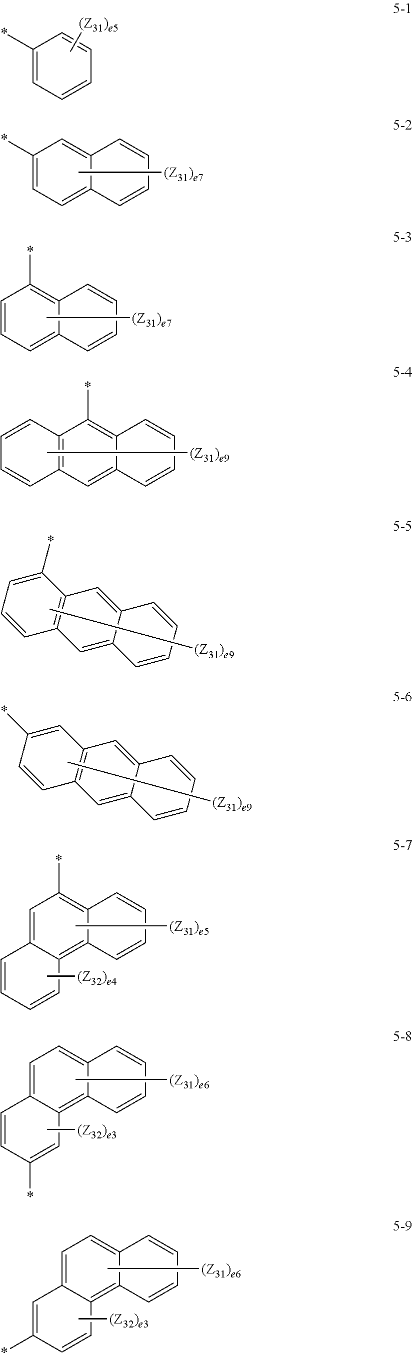

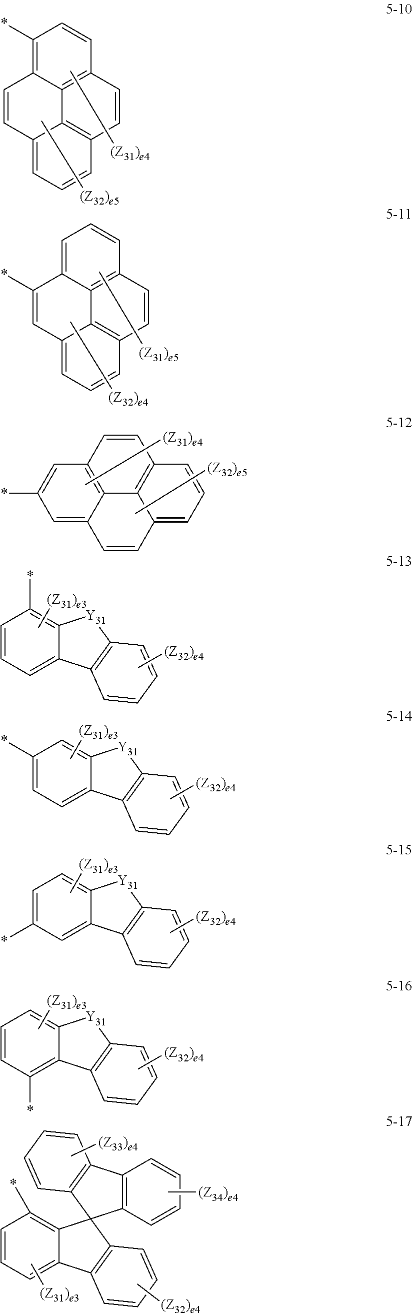

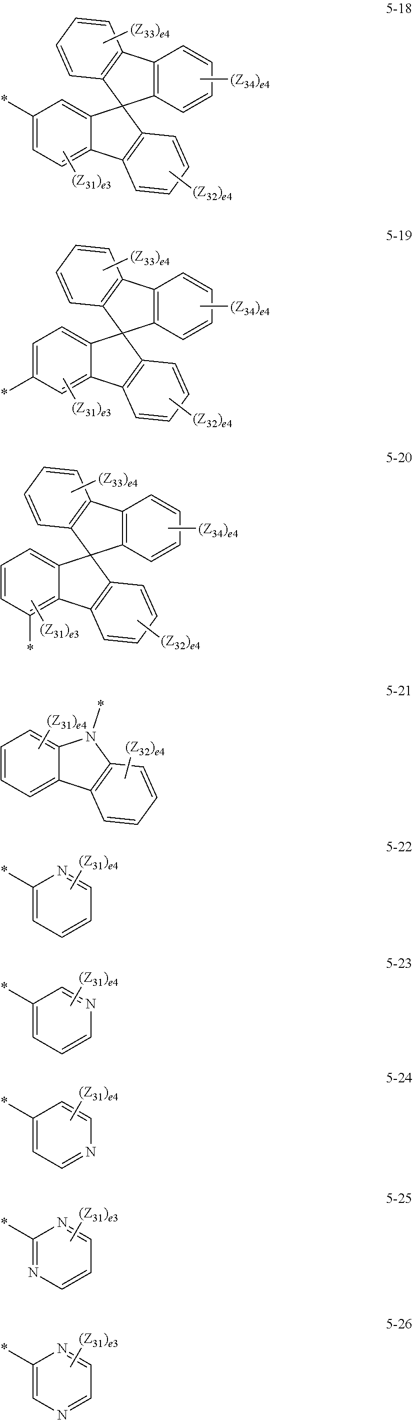

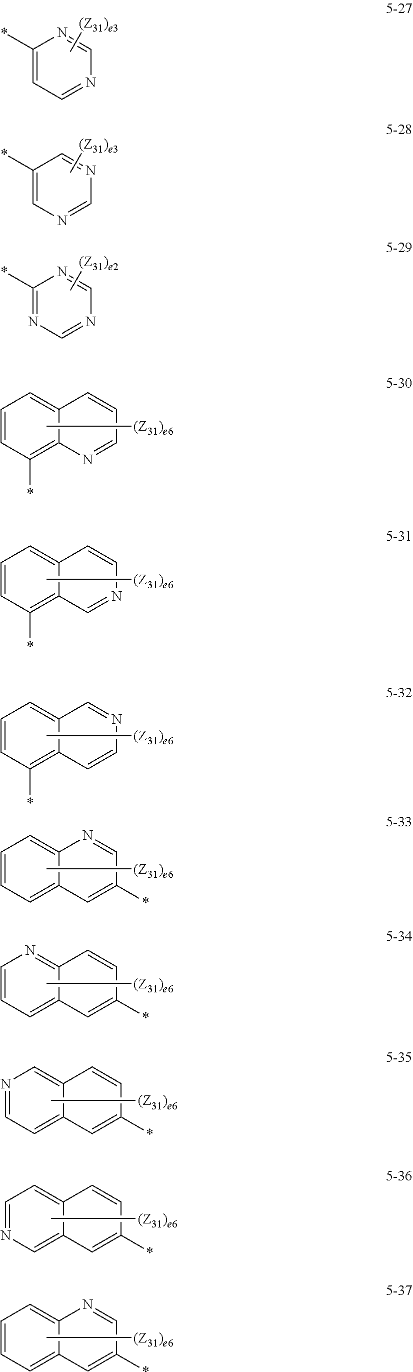

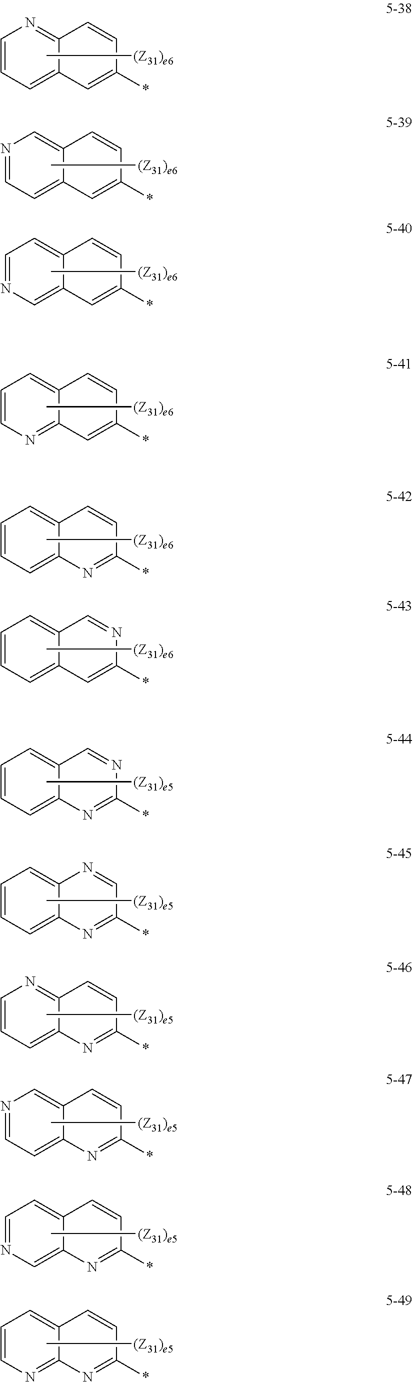

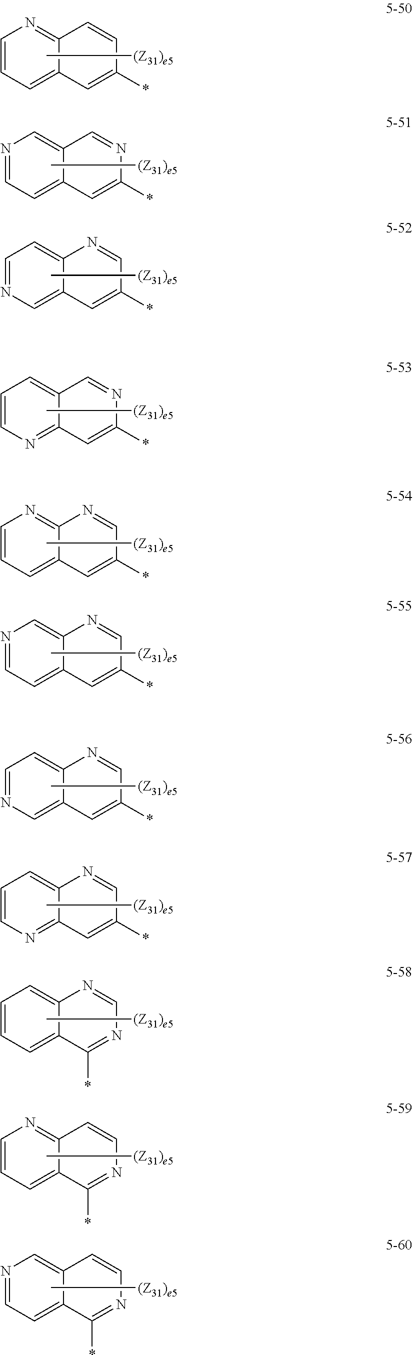

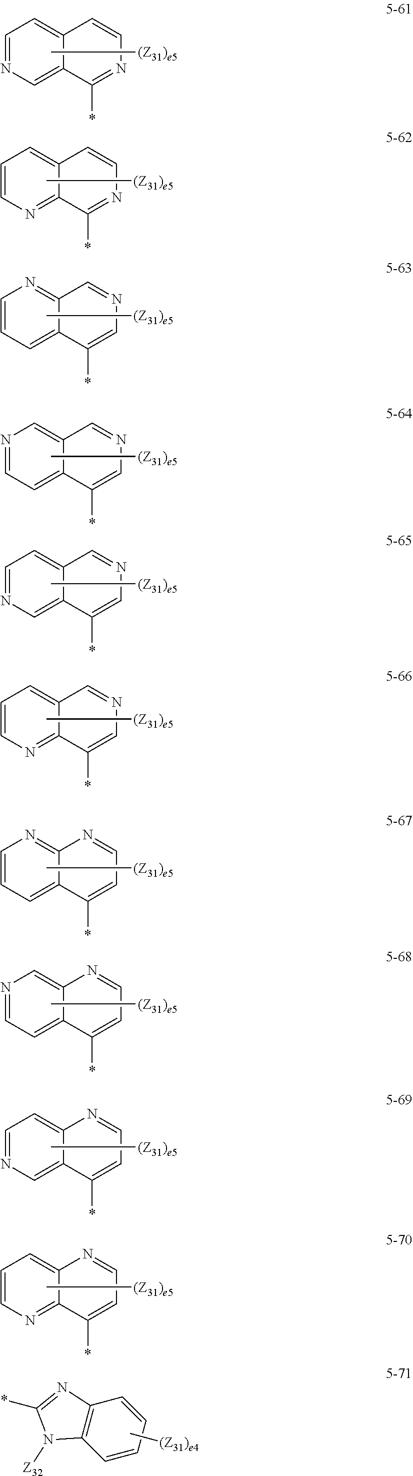

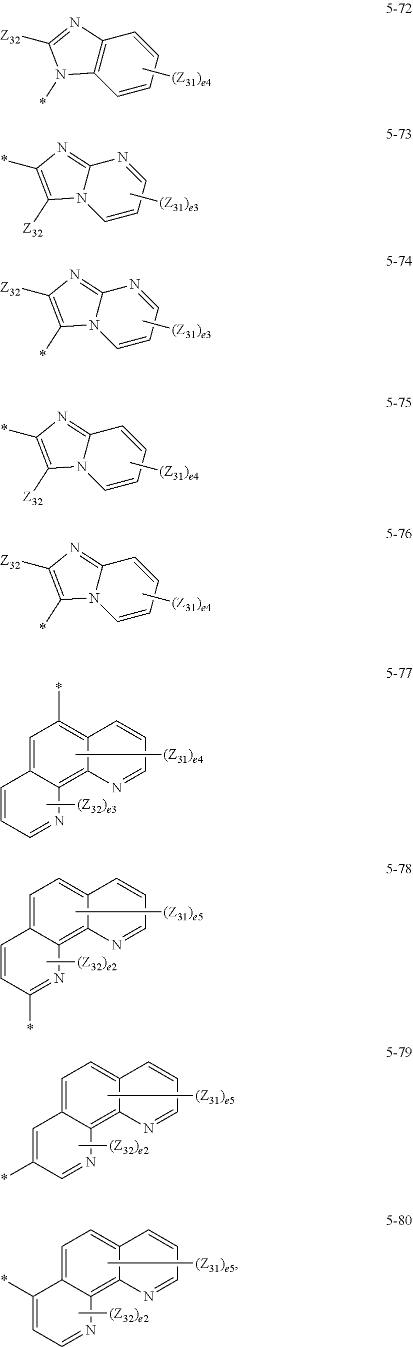

wherein, in Formulae 5-1 to 5-80,

Y₃₁ may be O, S, C(Z₃₃)(Z₃₄), N(Z₃₅), or Si(Z₃₆)(Z₃₇),

Z₃₁ to Z₃₇ may each independently be hydrogen, deuterium, —F, —Cl, —Br, —I, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, a dibenzosilolyl group, —Si(Q₃₁)(Q₃₂)(Q₃₃), or —B(Q₃₁)(Q₃₂),

e2 may be an integer from 0 to 2,

e3 may be an integer from 0 to 3,

e4 may be an integer from 0 to 4,

e5 may be an integer from 0 to 5,

e6 may be an integer from 0 to 6,

e7 may be an integer from 0 to 7,

e9 may be an integer from 0 to 9, and

* indicates a binding site to an adjacent atom.

In one or more embodiments, Ar₁ and Ar₂ may each independently be a group represented by one of Formulae 6-1 to 6-40:

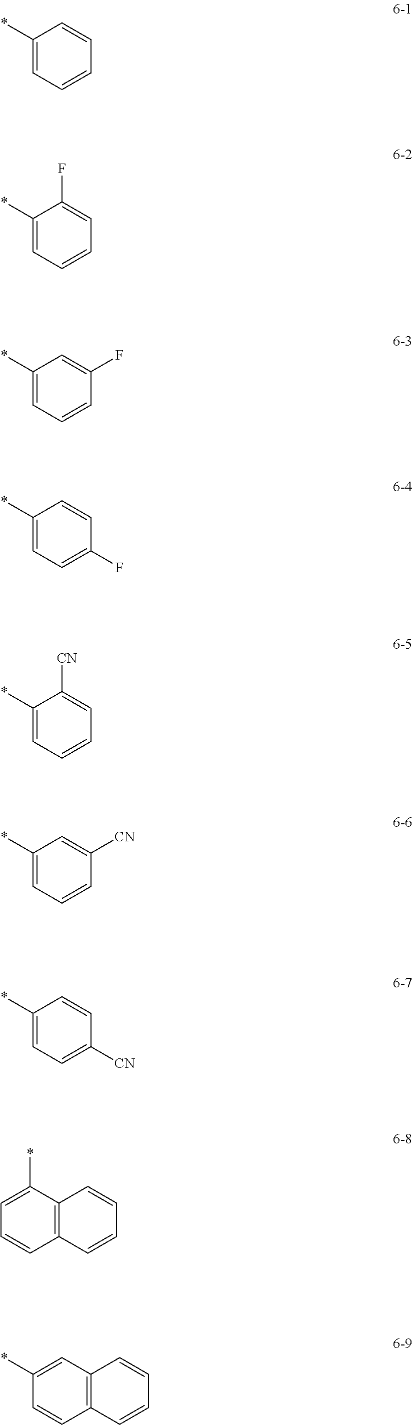

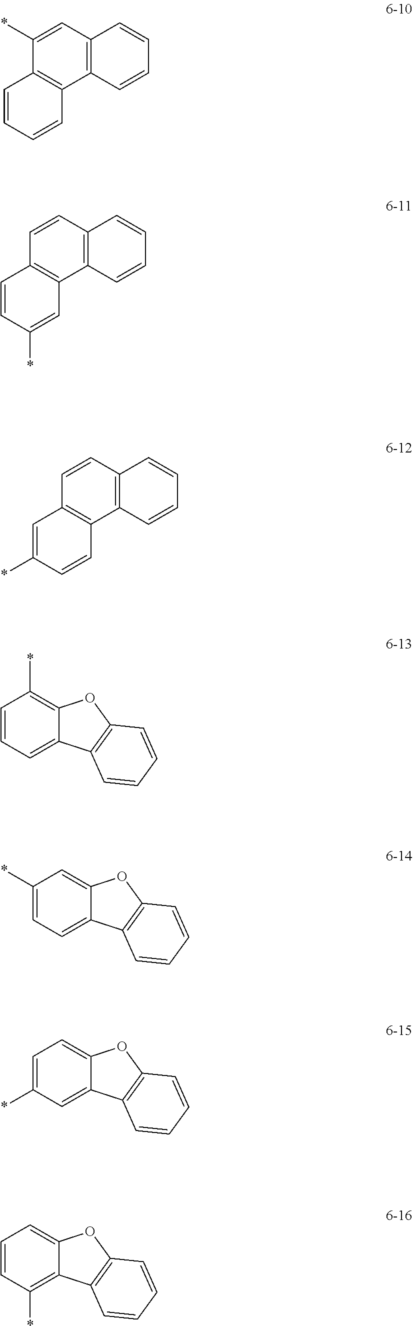

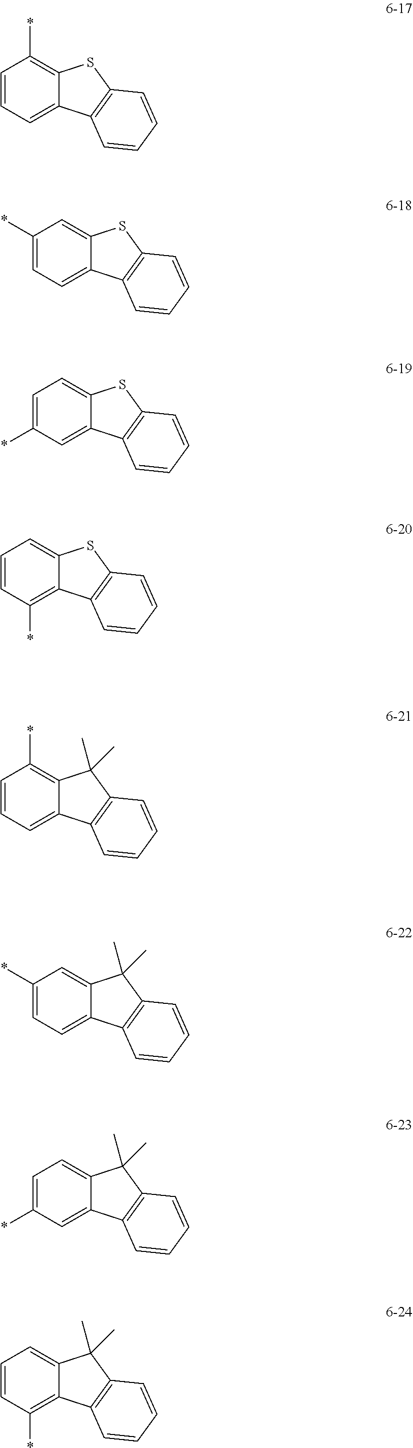

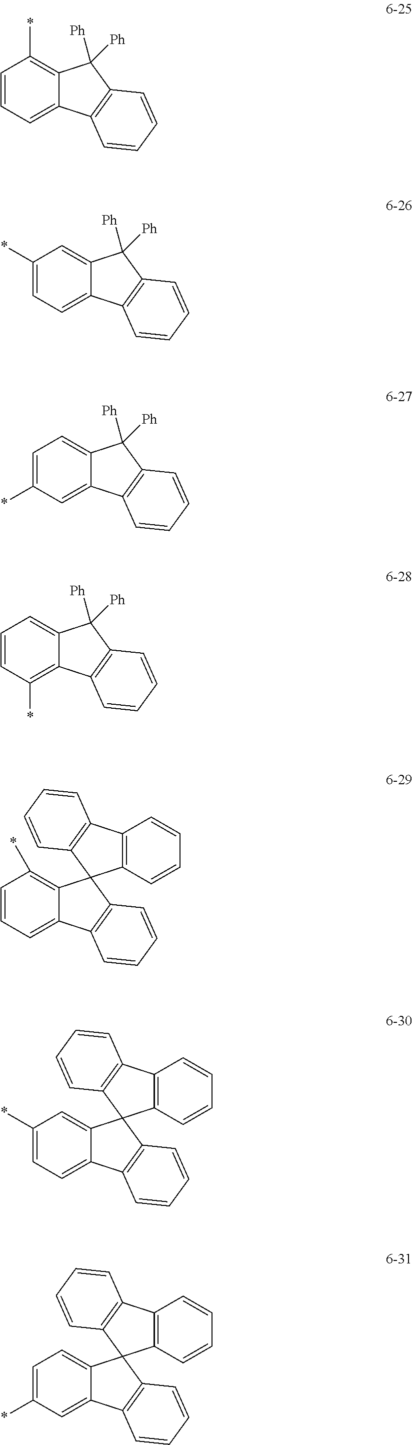

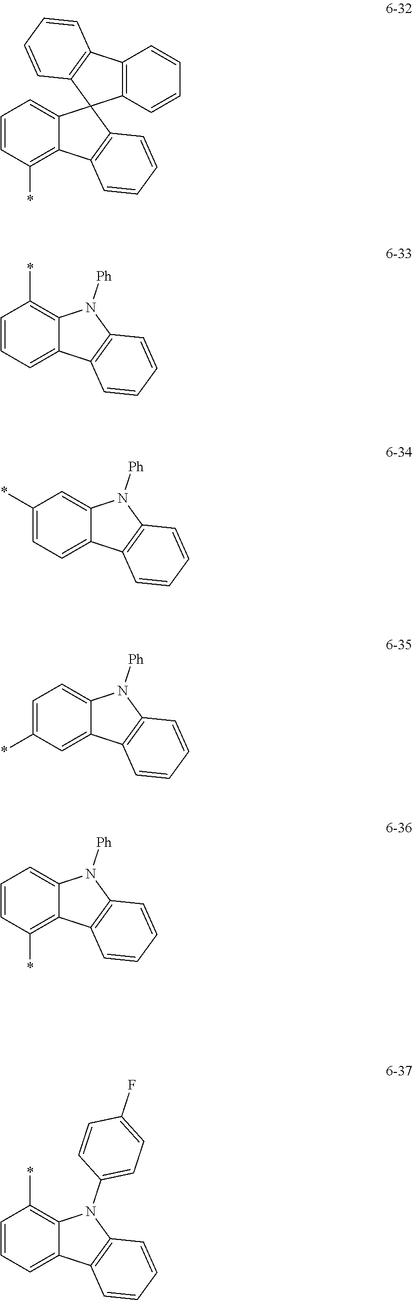

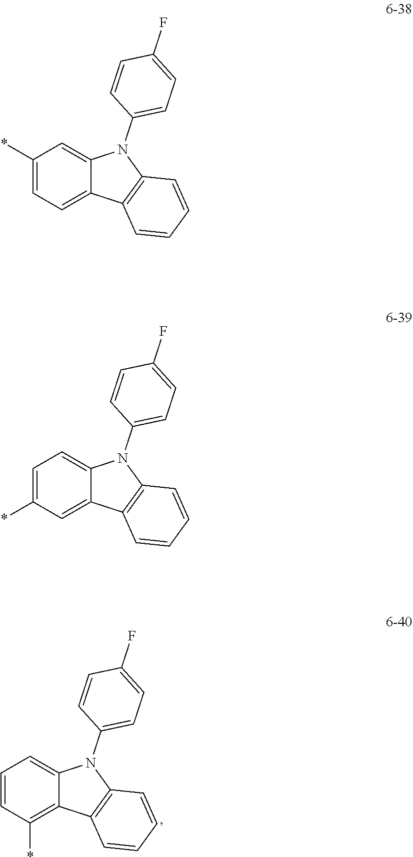

wherein, in Formulae 6-1 to 6-40,

“Ph” indicates a phenyl group, and

* indicates a binding site to an adjacent atom.

In an embodiment, b1 and b2 may each independently be an integer from 1 to 5.

In an embodiment, b1 and b2 may each independently be 1 or 2.

For example, b1 and b2 may each be 1.

In an embodiment, a group represented by *-(L₃)ₐ₃-*′ in Formula 2 may be a single bond.

In an embodiment, a group represented by *-(L₁)ₐ₁-(Ar₁)_{b1} and a group represented by *-(L₂)ₐ₂-(Ar₂)_{b2} in Formula 2 may be different from each other.

In an embodiment, a group represented by *-(L₁)ₐ₁-(Ar₁)_{b1} and a group represented by *-(L₂)ₐ₂-(Ar₂)_{b2} may each independently be a group represented by one of Formulae 7-1 to 7-10:

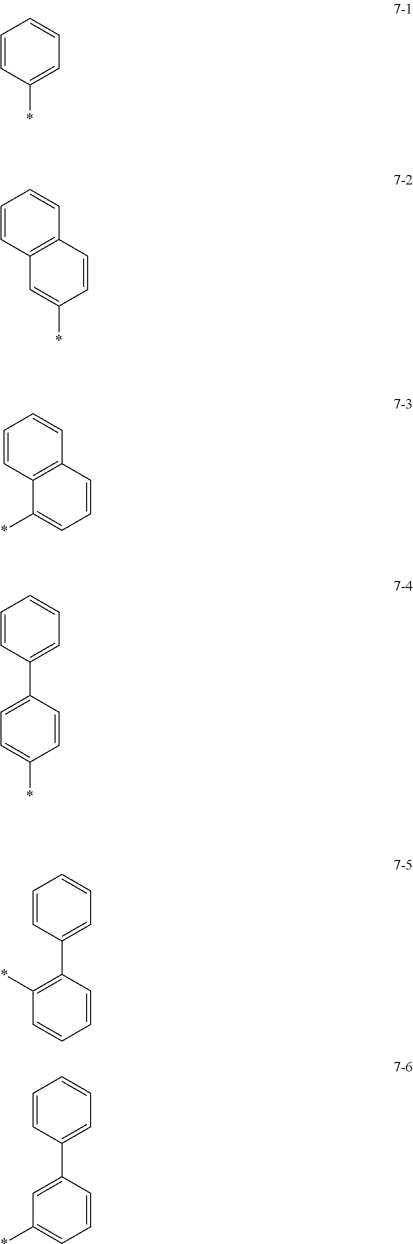

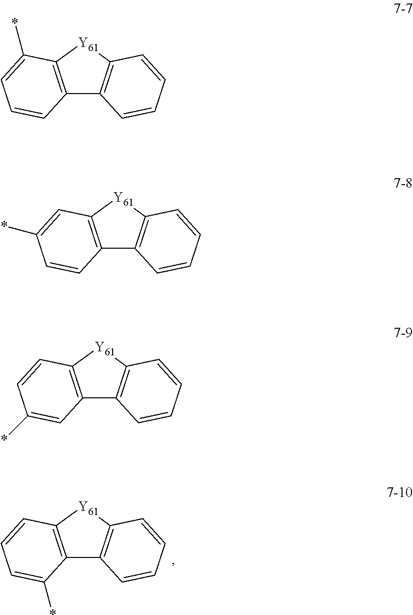

wherein, in Formulae 7-1 to 7-10,

Y₆₁ may be O, S, N(R₆₁), or C(R₆₁)(R₆₂),

R₆₁ and R₆₂ may each independently be: hydrogen; deuterium; —F; a cyano group; or a C₁-C₂₀ alkyl group or a phenyl group, each unsubstituted or substituted with deuterium, —F, a cyano group, a C₁-C₂₀ alkyl group, a phenyl group, or any combination thereof, and

* and *′ each indicate a binding site to an adjacent atom.

For example, R₆₁ and R₆₂ may each independently be a C₁-C₂₀ alkyl group or a phenyl group.

In an embodiment, the condensed cyclic compound represented by Formula 1 may be represented by one of Formulae 1A-1 to 1A-12:

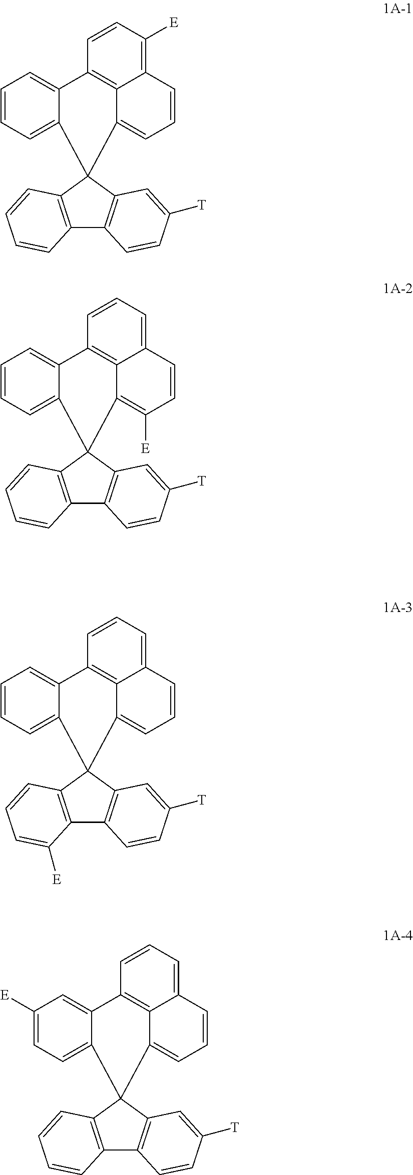

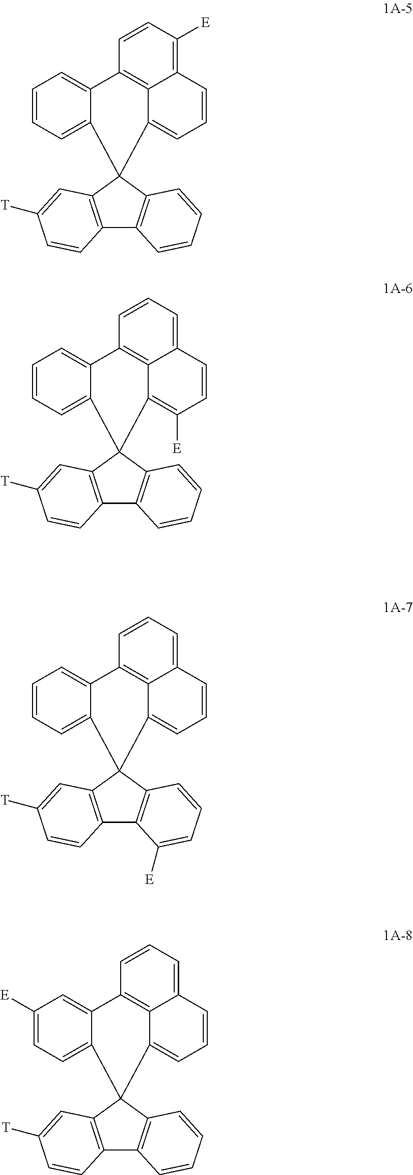

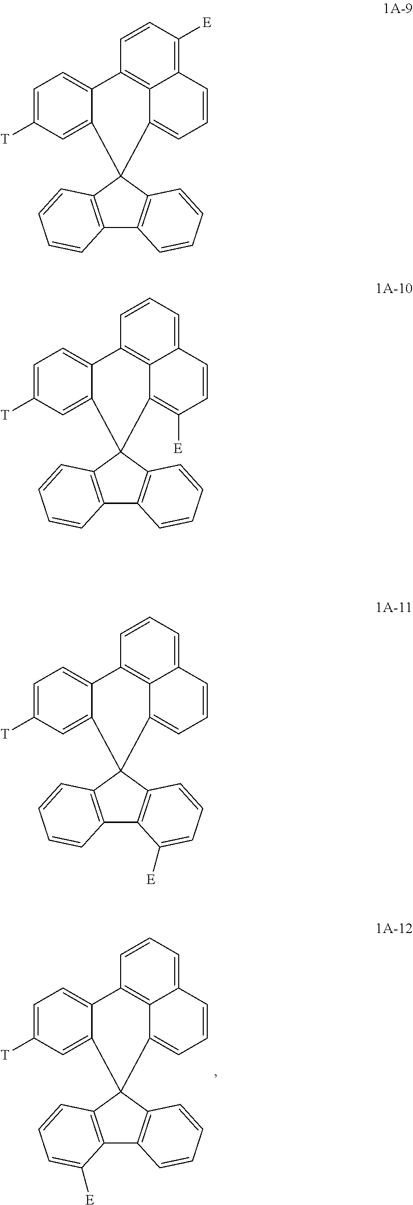

wherein, in Formulae 1A-1 to 1A-12,

T may be a group represented by Formula 2, and

E is the same as in the description of R₁₀ₐ. “R₁₀ₐ” may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂),

wherein Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ aryl alkyl group, or a C₂-C₆₀ heteroaryl alkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the condensed cyclic compound represented by Formula 1 may be a group represented by one of Compounds 1 to 375, but embodiments are not limited thereto:

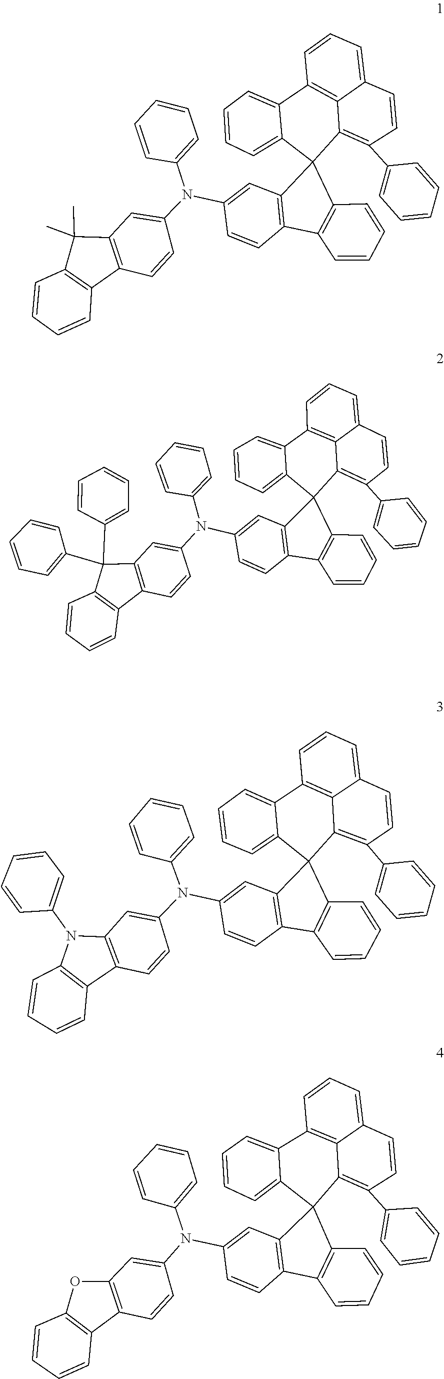 [SMILES: CC1(C)c2ccccc2-c2ccc(N(c3ccccc3)c3ccc4c(c3)C3(c5ccccc5-4)c4ccccc4-c4cccc5ccc(-c6ccccc6)c3c45)cc21 ; c1ccc(-c2ccc3cccc4c3c2C2(c3ccccc3-c3ccc(N(c5ccccc5)c5ccc6c(c5)C(c5ccccc5)(c5ccccc5)c5ccccc5-6)cc32)c2ccccc2-4)cc1 ; c1ccc(-c2ccc3cccc4c3c2C2(c3ccccc3-c3ccc(N(c5ccccc5)c5ccc6c7ccccc7n(-c7ccccc7)c6c5)cc32)c2ccccc2-4)cc1 ; c1ccc(-c2ccc3cccc4c3c2C2(c3ccccc3-c3ccc(N(c5ccccc5)c5ccc6c(c5)oc5ccccc56)cc32)c2ccccc2-4)cc1]

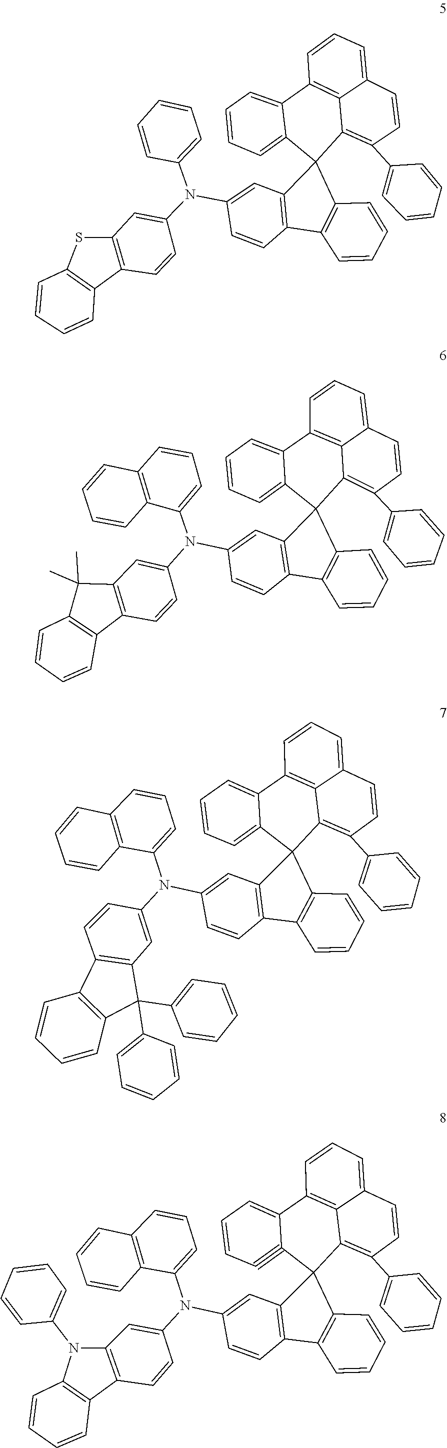

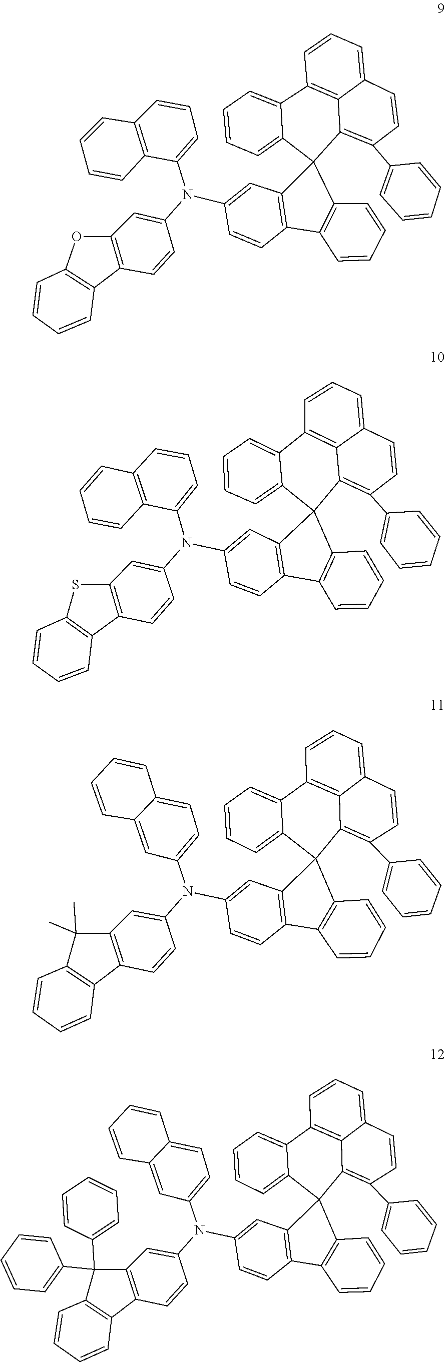 [SMILES: c1ccc(-c2ccc3cccc4c3c2C2(c3ccccc3-c3ccc(N(c5ccc6c(c5)oc5ccccc56)c5cccc6ccccc56)cc32)c2ccccc2-4)cc1 ; c1ccc(-c2ccc3cccc4c3c2C2(c3ccccc3-c3ccc(N(c5ccc6c(c5)sc5ccccc56)c5cccc6ccccc56)cc32)c2ccccc2-4)cc1 ; CC1(C)c2ccccc2-c2ccc(N(c3ccc4c(c3)C3(c5ccccc5-4)c4ccccc4-c4cccc5ccc(-c6ccccc6)c3c45)c3ccc4ccccc4c3)cc21 ; c1ccc(-c2ccc3cccc4c3c2C2(c3ccccc3-c3ccc(N(c5ccc6c(c5)C(c5ccccc5)(c5ccccc5)c5ccccc5-6)c5ccc6ccccc6c5)cc32)c2ccccc2-4)cc1]

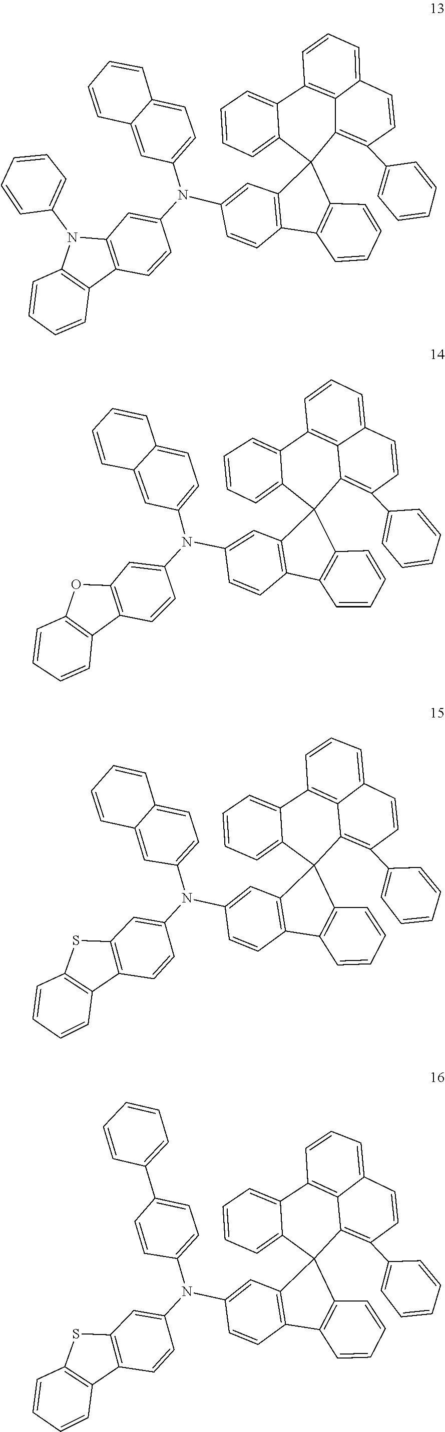 [SMILES: c1ccc(-c2ccc3cccc4c3c2C2(c3ccccc3-c3ccc(N(c5ccc6ccccc6c5)c5ccc6c7ccccc7n(-c7ccccc7)c6c5)cc32)c2ccccc2-4)cc1 ; c1ccc(-c2ccc3cccc4c3c2C2(c3ccccc3-c3ccc(N(c5ccc6ccccc6c5)c5ccc6c(c5)oc5ccccc56)cc32)c2ccccc2-4)cc1 ; c1ccc(-c2ccc3cccc4c3c2C2(c3ccccc3-c3ccc(N(c5ccc6ccccc6c5)c5ccc6c(c5)sc5ccccc56)cc32)c2ccccc2-4)cc1 ; c1ccc(-c2ccc(N(c3ccc4c(c3)C3(c5ccccc5-4)c4ccccc4-c4cccc5ccc(-c6ccccc6)c3c45)c3ccc4c(c3)sc3ccccc34)cc2)cc1]

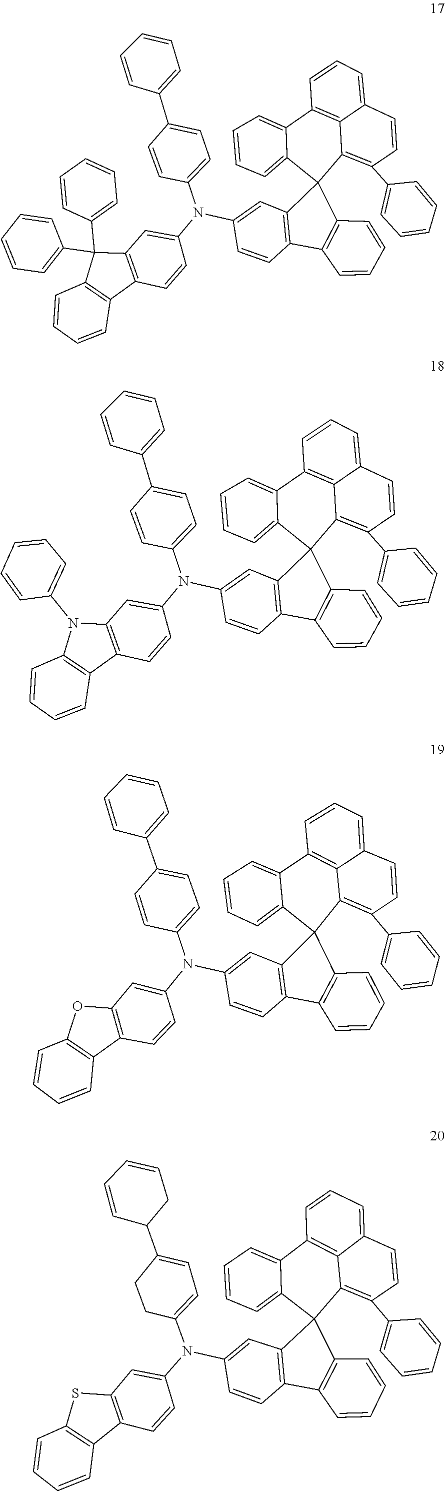 [SMILES: c1ccc(-c2ccc(N(c3ccc4c(c3)C(c3ccccc3)(c3ccccc3)c3ccccc3-4)c3ccc4c(c3)C3(c5ccccc5-4)c4ccccc4-c4cccc5ccc(-c6ccccc6)c3c45)cc2)cc1 ; c1ccc(-c2ccc(N(c3ccc4c(c3)C3(c5ccccc5-4)c4ccccc4-c4cccc5ccc(-c6ccccc6)c3c45)c3ccc4c5ccccc5n(-c5ccccc5)c4c3)cc2)cc1 ; c1ccc(-c2ccc(N(c3ccc4c(c3)C3(c5ccccc5-4)c4ccccc4-c4cccc5ccc(-c6ccccc6)c3c45)c3ccc4c(c3)oc3ccccc34)cc2)cc1 ; C1=CCC(C2=CC=C(N(c3ccc4c(c3)C3(c5ccccc5-4)c4ccccc4-c4cccc5ccc(-c6ccccc6)c3c45)c3ccc4c(c3)sc3ccccc34)CC2)C=C1]

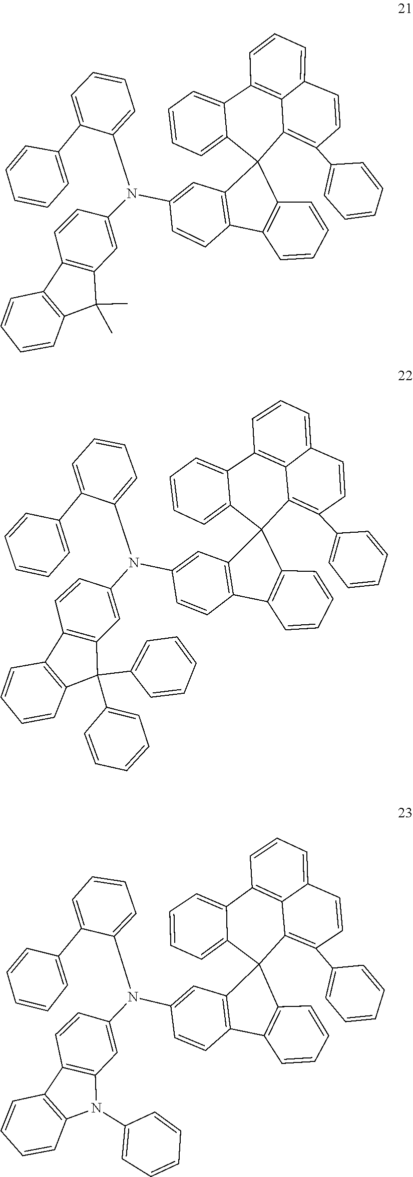 [SMILES: CC1(C)c2ccccc2-c2ccc(N(c3ccc4c(c3)C3(c5ccccc5-4)c4ccccc4-c4cccc5ccc(-c6ccccc6)c3c45)c3ccccc3-c3ccccc3)cc21 ; c1ccc(-c2ccccc2N(c2ccc3c(c2)C(c2ccccc2)(c2ccccc2)c2ccccc2-3)c2ccc3c(c2)C2(c4ccccc4-3)c3ccccc3-c3cccc4ccc(-c5ccccc5)c2c34)cc1 ; c1ccc(-c2ccccc2N(c2ccc3c(c2)C2(c4ccccc4-3)c3ccccc3-c3cccc4ccc(-c5ccccc5)c2c34)c2ccc3c4ccccc4n(-c4ccccc4)c3c2)cc1]

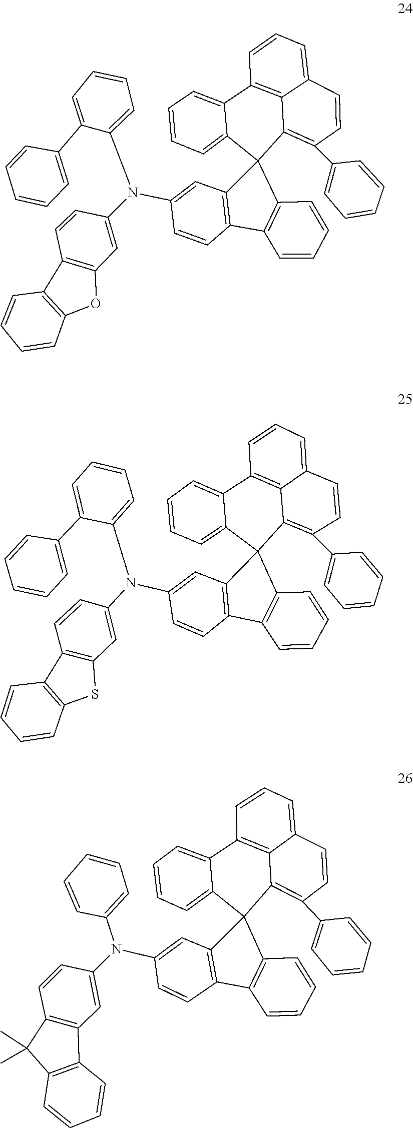 [SMILES: c1ccc(-c2ccccc2N(c2ccc3c(c2)C2(c4ccccc4-3)c3ccccc3-c3cccc4ccc(-c5ccccc5)c2c34)c2ccc3c(c2)oc2ccccc23)cc1 ; c1ccc(-c2ccccc2N(c2ccc3c(c2)C2(c4ccccc4-3)c3ccccc3-c3cccc4ccc(-c5ccccc5)c2c34)c2ccc3c(c2)sc2ccccc23)cc1 ; CC1(C)c2ccccc2-c2cc(N(c3ccccc3)c3ccc4c(c3)C3(c5ccccc5-4)c4ccccc4-c4cccc5ccc(-c6ccccc6)c3c45)ccc21]

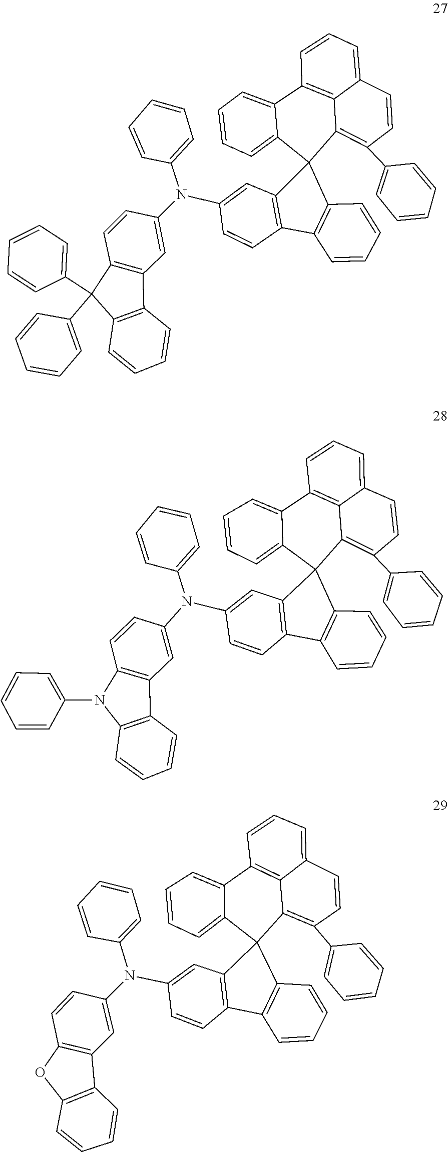 [SMILES: c1ccc(-c2ccc3cccc4c3c2C2(c3ccccc3-c3ccc(N(c5ccccc5)c5ccc6c(c5)-c5ccccc5C6(c5ccccc5)c5ccccc5)cc32)c2ccccc2-4)cc1 ; c1ccc(-c2ccc3cccc4c3c2C2(c3ccccc3-c3ccc(N(c5ccccc5)c5ccc6c(c5)c5ccccc5n6-c5ccccc5)cc32)c2ccccc2-4)cc1 ; c1ccc(-c2ccc3cccc4c3c2C2(c3ccccc3-c3ccc(N(c5ccccc5)c5ccc6oc7ccccc7c6c5)cc32)c2ccccc2-4)cc1]

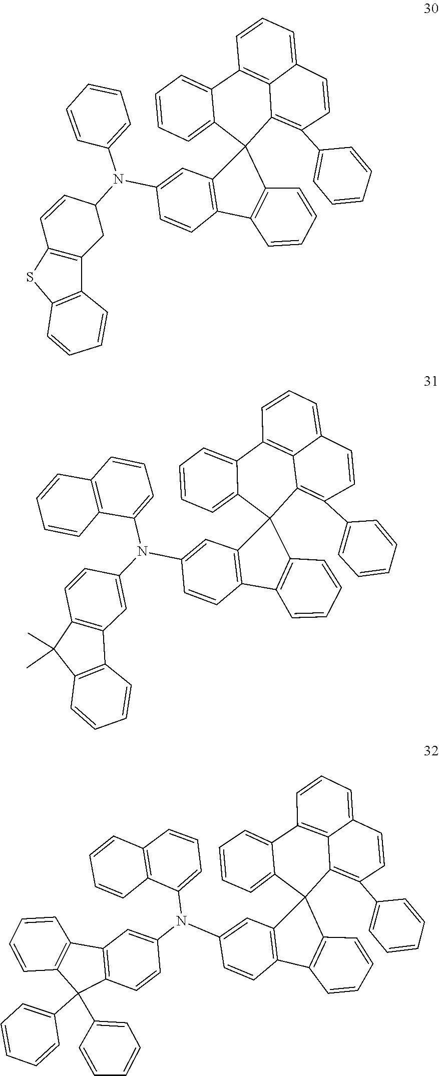 [SMILES: C1=CC(N(c2ccccc2)c2ccc3c(c2)C2(c4ccccc4-3)c3ccccc3-c3cccc4ccc(-c5ccccc5)c2c34)Cc2c1sc1ccccc21 ; CC1(C)c2ccccc2-c2cc(N(c3ccc4c(c3)C3(c5ccccc5-4)c4ccccc4-c4cccc5ccc(-c6ccccc6)c3c45)c3cccc4ccccc34)ccc21 ; c1ccc(-c2ccc3cccc4c3c2C2(c3ccccc3-c3ccc(N(c5ccc6c(c5)-c5ccccc5C6(c5ccccc5)c5ccccc5)c5cccc6ccccc56)cc32)c2ccccc2-4)cc1]

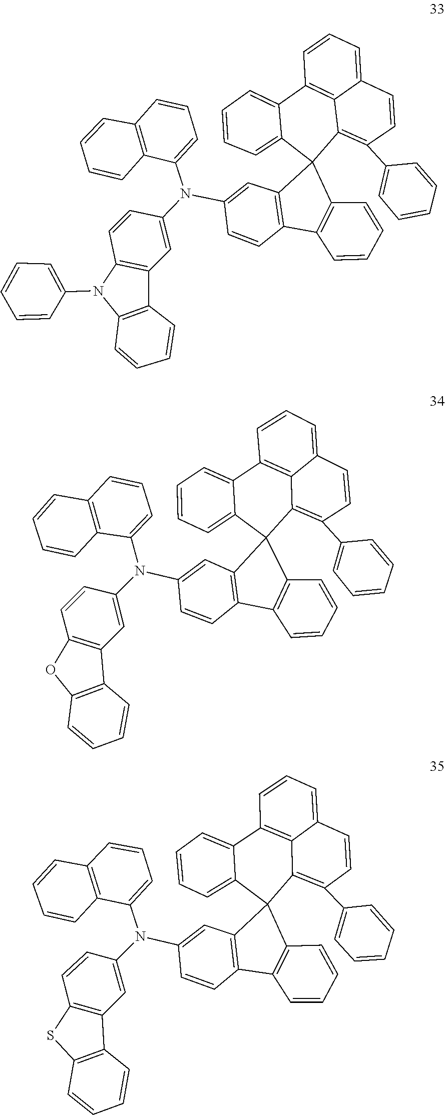 [SMILES: c1ccc(-c2ccc3cccc4c3c2C2(c3ccccc3-c3ccc(N(c5ccc6c(c5)c5ccccc5n6-c5ccccc5)c5cccc6ccccc56)cc32)c2ccccc2-4)cc1 ; c1ccc(-c2ccc3cccc4c3c2C2(c3ccccc3-c3ccc(N(c5ccc6oc7ccccc7c6c5)c5cccc6ccccc56)cc32)c2ccccc2-4)cc1 ; c1ccc(-c2ccc3cccc4c3c2C2(c3ccccc3-c3ccc(N(c5ccc6sc7ccccc7c6c5)c5cccc6ccccc56)cc32)c2ccccc2-4)cc1]

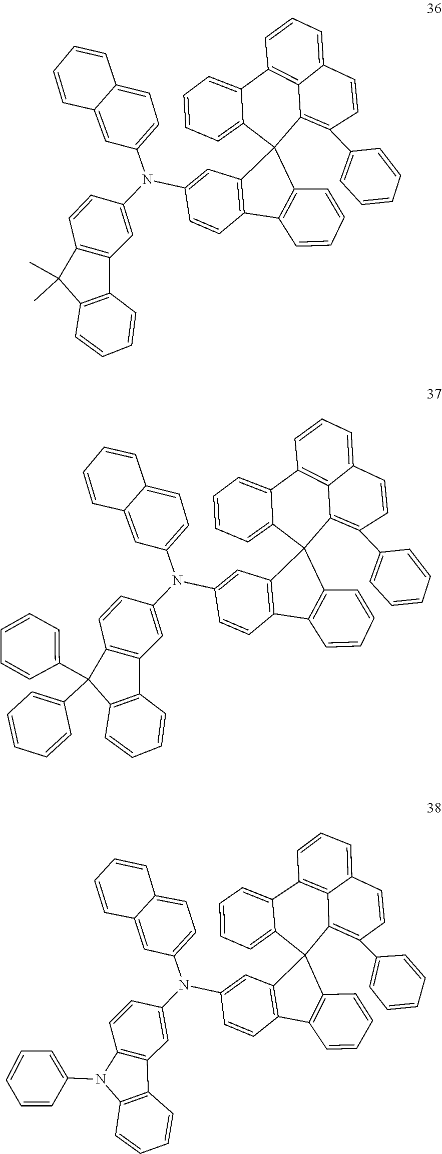 [SMILES: CC1(C)c2ccccc2-c2cc(N(c3ccc4c(c3)C3(c5ccccc5-4)c4ccccc4-c4cccc5ccc(-c6ccccc6)c3c45)c3ccc4ccccc4c3)ccc21 ; c1ccc(-c2ccc3cccc4c3c2C2(c3ccccc3-c3ccc(N(c5ccc6c(c5)-c5ccccc5C6(c5ccccc5)c5ccccc5)c5ccc6ccccc6c5)cc32)c2ccccc2-4)cc1 ; c1ccc(-c2ccc3cccc4c3c2C2(c3ccccc3-c3ccc(N(c5ccc6ccccc6c5)c5ccc6c(c5)c5ccccc5n6-c5ccccc5)cc32)c2ccccc2-4)cc1]

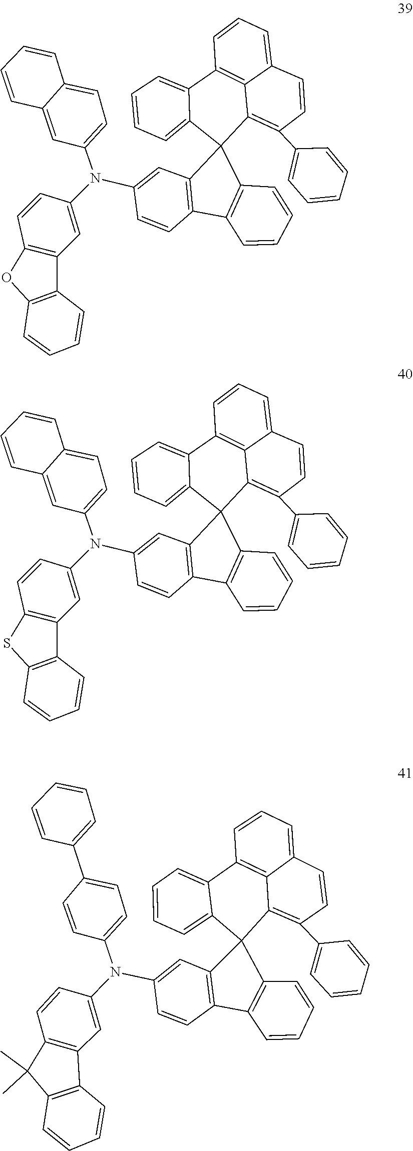 [SMILES: c1ccc(-c2ccc3cccc4c3c2C2(c3ccccc3-c3ccc(N(c5ccc6ccccc6c5)c5ccc6oc7ccccc7c6c5)cc32)c2ccccc2-4)cc1 ; c1ccc(-c2ccc3cccc4c3c2C2(c3ccccc3-c3ccc(N(c5ccc6ccccc6c5)c5ccc6sc7ccccc7c6c5)cc32)c2ccccc2-4)cc1 ; CC1(C)c2ccccc2-c2cc(N(c3ccc(-c4ccccc4)cc3)c3ccc4c(c3)C3(c5ccccc5-4)c4ccccc4-c4cccc5ccc(-c6ccccc6)c3c45)ccc21]

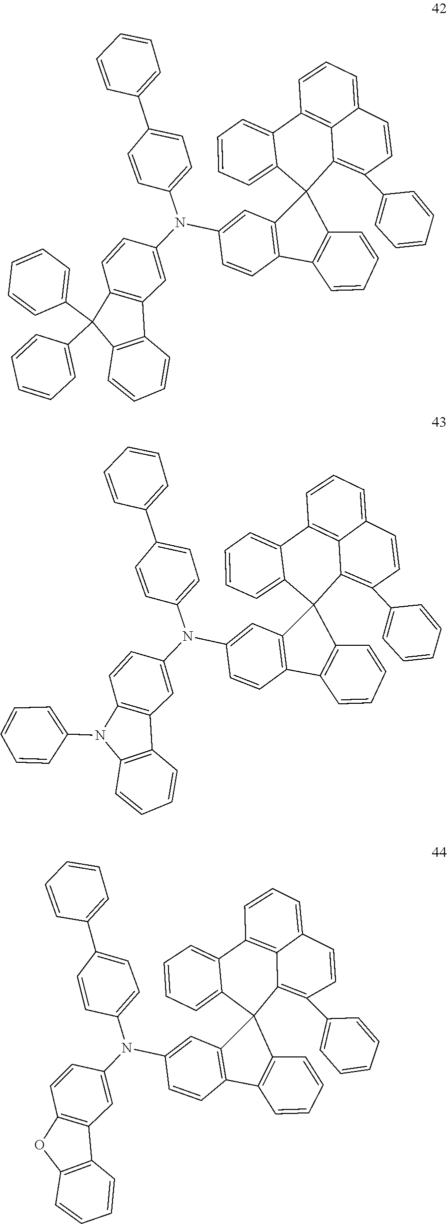 [SMILES: c1ccc(-c2ccc(N(c3ccc4c(c3)-c3ccccc3C4(c3ccccc3)c3ccccc3)c3ccc4c(c3)C3(c5ccccc5-4)c4ccccc4-c4cccc5ccc(-c6ccccc6)c3c45)cc2)cc1 ; c1ccc(-c2ccc(N(c3ccc4c(c3)C3(c5ccccc5-4)c4ccccc4-c4cccc5ccc(-c6ccccc6)c3c45)c3ccc4c(c3)c3ccccc3n4-c3ccccc3)cc2)cc1 ; c1ccc(-c2ccc(N(c3ccc4c(c3)C3(c5ccccc5-4)c4ccccc4-c4cccc5ccc(-c6ccccc6)c3c45)c3ccc4oc5ccccc5c4c3)cc2)cc1]

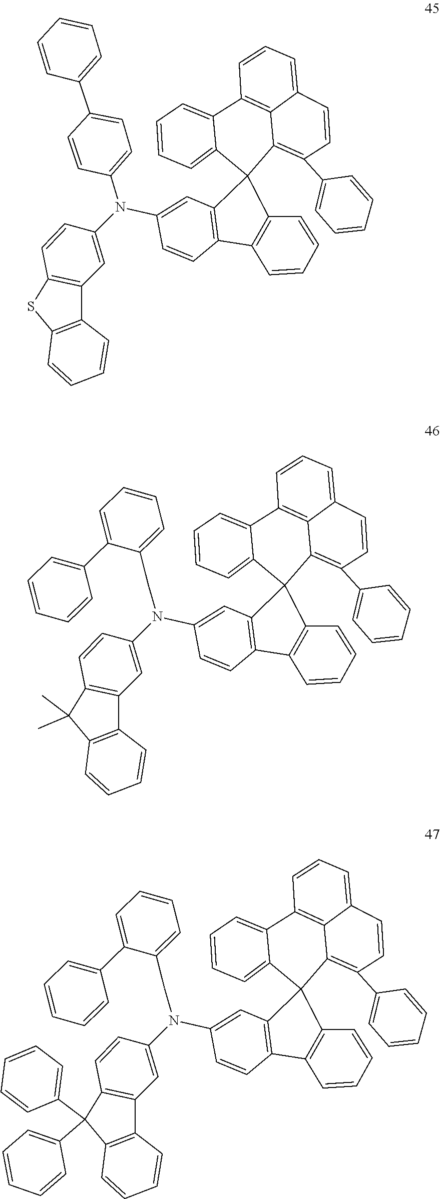 [SMILES: c1ccc(-c2ccc(N(c3ccc4c(c3)C3(c5ccccc5-4)c4ccccc4-c4cccc5ccc(-c6ccccc6)c3c45)c3ccc4sc5ccccc5c4c3)cc2)cc1 ; CC1(C)c2ccccc2-c2cc(N(Cc3ccccc3-c3ccccc3)c3ccc4c(c3)C3(c5ccccc5-4)c4ccccc4-c4cccc5ccc(-c6ccccc6)c3c45)ccc21 ; c1ccc(-c2ccccc2CN(c2ccc3c(c2)-c2ccccc2C3(c2ccccc2)c2ccccc2)c2ccc3c(c2)C2(c4ccccc4-3)c3ccccc3-c3cccc4ccc(-c5ccccc5)c2c34)cc1]

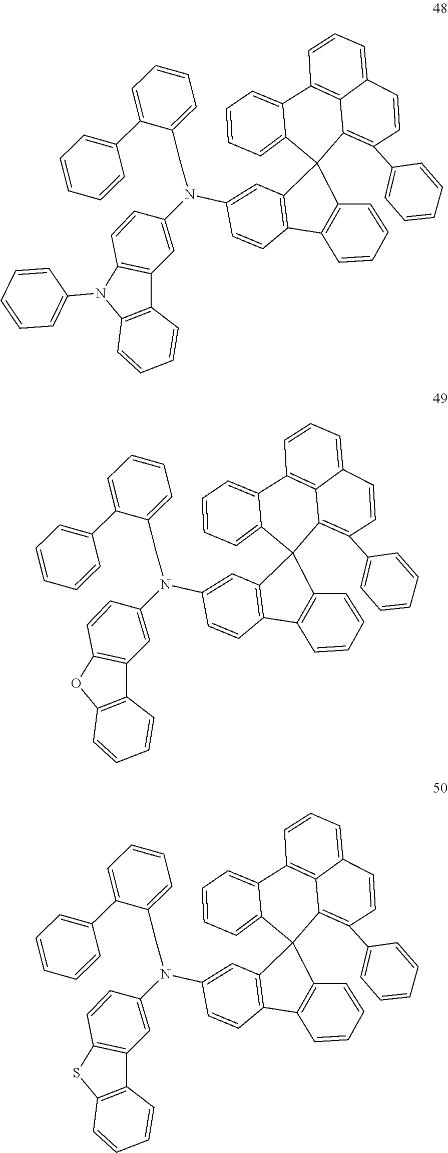 [SMILES: c1ccc(-c2ccccc2CN(c2ccc3c(c2)C2(c4ccccc4-3)c3ccccc3-c3cccc4ccc(-c5ccccc5)c2c34)c2ccc3c(c2)c2ccccc2n3-c2ccccc2)cc1 ; c1ccc(-c2ccccc2CN(c2ccc3c(c2)C2(c4ccccc4-3)c3ccccc3-c3cccc4ccc(-c5ccccc5)c2c34)c2ccc3oc4ccccc4c3c2)cc1 ; c1ccc(-c2ccccc2CN(c2ccc3c(c2)C2(c4ccccc4-3)c3ccccc3-c3cccc4ccc(-c5ccccc5)c2c34)c2ccc3sc4ccccc4c3c2)cc1]

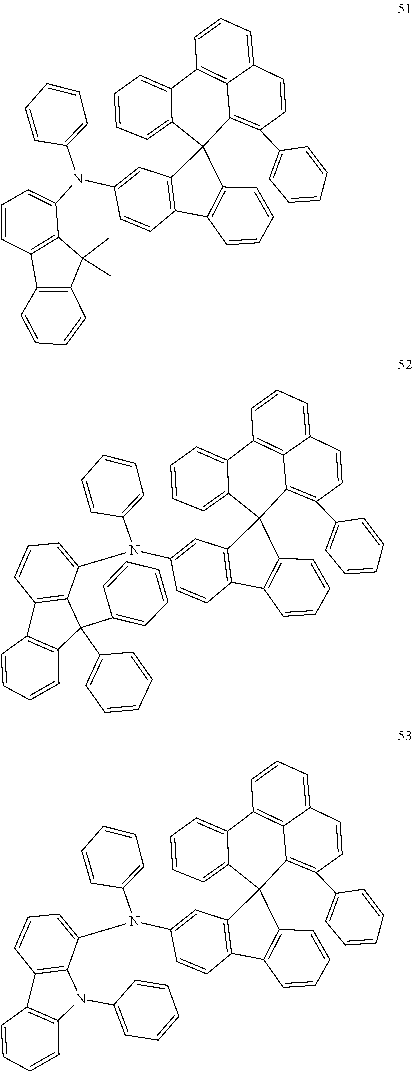 [SMILES: CC1(C)c2ccccc2-c2cccc(N(c3ccccc3)c3ccc4c(c3)C3(c5ccccc5-4)c4ccccc4-c4cccc5ccc(-c6ccccc6)c3c45)c21 ; c1ccc(-c2ccc3cccc4c3c2C2(c3ccccc3-c3ccc(N(c5ccccc5)c5cccc6c5C(c5ccccc5)(c5ccccc5)c5ccccc5-6)cc32)c2ccccc2-4)cc1 ; c1ccc(-c2ccc3cccc4c3c2C2(c3ccccc3-c3ccc(N(c5ccccc5)c5cccc6c7ccccc7n(-c7ccccc7)c56)cc32)c2ccccc2-4)cc1]

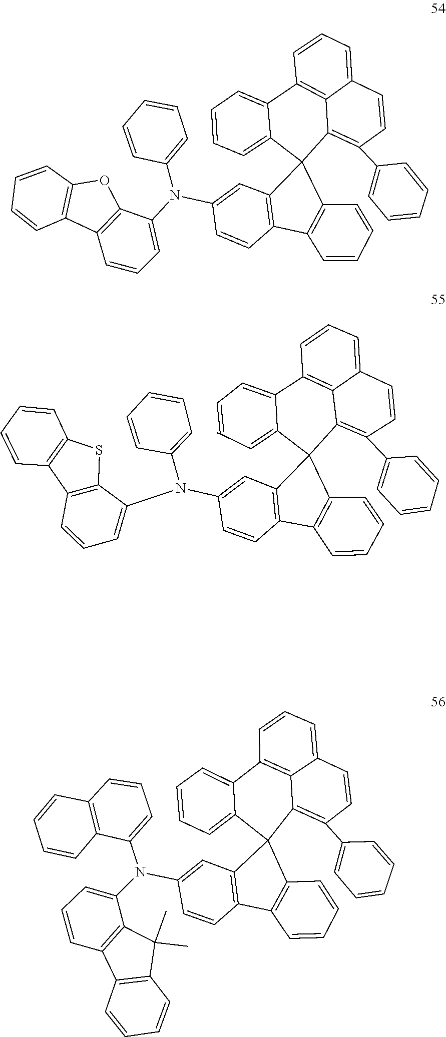 [SMILES: c1ccc(-c2ccc3cccc4c3c2C2(c3ccccc3-c3ccc(N(c5ccccc5)c5cccc6c5oc5ccccc56)cc32)c2ccccc2-4)cc1 ; Cc1ccc2c(c1)C1(c3ccccc3-c3ccc(N(c4ccccc4)c4cccc5c4sc4ccccc45)cc31)c1c(-c3ccccc3)ccc3cccc-2c13 ; CC1(C)c2ccccc2-c2cccc(N(c3ccc4c(c3)C3(c5ccccc5-4)c4ccccc4-c4cccc5ccc(-c6ccccc6)c3c45)c3cccc4ccccc34)c21]

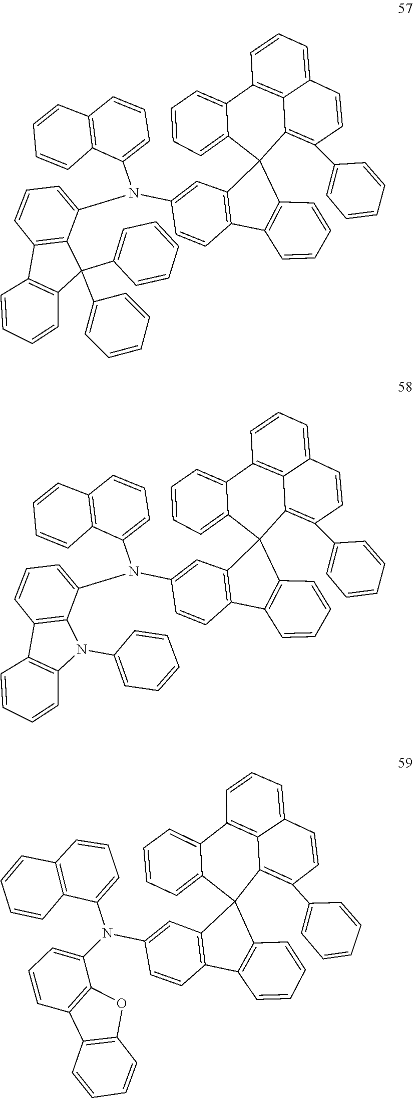 [SMILES: c1ccc(-c2ccc3cccc4c3c2C2(c3ccccc3-c3ccc(N(c5cccc6c5C(c5ccccc5)(c5ccccc5)c5ccccc5-6)c5cccc6ccccc56)cc32)c2ccccc2-4)cc1 ; c1ccc(-c2ccc3cccc4c3c2C2(c3ccccc3-c3ccc(N(c5cccc6ccccc56)c5cccc6c7ccccc7n(-c7ccccc7)c56)cc32)c2ccccc2-4)cc1 ; c1ccc(-c2ccc3cccc4c3c2C2(c3ccccc3-c3ccc(N(c5cccc6ccccc56)c5cccc6c5oc5ccccc56)cc32)c2ccccc2-4)cc1]

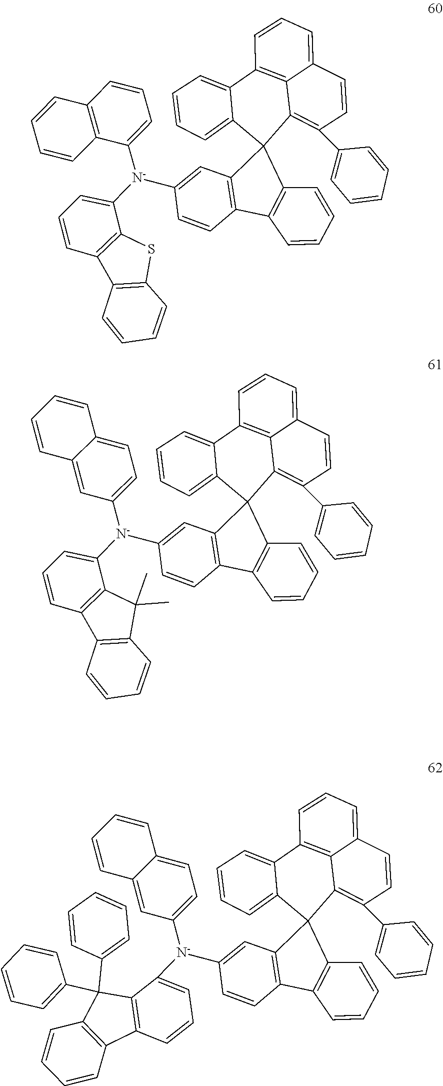

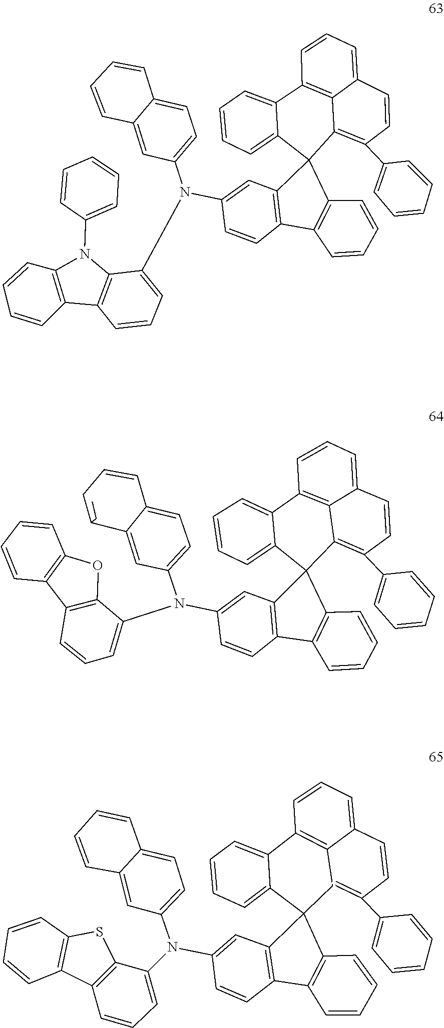

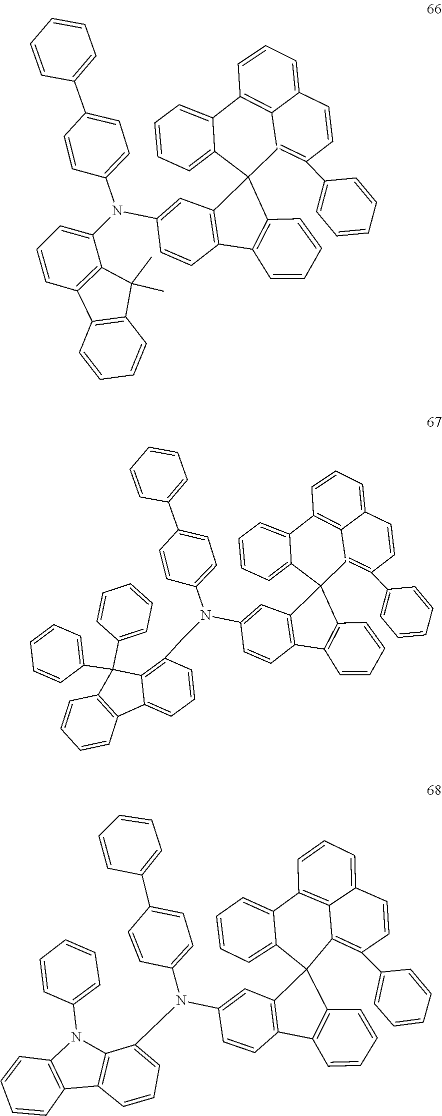 [SMILES: CC1(C)c2ccccc2-c2cccc(N(c3ccc(-c4ccccc4)cc3)c3ccc4c(c3)C3(Cc5c(-c6ccccc6)ccc6cccc(c56)-c5ccccc53)c3ccccc3-4)c21 ; c1ccc(-c2ccc(N(Cc3cccc4c3C(c3ccccc3)(c3ccccc3)c3ccccc3-4)c3ccc4c(c3)C3(Cc5c(-c6ccccc6)ccc6cccc(c56)-c5ccccc53)c3ccccc3-4)cc2)cc1 ; c1ccc(-c2ccc(N(c3ccc4c(c3)C3(c5ccccc5-4)c4ccccc4-c4cccc5ccc(-c6ccccc6)c3c45)c3cccc4c5ccccc5n(-c5ccccc5)c34)cc2)cc1]

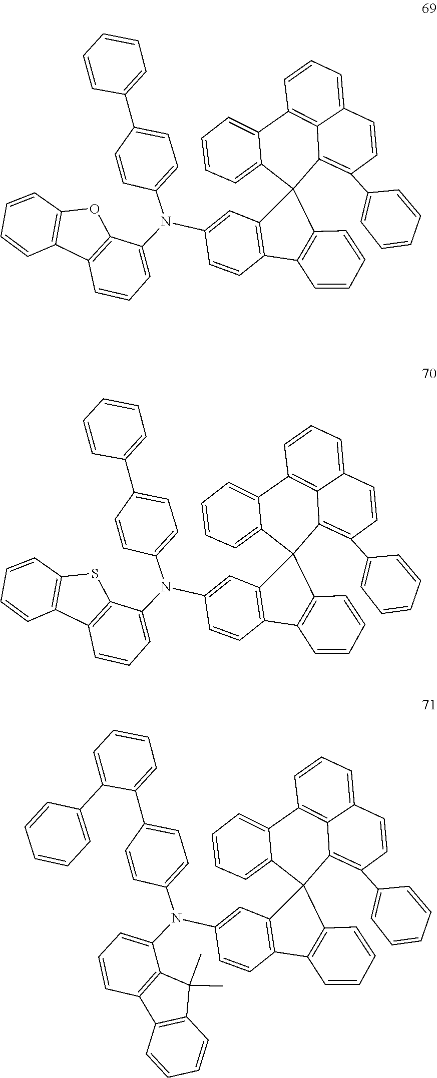 [SMILES: c1ccc(-c2ccc(N(c3ccc4c(c3)C3(c5ccccc5-4)c4ccccc4-c4cccc5ccc(-c6ccccc6)c3c45)c3cccc4c3oc3ccccc34)cc2)cc1 ; c1ccc(-c2ccc(N(c3ccc4c(c3)C3(c5ccccc5-4)c4ccccc4-c4cccc5ccc(-c6ccccc6)c3c45)c3cccc4c3sc3ccccc34)cc2)cc1 ; CC1(C)c2ccccc2-c2cccc(N(c3ccc(-c4ccccc4-c4ccccc4)cc3)c3ccc4c(c3)C3(c5ccccc5-4)c4ccccc4-c4cccc5ccc(-c6ccccc6)c3c45)c21]

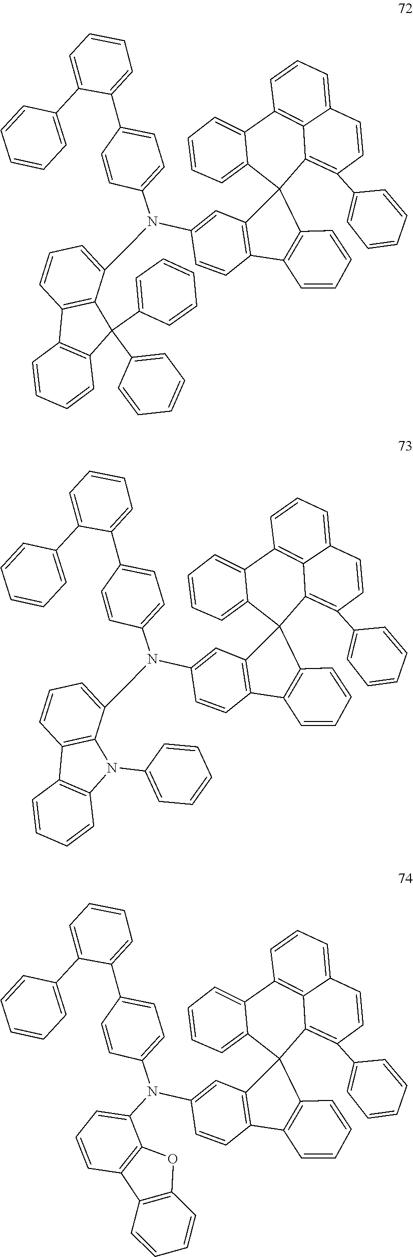 [SMILES: c1ccc(-c2ccccc2-c2ccc(N(Cc3cccc4c3C(c3ccccc3)(c3ccccc3)c3ccccc3-4)c3ccc4c(c3)C3(c5ccccc5-4)c4ccccc4-c4cccc5ccc(-c6ccccc6)c3c45)cc2)cc1 ; c1ccc(-c2ccccc2-c2ccc(N(Cc3cccc4c5ccccc5n(-c5ccccc5)c34)c3ccc4c(c3)C3(c5ccccc5-4)c4ccccc4-c4cccc5ccc(-c6ccccc6)c3c45)cc2)cc1 ; c1ccc(-c2ccccc2-c2ccc(N(c3ccc4c(c3)C3(c5ccccc5-4)c4ccccc4-c4cccc5ccc(-c6ccccc6)c3c45)c3cccc4c3oc3ccccc34)cc2)cc1]

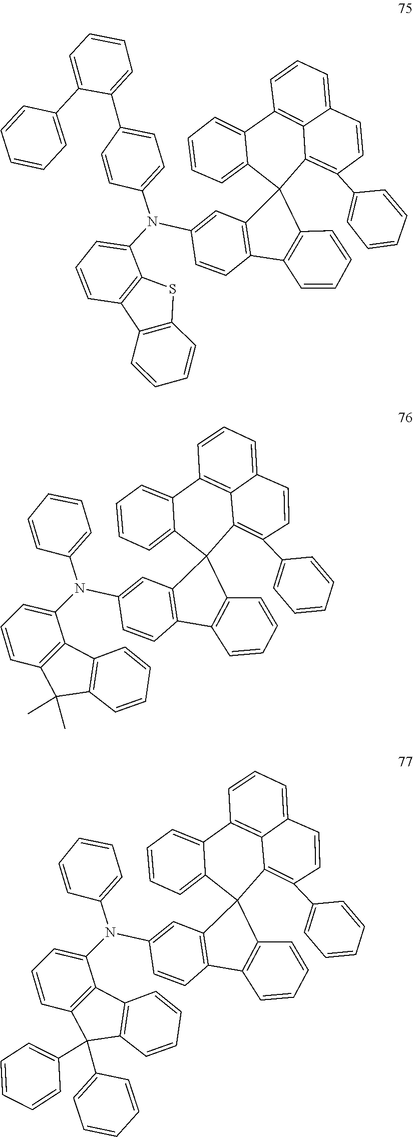 [SMILES: c1ccc(-c2ccccc2-c2ccc(N(c3ccc4c(c3)C3(c5ccccc5-4)c4ccccc4-c4cccc5ccc(-c6ccccc6)c3c45)c3cccc4c3sc3ccccc34)cc2)cc1 ; CC1(C)c2ccccc2-c2c(N(c3ccccc3)c3ccc4c(c3)C3(c5ccccc5-4)c4ccccc4-c4cccc5ccc(-c6ccccc6)c3c45)cccc21 ; c1ccc(-c2ccc3cccc4c3c2C2(c3ccccc3-c3ccc(N(c5ccccc5)c5cccc6c5-c5ccccc5C6(c5ccccc5)c5ccccc5)cc32)c2ccccc2-4)cc1]

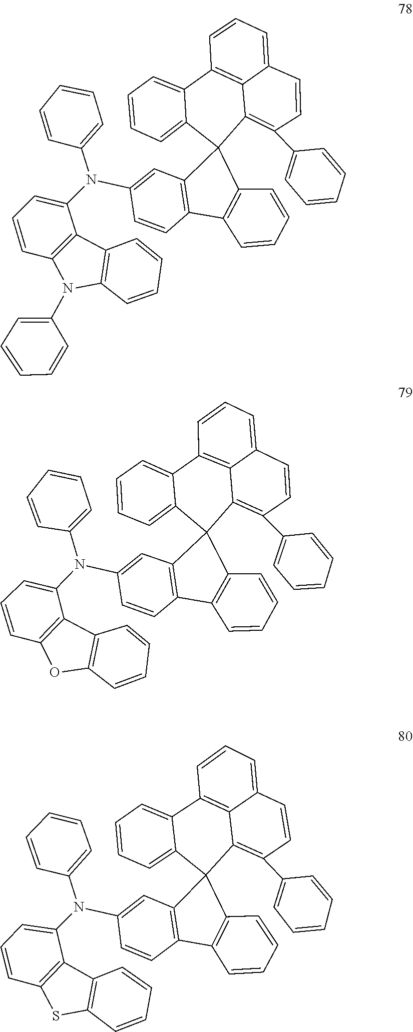 [SMILES: c1ccc(-c2ccc3cccc4c3c2C2(c3ccccc3-c3ccc(N(c5ccccc5)c5cccc6c5c5ccccc5n6-c5ccccc5)cc32)c2ccccc2-4)cc1 ; c1ccc(-c2ccc3cccc4c3c2C2(c3ccccc3-c3ccc(N(c5ccccc5)c5cccc6oc7ccccc7c56)cc32)c2ccccc2-4)cc1 ; c1ccc(-c2ccc3cccc4c3c2C2(c3ccccc3-c3ccc(N(c5ccccc5)c5cccc6sc7ccccc7c56)cc32)c2ccccc2-4)cc1]

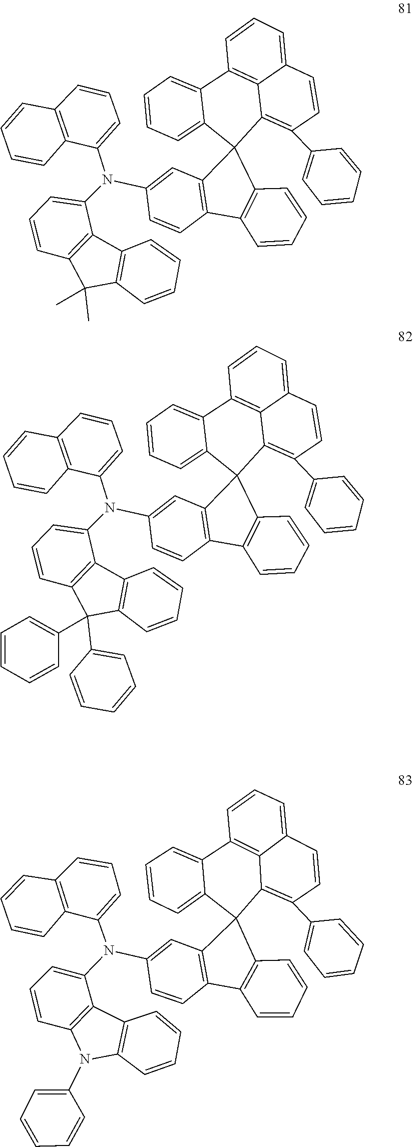 [SMILES: CC1(C)c2ccccc2-c2c(N(c3ccc4c(c3)C3(c5ccccc5-4)c4ccccc4-c4cccc5ccc(-c6ccccc6)c3c45)c3cccc4ccccc34)cccc21 ; c1ccc(-c2ccc3cccc4c3c2C2(c3ccccc3-c3ccc(N(c5cccc6c5-c5ccccc5C6(c5ccccc5)c5ccccc5)c5cccc6ccccc56)cc32)c2ccccc2-4)cc1 ; c1ccc(-c2ccc3cccc4c3c2C2(c3ccccc3-c3ccc(N(c5cccc6ccccc56)c5cccc6c5c5ccccc5n6-c5ccccc5)cc32)c2ccccc2-4)cc1]

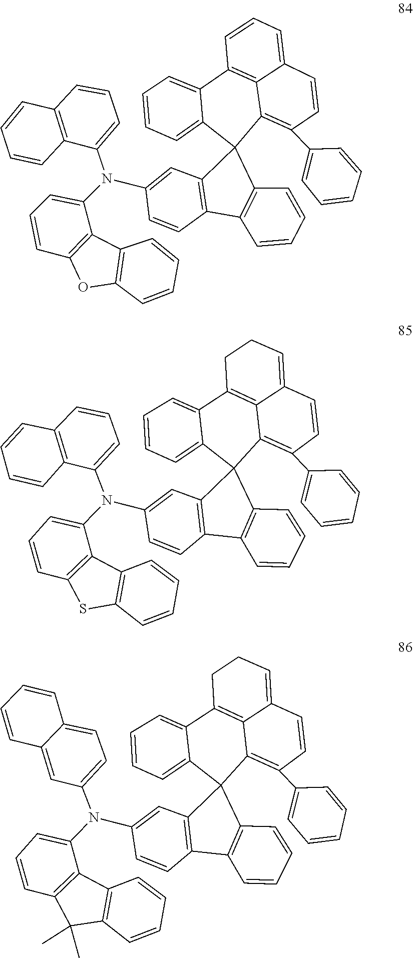 [SMILES: c1ccc(-c2ccc3cccc4c3c2C2(c3ccccc3-c3ccc(N(c5cccc6ccccc56)c5cccc6oc7ccccc7c56)cc32)c2ccccc2-4)cc1 ; C1=c2ccc(-c3ccccc3)c3c2=C(CC1)c1ccccc1C31c2ccccc2-c2ccc(N(c3cccc4ccccc34)c3cccc4sc5ccccc5c34)cc21 ; CC1(C)c2ccccc2-c2c(N(c3ccc4c(c3)C3(c5ccccc5C5=c6c3c(-c3ccccc3)ccc6=CCC5)c3ccccc3-4)c3ccc4ccccc4c3)cccc21]

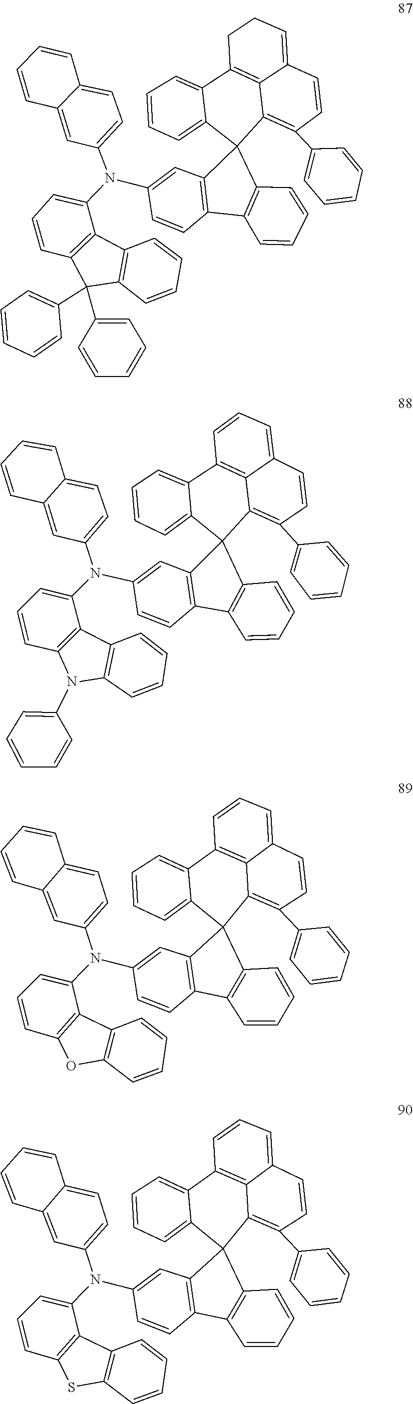 [SMILES: C1=c2ccc(-c3ccccc3)c3c2=C(CC1)c1ccccc1C31c2ccccc2-c2ccc(N(c3ccc4ccccc4c3)c3cccc4c3-c3ccccc3C4(c3ccccc3)c3ccccc3)cc21 ; c1ccc(-c2ccc3cccc4c3c2C2(c3ccccc3-c3ccc(N(c5ccc6ccccc6c5)c5cccc6c5c5ccccc5n6-c5ccccc5)cc32)c2ccccc2-4)cc1 ; c1ccc(-c2ccc3cccc4c3c2C2(c3ccccc3-c3ccc(N(c5ccc6ccccc6c5)c5cccc6oc7ccccc7c56)cc32)c2ccccc2-4)cc1 ; c1ccc(-c2ccc3cccc4c3c2C2(c3ccccc3-c3ccc(N(c5ccc6ccccc6c5)c5cccc6sc7ccccc7c56)cc32)c2ccccc2-4)cc1]

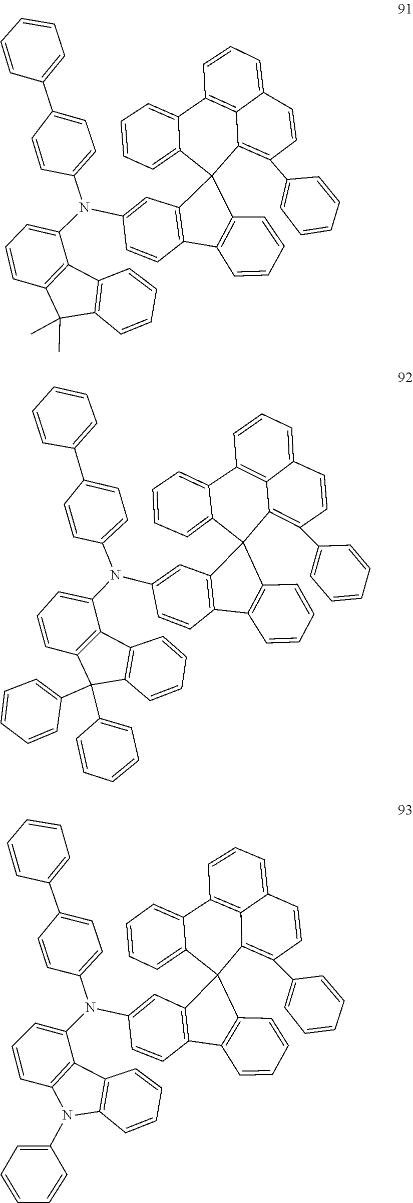

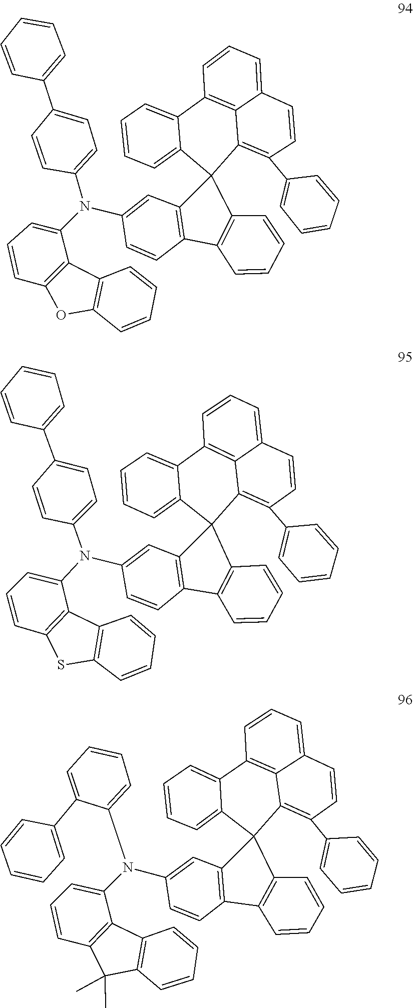 [SMILES: c1ccc(-c2ccc(N(c3ccc4c(c3)C3(c5ccccc5-4)c4ccccc4-c4cccc5ccc(-c6ccccc6)c3c45)c3cccc4oc5ccccc5c34)cc2)cc1 ; c1ccc(-c2ccc(N(c3ccc4c(c3)C3(c5ccccc5-4)c4ccccc4-c4cccc5ccc(-c6ccccc6)c3c45)c3cccc4sc5ccccc5c34)cc2)cc1 ; CC1(C)c2ccccc2-c2c(N(c3ccc4c(c3)C3(c5ccccc5-4)c4ccccc4-c4cccc5ccc(-c6ccccc6)c3c45)c3ccccc3-c3ccccc3)cccc21]

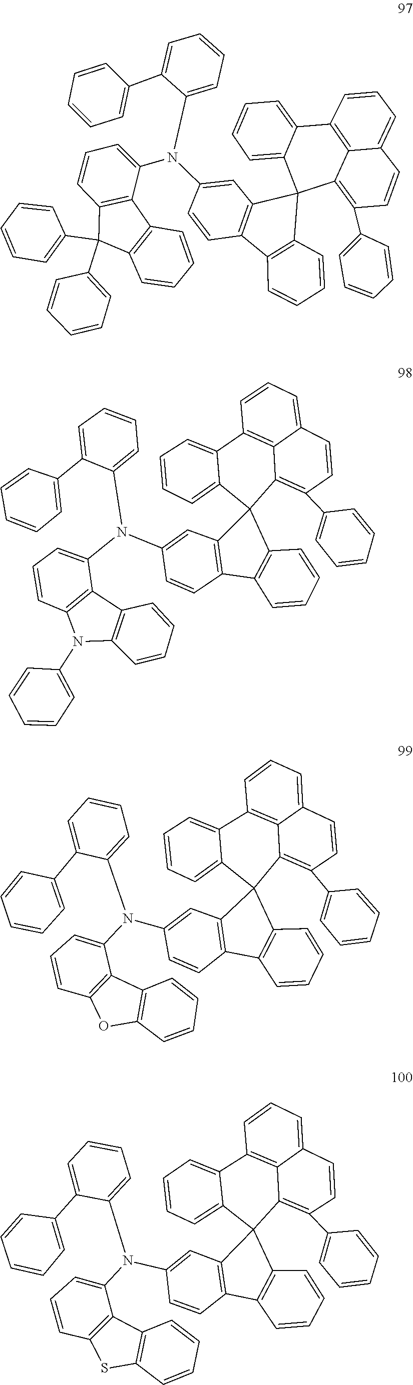 [SMILES: c1ccc(-c2ccccc2N(c2ccc3c(c2)C2(c4ccccc4-3)c3ccccc3-c3cccc4ccc(-c5ccccc5)c2c34)c2cccc3c2-c2ccccc2C3(c2ccccc2)c2ccccc2)cc1 ; c1ccc(-c2ccccc2N(c2ccc3c(c2)C2(c4ccccc4-3)c3ccccc3-c3cccc4ccc(-c5ccccc5)c2c34)c2cccc3c2c2ccccc2n3-c2ccccc2)cc1 ; c1ccc(-c2ccccc2N(c2ccc3c(c2)C2(c4ccccc4-3)c3ccccc3-c3cccc4ccc(-c5ccccc5)c2c34)c2cccc3oc4ccccc4c23)cc1 ; c1ccc(-c2ccccc2N(c2ccc3c(c2)C2(c4ccccc4-3)c3ccccc3-c3cccc4ccc(-c5ccccc5)c2c34)c2cccc3sc4ccccc4c23)cc1]

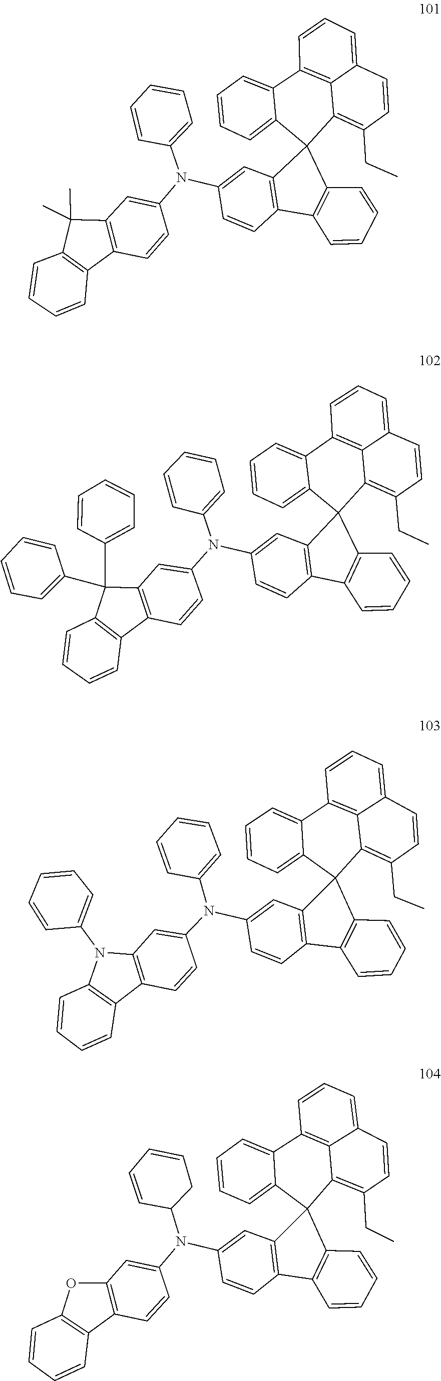 [SMILES: CCc1ccc2cccc3c2c1C1(c2ccccc2-c2ccc(N(c4ccccc4)c4ccc5c(c4)C(C)(C)c4ccccc4-5)cc21)c1ccccc1-3 ; CCc1ccc2cccc3c2c1C1(c2ccccc2-c2ccc(N(c4ccccc4)c4ccc5c(c4)C(c4ccccc4)(c4ccccc4)c4ccccc4-5)cc21)c1ccccc1-3 ; CCc1ccc2cccc3c2c1C1(c2ccccc2-c2ccc(N(c4ccccc4)c4ccc5c6ccccc6n(-c6ccccc6)c5c4)cc21)c1ccccc1-3 ; CCc1ccc2cccc3c2c1C1(c2ccccc2-c2ccc(N(c4ccc5c(c4)oc4ccccc45)C4C=CC=CC4)cc21)c1ccccc1-3]

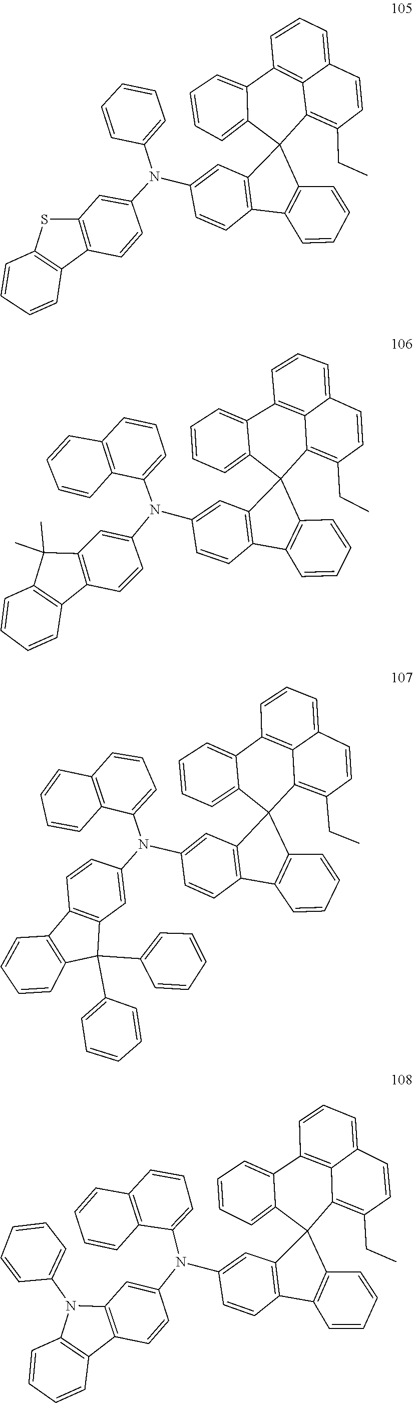 [SMILES: CCc1ccc2cccc3c2c1C1(c2ccccc2-c2ccc(N(c4ccccc4)c4ccc5c(c4)sc4ccccc45)cc21)c1ccccc1-3 ; CCc1ccc2cccc3c2c1C1(c2ccccc2-c2ccc(N(c4ccc5c(c4)C(C)(C)c4ccccc4-5)c4cccc5ccccc45)cc21)c1ccccc1-3 ; CCc1ccc2cccc3c2c1C1(c2ccccc2-c2ccc(N(c4ccc5c(c4)C(c4ccccc4)(c4ccccc4)c4ccccc4-5)c4cccc5ccccc45)cc21)c1ccccc1-3 ; CCc1ccc2cccc3c2c1C1(c2ccccc2-c2ccc(N(c4ccc5c6ccccc6n(-c6ccccc6)c5c4)c4cccc5ccccc45)cc21)c1ccccc1-3]

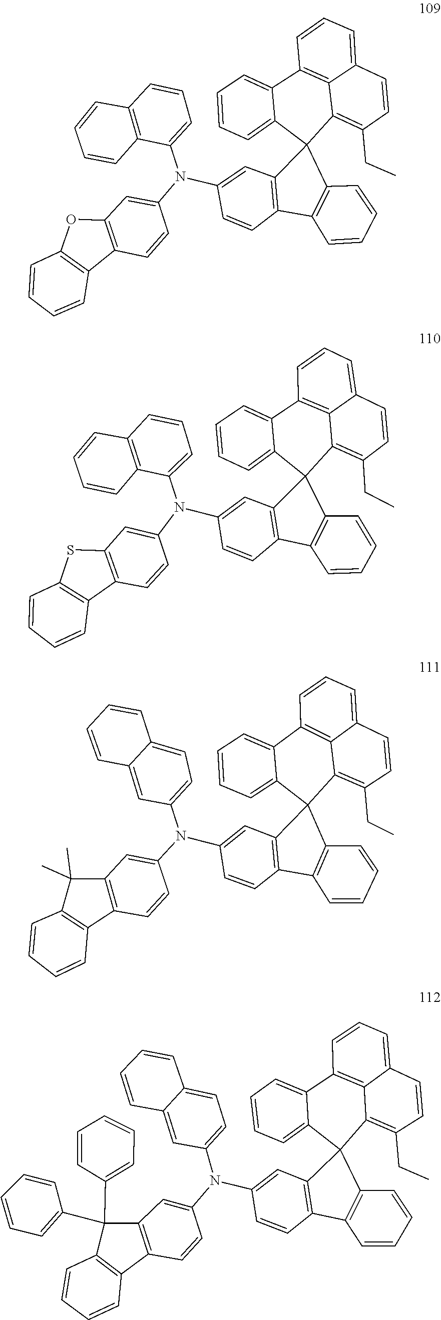 [SMILES: CCc1ccc2cccc3c2c1C1(c2ccccc2-c2ccc(N(c4ccc5c(c4)oc4ccccc45)c4cccc5ccccc45)cc21)c1ccccc1-3 ; CCc1ccc2cccc3c2c1C1(c2ccccc2-c2ccc(N(c4ccc5c(c4)sc4ccccc45)c4cccc5ccccc45)cc21)c1ccccc1-3 ; CCc1ccc2cccc3c2c1C1(c2ccccc2-c2ccc(N(c4ccc5c(c4)C(C)(C)c4ccccc4-5)c4ccc5ccccc5c4)cc21)c1ccccc1-3 ; CCc1ccc2cccc3c2c1C1(c2ccccc2-c2ccc(N(c4ccc5c(c4)C(c4ccccc4)(c4ccccc4)c4ccccc4-5)c4ccc5ccccc5c4)cc21)c1ccccc1-3]

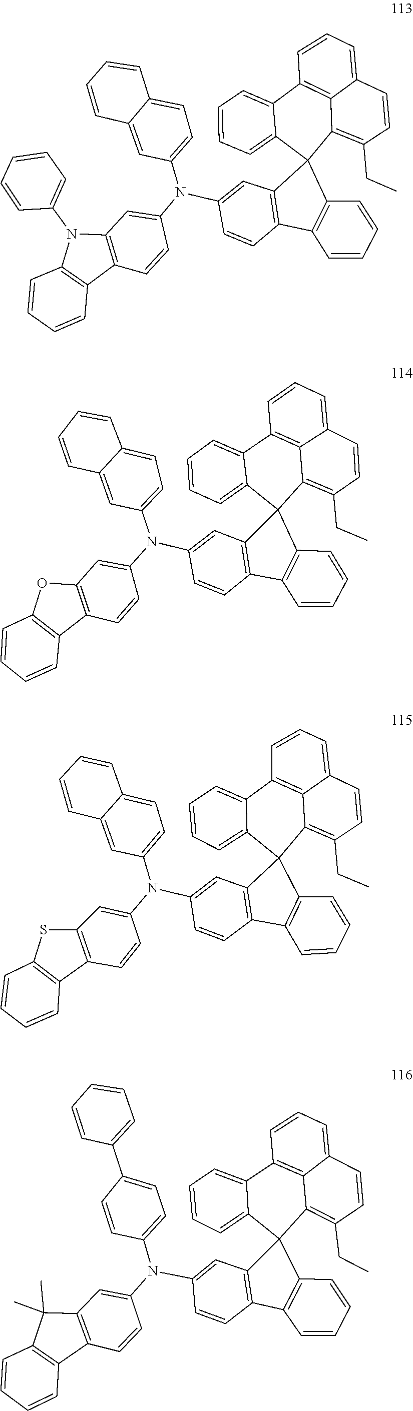 [SMILES: CCc1ccc2cccc3c2c1C1(c2ccccc2-c2ccc(N(c4ccc5ccccc5c4)c4ccc5c6ccccc6n(-c6ccccc6)c5c4)cc21)c1ccccc1-3 ; CCc1ccc2cccc3c2c1C1(c2ccccc2-c2ccc(N(c4ccc5ccccc5c4)c4ccc5c(c4)oc4ccccc45)cc21)c1ccccc1-3 ; CCc1ccc2cccc3c2c1C1(c2ccccc2-c2ccc(N(c4ccc5ccccc5c4)c4ccc5c(c4)sc4ccccc45)cc21)c1ccccc1-3 ; CCc1ccc2cccc3c2c1C1(c2ccccc2-c2ccc(N(c4ccc(-c5ccccc5)cc4)c4ccc5c(c4)C(C)(C)c4ccccc4-5)cc21)c1ccccc1-3]

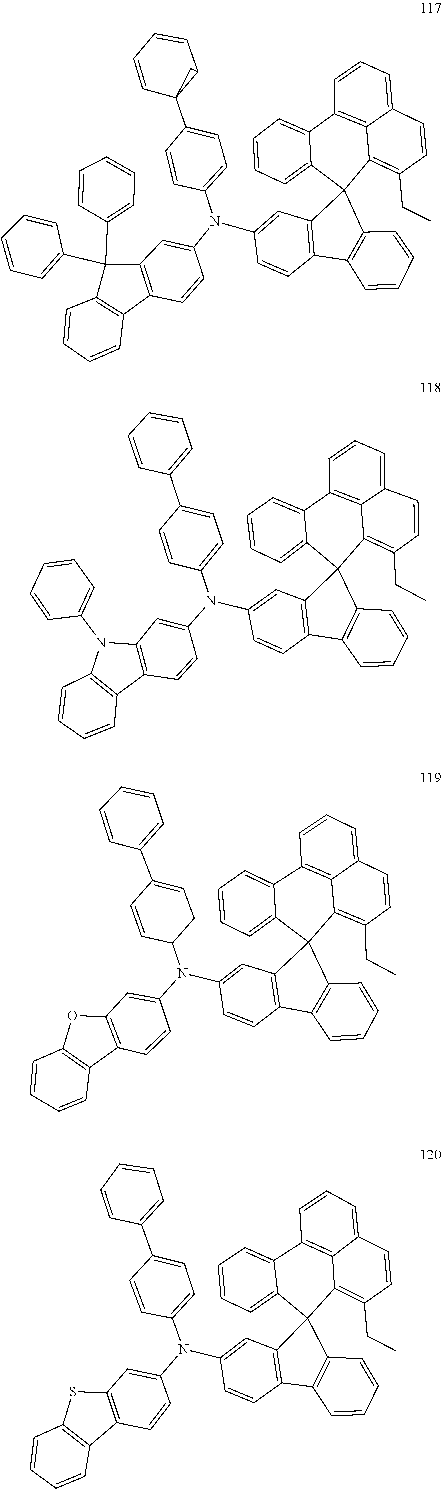 [SMILES: CCc1ccc2cccc3c2c1C1(c2ccccc2-c2ccc(N(c4ccc(C56C=CC=CC5C6)cc4)c4ccc5c(c4)C(c4ccccc4)(c4ccccc4)c4ccccc4-5)cc21)c1ccccc1-3 ; CCc1ccc2cccc3c2c1C1(c2ccccc2-c2ccc(N(c4ccc(-c5ccccc5)cc4)c4ccc5c6ccccc6n(-c6ccccc6)c5c4)cc21)c1ccccc1-3 ; CCc1ccc2cccc3c2c1C1(c2ccccc2-c2ccc(N(c4ccc5c(c4)oc4ccccc45)C4C=CC(c5ccccc5)=CC4)cc21)c1ccccc1-3 ; CCc1ccc2cccc3c2c1C1(c2ccccc2-c2ccc(N(c4ccc(-c5ccccc5)cc4)c4ccc5c(c4)sc4ccccc45)cc21)c1ccccc1-3]

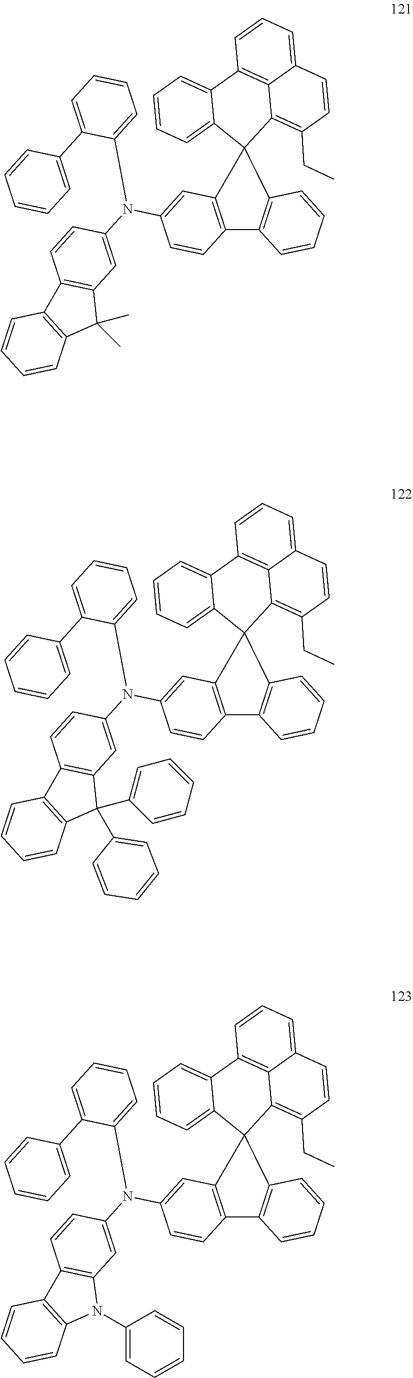 [SMILES: CCc1ccc2cccc3c2c1C1(c2ccccc2-c2ccc(N(c4ccc5c(c4)C(C)(C)c4ccccc4-5)c4ccccc4-c4ccccc4)cc21)c1ccccc1-3 ; CCc1ccc2cccc3c2c1C1(c2ccccc2-c2ccc(N(c4ccc5c(c4)C(c4ccccc4)(c4ccccc4)c4ccccc4-5)c4ccccc4-c4ccccc4)cc21)c1ccccc1-3 ; CCc1ccc2cccc3c2c1C1(c2ccccc2-c2ccc(N(c4ccc5c6ccccc6n(-c6ccccc6)c5c4)c4ccccc4-c4ccccc4)cc21)c1ccccc1-3]

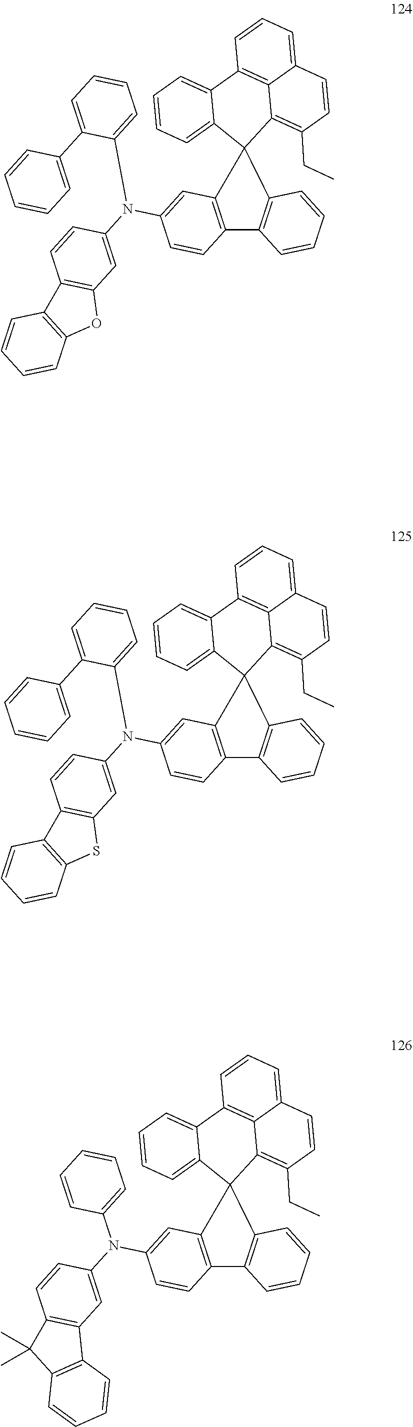

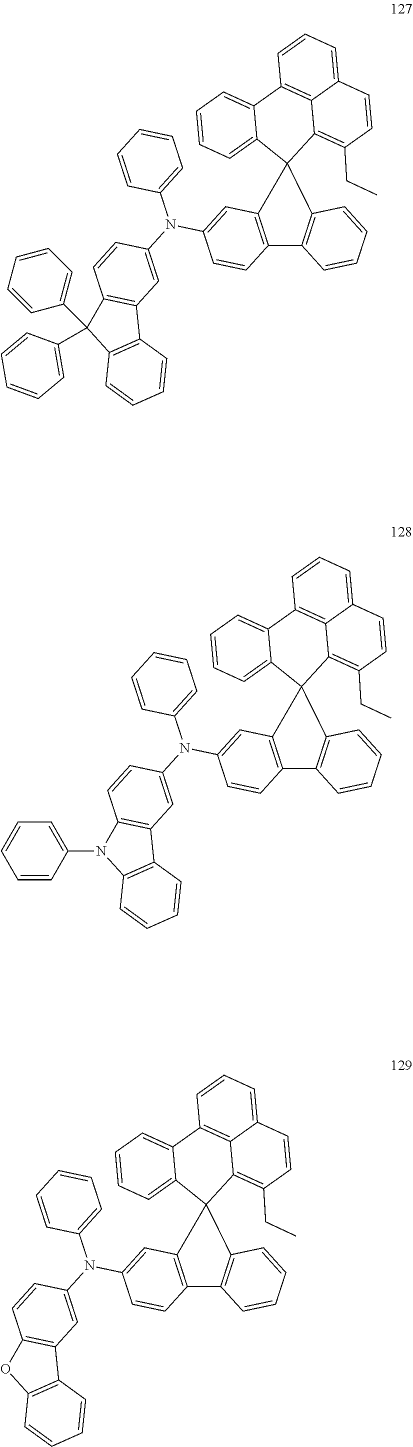 [SMILES: CCc1ccc2cccc3c2c1C1(c2ccccc2-c2ccc(N(c4ccccc4)c4ccc5c(c4)-c4ccccc4C5(c4ccccc4)c4ccccc4)cc21)c1ccccc1-3 ; CCc1ccc2cccc3c2c1C1(c2ccccc2-c2ccc(N(c4ccccc4)c4ccc5c(c4)c4ccccc4n5-c4ccccc4)cc21)c1ccccc1-3 ; CCc1ccc2cccc3c2c1C1(c2ccccc2-c2ccc(N(c4ccccc4)c4ccc5oc6ccccc6c5c4)cc21)c1ccccc1-3]

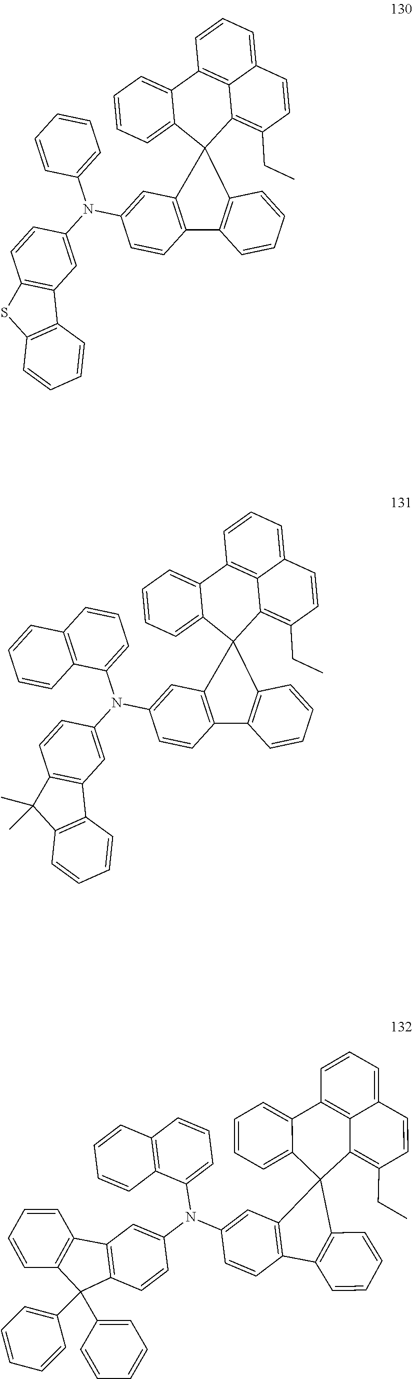

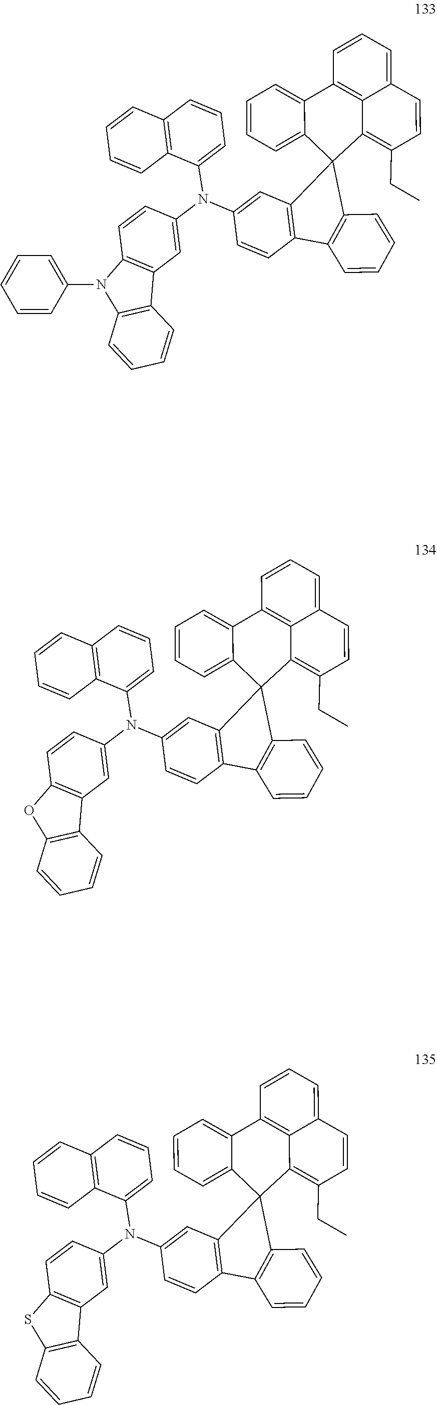 [SMILES: CCc1ccc2cccc3c2c1C1(c2ccccc2-c2ccc(N(c4ccc5c(c4)c4ccccc4n5-c4ccccc4)c4cccc5ccccc45)cc21)c1ccccc1-3 ; CCc1ccc2cccc3c2c1C1(c2ccccc2-c2ccc(N(c4ccc5oc6ccccc6c5c4)c4cccc5ccccc45)cc21)c1ccccc1-3 ; CCc1ccc2cccc3c2c1C1(c2ccccc2-c2ccc(N(c4ccc5sc6ccccc6c5c4)c4cccc5ccccc45)cc21)c1ccccc1-3]

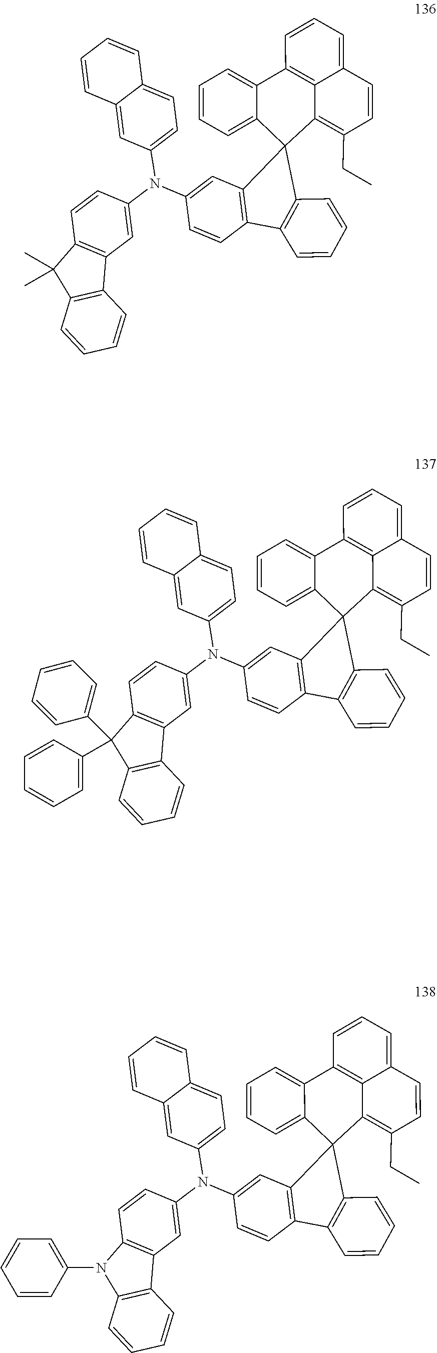 [SMILES: CCc1ccc2cccc3c2c1C1(c2ccccc2-c2ccc(N(c4ccc5c(c4)-c4ccccc4C5(C)C)c4ccc5ccccc5c4)cc21)c1ccccc1-3 ; CCc1ccc2cccc3c2c1C1(c2ccccc2-c2ccc(N(c4ccc5c(c4)-c4ccccc4C5(c4ccccc4)c4ccccc4)c4ccc5ccccc5c4)cc21)c1ccccc1-3 ; CCc1ccc2cccc3c2c1C1(c2ccccc2-c2ccc(N(c4ccc5ccccc5c4)c4ccc5c(c4)c4ccccc4n5-c4ccccc4)cc21)c1ccccc1-3]

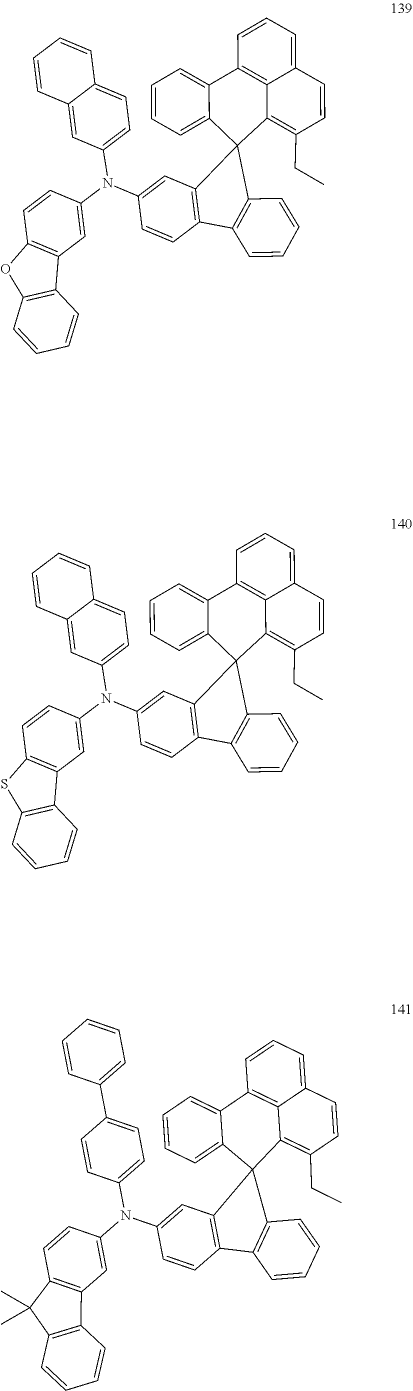 [SMILES: CCc1ccc2cccc3c2c1C1(c2ccccc2-c2ccc(N(c4ccc5ccccc5c4)c4ccc5oc6ccccc6c5c4)cc21)c1ccccc1-3 ; CCc1ccc2cccc3c2c1C1(c2ccccc2-c2ccc(N(c4ccc5ccccc5c4)c4ccc5sc6ccccc6c5c4)cc21)c1ccccc1-3 ; CCc1ccc2cccc3c2c1C1(c2ccccc2-c2ccc(N(c4ccc(-c5ccccc5)cc4)c4ccc5c(c4)-c4ccccc4C5(C)C)cc21)c1ccccc1-3]

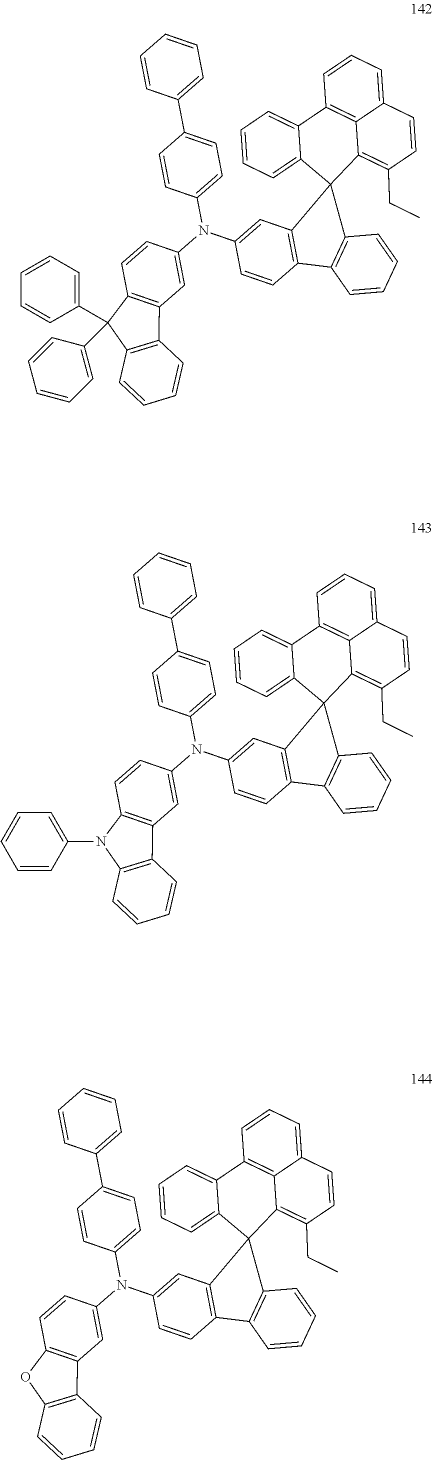 [SMILES: CCc1ccc2cccc3c2c1C1(c2ccccc2-c2ccc(N(c4ccc(-c5ccccc5)cc4)c4ccc5c(c4)-c4ccccc4C5(c4ccccc4)c4ccccc4)cc21)c1ccccc1-3 ; CCc1ccc2cccc3c2c1C1(c2ccccc2-c2ccc(N(c4ccc(-c5ccccc5)cc4)c4ccc5c(c4)c4ccccc4n5-c4ccccc4)cc21)c1ccccc1-3 ; CCc1ccc2cccc3c2c1C1(c2ccccc2-c2ccc(N(c4ccc(-c5ccccc5)cc4)c4ccc5oc6ccccc6c5c4)cc21)c1ccccc1-3]

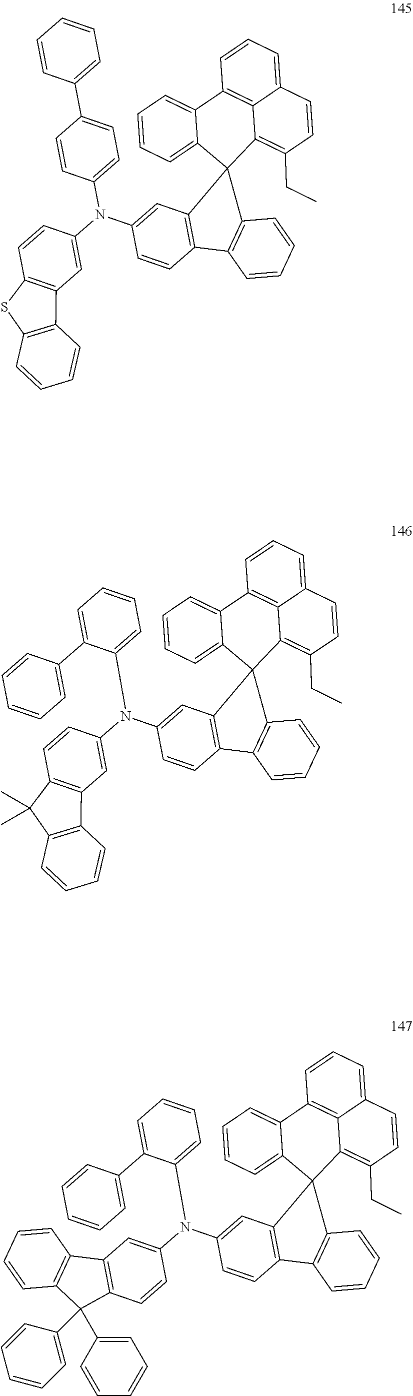

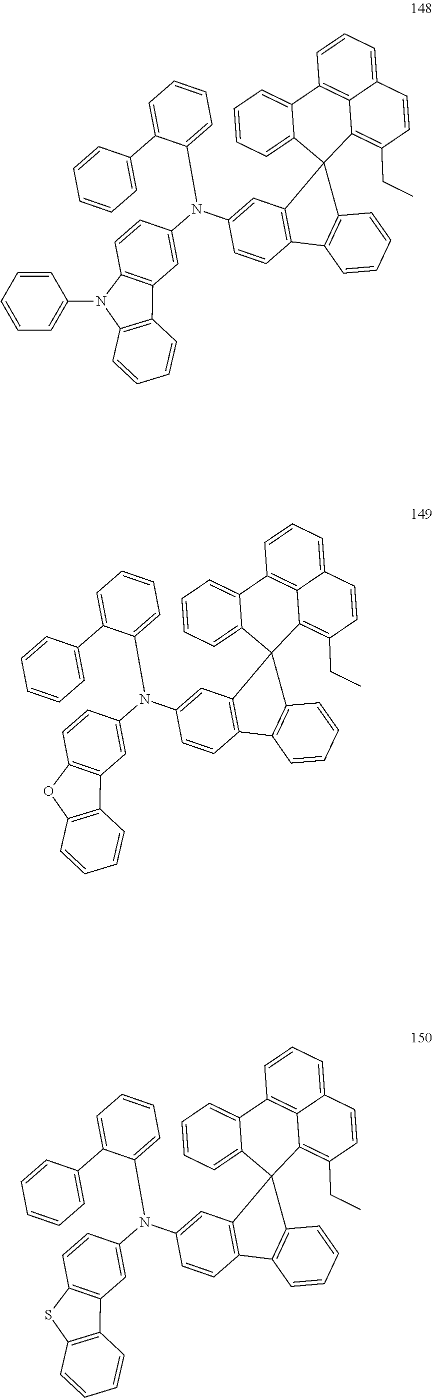 [SMILES: CCc1ccc2cccc3c2c1C1(c2ccccc2-c2ccc(N(c4ccc5c(c4)c4ccccc4n5-c4ccccc4)c4ccccc4-c4ccccc4)cc21)c1ccccc1-3 ; CCc1ccc2cccc3c2c1C1(c2ccccc2-c2ccc(N(c4ccc5oc6ccccc6c5c4)c4ccccc4-c4ccccc4)cc21)c1ccccc1-3 ; CCc1ccc2cccc3c2c1C1(c2ccccc2-c2ccc(N(c4ccc5sc6ccccc6c5c4)c4ccccc4-c4ccccc4)cc21)c1ccccc1-3]

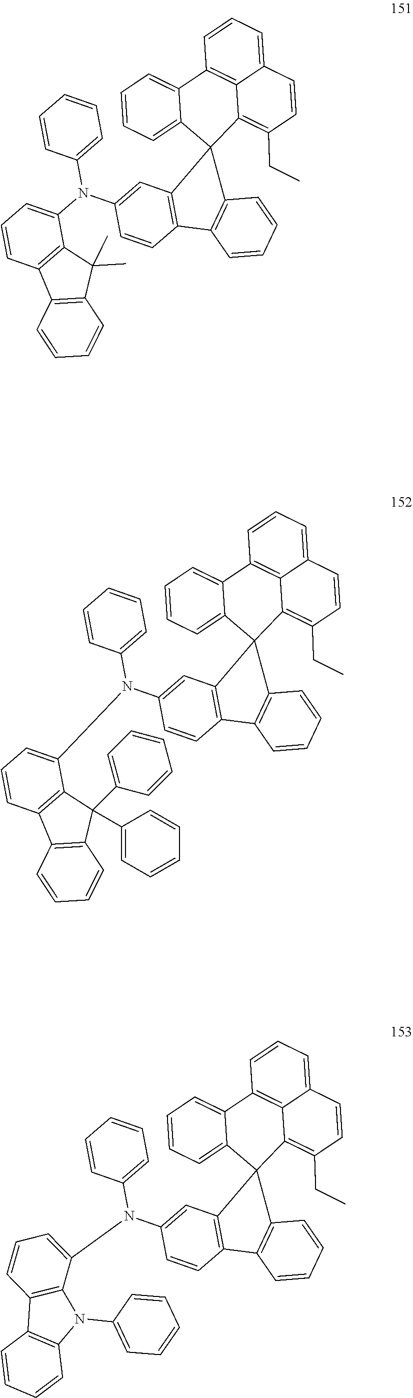 [SMILES: CCc1ccc2cccc3c2c1C1(c2ccccc2-c2ccc(N(c4ccccc4)c4cccc5c4C(C)(C)c4ccccc4-5)cc21)c1ccccc1-3 ; CCc1ccc2cccc3c2c1C1(c2ccccc2-c2ccc(N(c4ccccc4)c4cccc5c4C(c4ccccc4)(c4ccccc4)c4ccccc4-5)cc21)c1ccccc1-3 ; CCc1ccc2cccc3c2c1C1(c2ccccc2-c2ccc(N(c4ccccc4)c4cccc5c6ccccc6n(-c6ccccc6)c45)cc21)c1ccccc1-3]

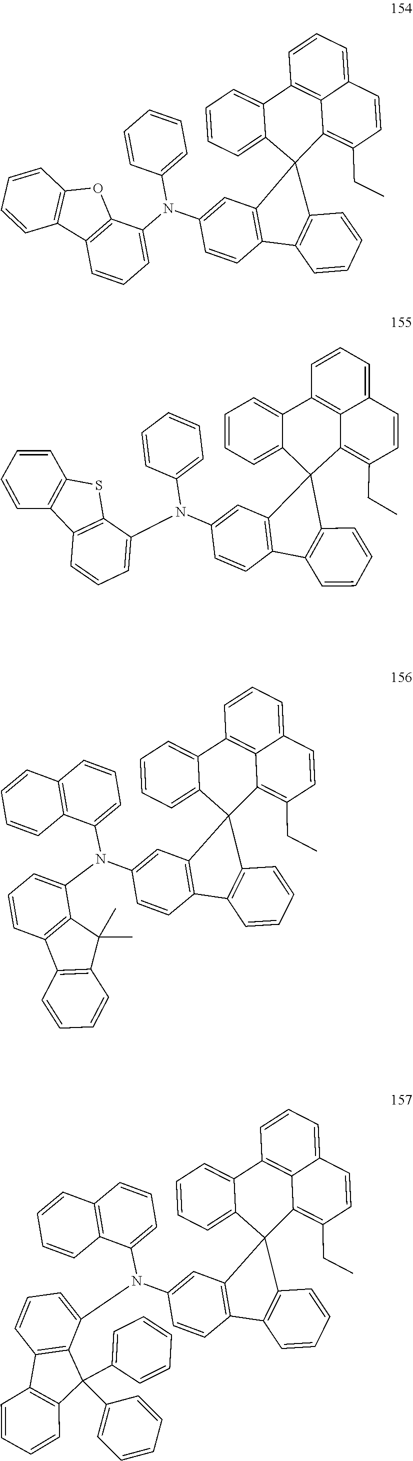 [SMILES: CCc1ccc2cccc3c2c1C1(c2ccccc2-c2ccc(N(c4ccccc4)c4cccc5c4oc4ccccc45)cc21)c1ccccc1-3 ; CCc1ccc2cccc3c2c1C1(c2ccccc2-c2ccc(N(c4ccccc4)c4cccc5c4sc4ccccc45)cc21)c1ccccc1-3 ; CCc1ccc2cccc3c2c1C1(c2ccccc2-c2ccc(N(c4cccc5c4C(C)(C)c4ccccc4-5)c4cccc5ccccc45)cc21)c1ccccc1-3 ; CCc1ccc2cccc3c2c1C1(c2ccccc2-c2ccc(N(c4cccc5c4C(c4ccccc4)(c4ccccc4)c4ccccc4-5)c4cccc5ccccc45)cc21)c1ccccc1-3]

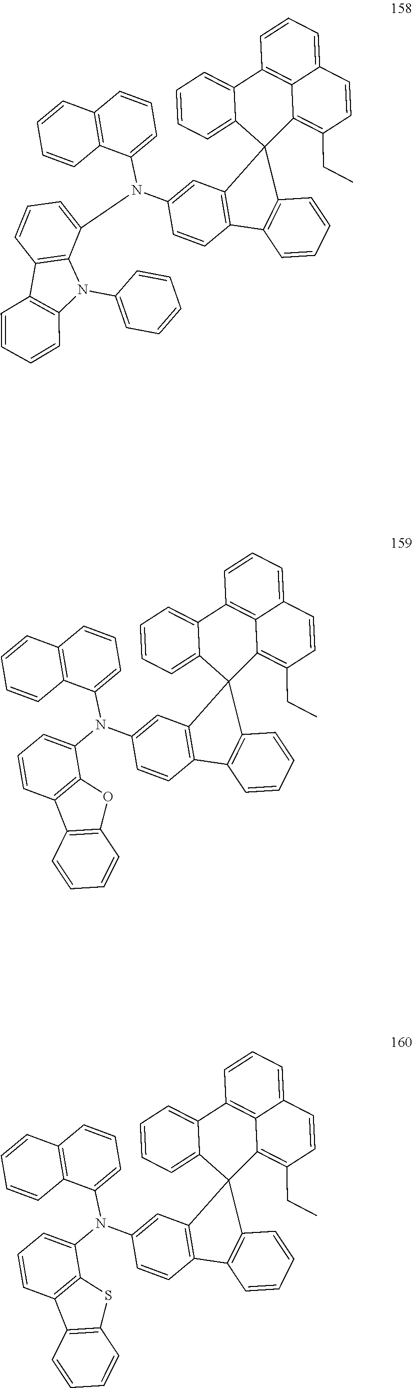 [SMILES: CCc1ccc2cccc3c2c1C1(c2ccccc2-c2ccc(N(c4cccc5ccccc45)c4cccc5c6ccccc6n(-c6ccccc6)c45)cc21)c1ccccc1-3 ; CCc1ccc2cccc3c2c1C1(c2ccccc2-c2ccc(N(c4cccc5ccccc45)c4cccc5c4oc4ccccc45)cc21)c1ccccc1-3 ; CCc1ccc2cccc3c2c1C1(c2ccccc2-c2ccc(N(c4cccc5ccccc45)c4cccc5c4sc4ccccc45)cc21)c1ccccc1-3]

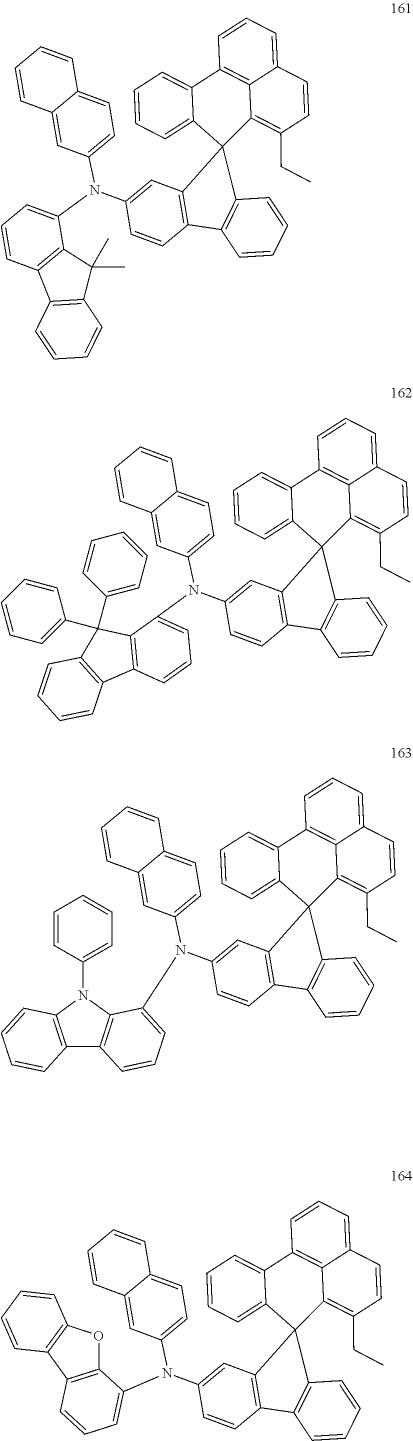 [SMILES: CCc1ccc2cccc3c2c1C1(c2ccccc2-c2ccc(N(c4ccc5ccccc5c4)c4cccc5c4C(C)(C)c4ccccc4-5)cc21)c1ccccc1-3 ; CCc1ccc2cccc3c2c1C1(c2ccccc2-c2ccc(N(c4ccc5ccccc5c4)c4cccc5c4C(c4ccccc4)(c4ccccc4)c4ccccc4-5)cc21)c1ccccc1-3 ; CCc1ccc2cccc3c2c1C1(c2ccccc2-c2ccc(N(c4ccc5ccccc5c4)c4cccc5c6ccccc6n(-c6ccccc6)c45)cc21)c1ccccc1-3 ; CCc1ccc2cccc3c2c1C1(c2ccccc2-c2ccc(N(c4ccc5ccccc5c4)c4cccc5c4oc4ccccc45)cc21)c1ccccc1-3]

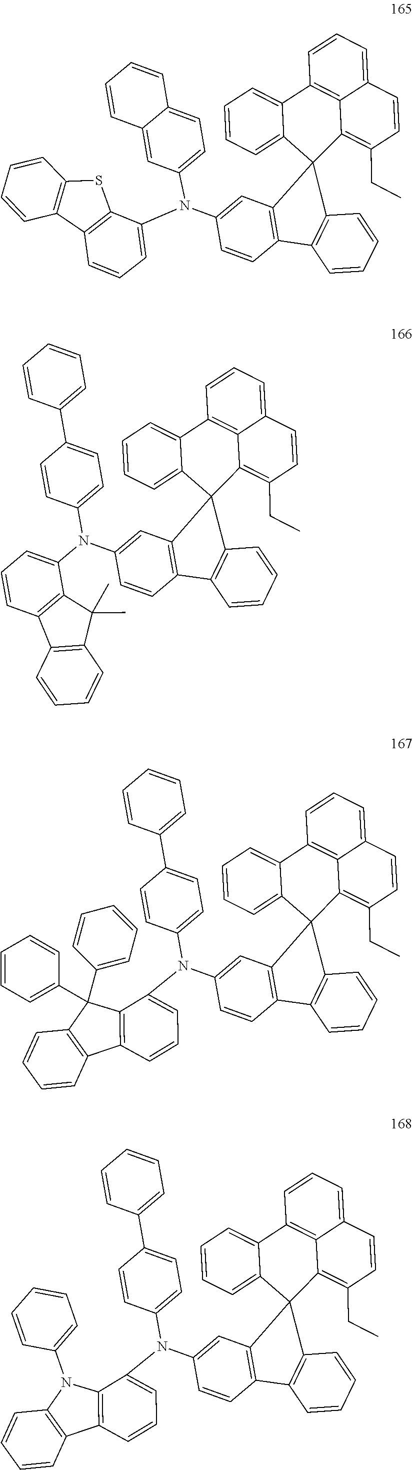 [SMILES: CCc1ccc2cccc3c2c1C1(c2ccccc2-c2ccc(N(c4ccc5ccccc5c4)c4cccc5c4sc4ccccc45)cc21)c1ccccc1-3 ; CCc1ccc2cccc3c2c1C1(c2ccccc2-c2ccc(N(c4ccc(-c5ccccc5)cc4)c4cccc5c4C(C)(C)c4ccccc4-5)cc21)c1ccccc1-3 ; CCc1ccc2cccc3c2c1C1(c2ccccc2-c2ccc(N(c4ccc(-c5ccccc5)cc4)c4cccc5c4C(c4ccccc4)(c4ccccc4)c4ccccc4-5)cc21)c1ccccc1-3 ; CCc1ccc2cccc3c2c1C1(c2ccccc2-c2ccc(N(c4ccc(-c5ccccc5)cc4)c4cccc5c6ccccc6n(-c6ccccc6)c45)cc21)c1ccccc1-3]

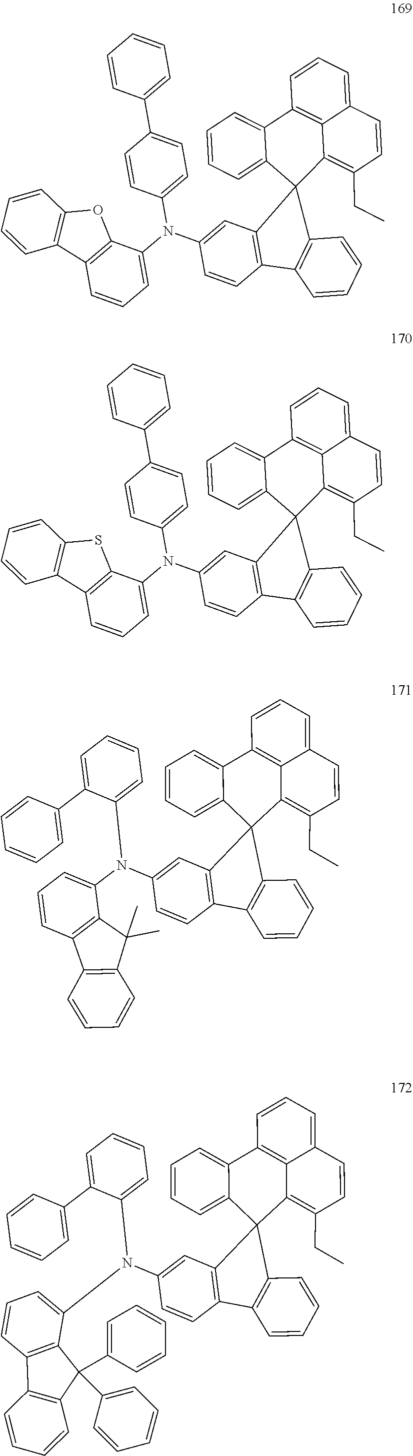 [SMILES: CCc1ccc2cccc3c2c1C1(c2ccccc2-c2ccc(N(c4ccc(-c5ccccc5)cc4)c4cccc5c4oc4ccccc45)cc21)c1ccccc1-3 ; CCc1ccc2cccc3c2c1C1(c2ccccc2-c2ccc(N(c4ccc(-c5ccccc5)cc4)c4cccc5c4sc4ccccc45)cc21)c1ccccc1-3 ; CCc1ccc2cccc3c2c1C1(c2ccccc2-c2ccc(N(c4ccccc4-c4ccccc4)c4cccc5c4C(C)(C)c4ccccc4-5)cc21)c1ccccc1-3 ; CCc1ccc2cccc3c2c1C1(c2ccccc2-c2ccc(N(c4ccccc4-c4ccccc4)c4cccc5c4C(c4ccccc4)(c4ccccc4)c4ccccc4-5)cc21)c1ccccc1-3]

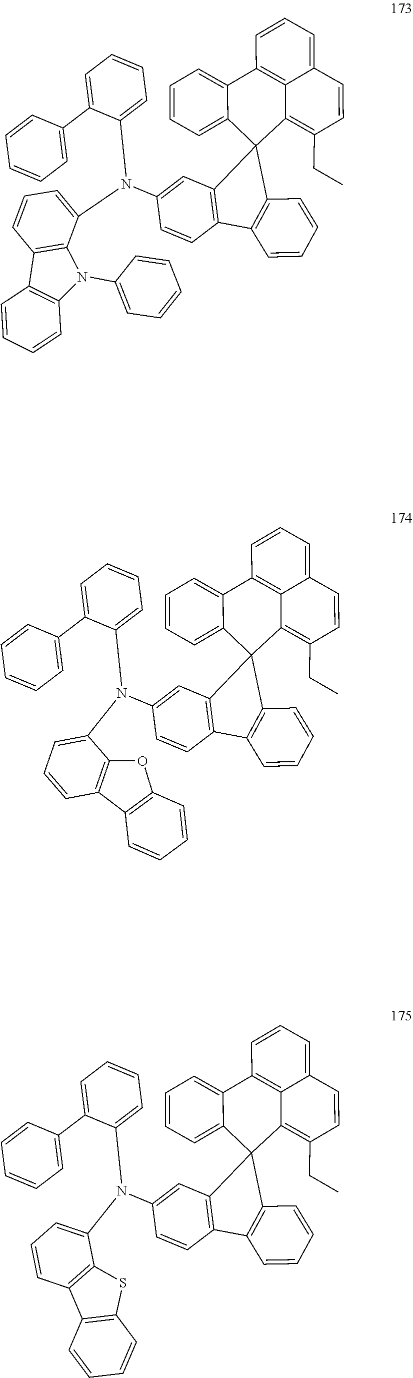 [SMILES: CCc1ccc2cccc3c2c1C1(c2ccccc2-c2ccc(N(c4ccccc4-c4ccccc4)c4cccc5c6ccccc6n(-c6ccccc6)c45)cc21)c1ccccc1-3 ; CCc1ccc2cccc3c2c1C1(c2ccccc2-c2ccc(N(c4ccccc4-c4ccccc4)c4cccc5c4oc4ccccc45)cc21)c1ccccc1-3 ; CCc1ccc2cccc3c2c1C1(c2ccccc2-c2ccc(N(c4ccccc4-c4ccccc4)c4cccc5c4sc4ccccc45)cc21)c1ccccc1-3]

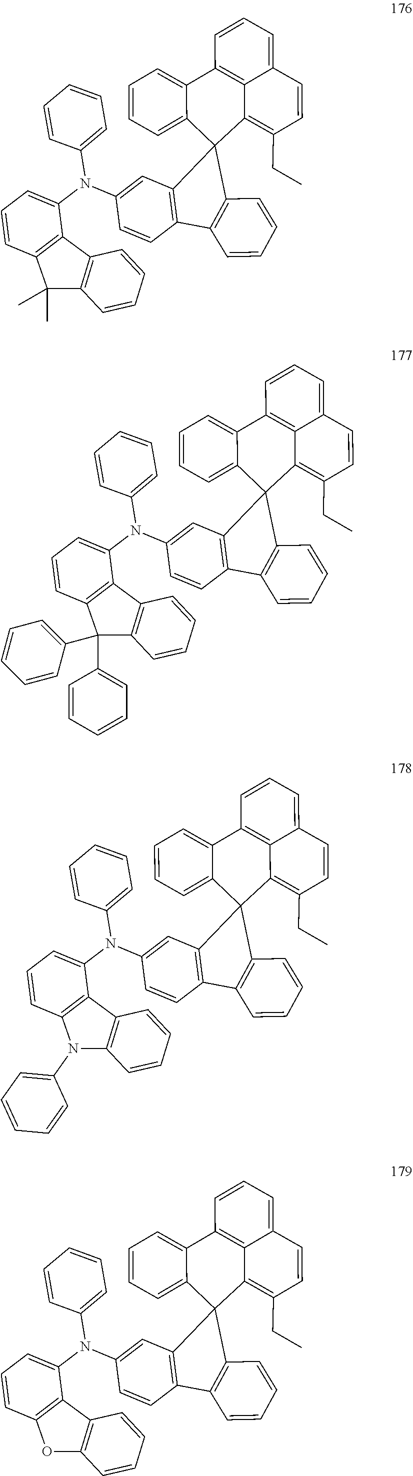 [SMILES: CCc1ccc2cccc3c2c1C1(c2ccccc2-c2ccc(N(c4ccccc4)c4cccc5c4-c4ccccc4C5(C)C)cc21)c1ccccc1-3 ; CCc1ccc2cccc3c2c1C1(c2ccccc2-c2ccc(N(c4ccccc4)c4cccc5c4-c4ccccc4C5(c4ccccc4)c4ccccc4)cc21)c1ccccc1-3 ; CCc1ccc2cccc3c2c1C1(c2ccccc2-c2ccc(N(c4ccccc4)c4cccc5c4c4ccccc4n5-c4ccccc4)cc21)c1ccccc1-3 ; CCc1ccc2cccc3c2c1C1(c2ccccc2-c2ccc(N(c4ccccc4)c4cccc5oc6ccccc6c45)cc21)c1ccccc1-3]

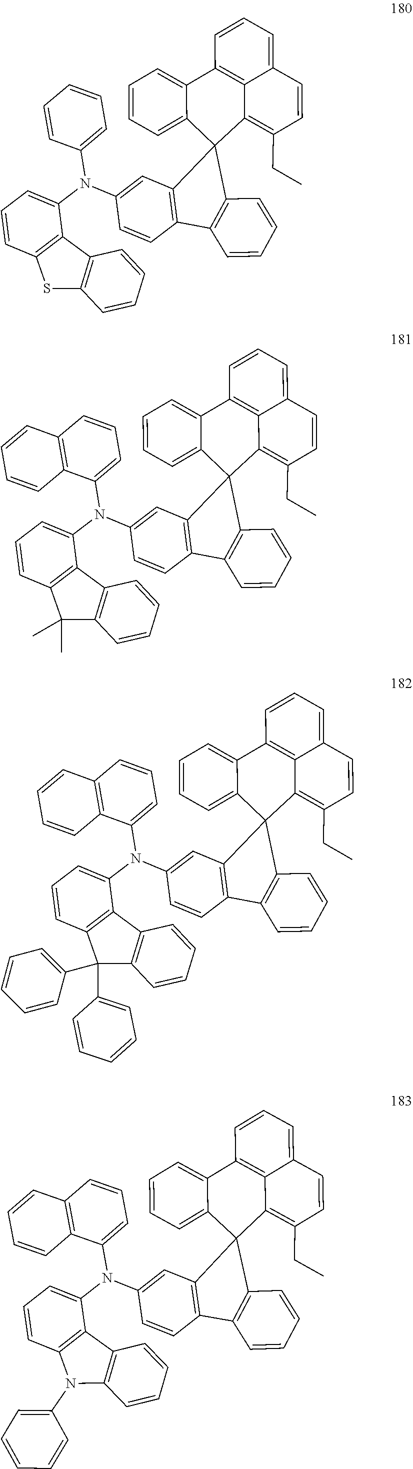 [SMILES: CCc1ccc2cccc3c2c1C1(c2ccccc2-c2ccc(N(c4ccccc4)c4cccc5sc6ccccc6c45)cc21)c1ccccc1-3 ; CCc1ccc2cccc3c2c1C1(c2ccccc2-c2ccc(N(c4cccc5c4-c4ccccc4C5(C)C)c4cccc5ccccc45)cc21)c1ccccc1-3 ; CCc1ccc2cccc3c2c1C1(c2ccccc2-c2ccc(N(c4cccc5c4-c4ccccc4C5(c4ccccc4)c4ccccc4)c4cccc5ccccc45)cc21)c1ccccc1-3 ; CCc1ccc2cccc3c2c1C1(c2ccccc2-c2ccc(N(c4cccc5ccccc45)c4cccc5c4c4ccccc4n5-c4ccccc4)cc21)c1ccccc1-3]

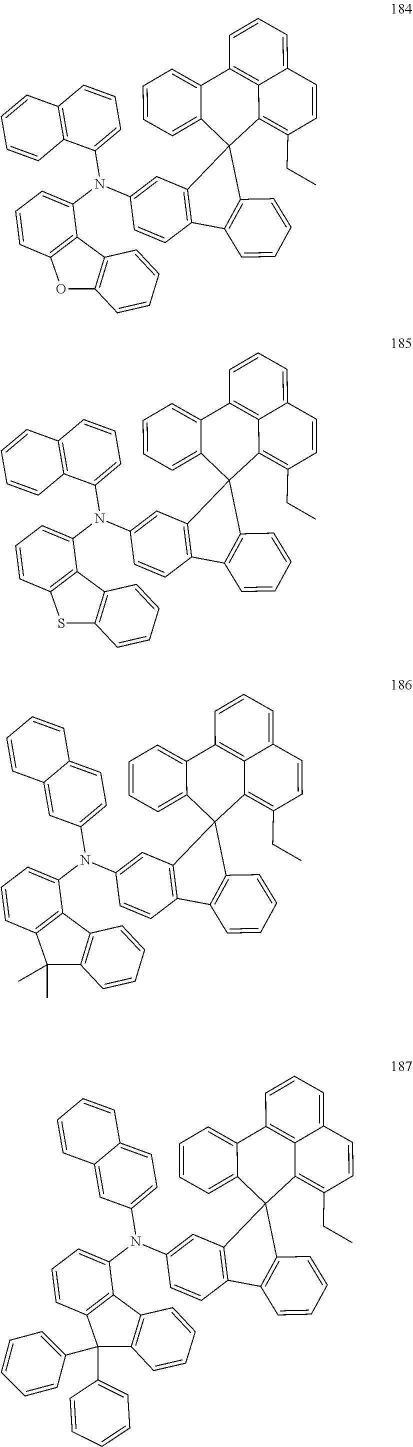 [SMILES: CCc1ccc2cccc3c2c1C1(c2ccccc2-c2ccc(N(c4cccc5ccccc45)c4cccc5oc6ccccc6c45)cc21)c1ccccc1-3 ; CCc1ccc2cccc3c2c1C1(c2ccccc2-c2ccc(N(c4cccc5ccccc45)c4cccc5sc6ccccc6c45)cc21)c1ccccc1-3 ; CCc1ccc2cccc3c2c1C1(c2ccccc2-c2ccc(N(c4ccc5ccccc5c4)c4cccc5c4-c4ccccc4C5(C)C)cc21)c1ccccc1-3 ; CCc1ccc2cccc3c2c1C1(c2ccccc2-c2ccc(N(c4ccc5ccccc5c4)c4cccc5c4-c4ccccc4C5(c4ccccc4)c4ccccc4)cc21)c1ccccc1-3]

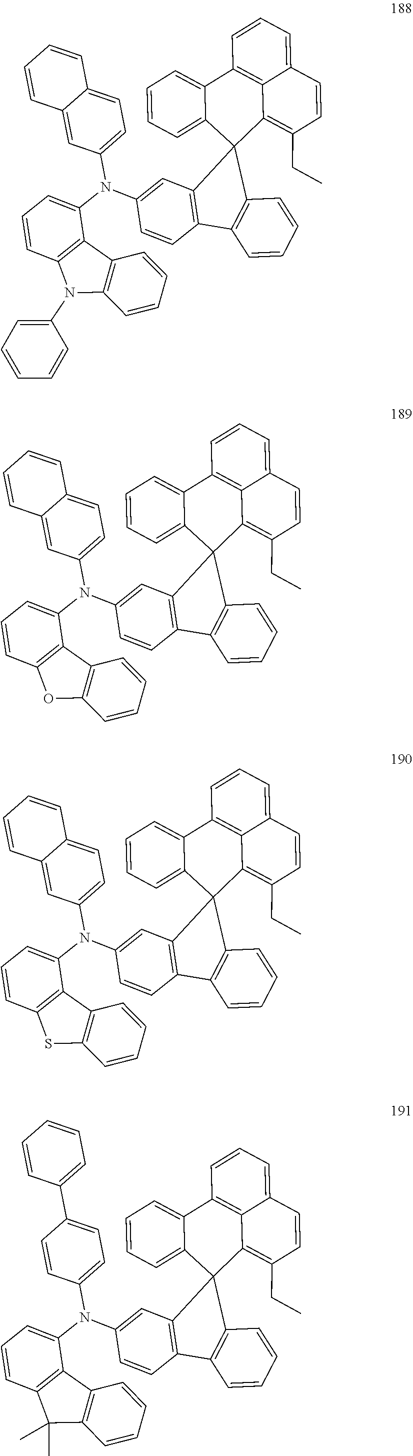 [SMILES: CCc1ccc2cccc3c2c1C1(c2ccccc2-c2ccc(N(c4ccc5ccccc5c4)c4cccc5c4c4ccccc4n5-c4ccccc4)cc21)c1ccccc1-3 ; CCc1ccc2cccc3c2c1C1(c2ccccc2-c2ccc(N(c4ccc5ccccc5c4)c4cccc5oc6ccccc6c45)cc21)c1ccccc1-3 ; CCc1ccc2cccc3c2c1C1(c2ccccc2-c2ccc(N(c4ccc5ccccc5c4)c4cccc5sc6ccccc6c45)cc21)c1ccccc1-3 ; CCc1ccc2cccc3c2c1C1(c2ccccc2-c2ccc(N(c4ccc(-c5ccccc5)cc4)c4cccc5c4-c4ccccc4C5(C)C)cc21)c1ccccc1-3]

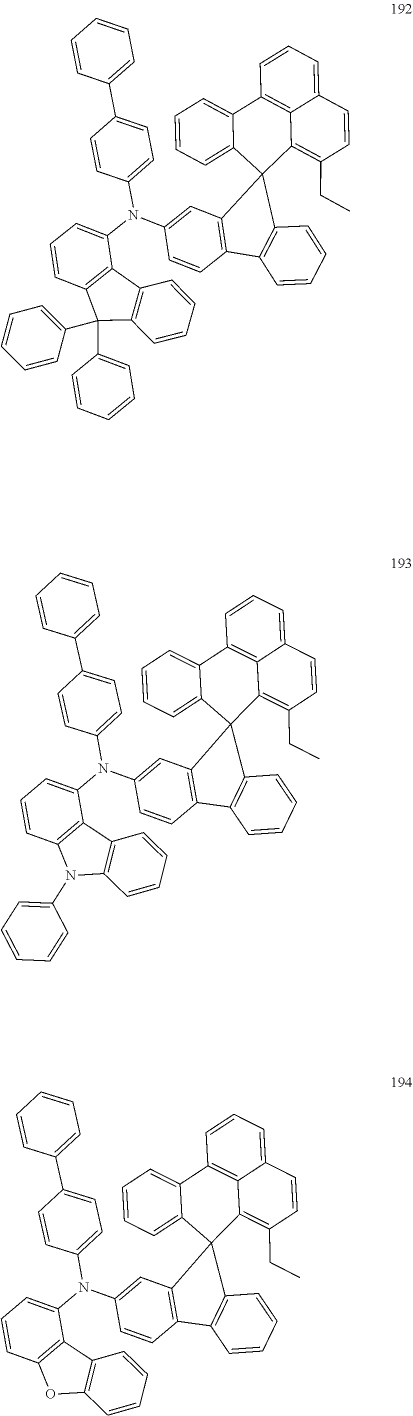 [SMILES: CCc1ccc2cccc3c2c1C1(c2ccccc2-c2ccc(N(c4ccc(-c5ccccc5)cc4)c4cccc5c4-c4ccccc4C5(c4ccccc4)c4ccccc4)cc21)c1ccccc1-3 ; CCc1ccc2cccc3c2c1C1(c2ccccc2-c2ccc(N(c4ccc(-c5ccccc5)cc4)c4cccc5c4c4ccccc4n5-c4ccccc4)cc21)c1ccccc1-3 ; CCc1ccc2cccc3c2c1C1(c2ccccc2-c2ccc(N(c4ccc(-c5ccccc5)cc4)c4cccc5oc6ccccc6c45)cc21)c1ccccc1-3]

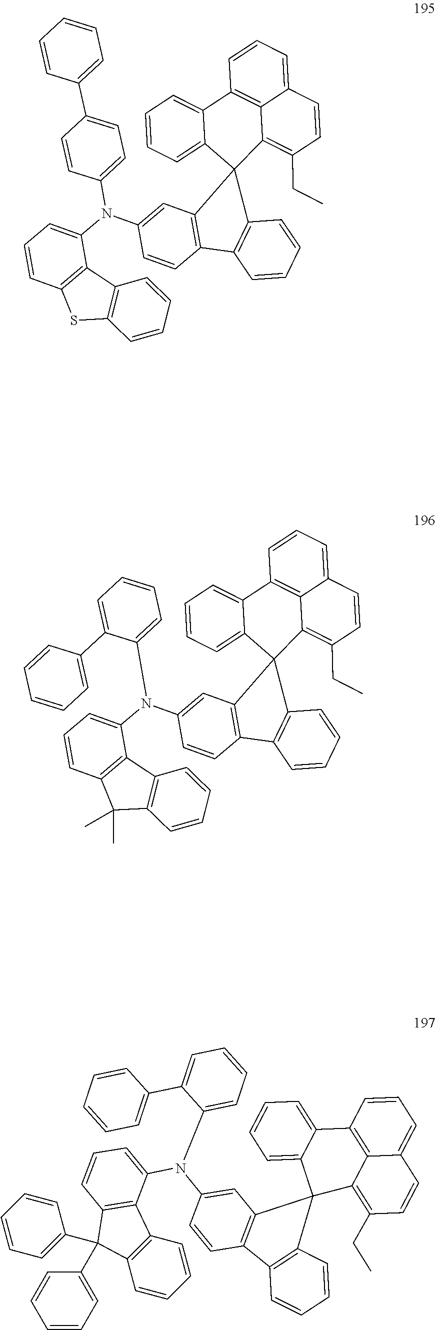 [SMILES: CCc1ccc2cccc3c2c1C1(c2ccccc2-c2ccc(N(c4ccc(-c5ccccc5)cc4)c4cccc5sc6ccccc6c45)cc21)c1ccccc1-3 ; CCc1ccc2cccc3c2c1C1(c2ccccc2-c2ccc(N(c4ccccc4-c4ccccc4)c4cccc5c4-c4ccccc4C5(C)C)cc21)c1ccccc1-3 ; CCc1ccc2cccc3c2c1C1(c2ccccc2-c2ccc(N(c4ccccc4-c4ccccc4)c4cccc5c4-c4ccccc4C5(c4ccccc4)c4ccccc4)cc21)c1ccccc1-3]

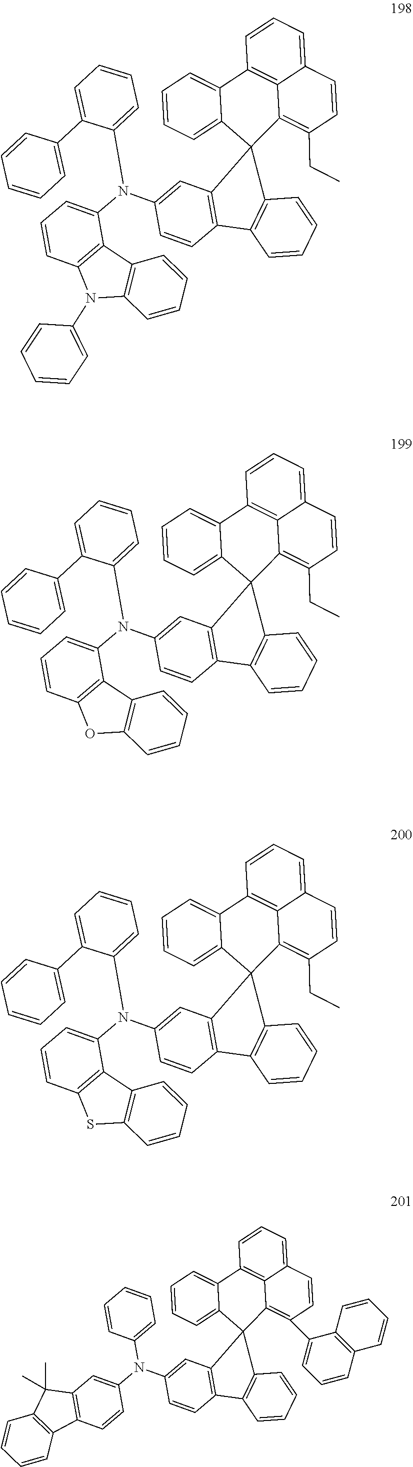 [SMILES: CCc1ccc2cccc3c2c1C1(c2ccccc2-c2ccc(N(c4ccccc4-c4ccccc4)c4cccc5c4c4ccccc4n5-c4ccccc4)cc21)c1ccccc1-3 ; CCc1ccc2cccc3c2c1C1(c2ccccc2-c2ccc(N(c4ccccc4-c4ccccc4)c4cccc5oc6ccccc6c45)cc21)c1ccccc1-3 ; CCc1ccc2cccc3c2c1C1(c2ccccc2-c2ccc(N(c4ccccc4-c4ccccc4)c4cccc5sc6ccccc6c45)cc21)c1ccccc1-3 ; CC1(C)c2ccccc2-c2ccc(N(c3ccccc3)c3ccc4c(c3)C3(c5ccccc5-4)c4ccccc4-c4cccc5ccc(-c6cccc7ccccc67)c3c45)cc21]

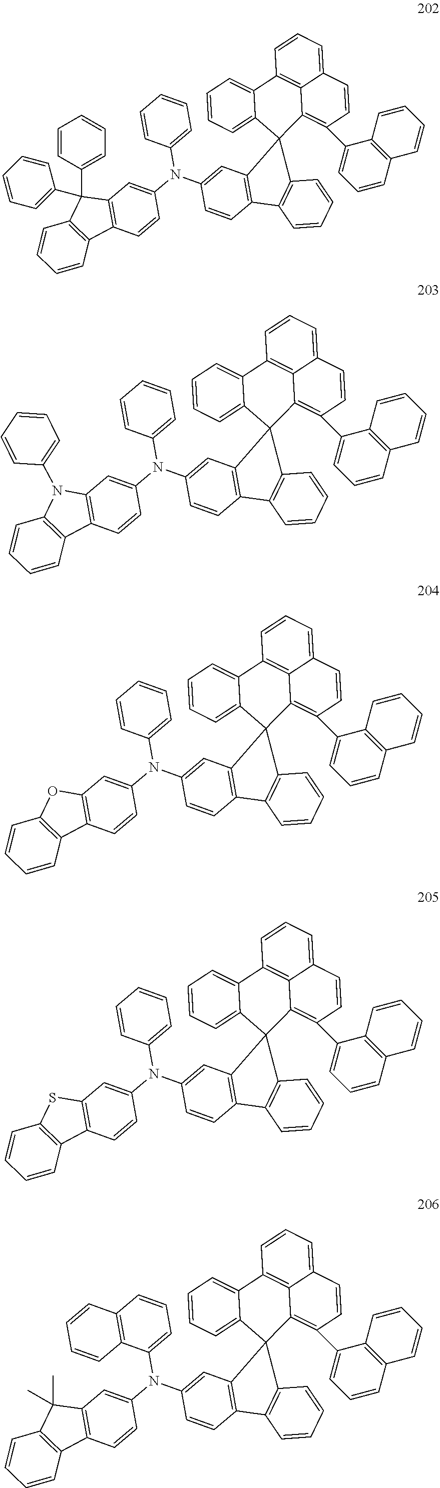

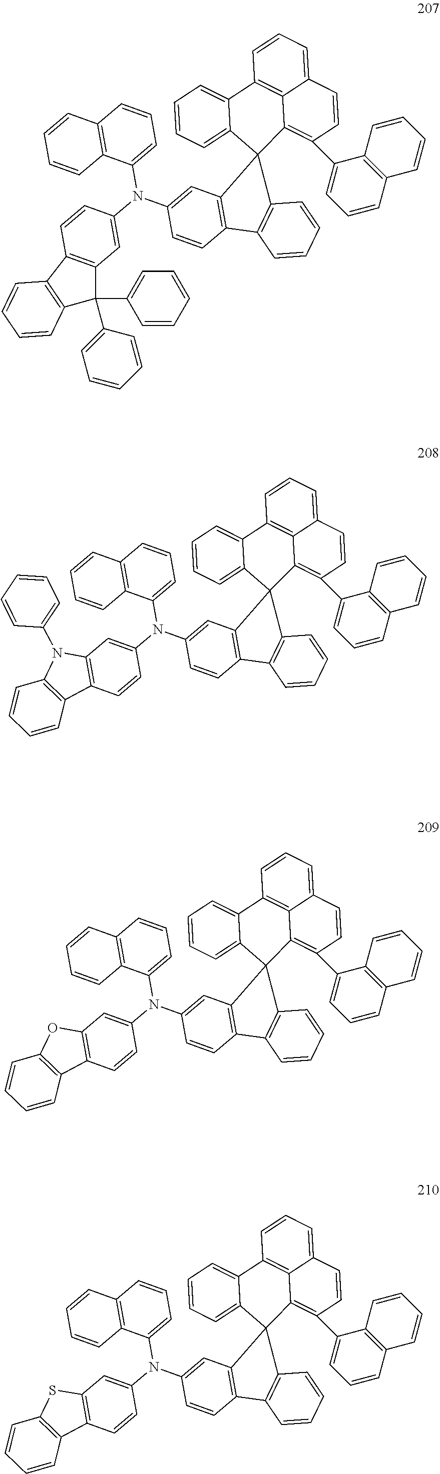 [SMILES: c1ccc(C2(c3ccccc3)c3ccccc3-c3ccc(N(c4ccc5c(c4)C4(c6ccccc6-5)c5ccccc5-c5cccc6ccc(-c7cccc8ccccc78)c4c56)c4cccc5ccccc45)cc32)cc1 ; c1ccc(-n2c3ccccc3c3ccc(N(c4ccc5c(c4)C4(c6ccccc6-5)c5ccccc5-c5cccc6ccc(-c7cccc8ccccc78)c4c56)c4cccc5ccccc45)cc32)cc1 ; c1ccc2c(c1)-c1ccc(N(c3ccc4c(c3)oc3ccccc34)c3cccc4ccccc34)cc1C21c2ccccc2-c2cccc3ccc(-c4cccc5ccccc45)c1c23 ; c1ccc2c(c1)-c1ccc(N(c3ccc4c(c3)sc3ccccc34)c3cccc4ccccc34)cc1C21c2ccccc2-c2cccc3ccc(-c4cccc5ccccc45)c1c23]

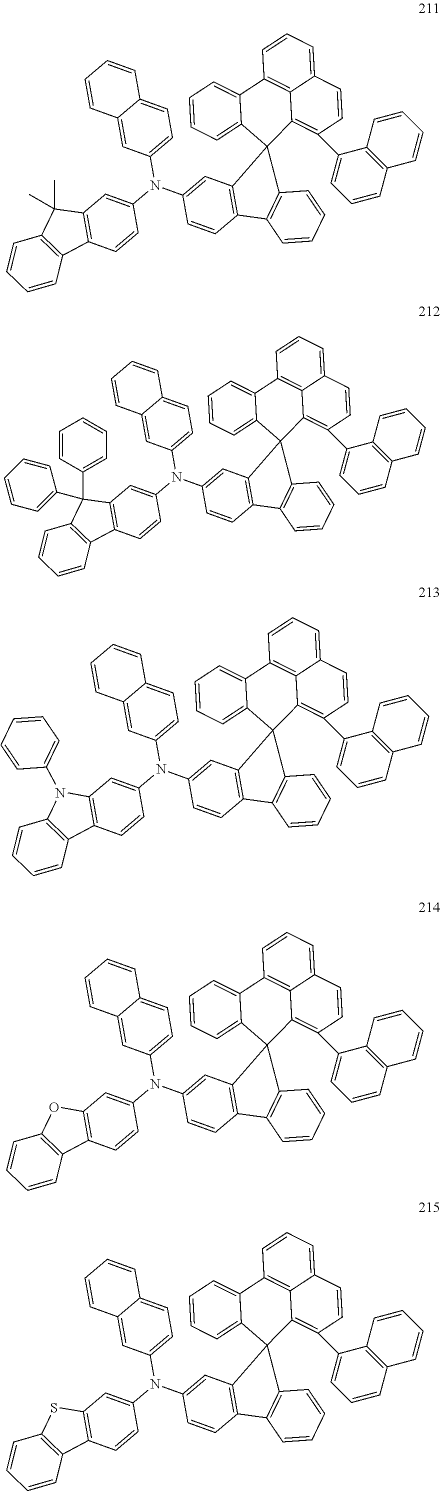

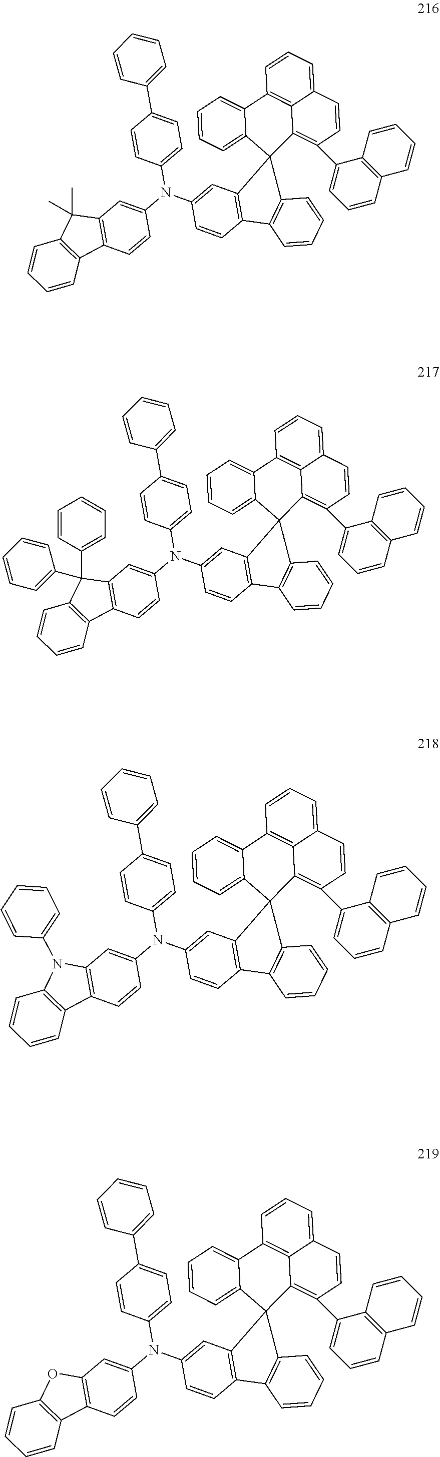 [SMILES: CC1(C)c2ccccc2-c2ccc(N(c3ccc(-c4ccccc4)cc3)c3ccc4c(c3)C3(c5ccccc5-4)c4ccccc4-c4cccc5ccc(-c6cccc7ccccc67)c3c45)cc21 ; c1ccc(-c2ccc(N(c3ccc4c(c3)C(c3ccccc3)(c3ccccc3)c3ccccc3-4)c3ccc4c(c3)C3(c5ccccc5-4)c4ccccc4-c4cccc5ccc(-c6cccc7ccccc67)c3c45)cc2)cc1 ; c1ccc(-c2ccc(N(c3ccc4c(c3)C3(c5ccccc5-4)c4ccccc4-c4cccc5ccc(-c6cccc7ccccc67)c3c45)c3ccc4c5ccccc5n(-c5ccccc5)c4c3)cc2)cc1 ; c1ccc(-c2ccc(N(c3ccc4c(c3)C3(c5ccccc5-4)c4ccccc4-c4cccc5ccc(-c6cccc7ccccc67)c3c45)c3ccc4c(c3)oc3ccccc34)cc2)cc1]

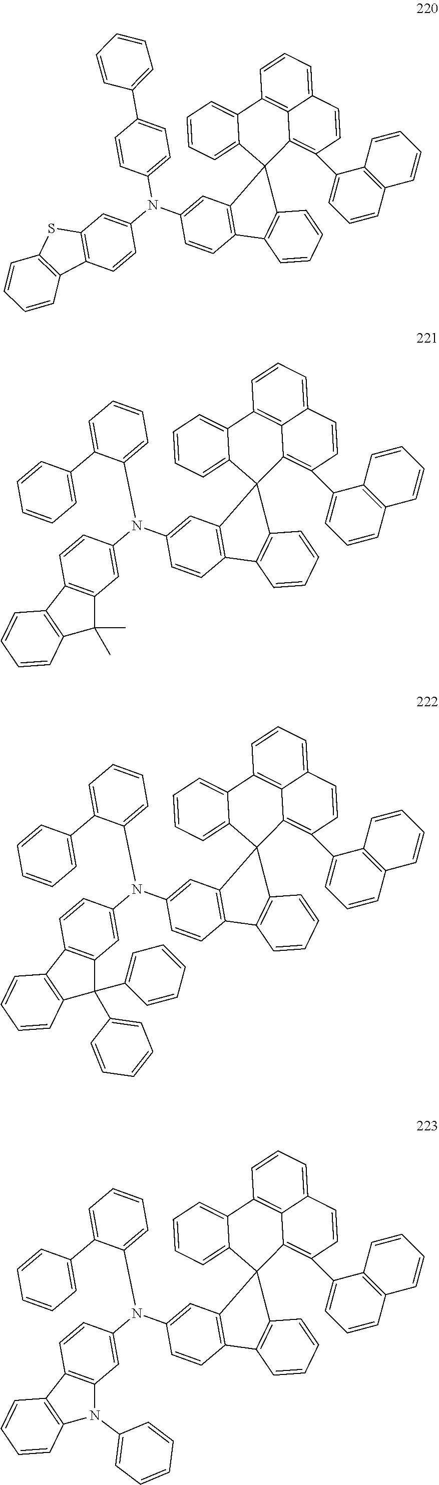 [SMILES: c1ccc(-c2ccc(N(c3ccc4c(c3)C3(c5ccccc5-4)c4ccccc4-c4cccc5ccc(-c6cccc7ccccc67)c3c45)c3ccc4c(c3)sc3ccccc34)cc2)cc1 ; CC1(C)c2ccccc2-c2ccc(N(c3ccc4c(c3)C3(c5ccccc5-4)c4ccccc4-c4cccc5ccc(-c6cccc7ccccc67)c3c45)c3ccccc3-c3ccccc3)cc21 ; c1ccc(-c2ccccc2N(c2ccc3c(c2)C(c2ccccc2)(c2ccccc2)c2ccccc2-3)c2ccc3c(c2)C2(c4ccccc4-3)c3ccccc3-c3cccc4ccc(-c5cccc6ccccc56)c2c34)cc1 ; c1ccc(-c2ccccc2N(c2ccc3c(c2)C2(c4ccccc4-3)c3ccccc3-c3cccc4ccc(-c5cccc6ccccc56)c2c34)c2ccc3c4ccccc4n(-c4ccccc4)c3c2)cc1]

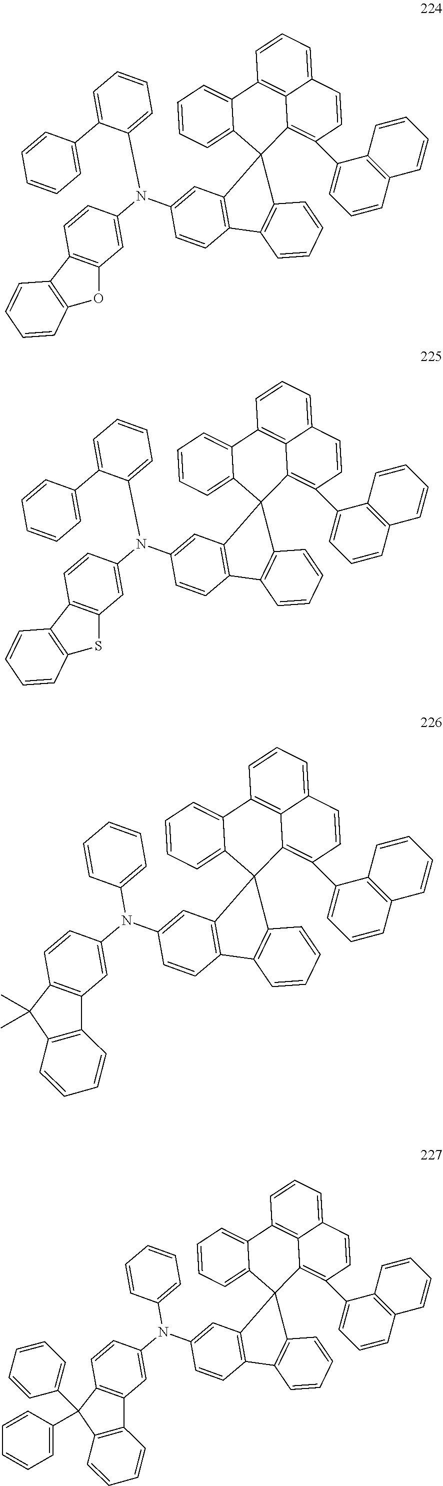 [SMILES: c1ccc(-c2ccccc2N(c2ccc3c(c2)C2(c4ccccc4-3)c3ccccc3-c3cccc4ccc(-c5cccc6ccccc56)c2c34)c2ccc3c(c2)oc2ccccc23)cc1 ; c1ccc(-c2ccccc2N(c2ccc3c(c2)C2(c4ccccc4-3)c3ccccc3-c3cccc4ccc(-c5cccc6ccccc56)c2c34)c2ccc3c(c2)sc2ccccc23)cc1 ; CC1(C)c2ccccc2-c2cc(N(c3ccccc3)c3ccc4c(c3)C3(c5ccccc5-4)c4ccccc4-c4cccc5ccc(-c6cccc7ccccc67)c3c45)ccc21 ; c1ccc(N(c2ccc3c(c2)-c2ccccc2C3(c2ccccc2)c2ccccc2)c2ccc3c(c2)C2(c4ccccc4-3)c3ccccc3-c3cccc4ccc(-c5cccc6ccccc56)c2c34)cc1]

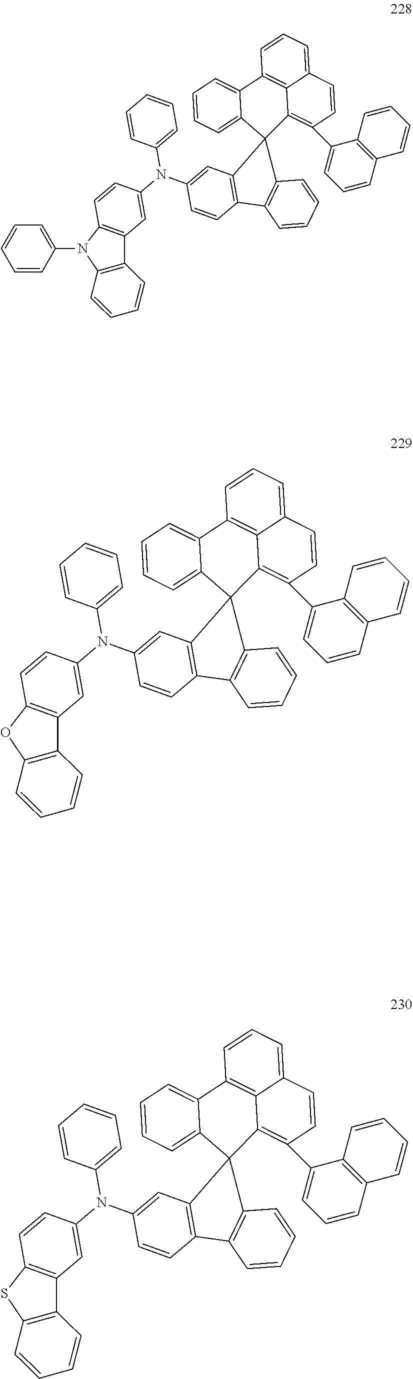 [SMILES: c1ccc(N(c2ccc3c(c2)C2(c4ccccc4-3)c3ccccc3-c3cccc4ccc(-c5cccc6ccccc56)c2c34)c2ccc3c(c2)c2ccccc2n3-c2ccccc2)cc1 ; c1ccc(N(c2ccc3c(c2)C2(c4ccccc4-3)c3ccccc3-c3cccc4ccc(-c5cccc6ccccc56)c2c34)c2ccc3oc4ccccc4c3c2)cc1 ; c1ccc(N(c2ccc3c(c2)C2(c4ccccc4-3)c3ccccc3-c3cccc4ccc(-c5cccc6ccccc56)c2c34)c2ccc3sc4ccccc4c3c2)cc1]

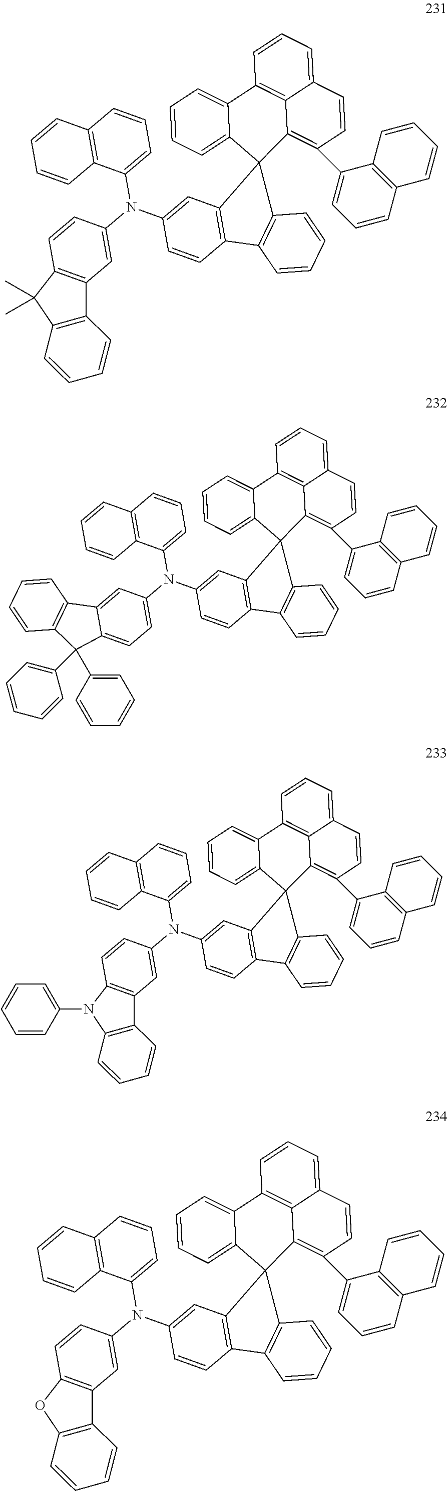 [SMILES: CC1(C)c2ccccc2-c2cc(N(c3ccc4c(c3)C3(c5ccccc5-4)c4ccccc4-c4cccc5ccc(-c6cccc7ccccc67)c3c45)c3cccc4ccccc34)ccc21 ; c1ccc(C2(c3ccccc3)c3ccccc3-c3cc(N(c4ccc5c(c4)C4(c6ccccc6-5)c5ccccc5-c5cccc6ccc(-c7cccc8ccccc78)c4c56)c4cccc5ccccc45)ccc32)cc1 ; c1ccc(-n2c3ccccc3c3cc(N(c4ccc5c(c4)C4(c6ccccc6-5)c5ccccc5-c5cccc6ccc(-c7cccc8ccccc78)c4c56)c4cccc5ccccc45)ccc32)cc1 ; c1ccc2c(c1)-c1ccc(N(c3ccc4oc5ccccc5c4c3)c3cccc4ccccc34)cc1C21c2ccccc2-c2cccc3ccc(-c4cccc5ccccc45)c1c23]

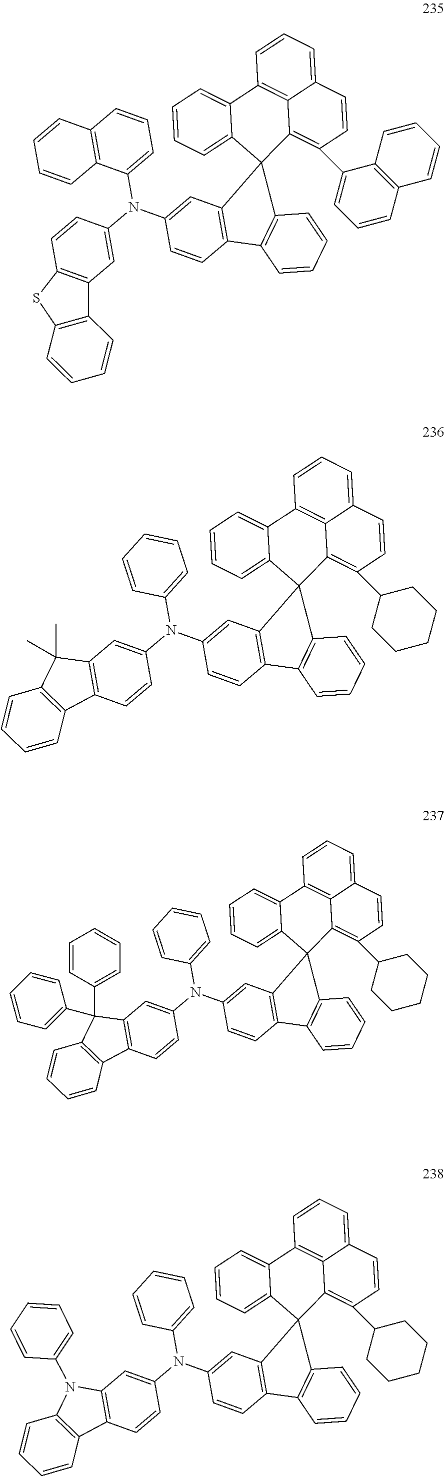 [SMILES: c1ccc2c(c1)-c1ccc(N(c3ccc4sc5ccccc5c4c3)c3cccc4ccccc34)cc1C21c2ccccc2-c2cccc3ccc(-c4cccc5ccccc45)c1c23 ; CC1(C)c2ccccc2-c2ccc(N(c3ccccc3)c3ccc4c(c3)C3(c5ccccc5-4)c4ccccc4-c4cccc5ccc(C6CCCCC6)c3c45)cc21 ; c1ccc(N(c2ccc3c(c2)C(c2ccccc2)(c2ccccc2)c2ccccc2-3)c2ccc3c(c2)C2(c4ccccc4-3)c3ccccc3-c3cccc4ccc(C5CCCCC5)c2c34)cc1 ; c1ccc(N(c2ccc3c(c2)C2(c4ccccc4-3)c3ccccc3-c3cccc4ccc(C5CCCCC5)c2c34)c2ccc3c4ccccc4n(-c4ccccc4)c3c2)cc1]

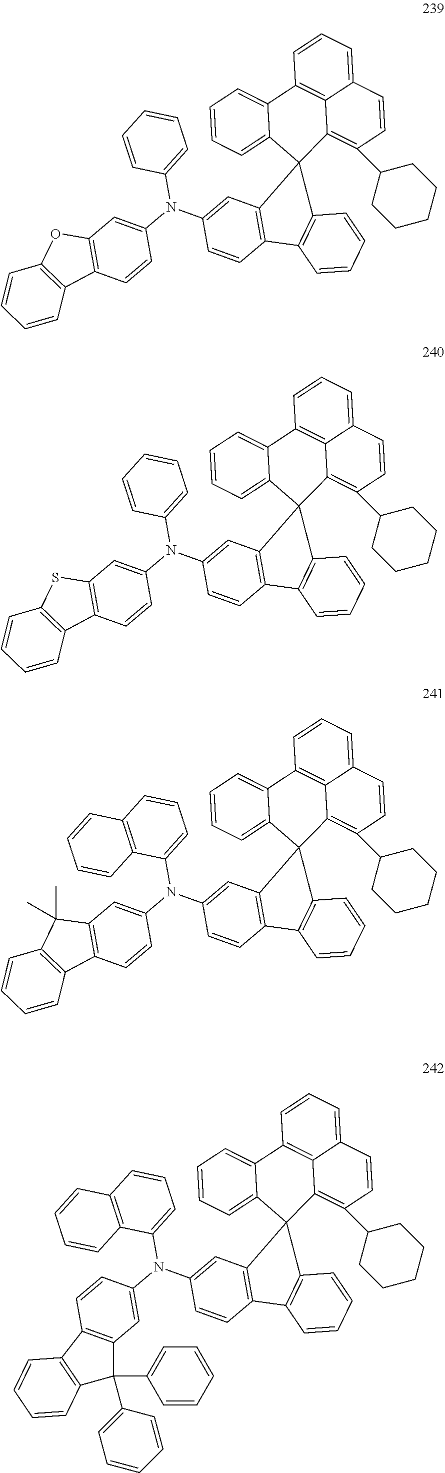 [SMILES: c1ccc(N(c2ccc3c(c2)C2(c4ccccc4-3)c3ccccc3-c3cccc4ccc(C5CCCCC5)c2c34)c2ccc3c(c2)oc2ccccc23)cc1 ; c1ccc(N(c2ccc3c(c2)C2(c4ccccc4-3)c3ccccc3-c3cccc4ccc(C5CCCCC5)c2c34)c2ccc3c(c2)sc2ccccc23)cc1 ; CC1(C)c2ccccc2-c2ccc(N(c3ccc4c(c3)C3(c5ccccc5-4)c4ccccc4-c4cccc5ccc(C6CCCCC6)c3c45)c3cccc4ccccc34)cc21 ; c1ccc(C2(c3ccccc3)c3ccccc3-c3ccc(N(c4ccc5c(c4)C4(c6ccccc6-5)c5ccccc5-c5cccc6ccc(C7CCCCC7)c4c56)c4cccc5ccccc45)cc32)cc1]

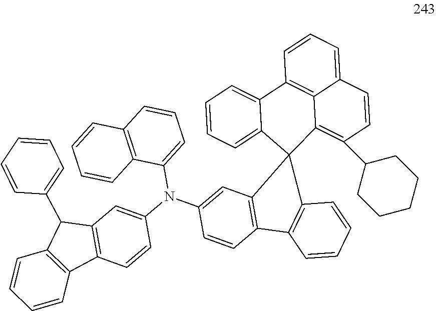 [SMILES: c1ccc(C2c3ccccc3-c3ccc(N(c4ccc5c(c4)C4(c6ccccc6-5)c5ccccc5-c5cccc6ccc(C7CCCCC7)c4c56)c4cccc5ccccc45)cc32)cc1]

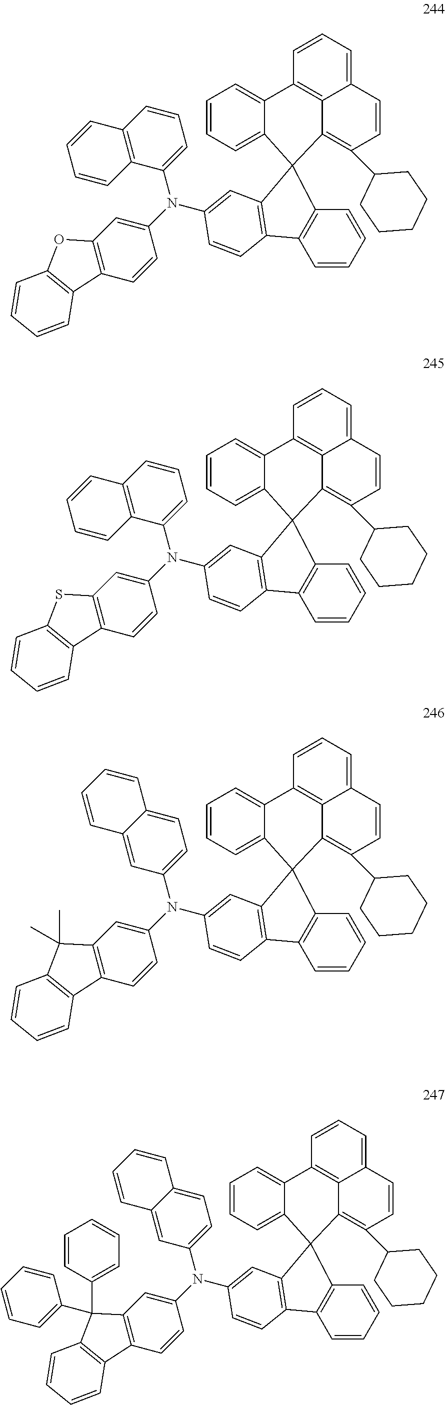 [SMILES: c1ccc2c(c1)-c1ccc(N(c3ccc4c(c3)oc3ccccc34)c3cccc4ccccc34)cc1C21c2ccccc2-c2cccc3ccc(C4CCCCC4)c1c23 ; c1ccc2c(c1)-c1ccc(N(c3ccc4c(c3)sc3ccccc34)c3cccc4ccccc34)cc1C21c2ccccc2-c2cccc3ccc(C4CCCCC4)c1c23 ; CC1(C)c2ccccc2-c2ccc(N(c3ccc4c(c3)C3(c5ccccc5-4)c4ccccc4-c4cccc5ccc(C6CCCCC6)c3c45)c3ccc4ccccc4c3)cc21 ; c1ccc(C2(c3ccccc3)c3ccccc3-c3ccc(N(c4ccc5c(c4)C4(c6ccccc6-5)c5ccccc5-c5cccc6ccc(C7CCCCC7)c4c56)c4ccc5ccccc5c4)cc32)cc1]

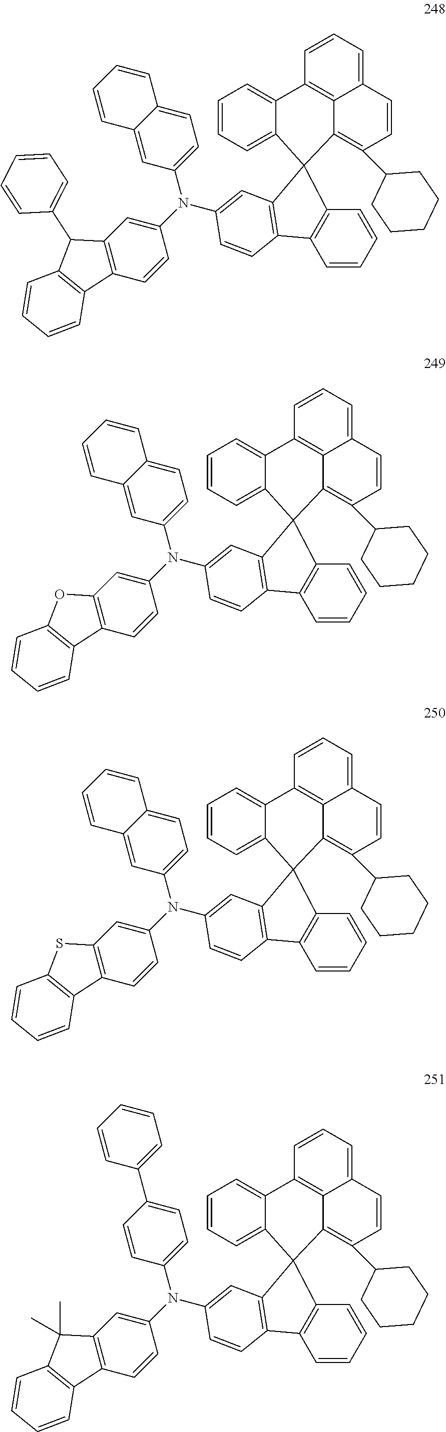 [SMILES: c1ccc(C2c3ccccc3-c3ccc(N(c4ccc5c(c4)C4(c6ccccc6-5)c5ccccc5-c5cccc6ccc(C7CCCCC7)c4c56)c4ccc5ccccc5c4)cc32)cc1 ; c1ccc2c(c1)-c1ccc(N(c3ccc4ccccc4c3)c3ccc4c(c3)oc3ccccc34)cc1C21c2ccccc2-c2cccc3ccc(C4CCCCC4)c1c23 ; c1ccc2c(c1)-c1ccc(N(c3ccc4ccccc4c3)c3ccc4c(c3)sc3ccccc34)cc1C21c2ccccc2-c2cccc3ccc(C4CCCCC4)c1c23 ; CC1(C)c2ccccc2-c2ccc(N(c3ccc(-c4ccccc4)cc3)c3ccc4c(c3)C3(c5ccccc5-4)c4ccccc4-c4cccc5ccc(C6CCCCC6)c3c45)cc21]

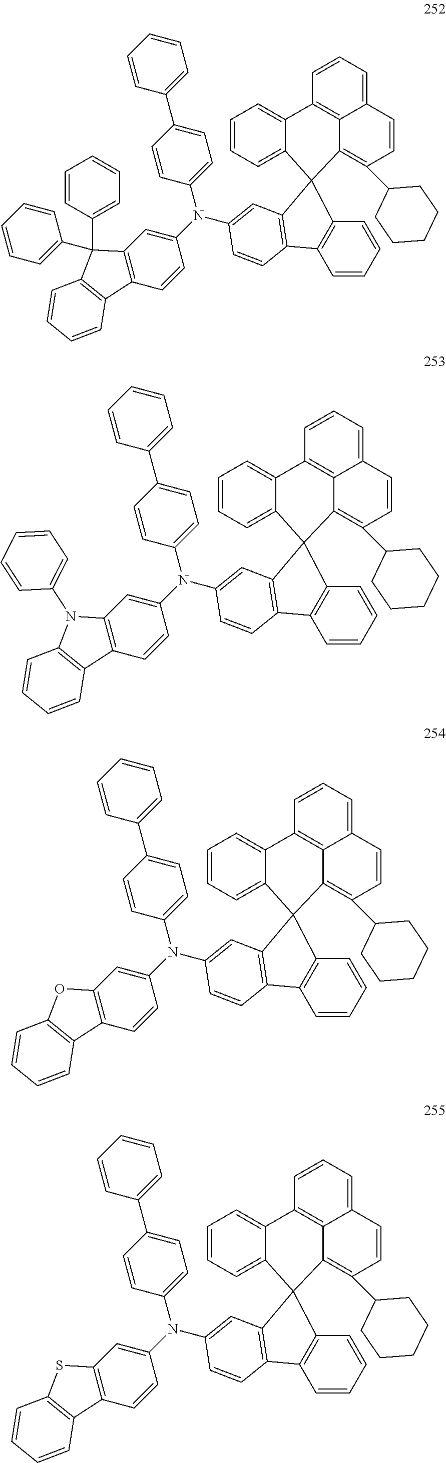 [SMILES: c1ccc(-c2ccc(N(c3ccc4c(c3)C(c3ccccc3)(c3ccccc3)c3ccccc3-4)c3ccc4c(c3)C3(c5ccccc5-4)c4ccccc4-c4cccc5ccc(C6CCCCC6)c3c45)cc2)cc1 ; c1ccc(-c2ccc(N(c3ccc4c(c3)C3(c5ccccc5-4)c4ccccc4-c4cccc5ccc(C6CCCCC6)c3c45)c3ccc4c5ccccc5n(-c5ccccc5)c4c3)cc2)cc1 ; c1ccc(-c2ccc(N(c3ccc4c(c3)C3(c5ccccc5-4)c4ccccc4-c4cccc5ccc(C6CCCCC6)c3c45)c3ccc4c(c3)oc3ccccc34)cc2)cc1 ; c1ccc(-c2ccc(N(c3ccc4c(c3)C3(c5ccccc5-4)c4ccccc4-c4cccc5ccc(C6CCCCC6)c3c45)c3ccc4c(c3)sc3ccccc34)cc2)cc1]

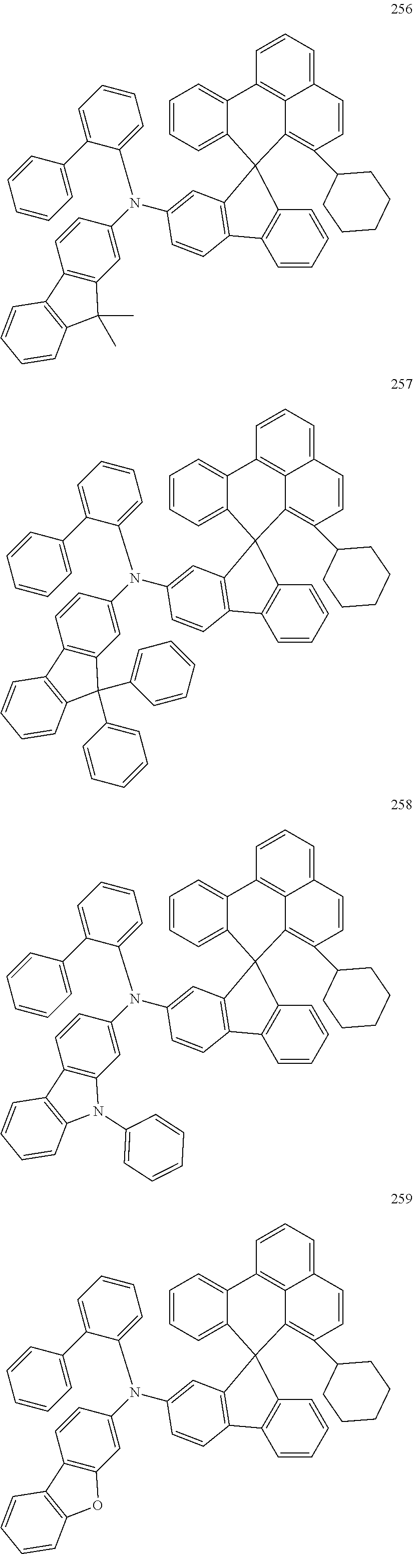 [SMILES: CC1(C)c2ccccc2-c2ccc(N(c3ccc4c(c3)C3(c5ccccc5-4)c4ccccc4-c4cccc5ccc(C6CCCCC6)c3c45)c3ccccc3-c3ccccc3)cc21 ; c1ccc(-c2ccccc2N(c2ccc3c(c2)C(c2ccccc2)(c2ccccc2)c2ccccc2-3)c2ccc3c(c2)C2(c4ccccc4-3)c3ccccc3-c3cccc4ccc(C5CCCCC5)c2c34)cc1 ; c1ccc(-c2ccccc2N(c2ccc3c(c2)C2(c4ccccc4-3)c3ccccc3-c3cccc4ccc(C5CCCCC5)c2c34)c2ccc3c4ccccc4n(-c4ccccc4)c3c2)cc1 ; c1ccc(-c2ccccc2N(c2ccc3c(c2)C2(c4ccccc4-3)c3ccccc3-c3cccc4ccc(C5CCCCC5)c2c34)c2ccc3c(c2)oc2ccccc23)cc1]

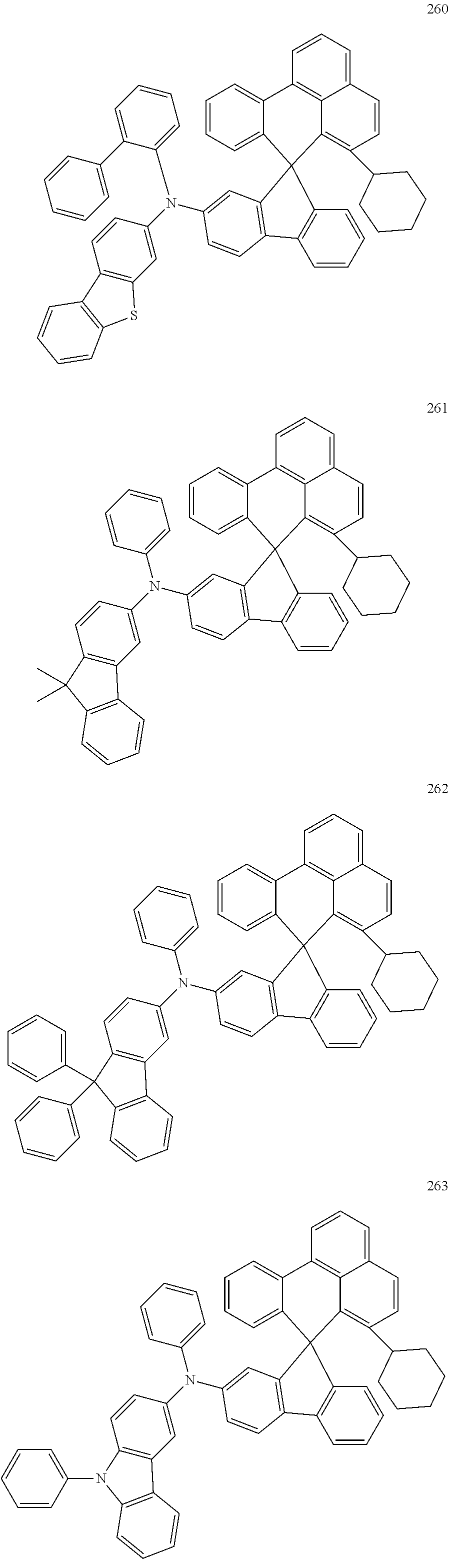 [SMILES: c1ccc(-c2ccccc2N(c2ccc3c(c2)C2(c4ccccc4-3)c3ccccc3-c3cccc4ccc(C5CCCCC5)c2c34)c2ccc3c(c2)sc2ccccc23)cc1 ; CC1(C)c2ccccc2-c2cc(N(c3ccccc3)c3ccc4c(c3)C3(c5ccccc5-4)c4ccccc4-c4cccc5ccc(C6CCCCC6)c3c45)ccc21 ; c1ccc(N(c2ccc3c(c2)-c2ccccc2C3(c2ccccc2)c2ccccc2)c2ccc3c(c2)C2(c4ccccc4-3)c3ccccc3-c3cccc4ccc(C5CCCCC5)c2c34)cc1 ; c1ccc(N(c2ccc3c(c2)C2(c4ccccc4-3)c3ccccc3-c3cccc4ccc(C5CCCCC5)c2c34)c2ccc3c(c2)c2ccccc2n3-c2ccccc2)cc1]

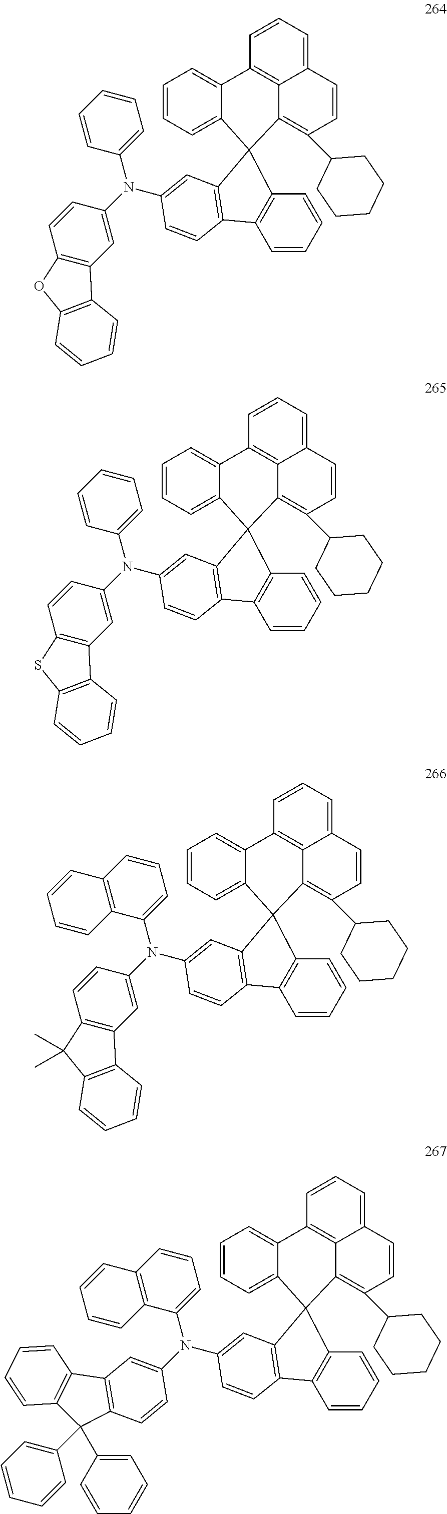 [SMILES: c1ccc(N(c2ccc3c(c2)C2(c4ccccc4-3)c3ccccc3-c3cccc4ccc(C5CCCCC5)c2c34)c2ccc3oc4ccccc4c3c2)cc1 ; c1ccc(N(c2ccc3c(c2)C2(c4ccccc4-3)c3ccccc3-c3cccc4ccc(C5CCCCC5)c2c34)c2ccc3sc4ccccc4c3c2)cc1 ; CC1(C)c2ccccc2-c2cc(N(c3ccc4c(c3)C3(c5ccccc5-4)c4ccccc4-c4cccc5ccc(C6CCCCC6)c3c45)c3cccc4ccccc34)ccc21 ; c1ccc(C2(c3ccccc3)c3ccccc3-c3cc(N(c4ccc5c(c4)C4(c6ccccc6-5)c5ccccc5-c5cccc6ccc(C7CCCCC7)c4c56)c4cccc5ccccc45)ccc32)cc1]

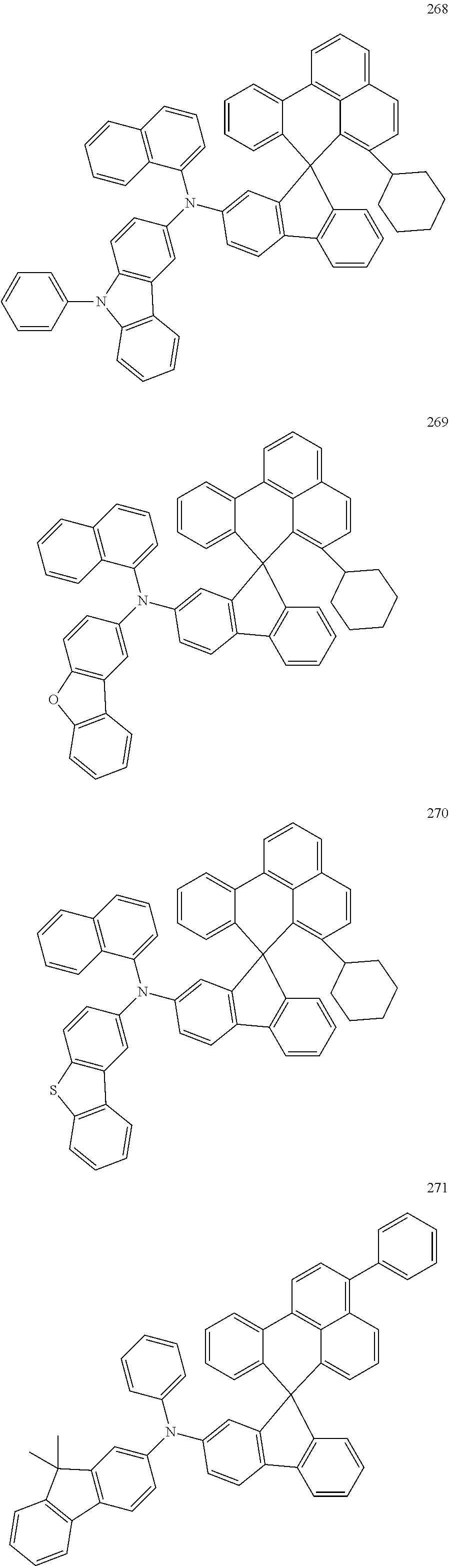 [SMILES: c1ccc(-n2c3ccccc3c3cc(N(c4ccc5c(c4)C4(c6ccccc6-5)c5ccccc5-c5cccc6ccc(C7CCCCC7)c4c56)c4cccc5ccccc45)ccc32)cc1 ; c1ccc2c(c1)-c1ccc(N(c3ccc4oc5ccccc5c4c3)c3cccc4ccccc34)cc1C21c2ccccc2-c2cccc3ccc(C4CCCCC4)c1c23 ; c1ccc2c(c1)-c1ccc(N(c3ccc4sc5ccccc5c4c3)c3cccc4ccccc34)cc1C21c2ccccc2-c2cccc3ccc(C4CCCCC4)c1c23 ; CC1(C)c2ccccc2-c2ccc(N(c3ccccc3)c3ccc4c(c3)C3(c5ccccc5-4)c4ccccc4-c4ccc(-c5ccccc5)c5cccc3c45)cc21]

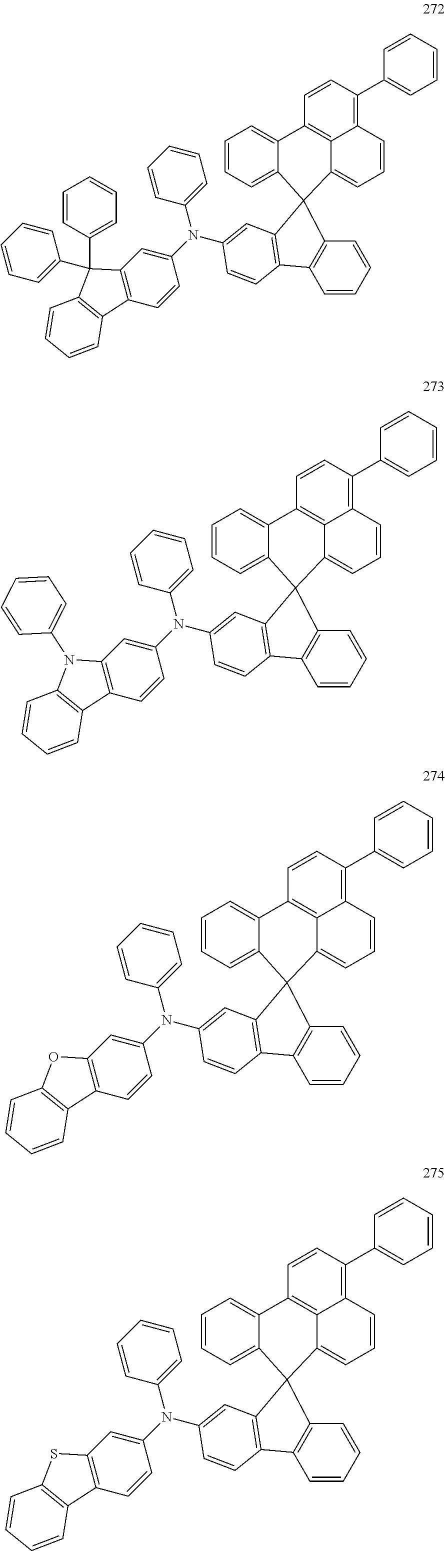 [SMILES: c1ccc(-c2ccc3c4c(cccc24)C2(c4ccccc4-c4ccc(N(c5ccccc5)c5ccc6c(c5)C(c5ccccc5)(c5ccccc5)c5ccccc5-6)cc42)c2ccccc2-3)cc1 ; c1ccc(-c2ccc3c4c(cccc24)C2(c4ccccc4-c4ccc(N(c5ccccc5)c5ccc6c7ccccc7n(-c7ccccc7)c6c5)cc42)c2ccccc2-3)cc1 ; c1ccc(-c2ccc3c4c(cccc24)C2(c4ccccc4-c4ccc(N(c5ccccc5)c5ccc6c(c5)oc5ccccc56)cc42)c2ccccc2-3)cc1 ; c1ccc(-c2ccc3c4c(cccc24)C2(c4ccccc4-c4ccc(N(c5ccccc5)c5ccc6c(c5)sc5ccccc56)cc42)c2ccccc2-3)cc1]

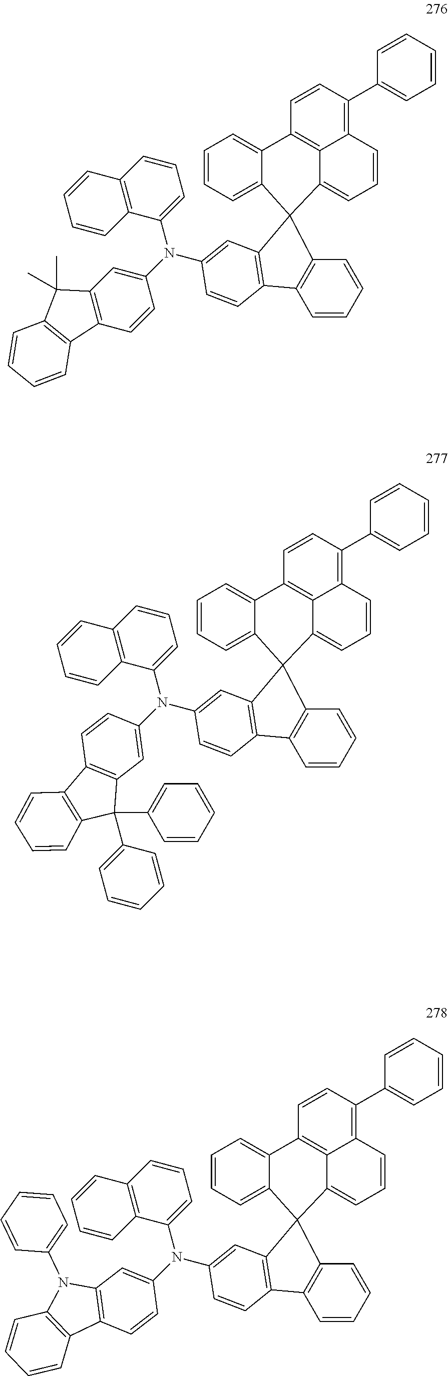 [SMILES: CC1(C)c2ccccc2-c2ccc(N(c3ccc4c(c3)C3(c5ccccc5-4)c4ccccc4-c4ccc(-c5ccccc5)c5cccc3c45)c3cccc4ccccc34)cc21 ; c1ccc(-c2ccc3c4c(cccc24)C2(c4ccccc4-c4ccc(N(c5ccc6c(c5)C(c5ccccc5)(c5ccccc5)c5ccccc5-6)c5cccc6ccccc56)cc42)c2ccccc2-3)cc1 ; c1ccc(-c2ccc3c4c(cccc24)C2(c4ccccc4-c4ccc(N(c5ccc6c7ccccc7n(-c7ccccc7)c6c5)c5cccc6ccccc56)cc42)c2ccccc2-3)cc1]

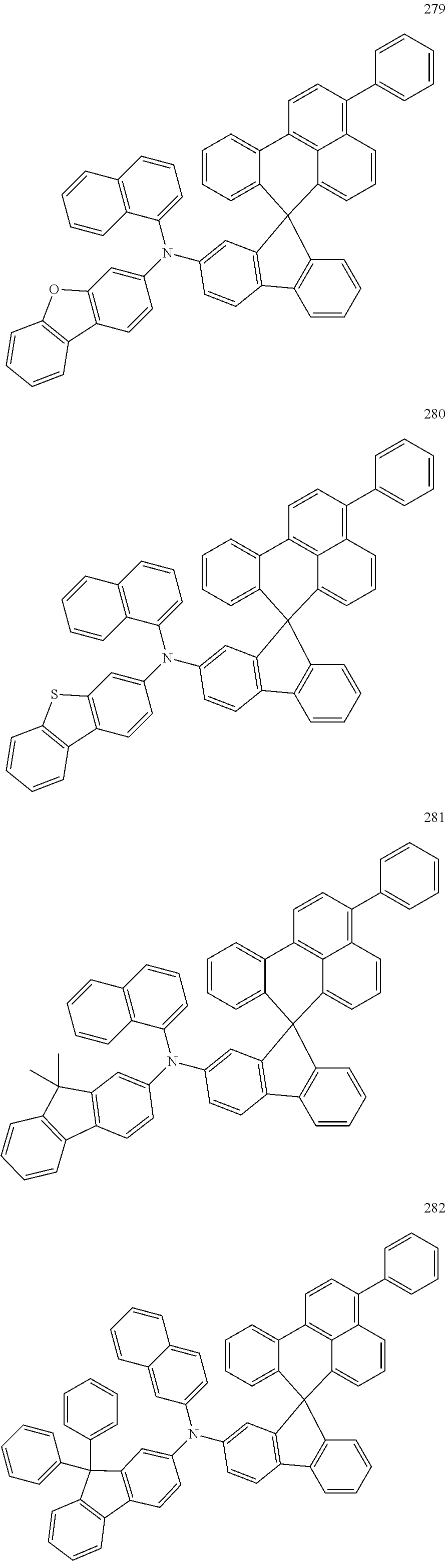 [SMILES: c1ccc(-c2ccc3c4c(cccc24)C2(c4ccccc4-c4ccc(N(c5ccc6c(c5)oc5ccccc56)c5cccc6ccccc56)cc42)c2ccccc2-3)cc1 ; c1ccc(-c2ccc3c4c(cccc24)C2(c4ccccc4-c4ccc(N(c5ccc6c(c5)sc5ccccc56)c5cccc6ccccc56)cc42)c2ccccc2-3)cc1 ; CC1(C)c2ccccc2-c2ccc(N(c3ccc4c(c3)C3(c5ccccc5-4)c4ccccc4-c4ccc(-c5ccccc5)c5cccc3c45)c3cccc4ccccc34)cc21 ; c1ccc(-c2ccc3c4c(cccc24)C2(c4ccccc4-c4ccc(N(c5ccc6c(c5)C(c5ccccc5)(c5ccccc5)c5ccccc5-6)c5ccc6ccccc6c5)cc42)c2ccccc2-3)cc1]

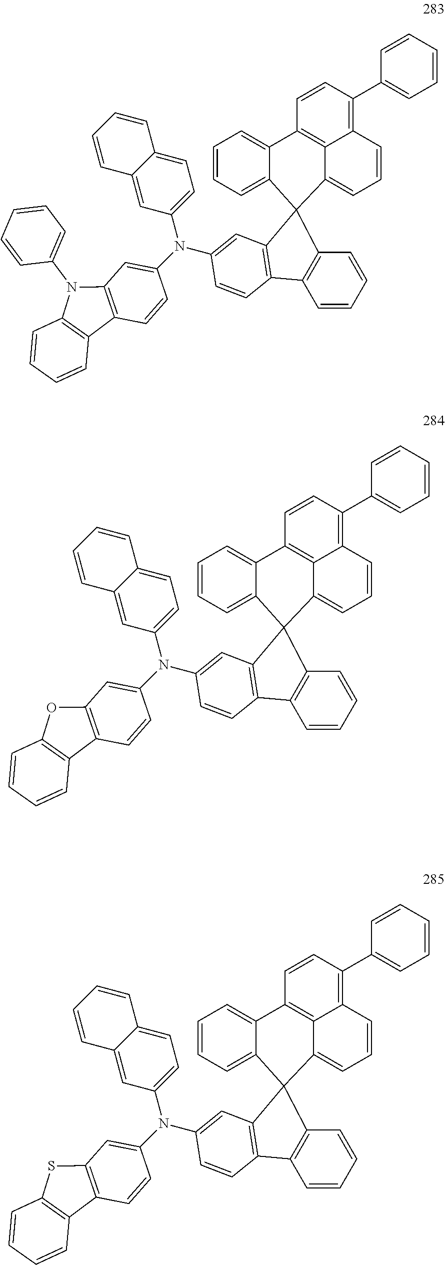 [SMILES: c1ccc(-c2ccc3c4c(cccc24)C2(c4ccccc4-c4ccc(N(c5ccc6ccccc6c5)c5ccc6c7ccccc7n(-c7ccccc7)c6c5)cc42)c2ccccc2-3)cc1 ; c1ccc(-c2ccc3c4c(cccc24)C2(c4ccccc4-c4ccc(N(c5ccc6ccccc6c5)c5ccc6c(c5)oc5ccccc56)cc42)c2ccccc2-3)cc1 ; c1ccc(-c2ccc3c4c(cccc24)C2(c4ccccc4-c4ccc(N(c5ccc6ccccc6c5)c5ccc6c(c5)sc5ccccc56)cc42)c2ccccc2-3)cc1]

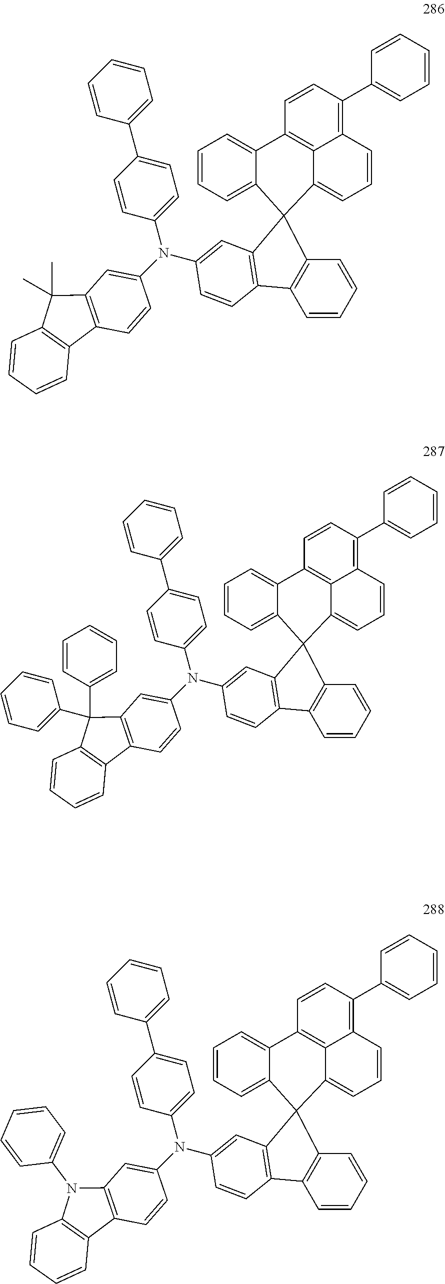 [SMILES: CC1(C)c2ccccc2-c2ccc(N(c3ccc(-c4ccccc4)cc3)c3ccc4c(c3)C3(c5ccccc5-4)c4ccccc4-c4ccc(-c5ccccc5)c5cccc3c45)cc21 ; c1ccc(-c2ccc(N(c3ccc4c(c3)C(c3ccccc3)(c3ccccc3)c3ccccc3-4)c3ccc4c(c3)C3(c5ccccc5-4)c4ccccc4-c4ccc(-c5ccccc5)c5cccc3c45)cc2)cc1 ; c1ccc(-c2ccc(N(c3ccc4c(c3)C3(c5ccccc5-4)c4ccccc4-c4ccc(-c5ccccc5)c5cccc3c45)c3ccc4c5ccccc5n(-c5ccccc5)c4c3)cc2)cc1]

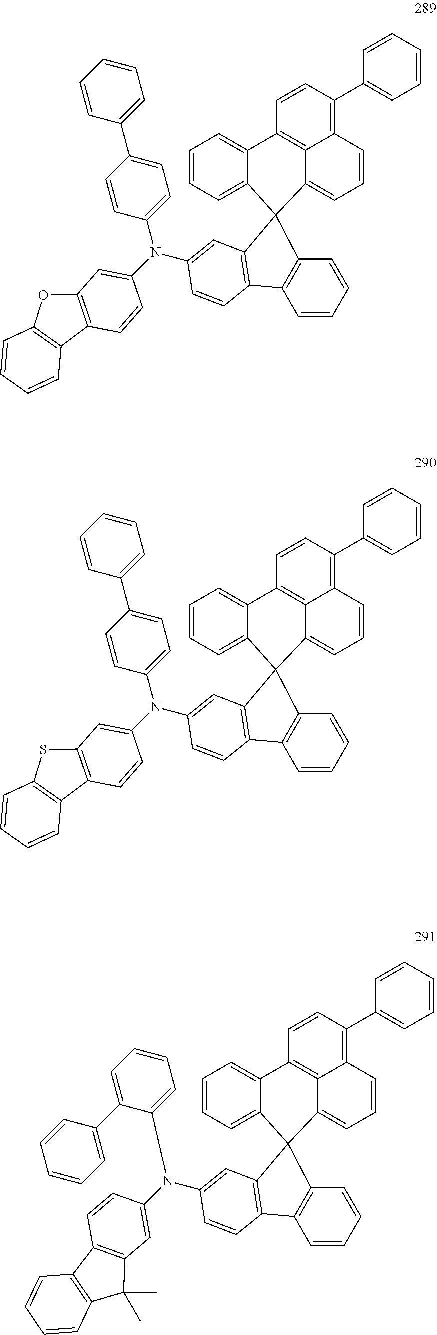 [SMILES: c1ccc(-c2ccc(N(c3ccc4c(c3)C3(c5ccccc5-4)c4ccccc4-c4ccc(-c5ccccc5)c5cccc3c45)c3ccc4c(c3)oc3ccccc34)cc2)cc1 ; c1ccc(-c2ccc(N(c3ccc4c(c3)C3(c5ccccc5-4)c4ccccc4-c4ccc(-c5ccccc5)c5cccc3c45)c3ccc4c(c3)sc3ccccc34)cc2)cc1 ; CC1(C)c2ccccc2-c2ccc(N(c3ccc4c(c3)C3(c5ccccc5-4)c4ccccc4-c4ccc(-c5ccccc5)c5cccc3c45)c3ccccc3-c3ccccc3)cc21]

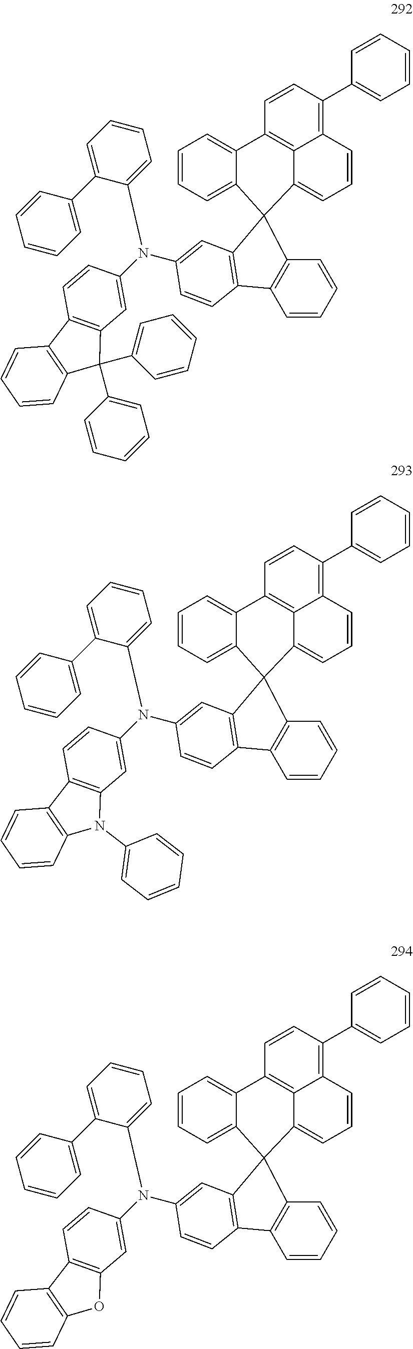 [SMILES: c1ccc(-c2ccccc2N(c2ccc3c(c2)C(c2ccccc2)(c2ccccc2)c2ccccc2-3)c2ccc3c(c2)C2(c4ccccc4-3)c3ccccc3-c3ccc(-c4ccccc4)c4cccc2c34)cc1 ; c1ccc(-c2ccccc2N(c2ccc3c(c2)C2(c4ccccc4-3)c3ccccc3-c3ccc(-c4ccccc4)c4cccc2c34)c2ccc3c4ccccc4n(-c4ccccc4)c3c2)cc1 ; c1ccc(-c2ccccc2N(c2ccc3c(c2)C2(c4ccccc4-3)c3ccccc3-c3ccc(-c4ccccc4)c4cccc2c34)c2ccc3c(c2)oc2ccccc23)cc1]

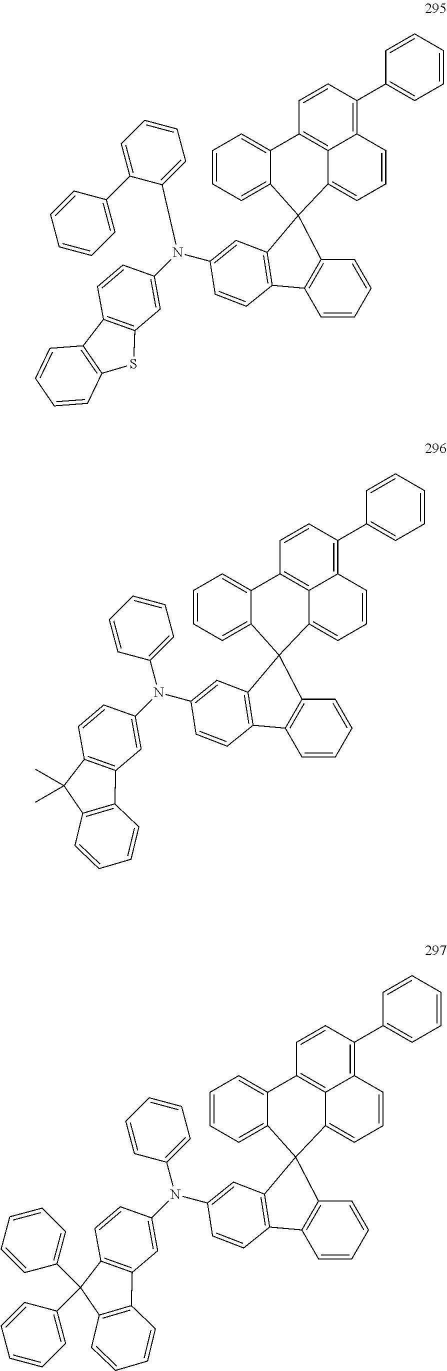 [SMILES: c1ccc(-c2ccccc2N(c2ccc3c(c2)C2(c4ccccc4-3)c3ccccc3-c3ccc(-c4ccccc4)c4cccc2c34)c2ccc3c(c2)sc2ccccc23)cc1 ; CC1(C)c2ccccc2-c2cc(N(c3ccccc3)c3ccc4c(c3)C3(c5ccccc5-4)c4ccccc4-c4ccc(-c5ccccc5)c5cccc3c45)ccc21 ; c1ccc(-c2ccc3c4c(cccc24)C2(c4ccccc4-c4ccc(N(c5ccccc5)c5ccc6c(c5)-c5ccccc5C6(c5ccccc5)c5ccccc5)cc42)c2ccccc2-3)cc1]

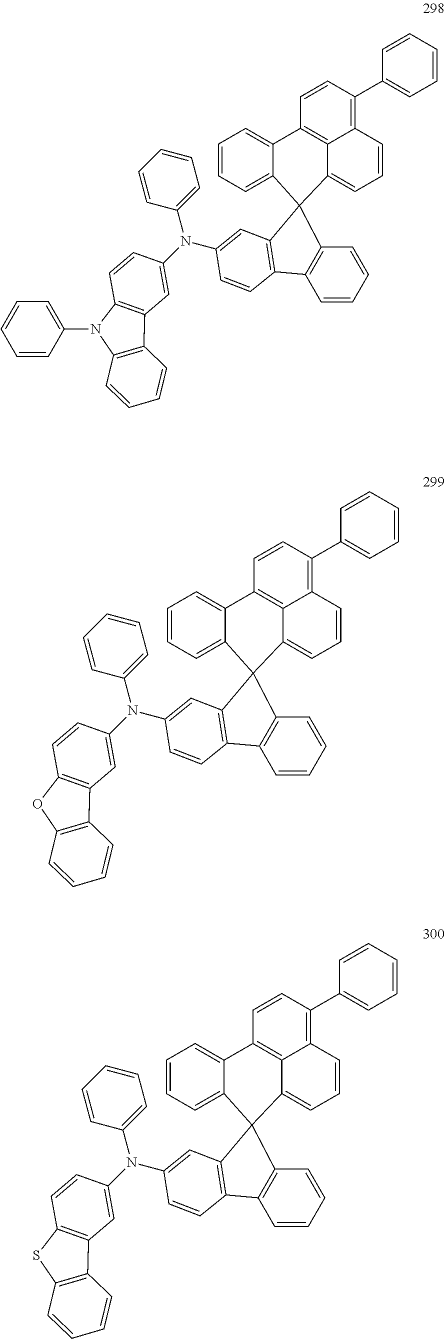 [SMILES: c1ccc(-c2ccc3c4c(cccc24)C2(c4ccccc4-c4ccc(N(c5ccccc5)c5ccc6c(c5)c5ccccc5n6-c5ccccc5)cc42)c2ccccc2-3)cc1 ; c1ccc(-c2ccc3c4c(cccc24)C2(c4ccccc4-c4ccc(N(c5ccccc5)c5ccc6oc7ccccc7c6c5)cc42)c2ccccc2-3)cc1 ; c1ccc(-c2ccc3c4c(cccc24)C2(c4ccccc4-c4ccc(N(c5ccccc5)c5ccc6sc7ccccc7c6c5)cc42)c2ccccc2-3)cc1]

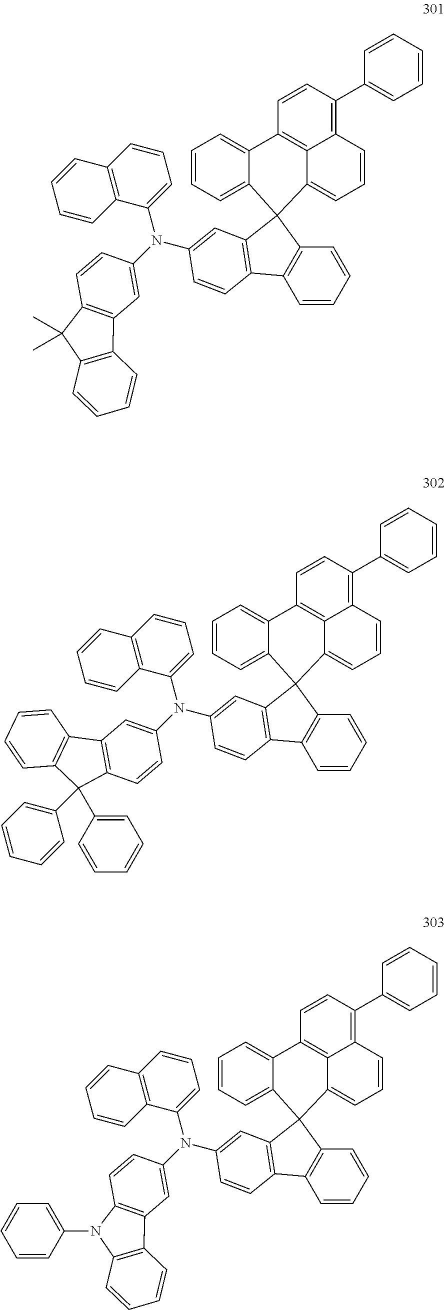 [SMILES: CC1(C)c2ccccc2-c2cc(N(c3ccc4c(c3)C3(c5ccccc5-4)c4ccccc4-c4ccc(-c5ccccc5)c5cccc3c45)c3cccc4ccccc34)ccc21 ; c1ccc(-c2ccc3c4c(cccc24)C2(c4ccccc4-c4ccc(N(c5ccc6c(c5)-c5ccccc5C6(c5ccccc5)c5ccccc5)c5cccc6ccccc56)cc42)c2ccccc2-3)cc1 ; c1ccc(-c2ccc3c4c(cccc24)C2(c4ccccc4-c4ccc(N(c5ccc6c(c5)c5ccccc5n6-c5ccccc5)c5cccc6ccccc56)cc42)c2ccccc2-3)cc1]

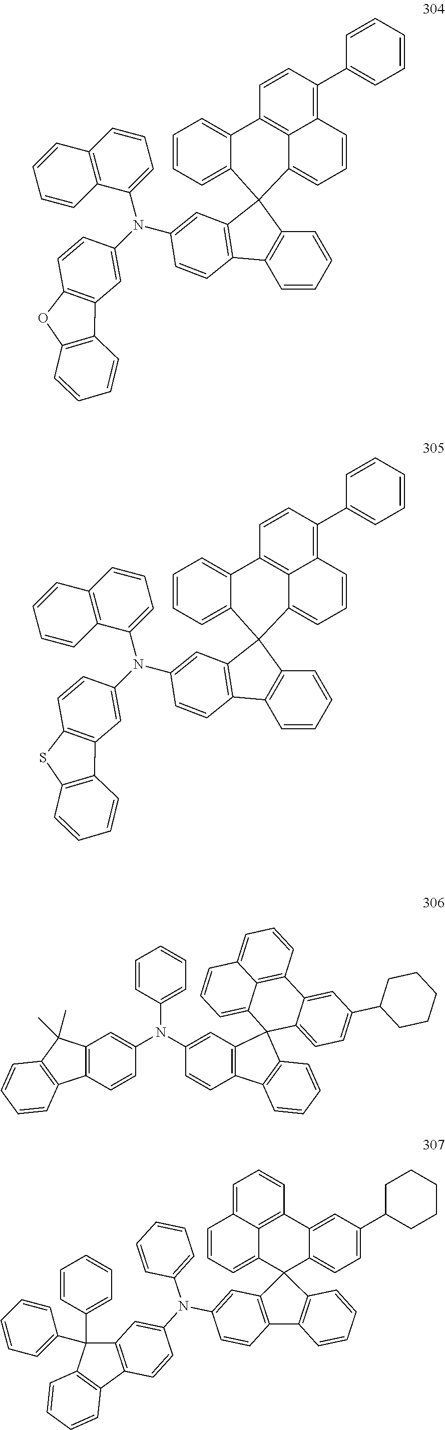 [SMILES: c1ccc(-c2ccc3c4c(cccc24)C2(c4ccccc4-c4ccc(N(c5ccc6oc7ccccc7c6c5)c5cccc6ccccc56)cc42)c2ccccc2-3)cc1 ; c1ccc(-c2ccc3c4c(cccc24)C2(c4ccccc4-c4ccc(N(c5ccc6sc7ccccc7c6c5)c5cccc6ccccc56)cc42)c2ccccc2-3)cc1 ; CC1(C)c2ccccc2-c2ccc(N(c3ccccc3)c3ccc4c(c3)C3(c5ccccc5-4)c4ccc(C5CCCCC5)cc4-c4cccc5cccc3c45)cc21 ; c1ccc(N(c2ccc3c(c2)C(c2ccccc2)(c2ccccc2)c2ccccc2-3)c2ccc3c(c2)C2(c4ccccc4-3)c3ccc(C4CCCCC4)cc3-c3cccc4cccc2c34)cc1]

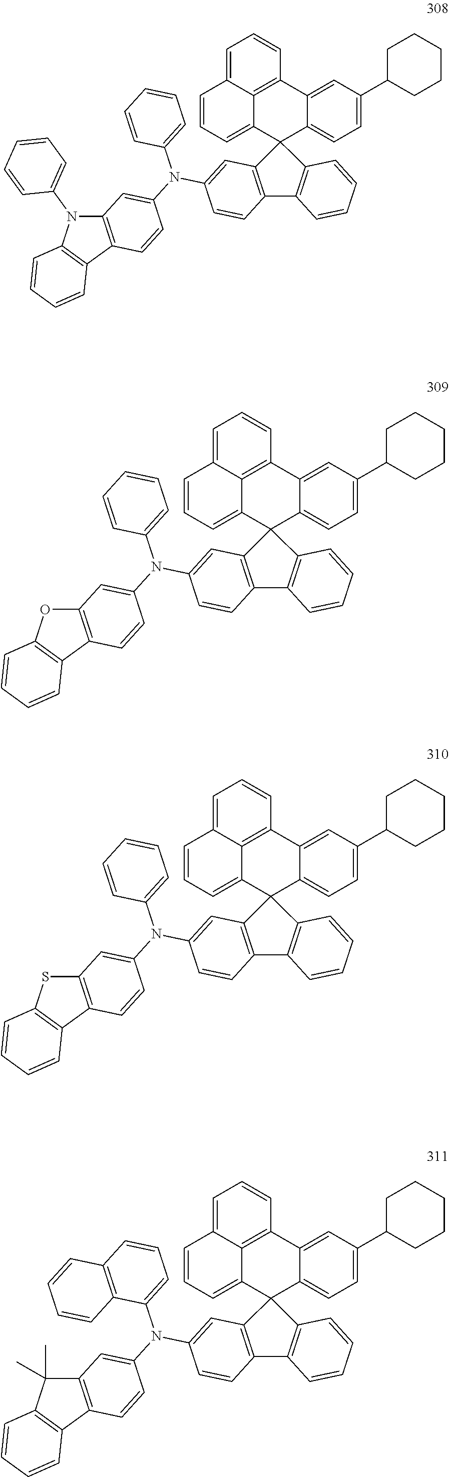 [SMILES: c1ccc(N(c2ccc3c(c2)C2(c4ccccc4-3)c3ccc(C4CCCCC4)cc3-c3cccc4cccc2c34)c2ccc3c4ccccc4n(-c4ccccc4)c3c2)cc1 ; c1ccc(N(c2ccc3c(c2)C2(c4ccccc4-3)c3ccc(C4CCCCC4)cc3-c3cccc4cccc2c34)c2ccc3c(c2)oc2ccccc23)cc1 ; c1ccc(N(c2ccc3c(c2)C2(c4ccccc4-3)c3ccc(C4CCCCC4)cc3-c3cccc4cccc2c34)c2ccc3c(c2)sc2ccccc23)cc1 ; CC1(C)c2ccccc2-c2ccc(N(c3ccc4c(c3)C3(c5ccccc5-4)c4ccc(C5CCCCC5)cc4-c4cccc5cccc3c45)c3cccc4ccccc34)cc21]

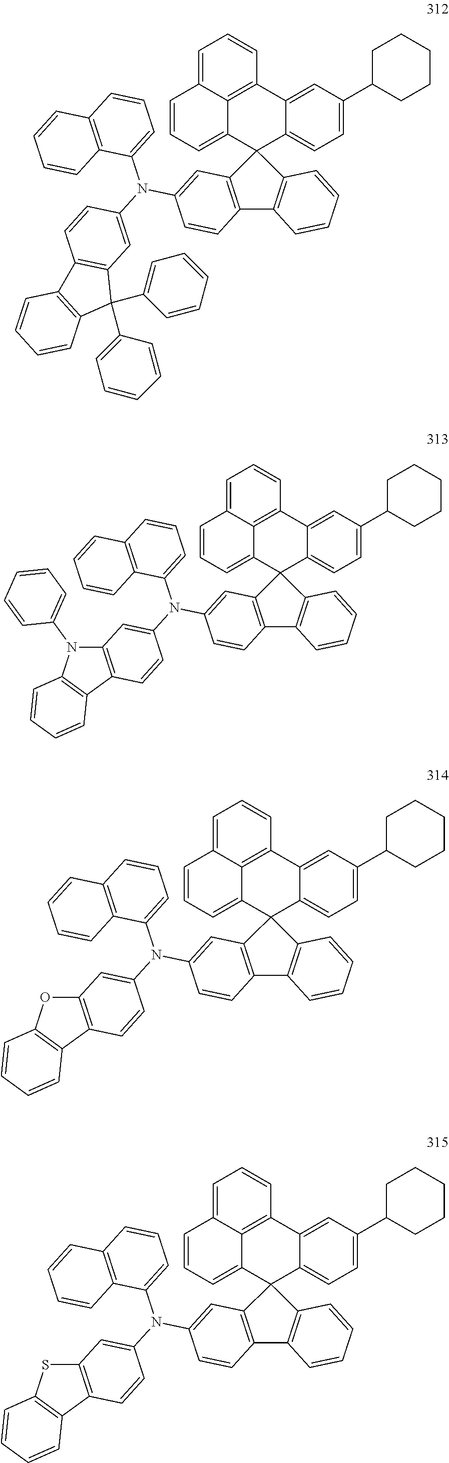 [SMILES: c1ccc(C2(c3ccccc3)c3ccccc3-c3ccc(N(c4ccc5c(c4)C4(c6ccccc6-5)c5ccc(C6CCCCC6)cc5-c5cccc6cccc4c56)c4cccc5ccccc45)cc32)cc1 ; c1ccc(-n2c3ccccc3c3ccc(N(c4ccc5c(c4)C4(c6ccccc6-5)c5ccc(C6CCCCC6)cc5-c5cccc6cccc4c56)c4cccc5ccccc45)cc32)cc1 ; c1ccc2c(c1)-c1ccc(N(c3ccc4c(c3)oc3ccccc34)c3cccc4ccccc34)cc1C21c2ccc(C3CCCCC3)cc2-c2cccc3cccc1c23 ; c1ccc2c(c1)-c1ccc(N(c3ccc4c(c3)sc3ccccc34)c3cccc4ccccc34)cc1C21c2ccc(C3CCCCC3)cc2-c2cccc3cccc1c23]

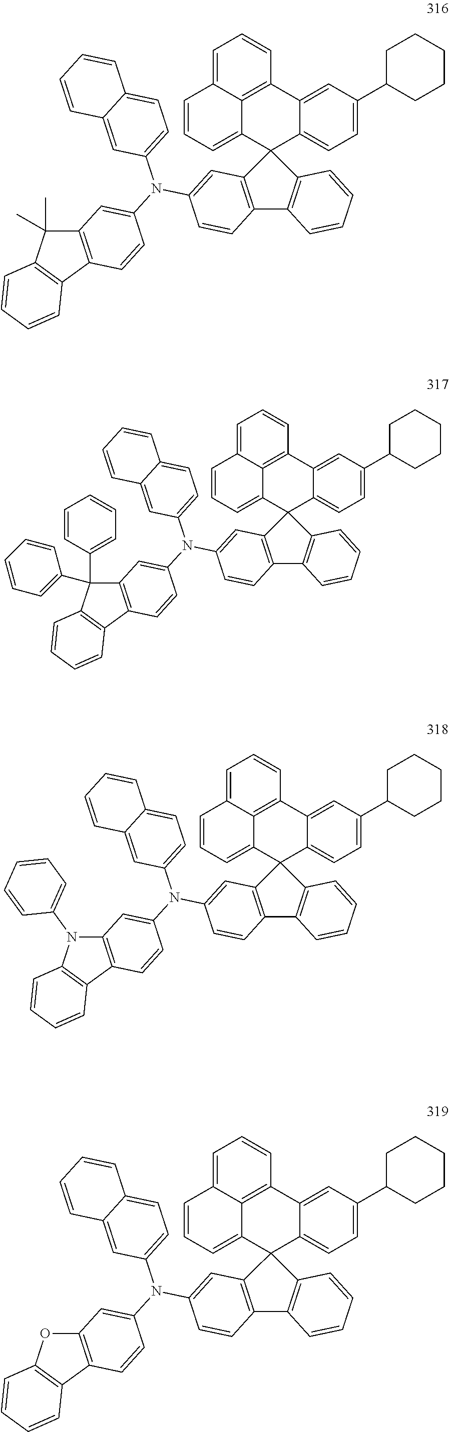 [SMILES: CC1(C)c2ccccc2-c2ccc(N(c3ccc4c(c3)C3(c5ccccc5-4)c4ccc(C5CCCCC5)cc4-c4cccc5cccc3c45)c3ccc4ccccc4c3)cc21 ; c1ccc(C2(c3ccccc3)c3ccccc3-c3ccc(N(c4ccc5c(c4)C4(c6ccccc6-5)c5ccc(C6CCCCC6)cc5-c5cccc6cccc4c56)c4ccc5ccccc5c4)cc32)cc1 ; c1ccc(-n2c3ccccc3c3ccc(N(c4ccc5c(c4)C4(c6ccccc6-5)c5ccc(C6CCCCC6)cc5-c5cccc6cccc4c56)c4ccc5ccccc5c4)cc32)cc1 ; c1ccc2c(c1)-c1ccc(N(c3ccc4ccccc4c3)c3ccc4c(c3)oc3ccccc34)cc1C21c2ccc(C3CCCCC3)cc2-c2cccc3cccc1c23]

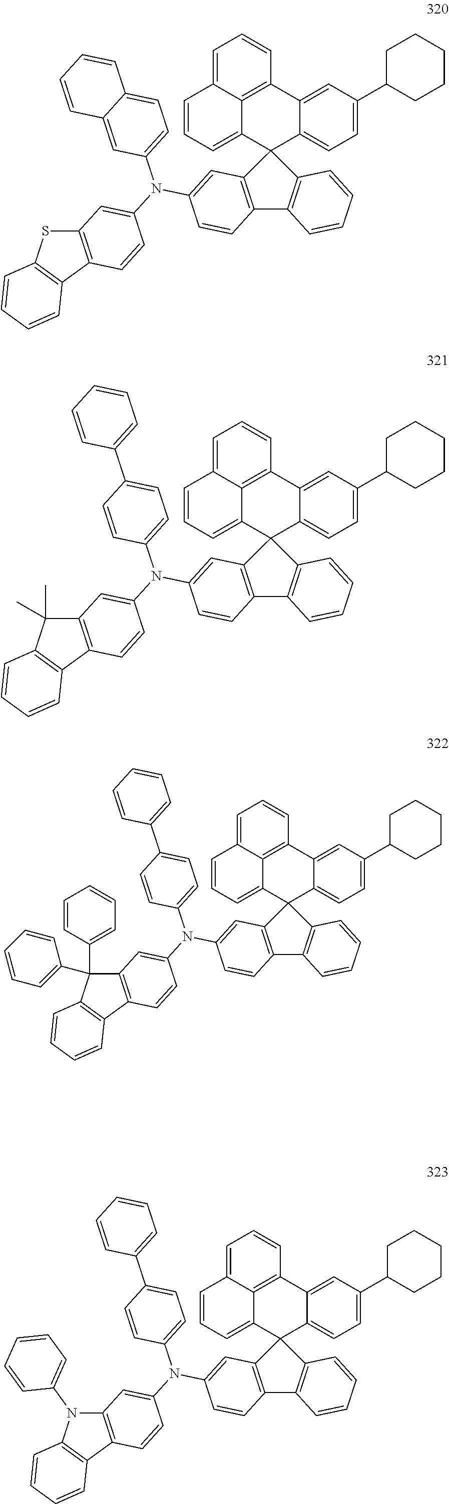 [SMILES: c1ccc2c(c1)-c1ccc(N(c3ccc4ccccc4c3)c3ccc4c(c3)sc3ccccc34)cc1C21c2ccc(C3CCCCC3)cc2-c2cccc3cccc1c23 ; CC1(C)c2ccccc2-c2ccc(N(c3ccc(-c4ccccc4)cc3)c3ccc4c(c3)C3(c5ccccc5-4)c4ccc(C5CCCCC5)cc4-c4cccc5cccc3c45)cc21 ; c1ccc(-c2ccc(N(c3ccc4c(c3)C(c3ccccc3)(c3ccccc3)c3ccccc3-4)c3ccc4c(c3)C3(c5ccccc5-4)c4ccc(C5CCCCC5)cc4-c4cccc5cccc3c45)cc2)cc1 ; c1ccc(-c2ccc(N(c3ccc4c(c3)C3(c5ccccc5-4)c4ccc(C5CCCCC5)cc4-c4cccc5cccc3c45)c3ccc4c5ccccc5n(-c5ccccc5)c4c3)cc2)cc1]

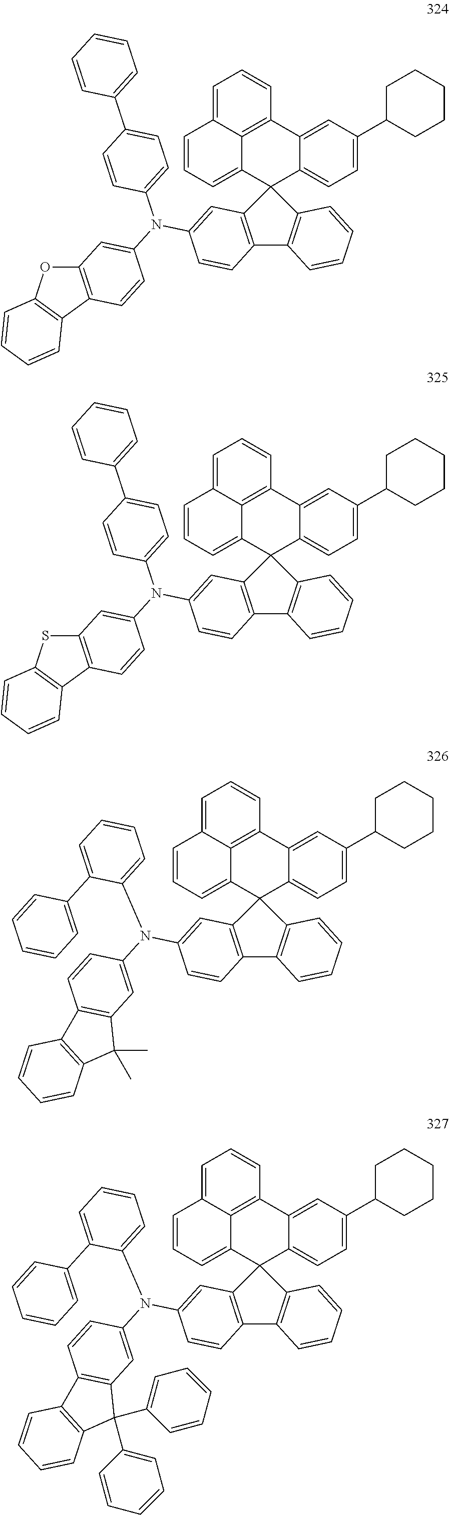 [SMILES: c1ccc(-c2ccc(N(c3ccc4c(c3)C3(c5ccccc5-4)c4ccc(C5CCCCC5)cc4-c4cccc5cccc3c45)c3ccc4c(c3)oc3ccccc34)cc2)cc1 ; c1ccc(-c2ccc(N(c3ccc4c(c3)C3(c5ccccc5-4)c4ccc(C5CCCCC5)cc4-c4cccc5cccc3c45)c3ccc4c(c3)sc3ccccc34)cc2)cc1 ; CC1(C)c2ccccc2-c2ccc(N(c3ccc4c(c3)C3(c5ccccc5-4)c4ccc(C5CCCCC5)cc4-c4cccc5cccc3c45)c3ccccc3-c3ccccc3)cc21 ; c1ccc(-c2ccccc2N(c2ccc3c(c2)C(c2ccccc2)(c2ccccc2)c2ccccc2-3)c2ccc3c(c2)C2(c4ccccc4-3)c3ccc(C4CCCCC4)cc3-c3cccc4cccc2c34)cc1]

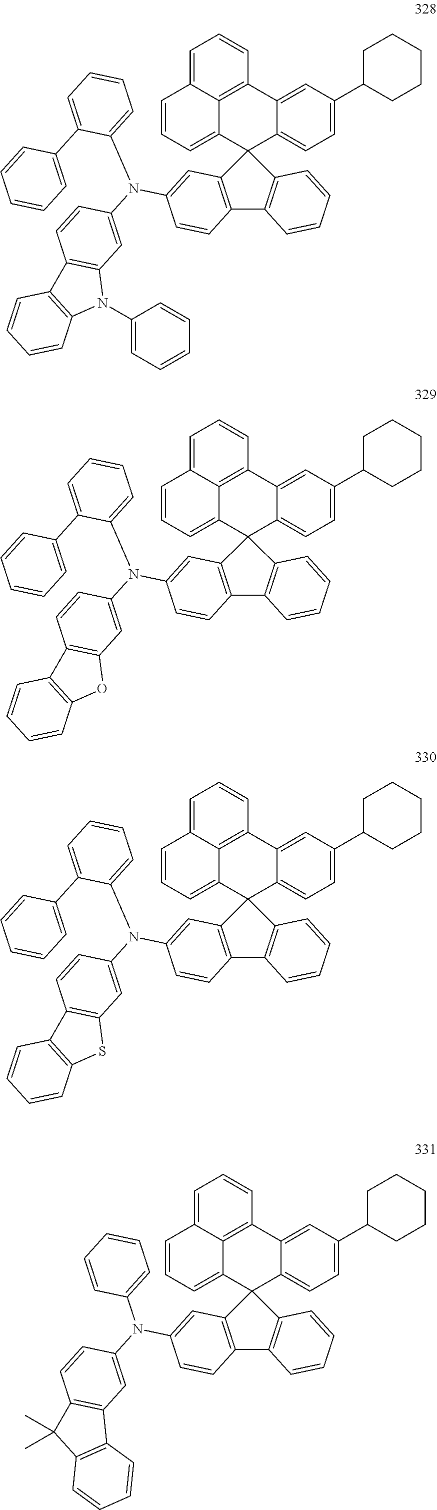 [SMILES: c1ccc(-c2ccccc2N(c2ccc3c(c2)C2(c4ccccc4-3)c3ccc(C4CCCCC4)cc3-c3cccc4cccc2c34)c2ccc3c4ccccc4n(-c4ccccc4)c3c2)cc1 ; c1ccc(-c2ccccc2N(c2ccc3c(c2)C2(c4ccccc4-3)c3ccc(C4CCCCC4)cc3-c3cccc4cccc2c34)c2ccc3c(c2)oc2ccccc23)cc1 ; c1ccc(-c2ccccc2N(c2ccc3c(c2)C2(c4ccccc4-3)c3ccc(C4CCCCC4)cc3-c3cccc4cccc2c34)c2ccc3c(c2)sc2ccccc23)cc1 ; CC1(C)c2ccccc2-c2cc(N(c3ccccc3)c3ccc4c(c3)C3(c5ccccc5-4)c4ccc(C5CCCCC5)cc4-c4cccc5cccc3c45)ccc21]

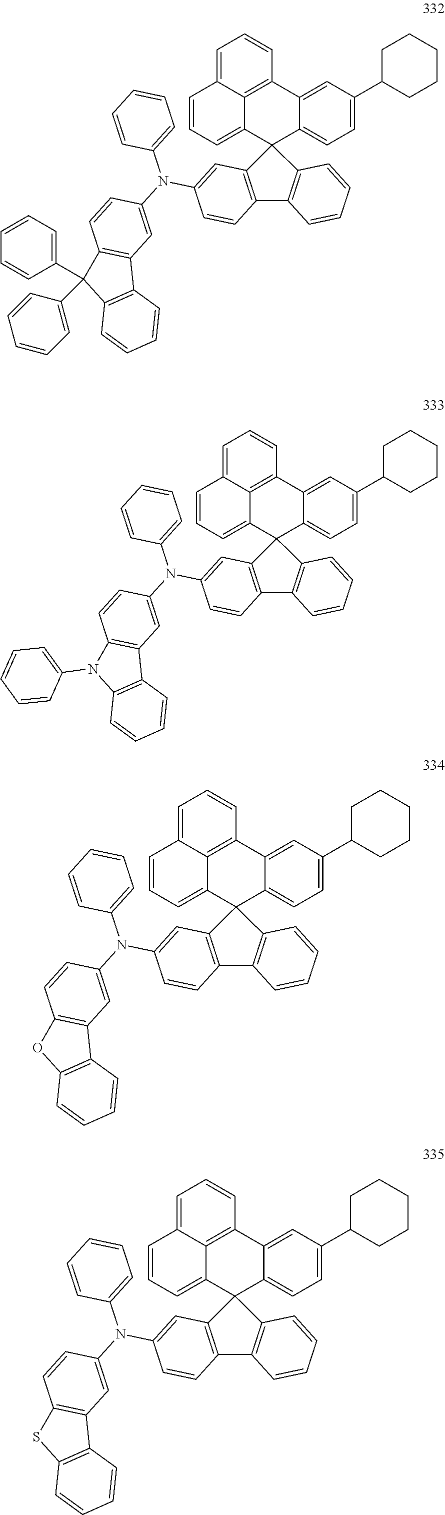 [SMILES: c1ccc(N(c2ccc3c(c2)-c2ccccc2C3(c2ccccc2)c2ccccc2)c2ccc3c(c2)C2(c4ccccc4-3)c3ccc(C4CCCCC4)cc3-c3cccc4cccc2c34)cc1 ; c1ccc(N(c2ccc3c(c2)C2(c4ccccc4-3)c3ccc(C4CCCCC4)cc3-c3cccc4cccc2c34)c2ccc3c(c2)c2ccccc2n3-c2ccccc2)cc1 ; c1ccc(N(c2ccc3c(c2)C2(c4ccccc4-3)c3ccc(C4CCCCC4)cc3-c3cccc4cccc2c34)c2ccc3oc4ccccc4c3c2)cc1 ; c1ccc(N(c2ccc3c(c2)C2(c4ccccc4-3)c3ccc(C4CCCCC4)cc3-c3cccc4cccc2c34)c2ccc3sc4ccccc4c3c2)cc1]

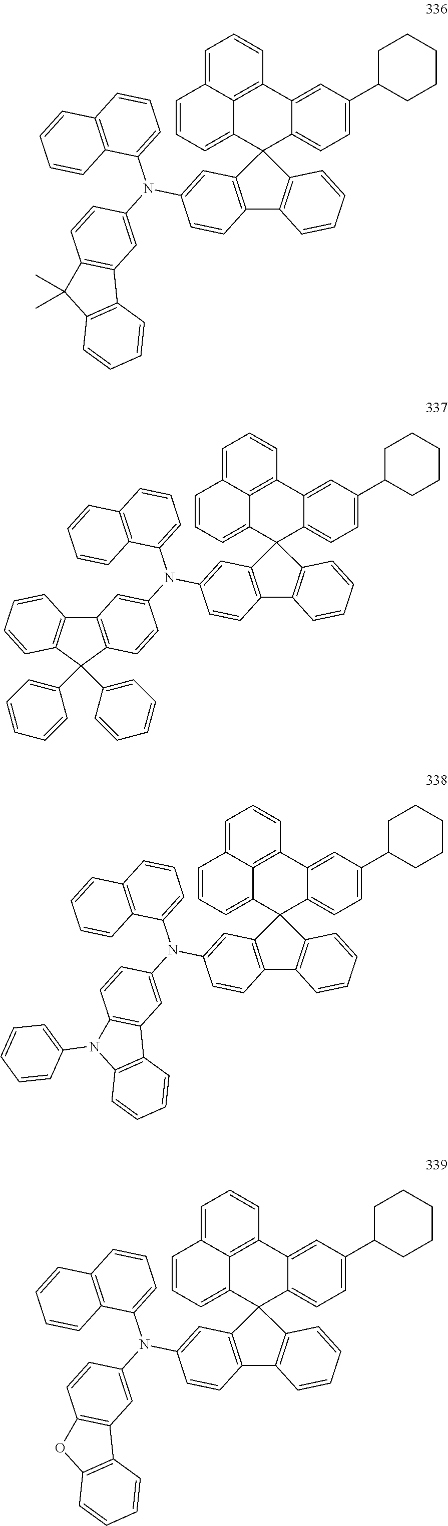 [SMILES: CC1(C)c2ccccc2-c2cc(N(c3ccc4c(c3)C3(c5ccccc5-4)c4ccc(C5CCCCC5)cc4-c4cccc5cccc3c45)c3cccc4ccccc34)ccc21 ; c1ccc(C2(c3ccccc3)c3ccccc3-c3cc(N(c4ccc5c(c4)C4(c6ccccc6-5)c5ccc(C6CCCCC6)cc5-c5cccc6cccc4c56)c4cccc5ccccc45)ccc32)cc1 ; c1ccc(-n2c3ccccc3c3cc(N(c4ccc5c(c4)C4(c6ccccc6-5)c5ccc(C6CCCCC6)cc5-c5cccc6cccc4c56)c4cccc5ccccc45)ccc32)cc1 ; c1ccc2c(c1)-c1ccc(N(c3ccc4oc5ccccc5c4c3)c3cccc4ccccc34)cc1C21c2ccc(C3CCCCC3)cc2-c2cccc3cccc1c23]

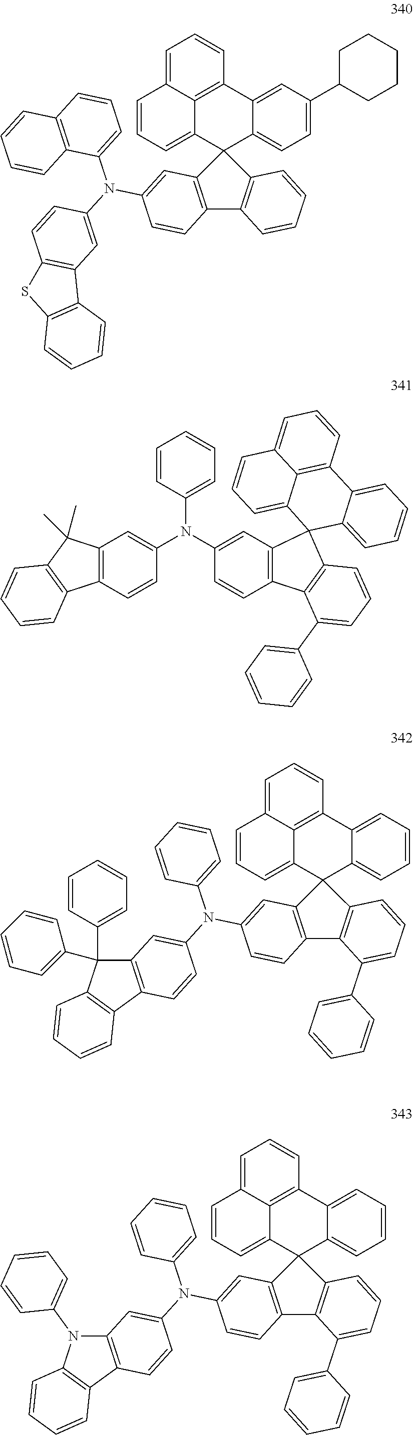 [SMILES: c1ccc2c(c1)-c1ccc(N(c3ccc4sc5ccccc5c4c3)c3cccc4ccccc34)cc1C21c2ccc(C3CCCCC3)cc2-c2cccc3cccc1c23 ; CC1(C)c2ccccc2-c2ccc(N(c3ccccc3)c3ccc4c(c3)C3(c5ccccc5-c5cccc6cccc3c56)c3cccc(-c5ccccc5)c3-4)cc21 ; c1ccc(-c2cccc3c2-c2ccc(N(c4ccccc4)c4ccc5c(c4)C(c4ccccc4)(c4ccccc4)c4ccccc4-5)cc2C32c3ccccc3-c3cccc4cccc2c34)cc1 ; c1ccc(-c2cccc3c2-c2ccc(N(c4ccccc4)c4ccc5c6ccccc6n(-c6ccccc6)c5c4)cc2C32c3ccccc3-c3cccc4cccc2c34)cc1]

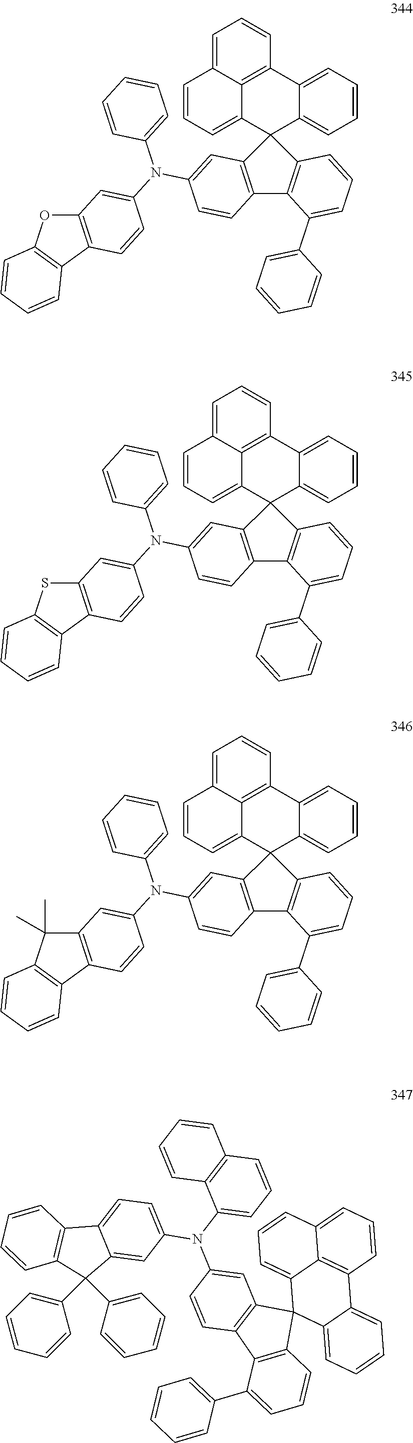 [SMILES: c1ccc(-c2cccc3c2-c2ccc(N(c4ccccc4)c4ccc5c(c4)oc4ccccc45)cc2C32c3ccccc3-c3cccc4cccc2c34)cc1 ; c1ccc(-c2cccc3c2-c2ccc(N(c4ccccc4)c4ccc5c(c4)sc4ccccc45)cc2C32c3ccccc3-c3cccc4cccc2c34)cc1 ; CC1(C)c2ccccc2-c2ccc(N(c3ccccc3)c3ccc4c(c3)C3(c5ccccc5-c5cccc6cccc3c56)c3cccc(-c5ccccc5)c3-4)cc21 ; c1ccc(-c2cccc3c2-c2ccc(N(c4ccc5c(c4)C(c4ccccc4)(c4ccccc4)c4ccccc4-5)c4cccc5ccccc45)cc2C32c3ccccc3-c3cccc4cccc2c34)cc1]

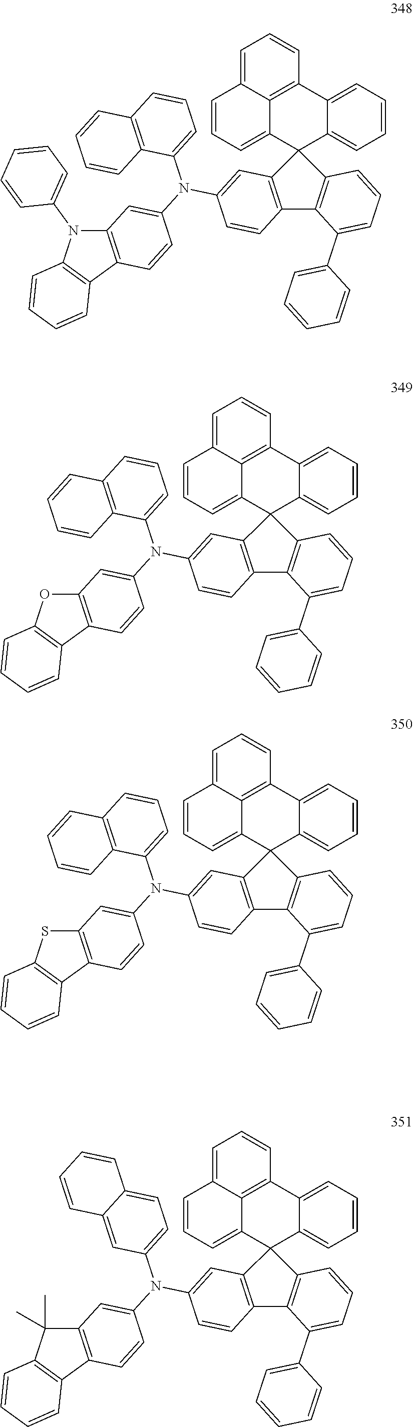 [SMILES: c1ccc(-c2cccc3c2-c2ccc(N(c4ccc5c6ccccc6n(-c6ccccc6)c5c4)c4cccc5ccccc45)cc2C32c3ccccc3-c3cccc4cccc2c34)cc1 ; c1ccc(-c2cccc3c2-c2ccc(N(c4ccc5c(c4)oc4ccccc45)c4cccc5ccccc45)cc2C32c3ccccc3-c3cccc4cccc2c34)cc1 ; c1ccc(-c2cccc3c2-c2ccc(N(c4ccc5c(c4)sc4ccccc45)c4cccc5ccccc45)cc2C32c3ccccc3-c3cccc4cccc2c34)cc1 ; CC1(C)c2ccccc2-c2ccc(N(c3ccc4c(c3)C3(c5ccccc5-c5cccc6cccc3c56)c3cccc(-c5ccccc5)c3-4)c3ccc4ccccc4c3)cc21]

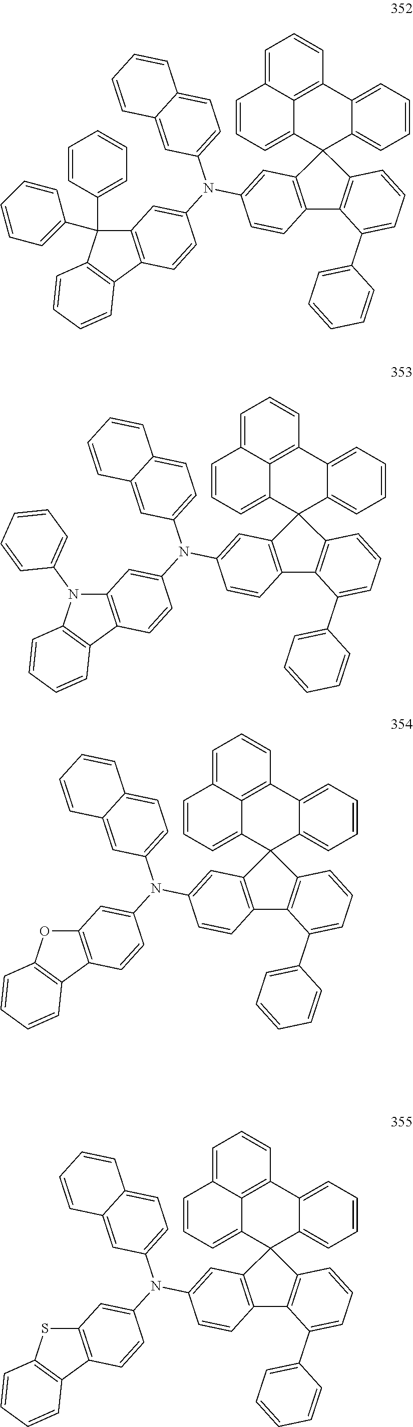 [SMILES: c1ccc(-c2cccc3c2-c2ccc(N(c4ccc5c(c4)C(c4ccccc4)(c4ccccc4)c4ccccc4-5)c4ccc5ccccc5c4)cc2C32c3ccccc3-c3cccc4cccc2c34)cc1 ; c1ccc(-c2cccc3c2-c2ccc(N(c4ccc5ccccc5c4)c4ccc5c6ccccc6n(-c6ccccc6)c5c4)cc2C32c3ccccc3-c3cccc4cccc2c34)cc1 ; c1ccc(-c2cccc3c2-c2ccc(N(c4ccc5ccccc5c4)c4ccc5c(c4)oc4ccccc45)cc2C32c3ccccc3-c3cccc4cccc2c34)cc1 ; c1ccc(-c2cccc3c2-c2ccc(N(c4ccc5ccccc5c4)c4ccc5c(c4)sc4ccccc45)cc2C32c3ccccc3-c3cccc4cccc2c34)cc1]

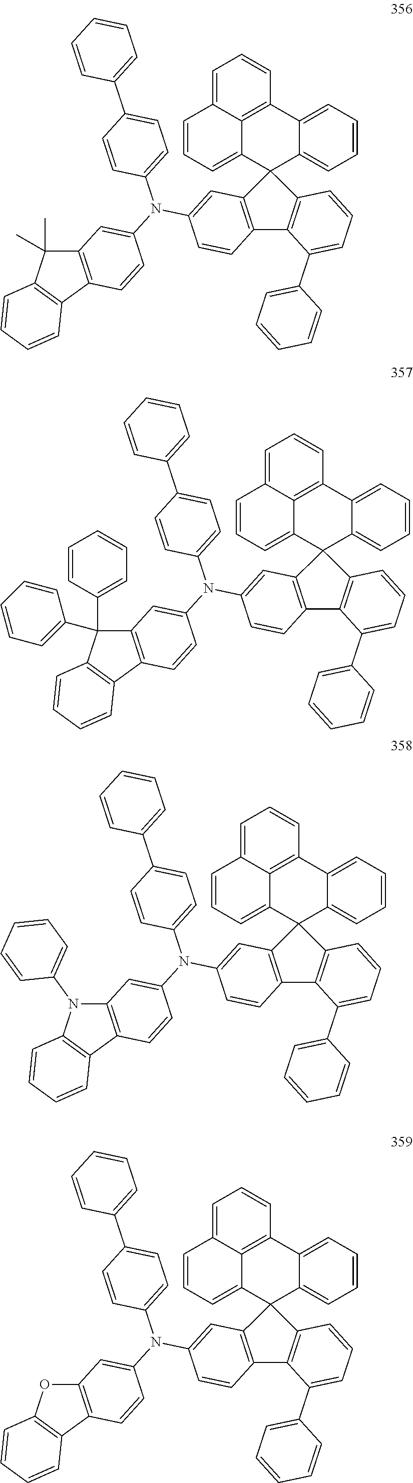 [SMILES: CC1(C)c2ccccc2-c2ccc(N(c3ccc(-c4ccccc4)cc3)c3ccc4c(c3)C3(c5ccccc5-c5cccc6cccc3c56)c3cccc(-c5ccccc5)c3-4)cc21 ; c1ccc(-c2ccc(N(c3ccc4c(c3)C(c3ccccc3)(c3ccccc3)c3ccccc3-4)c3ccc4c(c3)C3(c5ccccc5-c5cccc6cccc3c56)c3cccc(-c5ccccc5)c3-4)cc2)cc1 ; c1ccc(-c2ccc(N(c3ccc4c(c3)C3(c5ccccc5-c5cccc6cccc3c56)c3cccc(-c5ccccc5)c3-4)c3ccc4c5ccccc5n(-c5ccccc5)c4c3)cc2)cc1 ; c1ccc(-c2ccc(N(c3ccc4c(c3)C3(c5ccccc5-c5cccc6cccc3c56)c3cccc(-c5ccccc5)c3-4)c3ccc4c(c3)oc3ccccc34)cc2)cc1]

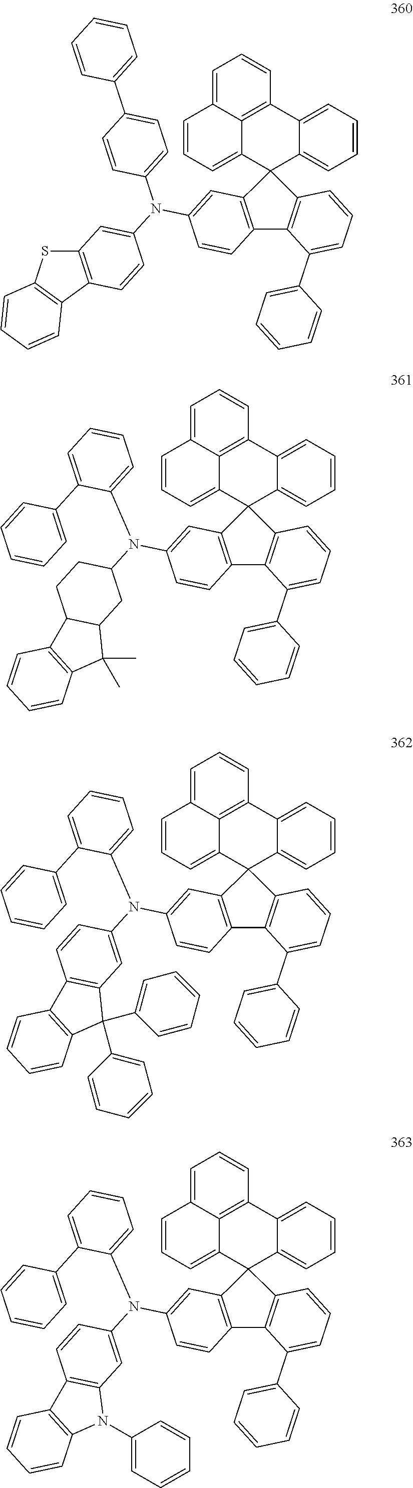 [SMILES: c1ccc(-c2ccc(N(c3ccc4c(c3)C3(c5ccccc5-c5cccc6cccc3c56)c3cccc(-c5ccccc5)c3-4)c3ccc4c(c3)sc3ccccc34)cc2)cc1 ; CC1(C)c2ccccc2C2CCC(N(c3ccc4c(c3)C3(c5ccccc5-c5cccc6cccc3c56)c3cccc(-c5ccccc5)c3-4)c3ccccc3-c3ccccc3)CC21 ; c1ccc(-c2ccccc2N(c2ccc3c(c2)C(c2ccccc2)(c2ccccc2)c2ccccc2-3)c2ccc3c(c2)C2(c4ccccc4-c4cccc5cccc2c45)c2cccc(-c4ccccc4)c2-3)cc1 ; c1ccc(-c2ccccc2N(c2ccc3c(c2)C2(c4ccccc4-c4cccc5cccc2c45)c2cccc(-c4ccccc4)c2-3)c2ccc3c4ccccc4n(-c4ccccc4)c3c2)cc1]

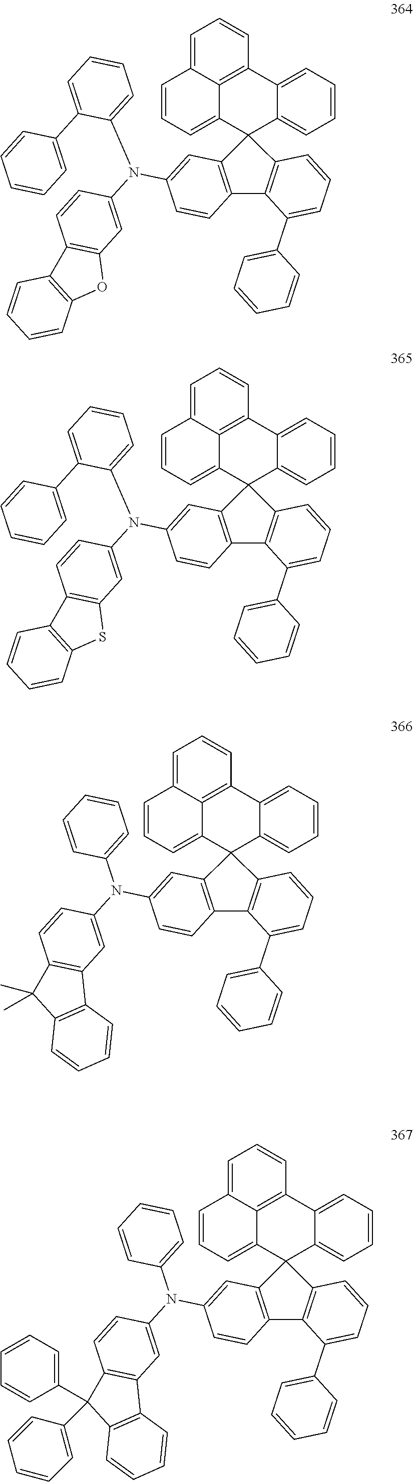 [SMILES: c1ccc(-c2ccccc2N(c2ccc3c(c2)C2(c4ccccc4-c4cccc5cccc2c45)c2cccc(-c4ccccc4)c2-3)c2ccc3c(c2)oc2ccccc23)cc1 ; c1ccc(-c2ccccc2N(c2ccc3c(c2)C2(c4ccccc4-c4cccc5cccc2c45)c2cccc(-c4ccccc4)c2-3)c2ccc3c(c2)sc2ccccc23)cc1 ; CC1(C)c2ccccc2-c2cc(N(c3ccccc3)c3ccc4c(c3)C3(c5ccccc5-c5cccc6cccc3c56)c3cccc(-c5ccccc5)c3-4)ccc21 ; c1ccc(-c2cccc3c2-c2ccc(N(c4ccccc4)c4ccc5c(c4)-c4ccccc4C5(c4ccccc4)c4ccccc4)cc2C32c3ccccc3-c3cccc4cccc2c34)cc1]

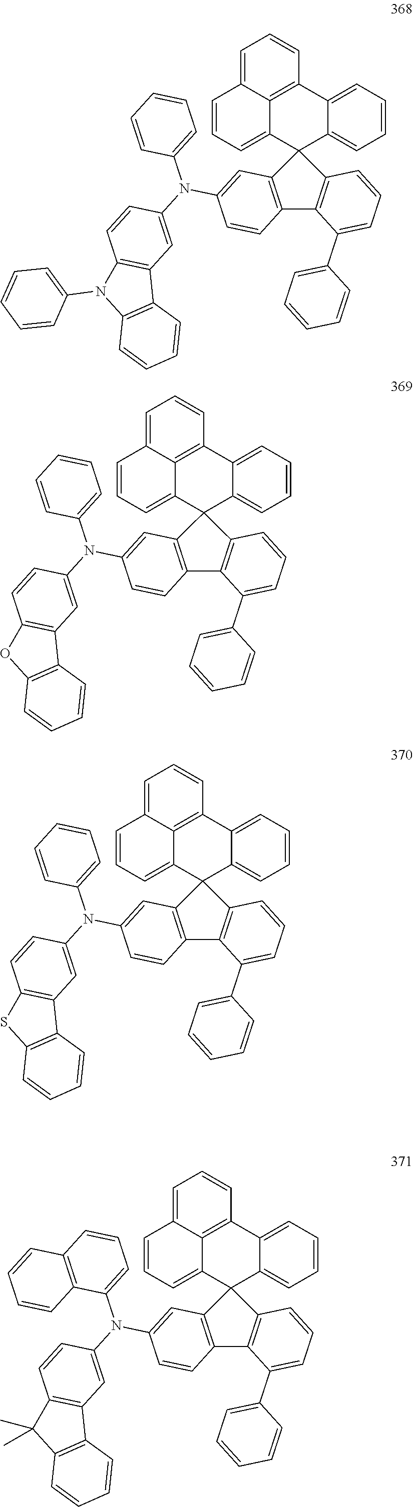

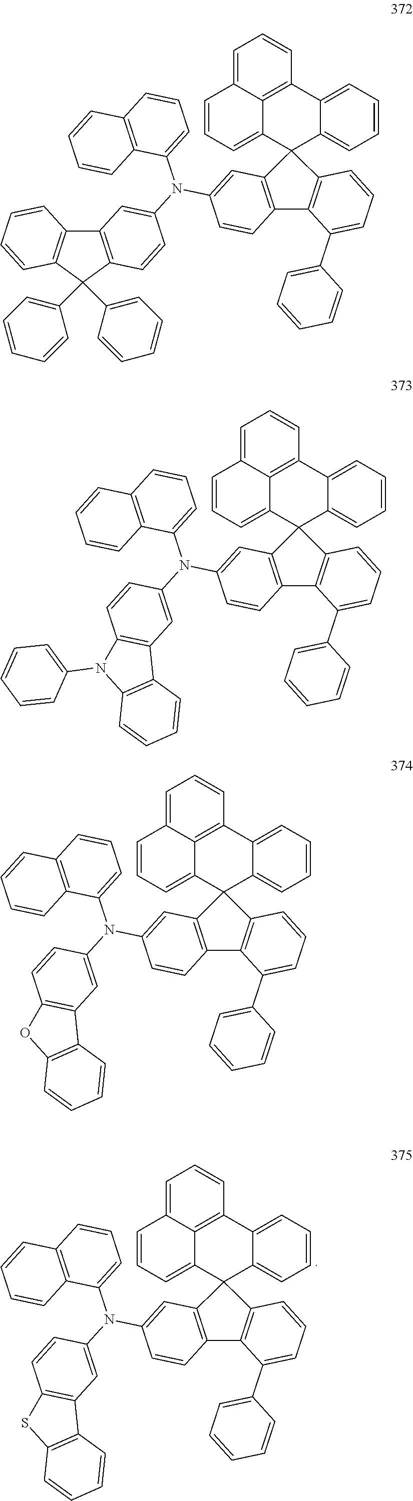 [SMILES: c1ccc(-c2cccc3c2-c2ccc(N(c4ccc5c(c4)-c4ccccc4C5(c4ccccc4)c4ccccc4)c4cccc5ccccc45)cc2C32c3ccccc3-c3cccc4cccc2c34)cc1 ; c1ccc(-c2cccc3c2-c2ccc(N(c4ccc5c(c4)c4ccccc4n5-c4ccccc4)c4cccc5ccccc45)cc2C32c3ccccc3-c3cccc4cccc2c34)cc1 ; c1ccc(-c2cccc3c2-c2ccc(N(c4ccc5oc6ccccc6c5c4)c4cccc5ccccc45)cc2C32c3ccccc3-c3cccc4cccc2c34)cc1 ; c1ccc(-c2cccc3c2-c2ccc(N(c4ccc5sc6ccccc6c5c4)c4cccc5ccccc45)cc2C32c3ccccc3-c3cccc4cccc2c34)cc1]

The condensed cyclic compound represented by Formula 1 may have a spiro[benzo[de]anthracene-7.9′-fluorene] structure as a core and may also (e.g., essentially) include an amine group (refer to the group represented by Formula 2) and substituents other than the amine group, wherein the physical and/or chemical properties of the compound may be controlled or selected according to the position and/or number and type or kind of such substituents.

First, because the condensed cyclic compound represented by Formula 1 usually has a large molecular weight, a glass transition temperature may be greatly increased. In some embodiments, by controlling the type or kind and/or position of the substituents other than the amine group, i) the molecular structure in the polaron state may be stabilized to improve device lifespan and efficiency through π-conjugation expansion, and/or ii) the intermolecular interactions may be controlled or selected and the packing density of molecules may be reduced through the introduction of substituents. Accordingly, by decreasing a sublimation temperature, processability may be improved.

Accordingly, the condensed cyclic compound represented by Formula 1 may have various suitable highest occupied molecular orbital (HOMO) energy level by variously suitably changing (e.g., the position of) the amine group and the substituents other than the amine group in the core. Accordingly, the hole injection barrier of the anode and the hole transport layer may be variously suitably changed and, by controlling an energy level between the hole transport layer and the emission layer to be appropriate or suitable, the exciton generation efficiency in the emission layer may be increased.

Thus, by utilizing the condensed cyclic compound represented by Formula 1, an electronic device (for example, an organic light-emitting device) having high efficiency, low voltage, high luminance, and long lifespan may be realized.

Methods of synthesizing the condensed cyclic compound represented by Formula 1 may be easily understood to those of ordinary skill in the art by referring to Synthesis Examples and Examples described herein.

At least one condensed cyclic compound represented by Formula 1 may be utilized in a light-emitting device (for example, an organic light-emitting device).

In an embodiment, a light-emitting device may include: a first electrode; a second electrode facing the first electrode; an interlayer located between the first electrode and the second electrode and including an emission layer; and the condensed cyclic compound represented by Formula 1 as described in the present specification.

In an embodiment,

the first electrode of the light-emitting device may be an anode,

the second electrode of the light-emitting device may be a cathode,

the interlayer may further include a hole transport region between the first electrode and the emission layer and an electron transport region between the emission layer and the second electrode,

the hole transport region may include a hole injection layer, a hole transport layer, an emission auxiliary layer, an electron blocking layer, or any combination thereof, and

the electron transport region may include a buffer layer, a hole blocking layer, an electron control layer, an electron transport layer, an electron injection layer, or any combination thereof.

In an embodiment, the condensed cyclic compound represented by Formula 1 may be included between the first electrode and the second electrode of the light-emitting device. The condensed cyclic compound represented by Formula 1 may be included in the interlayer of the light-emitting device, for example, at least one selected from the group consisting of the hole transport region, the electron transport region, and the emission layer.

In an embodiment, the condensed cyclic compound represented by Formula 1 may be included in the hole transport region of the interlayer.

In an embodiment, the hole transport region may include at least one selected from the group consisting of the hole injection layer, the hole transport layer, and the electron blocking layer, and

at least one selected from the group consisting of the hole injection layer, the hole transport layer, and the electron blocking layer may include the condensed cyclic compound represented by Formula 1.

For example, the hole transport layer may include the condensed cyclic compound represented by Formula 1.

In an embodiment, the hole transport region of the light-emitting device may further include a p-dopant having a lowest unoccupied molecular orbital (LUMO) energy level of −3.5 eV or less.

For example, the p-dopant may include at least one selected from one or more quinone derivatives, metal oxides and cyano group-containing compounds.

For example, the hole transport region may include a hole transport layer and the hole transport layer may include the p-dopant, or the hole transport region may include a hole injection layer and the hole injection layer may include the p-dopant.

In an embodiment, the emission layer may include a host and a dopant. For example, the host may include an anthracene compound, but embodiments of the present disclosure are not limited thereto, and the dopant may include an aryl amine compound and/or a styryl aryl compound, but embodiments of the present disclosure are not limited thereto. In some embodiments, the amount of the host may be greater than the amount of the dopant. For example, the amount of the dopant in the emission layer may be about 0.01 parts by weight to about 5 parts by weight or about 0.01 parts by weight to about 3 parts by weight, based on the total weight of 100 parts by weight of the host and the dopant, but embodiments of the present disclosure are not limited thereto.

In one or more embodiments, the emission layer in the interlayer of the light-emitting device may include a dopant and a host, and the condensed cyclic compound represented by Formula 1 may be included in the host. For example, the condensed cyclic compound represented by Formula 1 may act (or serve) as a host.

The emission layer may be to emit red light, green light, blue light, and/or white light. For example, the emission layer may be to emit blue light. The blue light may have a maximum emission wavelength of, for example, about 400 nm to about 490 nm, or about 430 nm to about 490 nm.

In an embodiment, the light-emitting device may include a capping layer located outside the first electrode or outside the second electrode.

In an embodiment, the light-emitting device may further include at least one of a first capping layer located outside the first electrode or a second capping layer located outside the second electrode, and at least one of the first capping layer or the second capping layer may include the condensed cyclic compound represented by Formula 1. More details for the first capping layer and/or second capping layer may each independently be the same as described in the present specification.

In an embodiment, the light-emitting device may include:

a first capping layer arranged outside the first electrode (e.g., facing away from the second electrode) and including the condensed cyclic compound represented by Formula 1;

a second capping layer arranged outside the second electrode (e.g., facing away from the first electrode) and including the condensed cyclic compound represented by Formula 1; or

both the first capping layer and the second capping layer.

The wording “(interlayer and/or capping layer) includes an condensed cyclic compound represented by Formula 1” as utilized herein may be understood as “(interlayer and/or capping layer) may include one kind of condensed cyclic compound represented by Formula 1 or two different kinds of condensed cyclic compounds, each represented by Formula 1”.

In an embodiment, the interlayer and/or the capping layer may include Compound 1 only as the condensed cyclic compound. In this regard, Compound 1 may be present in the emission layer of the light-emitting device. In one or more embodiments, the interlayer may include, as the condensed cyclic compound, Compound 1 and Compound 2. In this regard, Compounds 1 and 2 may be present in substantially the same layer (for example, both (e.g., simultaneously) Compounds 1 and 2 may be present in an emission layer), or may be present in different layers (for example, Compound 1 may be present in an emission layer, and Compound 2 may be present in a hole transport region).

The term “interlayer” as used herein refers to a single layer and/or all of a plurality of layers located between the first electrode and the second electrode of the light-emitting device.

Another aspect provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In an embodiment, the electronic apparatus may further include a color filter, a color conversion layer, a touch screen layer, a polarizing layer, or any combination thereof. For more details on the electronic apparatus, related descriptions provided herein may be referred to.

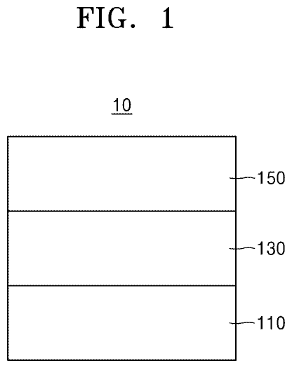

### Description of FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a substrate may be additionally located under the first electrode 110 and/or on the second electrode 150. As the substrate, a glass substrate and/or a plastic substrate may be utilized. In one or more embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be located on the first electrode 110. The interlayer 130 may include an emission layer.

The interlayer 130 may further include a hole transport region located between the first electrode 110 and the emission layer, and an electron transport region located between the emission layer and the second electrode 150.

The interlayer 130 may further include, in addition to one or more suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer located between the two or more emitting units. When the interlayer 130 includes the two or more emitting units and the charge generation layer as described above, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, with constituting layers of each structure being stacked sequentially from the first electrode 110 in the respective stated order.

The hole transport region may include a compound represented by Formula 201, a compound represented by Formula 202, or any combination thereof:

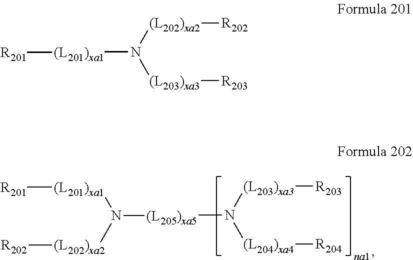

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, each of Formulae 201 and 202 may include at least one of the groups represented by Formulae CY201 to CY217:

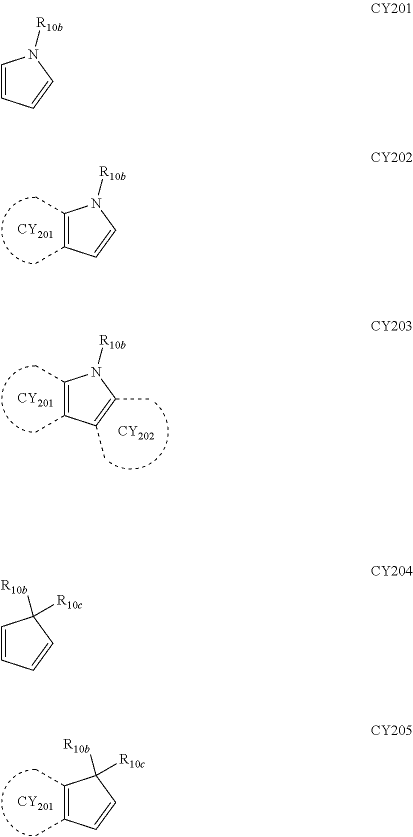

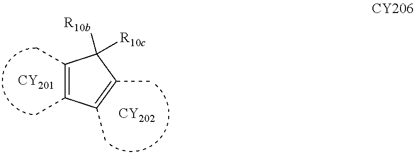

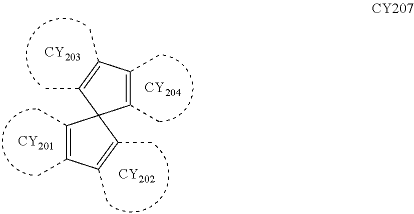

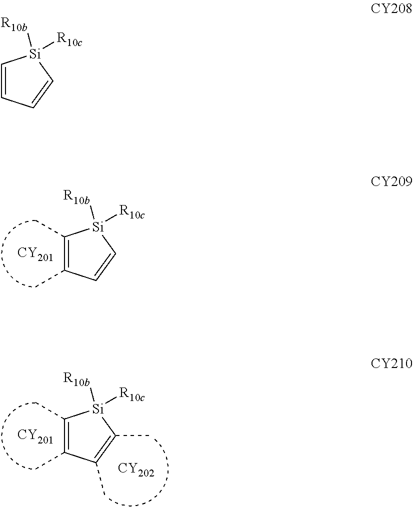

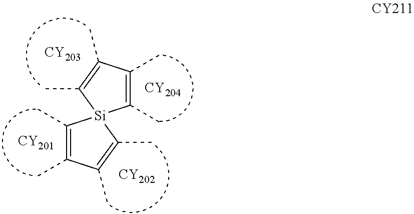

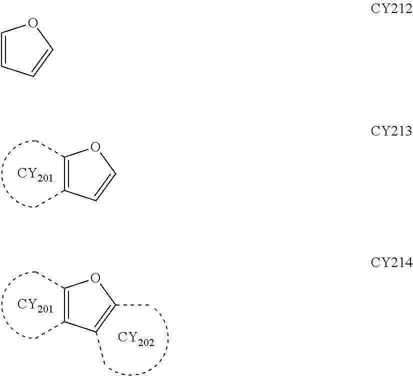

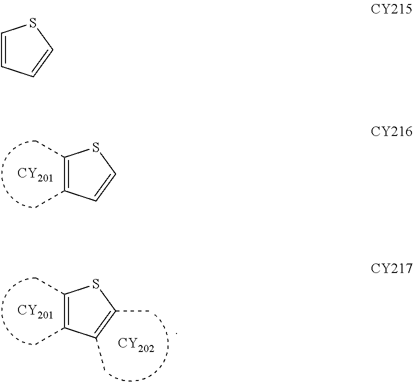

In Formulae CY201 to CY217, R_{10b} and R_{10c} may each independently be the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described above.

In an embodiment, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one of the groups represented by Formulae CY201 to CY203.

In one or more embodiments, Formula 201 may include at least one of the groups represented by Formulae CY201 to CY203 and at least one of the groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include (e.g., may exclude) any of the groups represented by Formulae CY201 to CY203.

In one or more embodiments, each of Formulae 201 and 202 may not include (e.g., may exclude) any of the groups represented by Formulae CY201 to CY203, and may include at least one of the groups represented by Formulae CY204 to CY217.

In one or more embodiments, each of Formulae 201 and 202 may not include(e.g., may exclude) any of the groups represented by Formulae CY201 to CY217.

In an embodiment, the hole transport region may include at least one of Compounds HT1 to HT46, m-MTDATA, TDATA, 2-TNATA, NPB(NPD), p-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

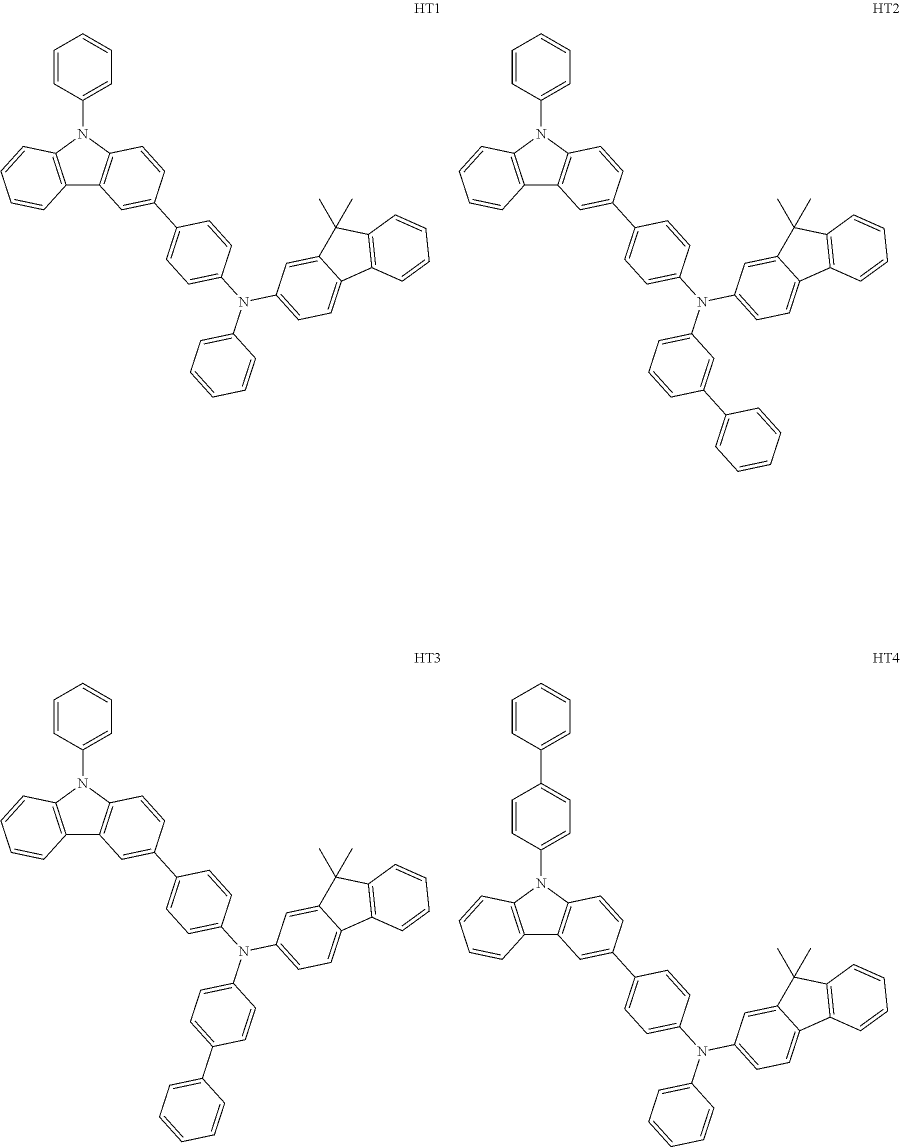

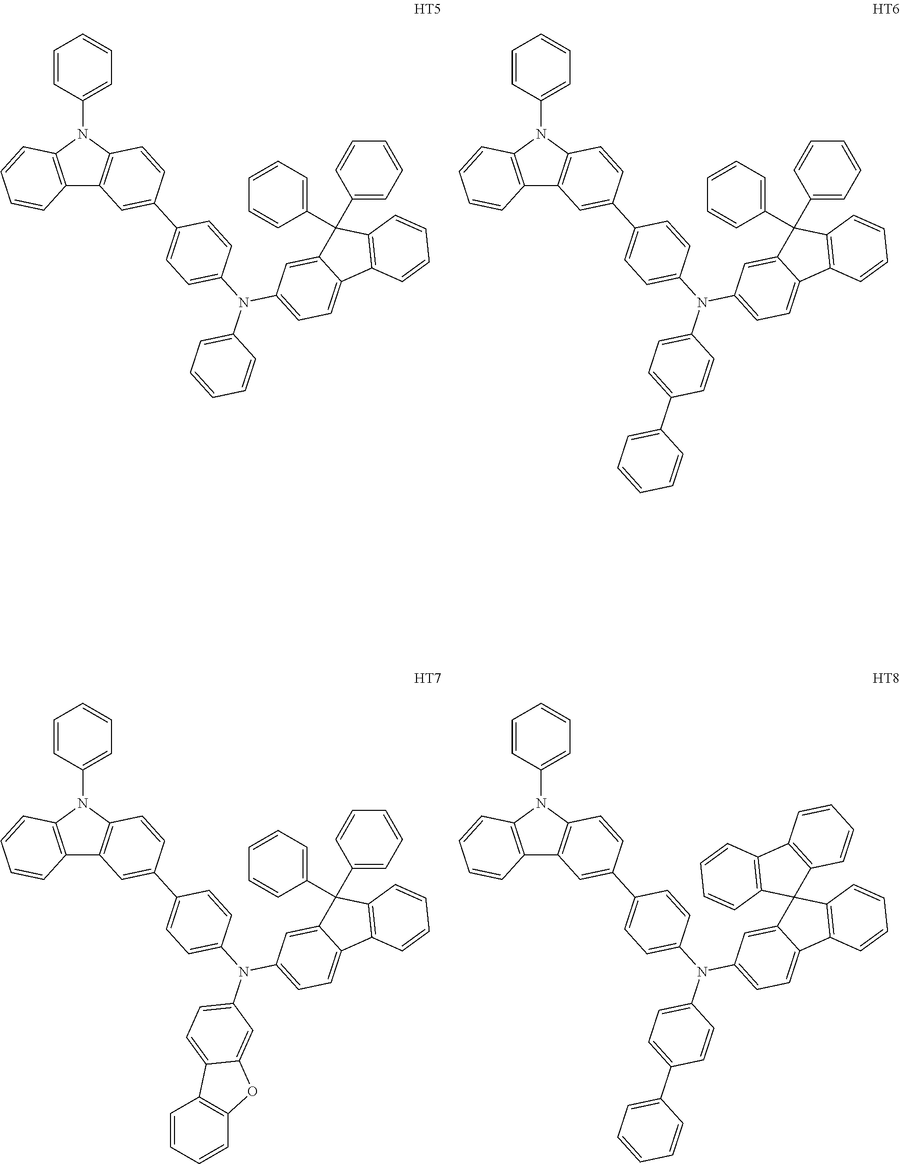 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

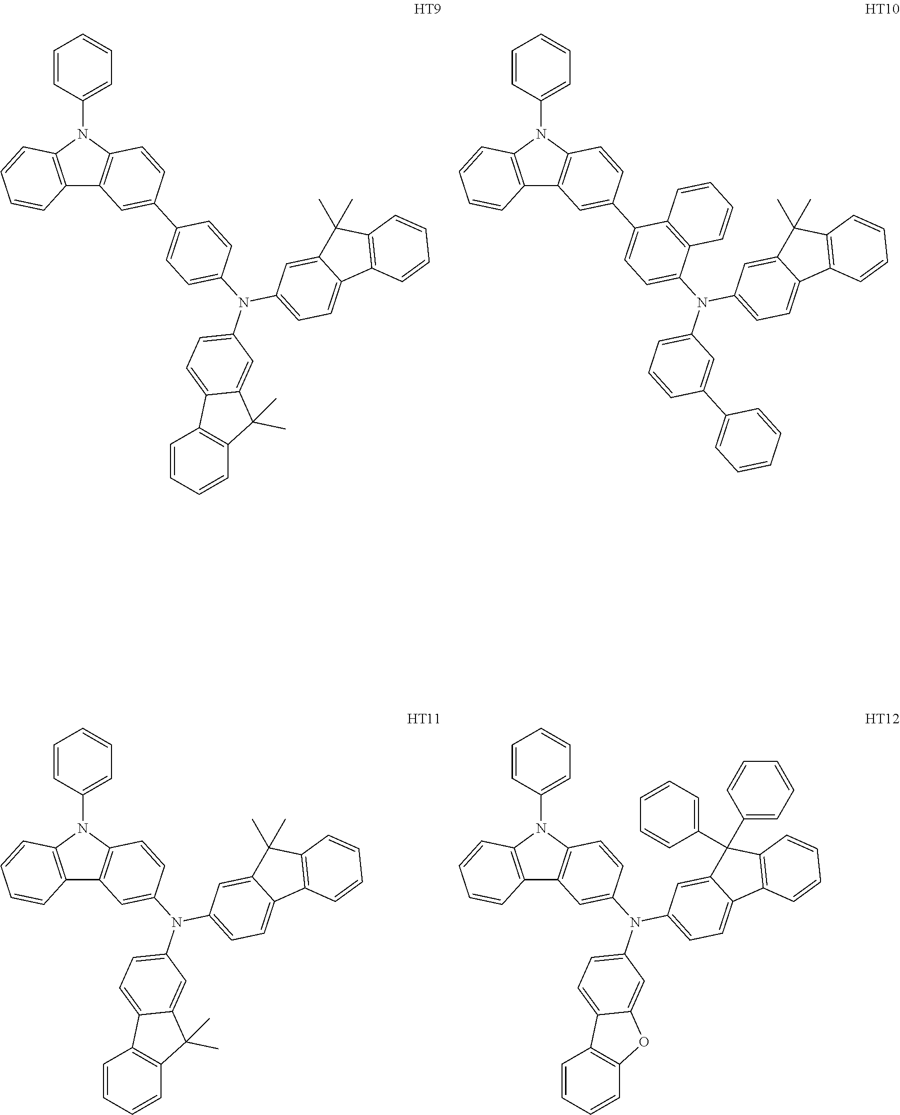 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

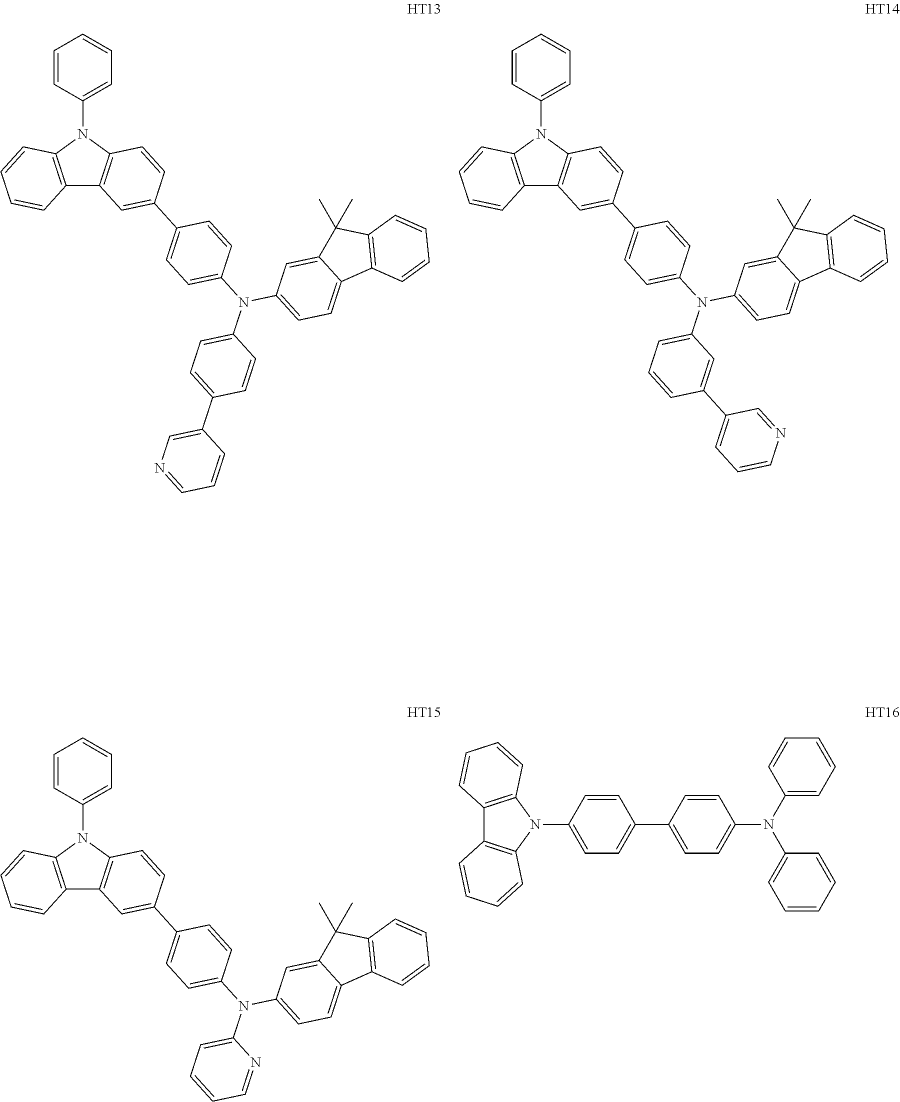 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

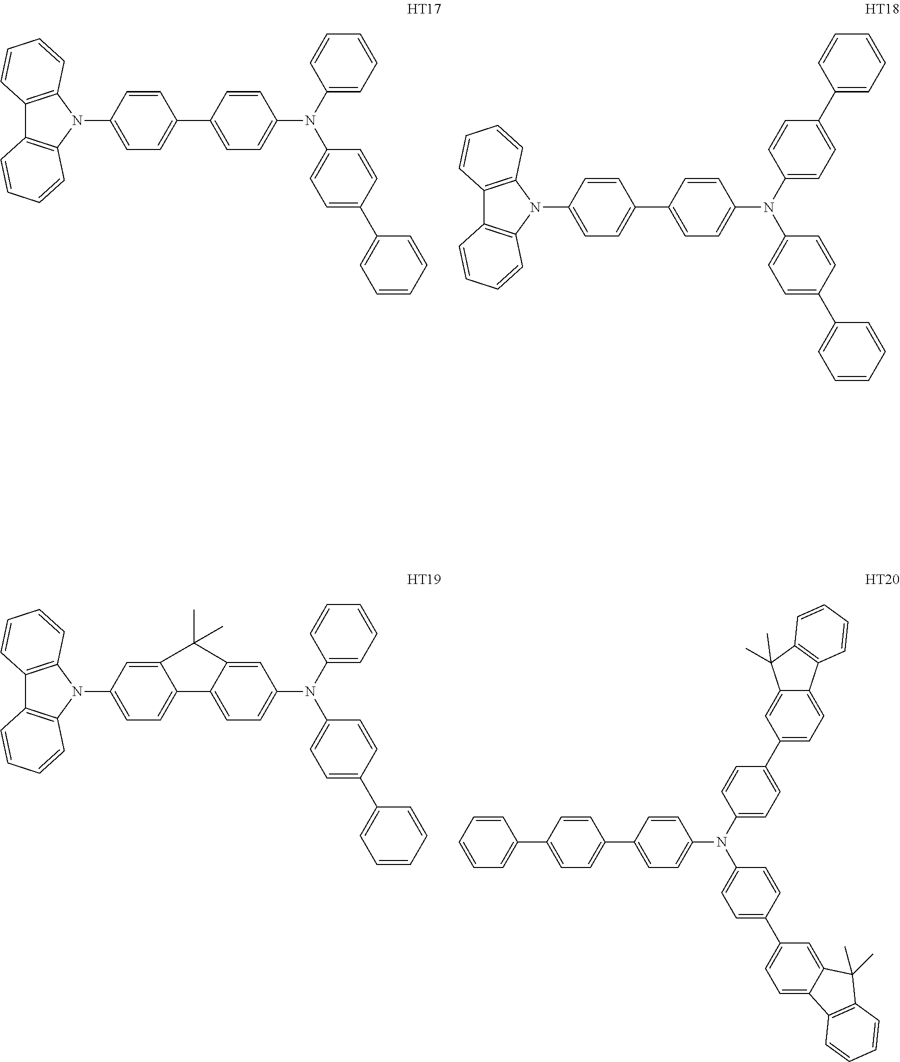

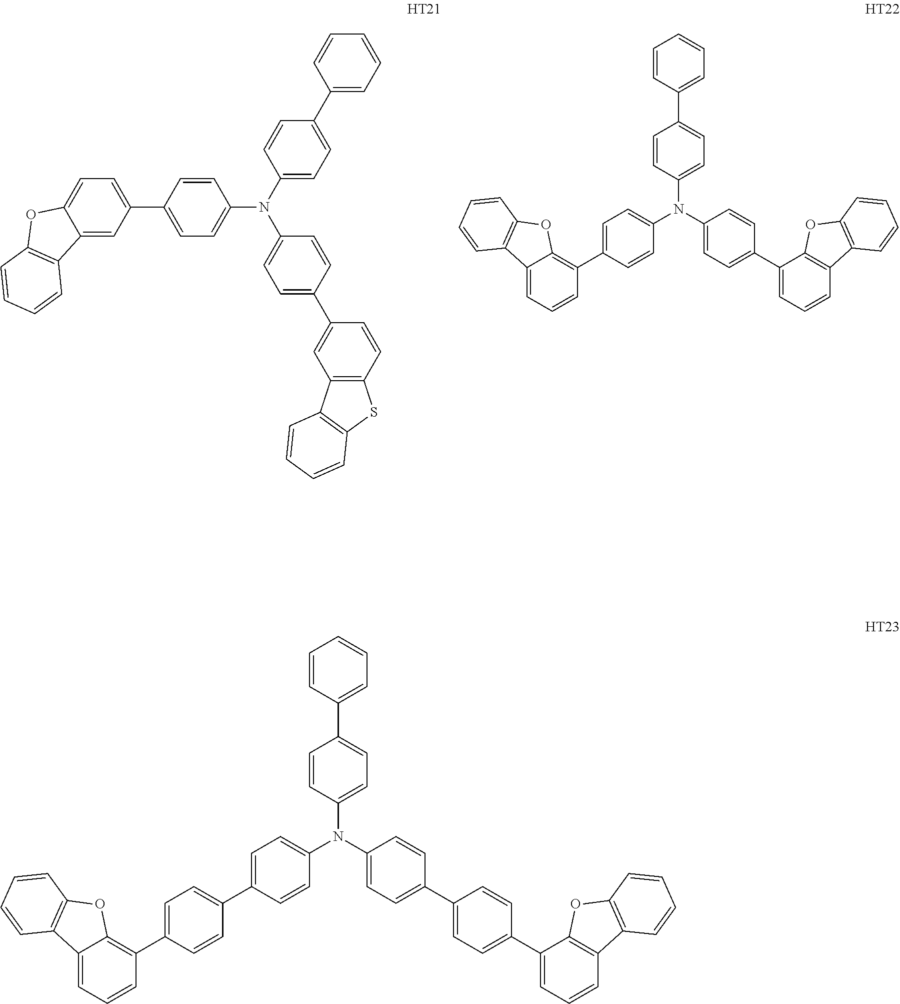 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

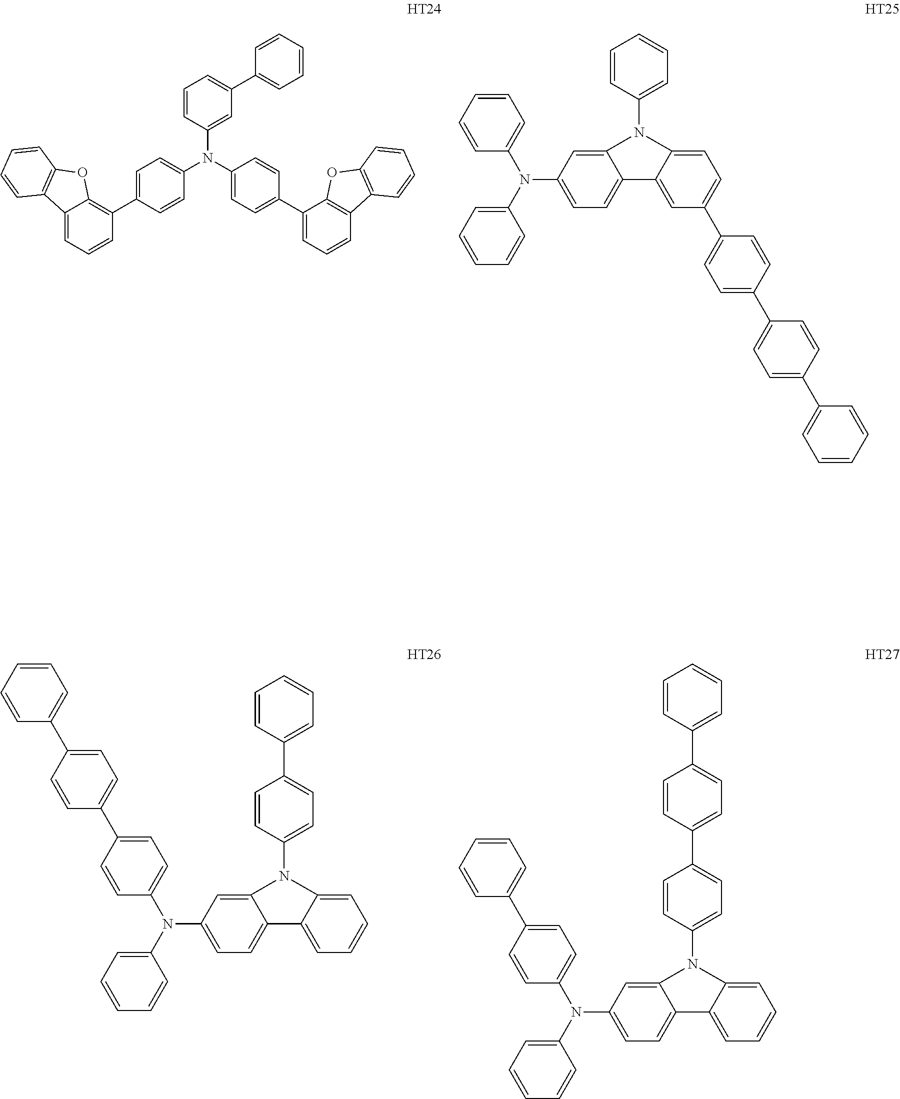 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

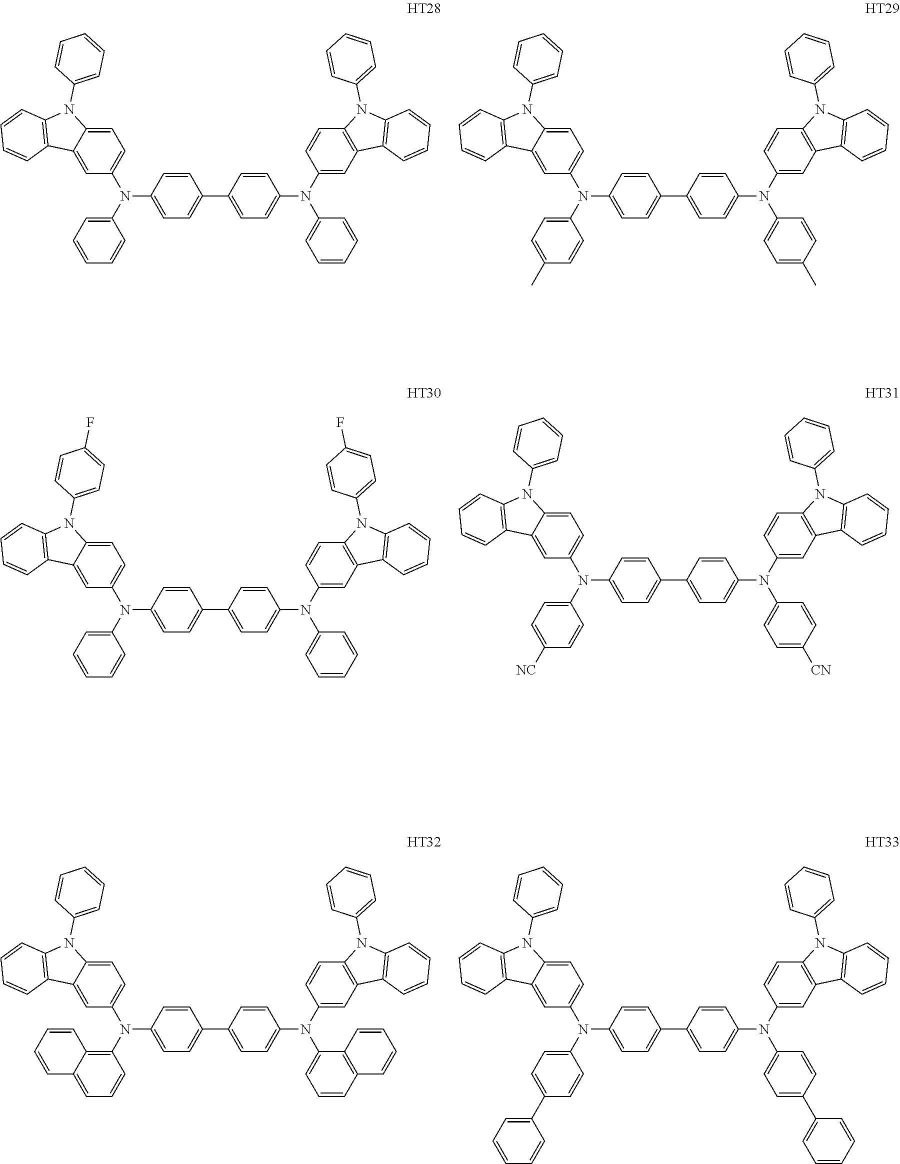

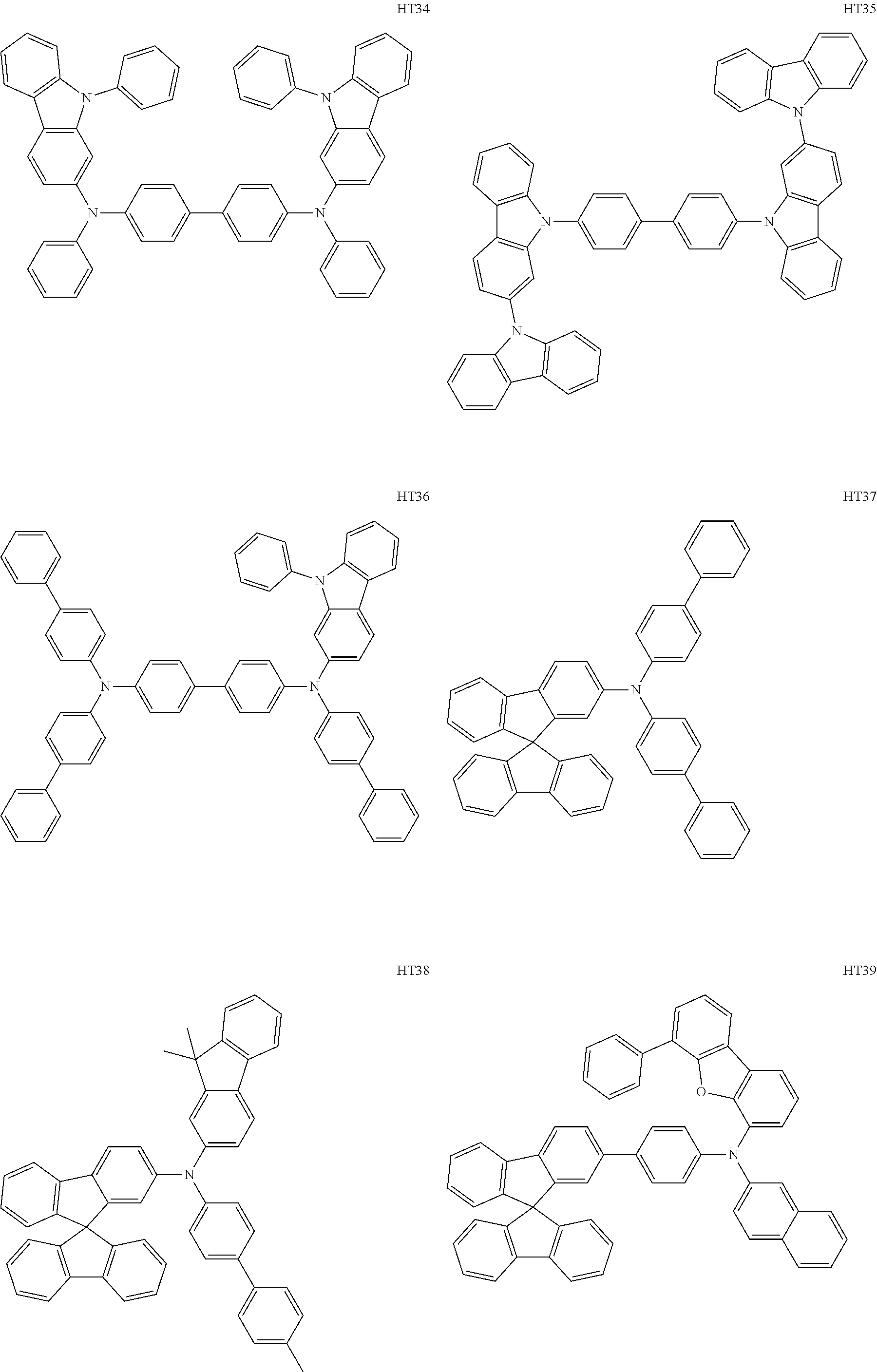

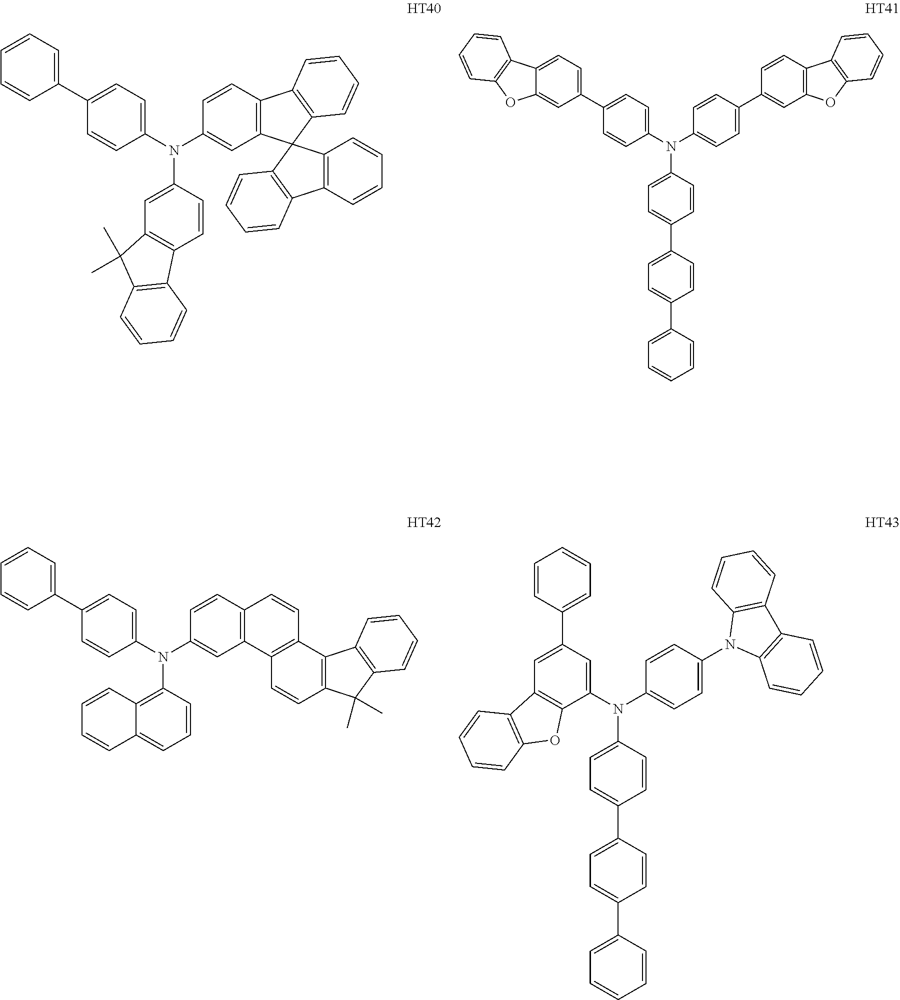 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

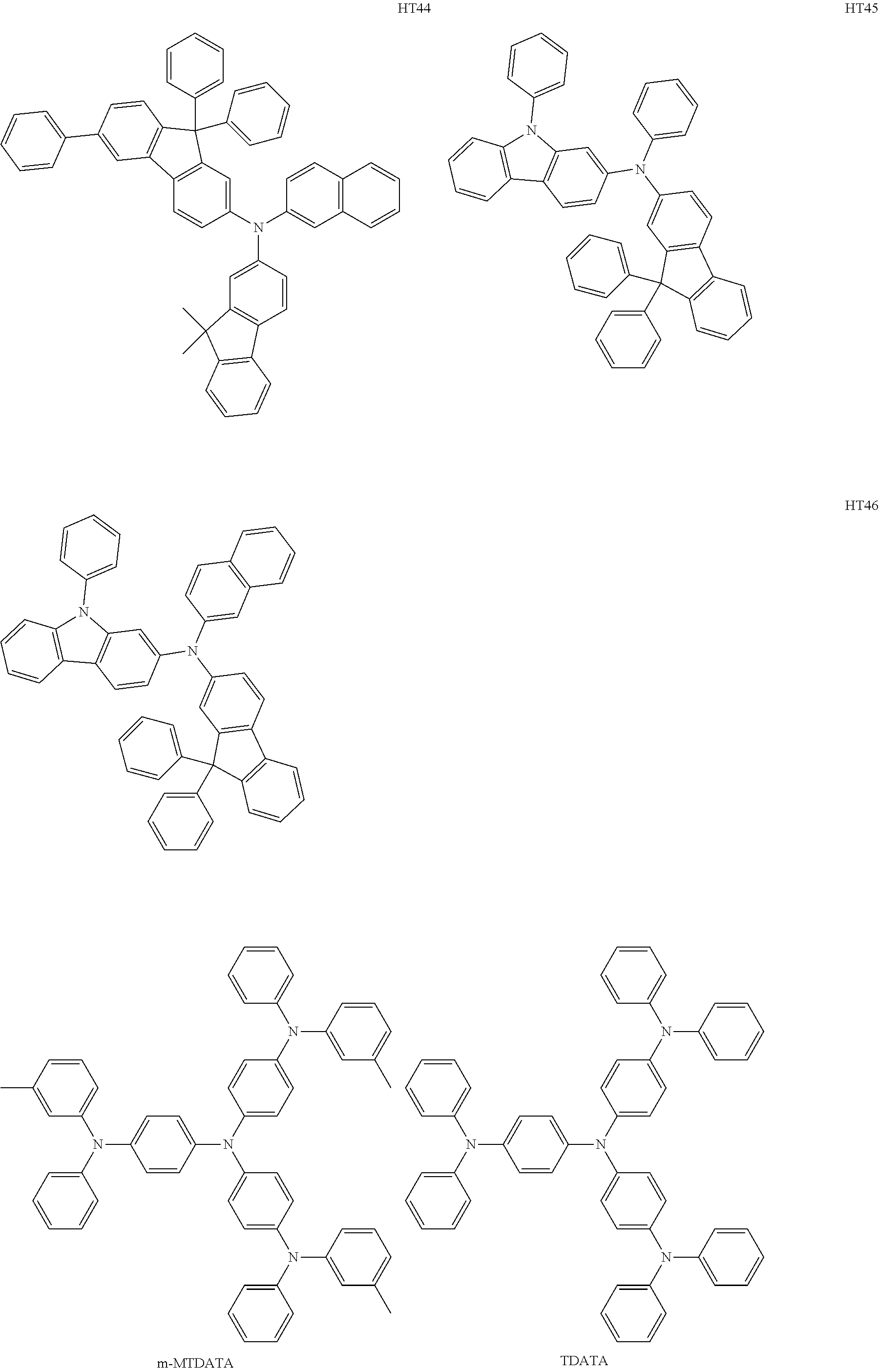

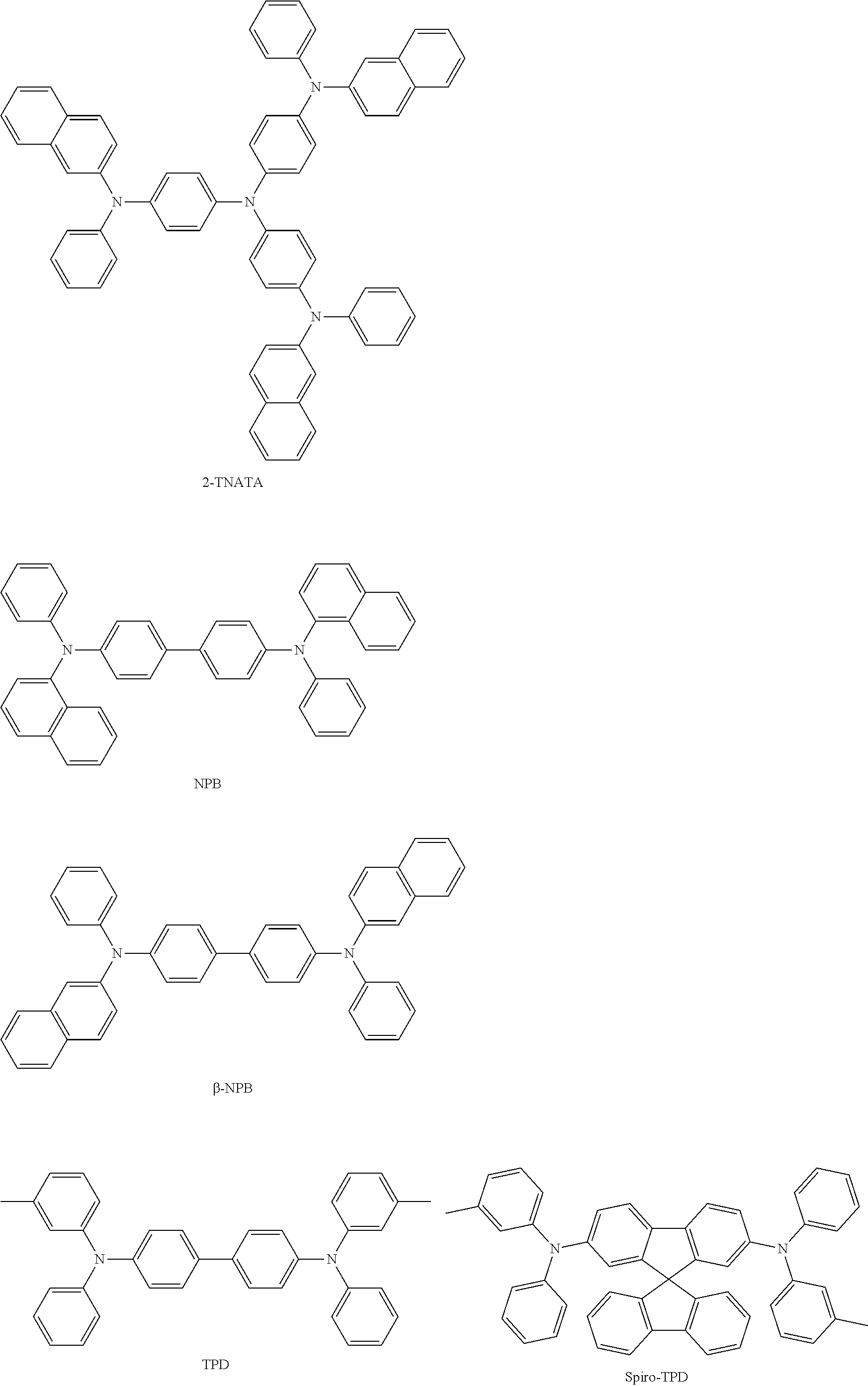

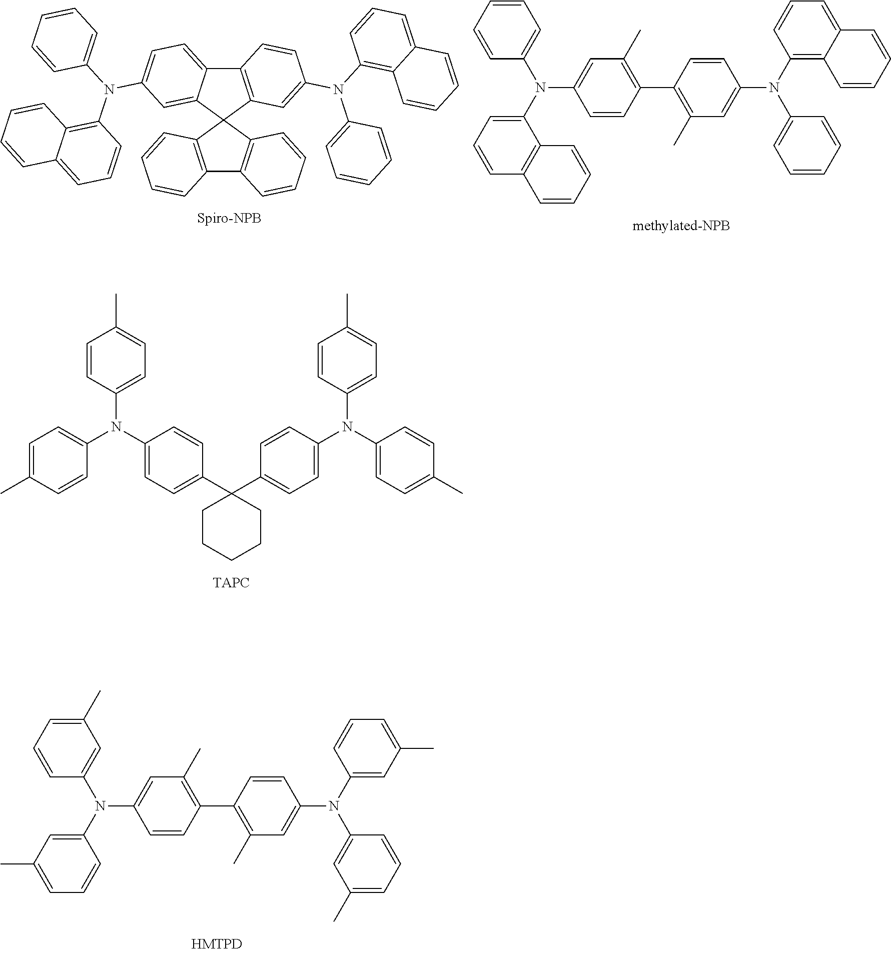

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may block or reduce the leakage of electrons from the emission layer to a hole transport region. Materials that may be included in the hole transport region may be included in the emission auxiliary layer and the electron blocking layer.

p-Dopant

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

For example, the lowest unoccupied molecular orbital (LUMO) energy level of the p-dopant may be −3.5 eV or less.

In one or more embodiments, the p-dopant may include a quinone derivative, a cyano group-containing compound, a compound including element EL1 and element EL2 (to be described in more detail below), or any combination thereof.

Examples of the quinone derivative may include (e.g., may be) TCNQ, F4-TCNQ, etc.

Examples of the cyano group-containing compound may include (e.g., may be) HAT-CN, and a compound represented by Formula 221.

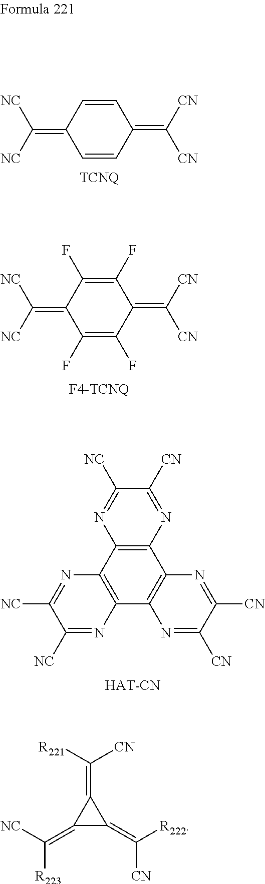

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be a metal, a metalloid, or any combination thereof, and element EL2 may be a non-metal, a metalloid, or any combination thereof.

Examples of the metal may include (e.g., may be) an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid may include (e.g., may be) silicon (Si), antimony (Sb), and tellurium (Te).

Examples of the non-metal may include (e.g., may be) oxygen (O) and halogen (for example, F, Cl, Br, I, etc.).

Examples of the compound including element EL1 and element EL2 may include (e.g., may be) a metal oxide, a metal halide (for example, a metal fluoride, a metal chloride, a metal bromide, and/or a metal iodide), a metalloid halide (for example, a metalloid fluoride, a metalloid chloride, a metalloid bromide, and/or a metalloid iodide), a metal telluride, or any combination thereof.

Examples of the metal oxide may include (e.g., may be) tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide may include (e.g., may be) an alkali metal halide, an alkaline earth metal halide, a transition metal halide, a post-transition metal halide, and a lanthanide metal halide.

Examples of the alkali metal halide may include (e.g., may be) LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include (e.g., may be) BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include (e.g., may be) titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, COCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and gold halide (for example, AuF, AuCl, AuBr, AuI, etc.).

Examples of the post-transition metal halide may include (e.g., may be) zinc halide (for example, ZnF₂, ZnCl₂, ZnBr₂, ZnI₂, etc.), indium halide (for example, InI₃, etc.), and tin halide (for example, SnI₂, etc.).

Examples of the lanthanide metal halide may include (e.g., may be) YbF, YbF₂, YbF₃, SmF₃, YbCl, YbCl₂, YbCl₃ SmCl₃, YbBr, YbBr₂, YbBr₃ SmBr₃, YbI, YbI₂, YbI₃, and SmI₃.

An example of the metalloid halide may include (e.g., may be) antimony halide (for example, SbCl₅, etc.).

Examples of the metal telluride may include (e.g., may be) an alkali metal telluride (for example, Li₂Te, Na₂Te, K₂Te, Rb₂Te, Cs₂Te, etc.), an alkaline earth metal telluride (for example, BeTe, MgTe, CaTe, SrTe, BaTe, etc.), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), a post-transition metal telluride (for example, ZnTe, etc.), and a lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other to emit white light. In one or more embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescence dopant, or any combination thereof.

The amount of the dopant in the emission layer may be from about 0.01 part by weight to about 15 parts by weight based on 100 parts by weight of the host.

In one or more embodiments, the emission layer may include a quantum dot.

In some embodiments, the emission layer may include a delayed fluorescence material. The delayed fluorescence material may act (e.g., serve) as a host or a dopant in the emission layer.

A thickness of the emission layer may be in a range of about 100 Å to about 1,000 Å, for example, about 200 Å to about 600 Å. When the thickness of the emission layer is within these ranges, excellent or suitable light-emission characteristics may be obtained without a substantial increase in driving voltage.

### Host

In one or more embodiments, the host may include a compound represented by Formula 301:

[Ar₃₀₁]_{xb11}-[(L₃₀₁)_{xb1}-R₃₀₁]_{xb21} Formula 301

In Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ may each independently be the same as described herein with respect to Q₁.

For example, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

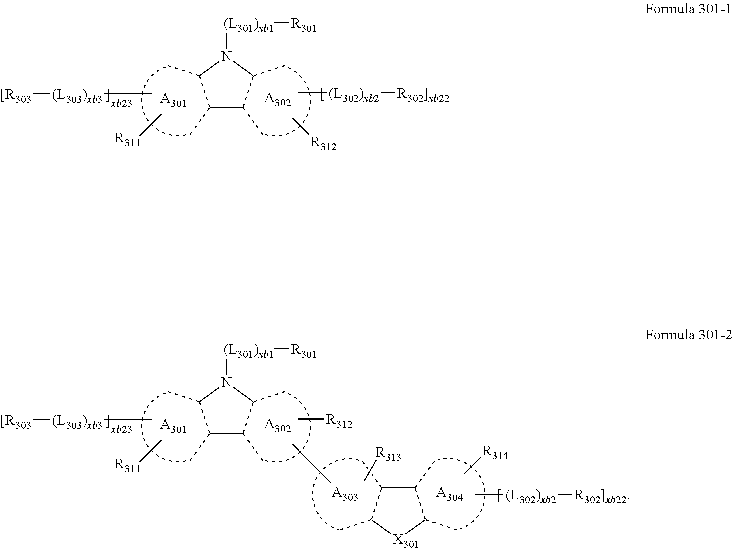

In Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each be the same as described herein,

L₃₀₂ to L₃₀₄ may each independently be the same as described herein with respect to with L₃₀₁,

xb2 to xb4 may each independently be the same as described herein with respect to xb1, and

R₃₀₂ to R₃₀₅ and R₃₁₁ to R₃₁₄ may each independently be the same as described herein with respect to R₃₀₁.

In one or more embodiments, the host may include an alkaline earth metal complex, a post-transition metal complex, or any combination thereof. For example, the host may include a Be complex (for example, Compound H55), an Mg complex, a Zn complex, or any combination thereof.

In an embodiment, the host may include at least one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

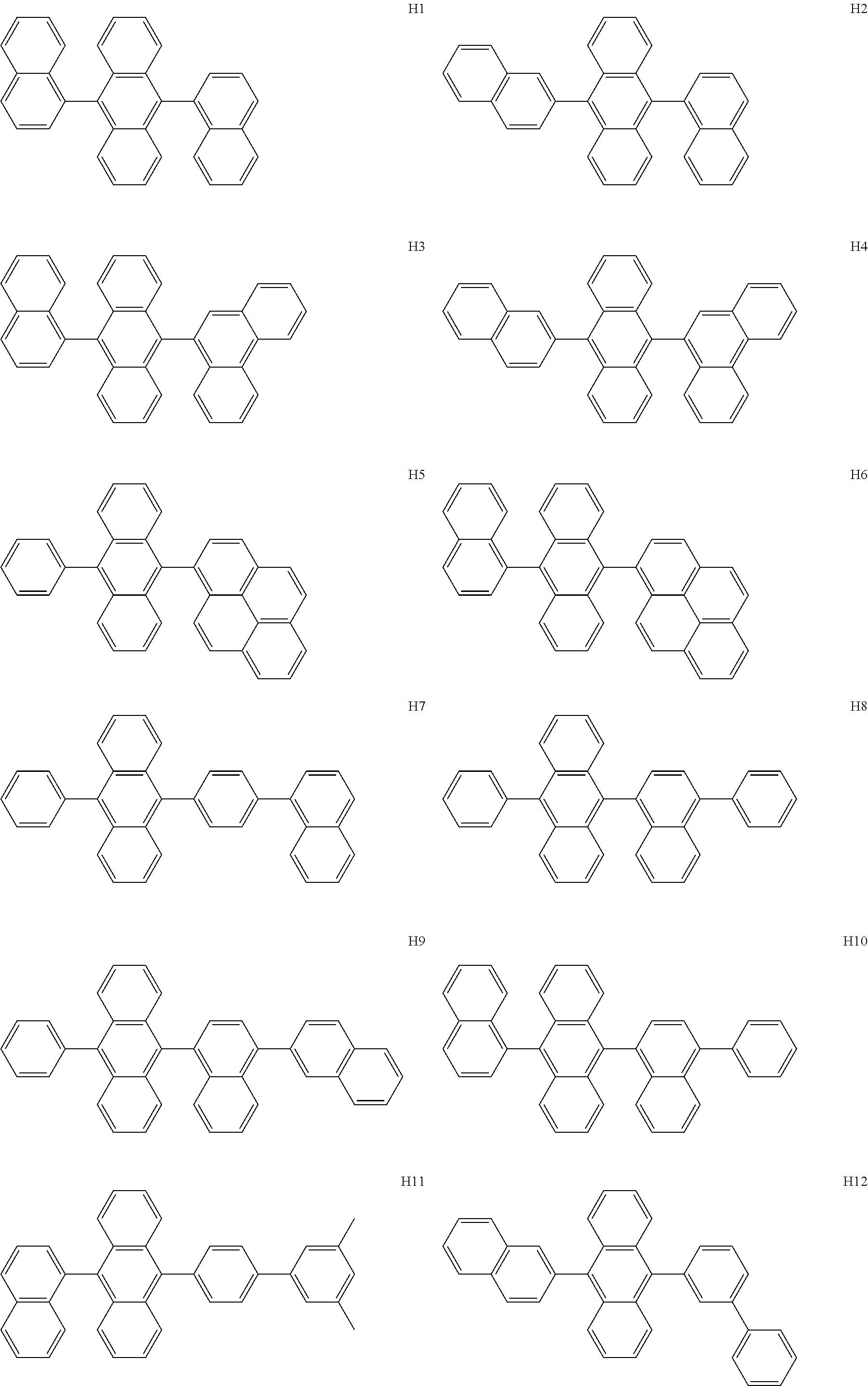

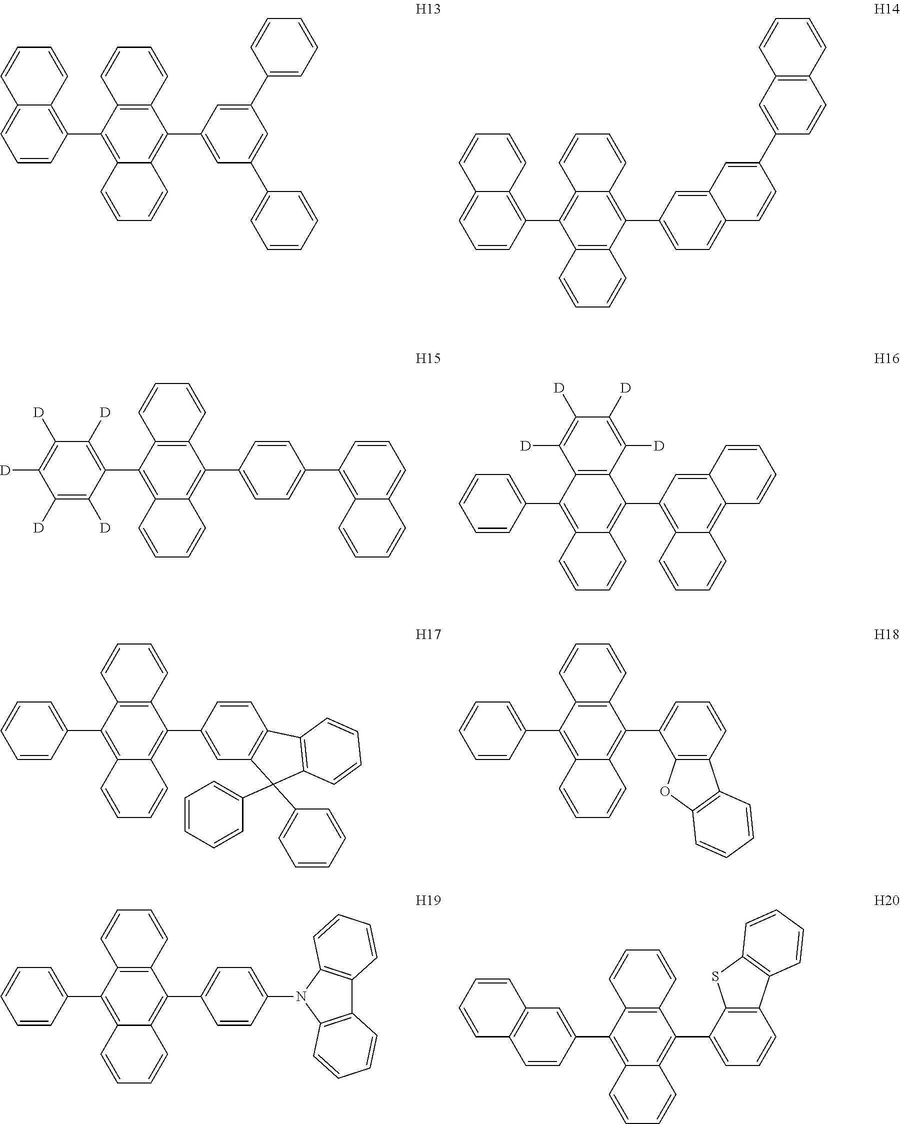

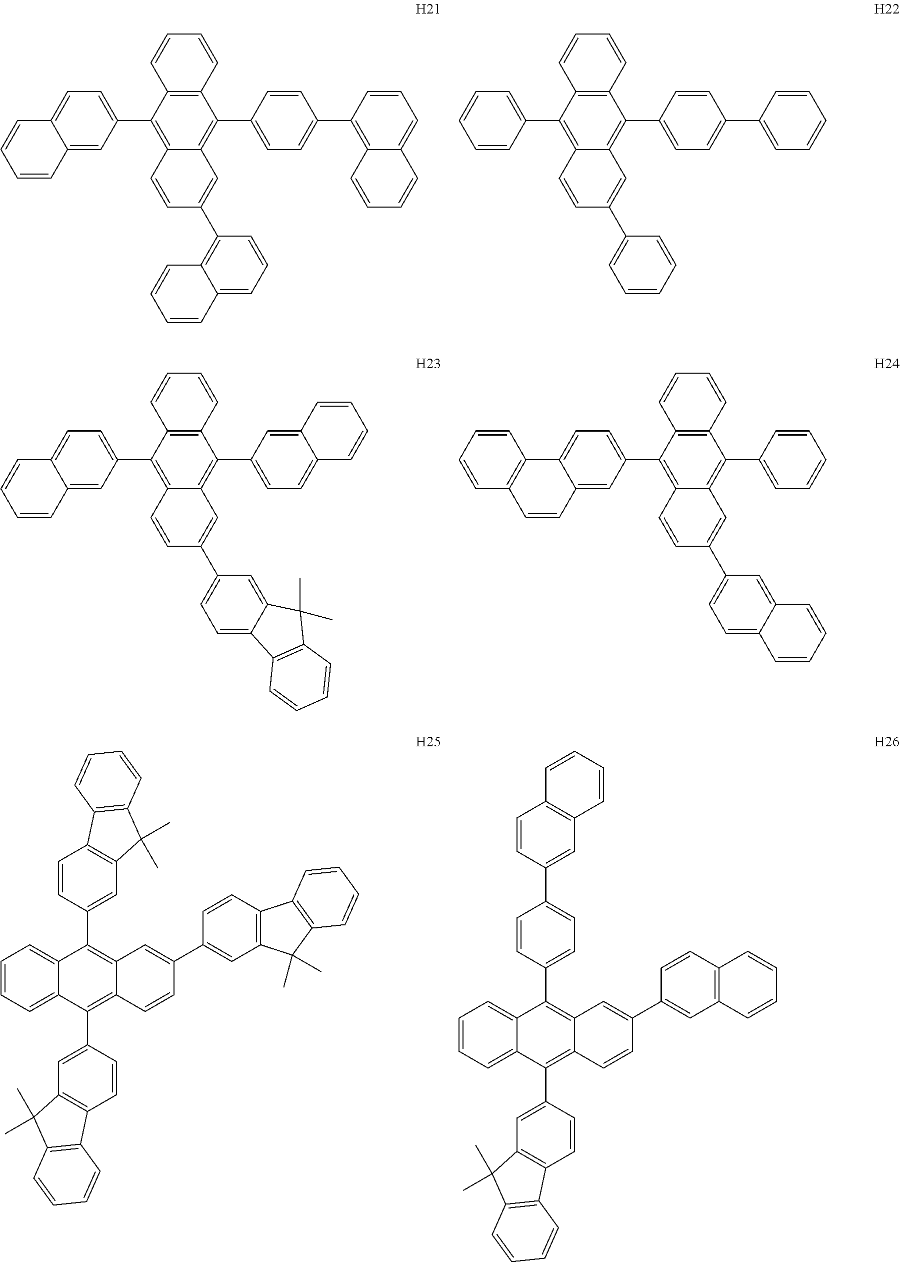

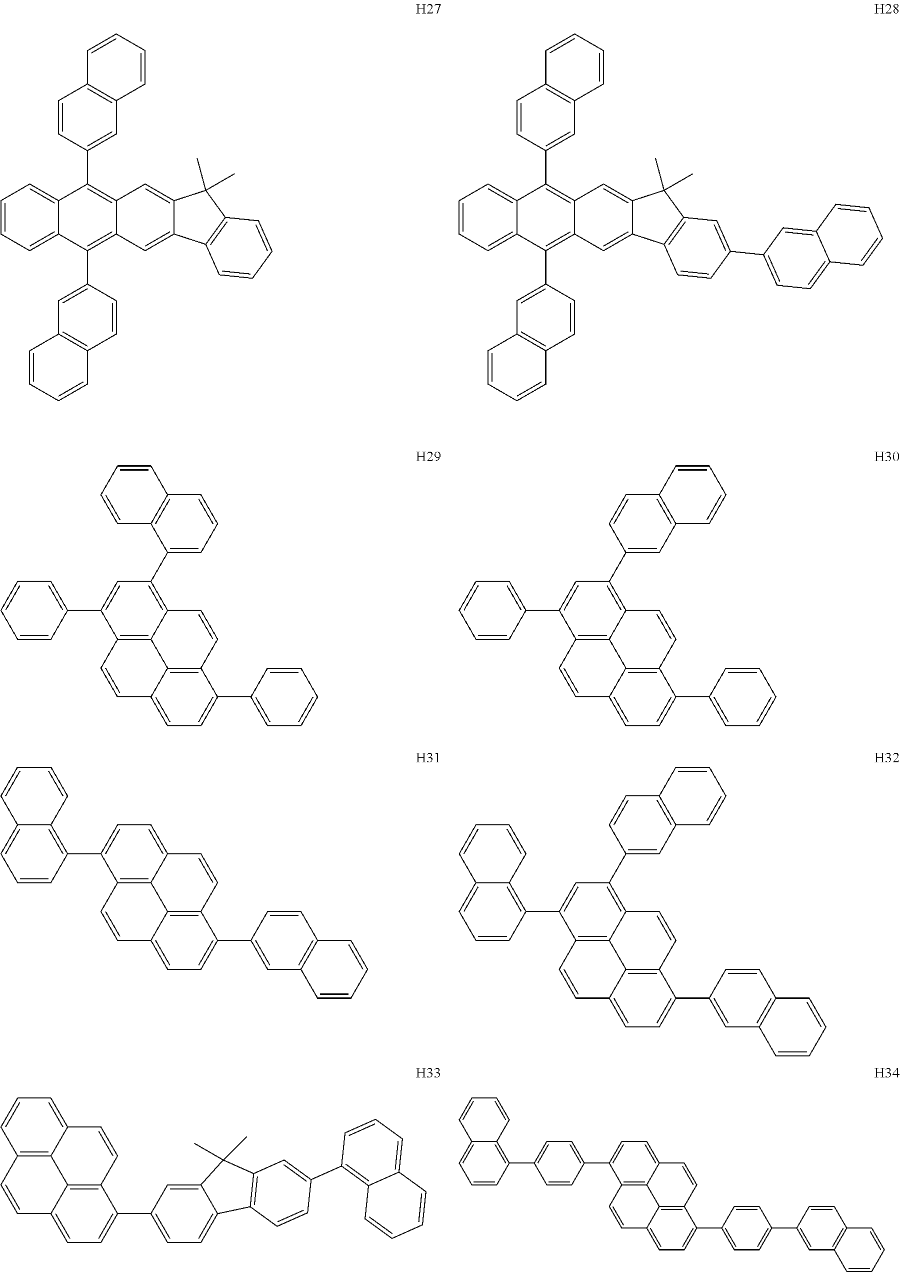

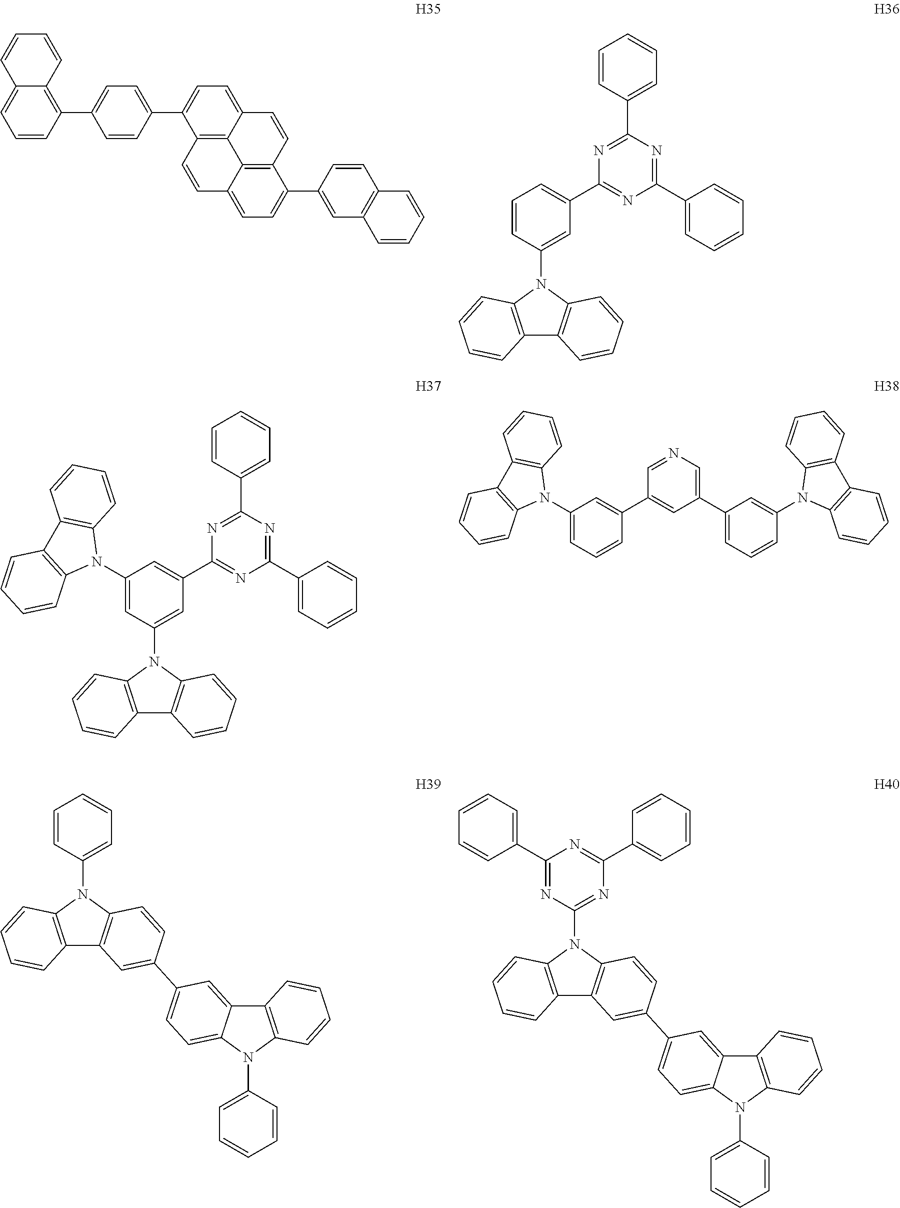

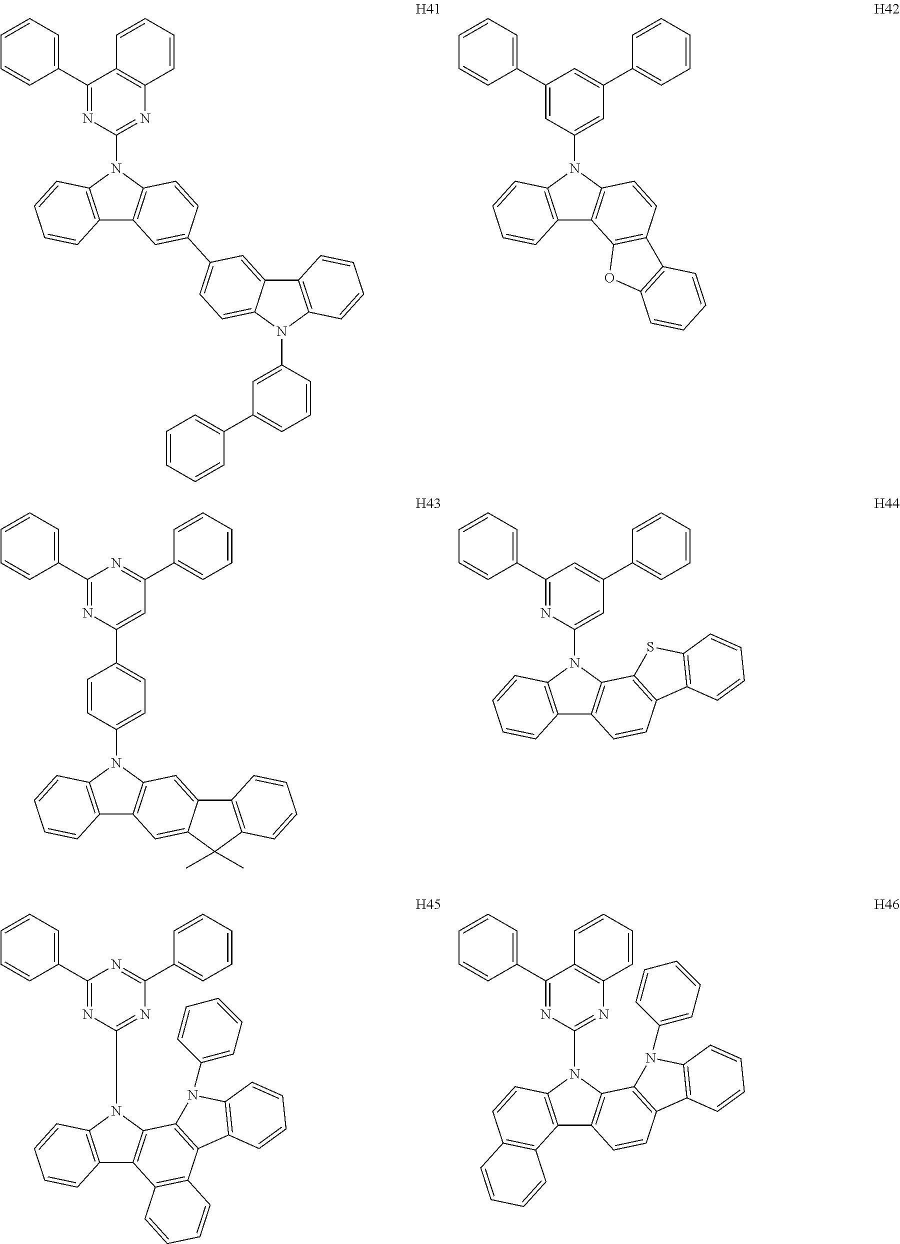

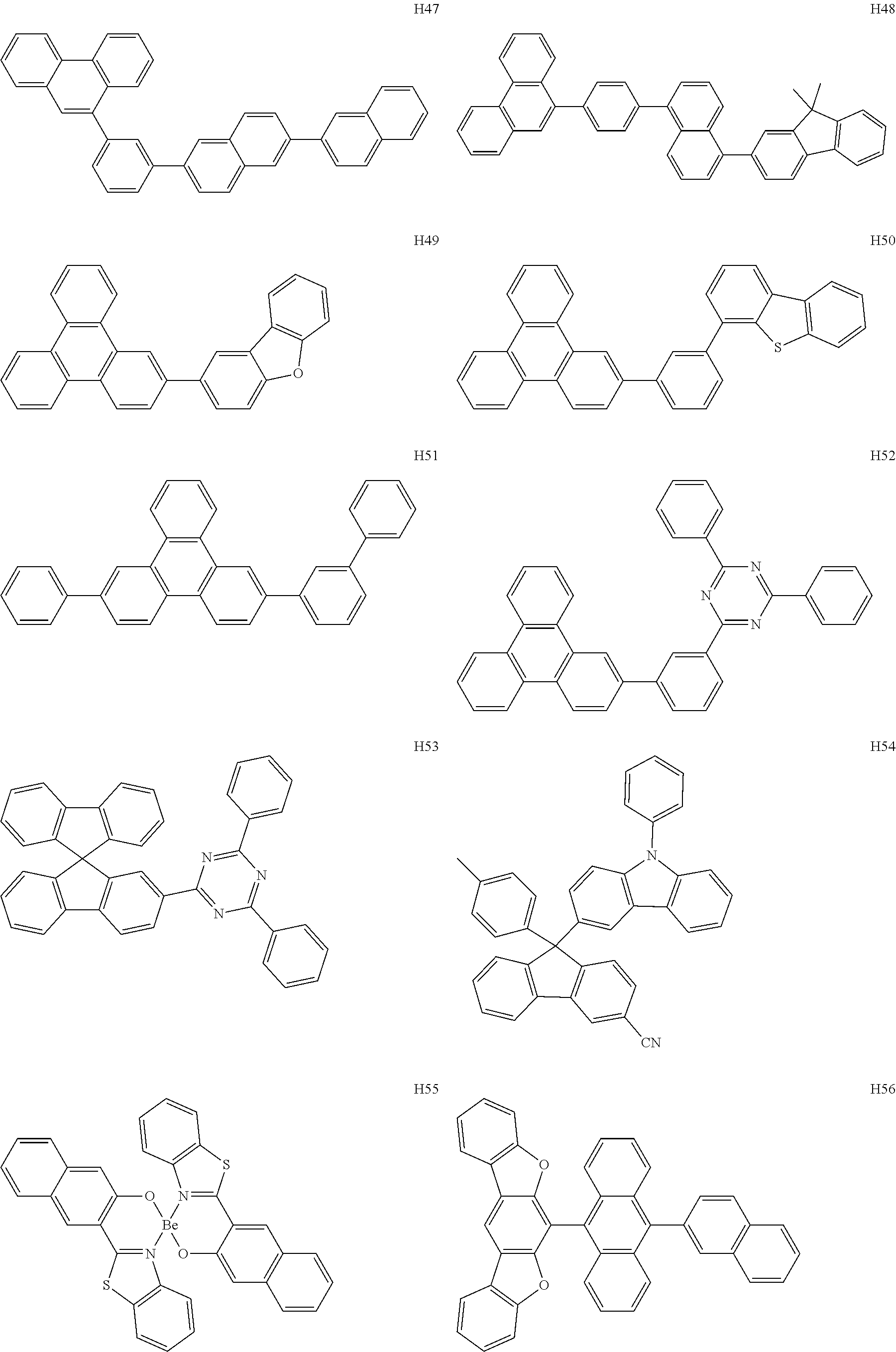

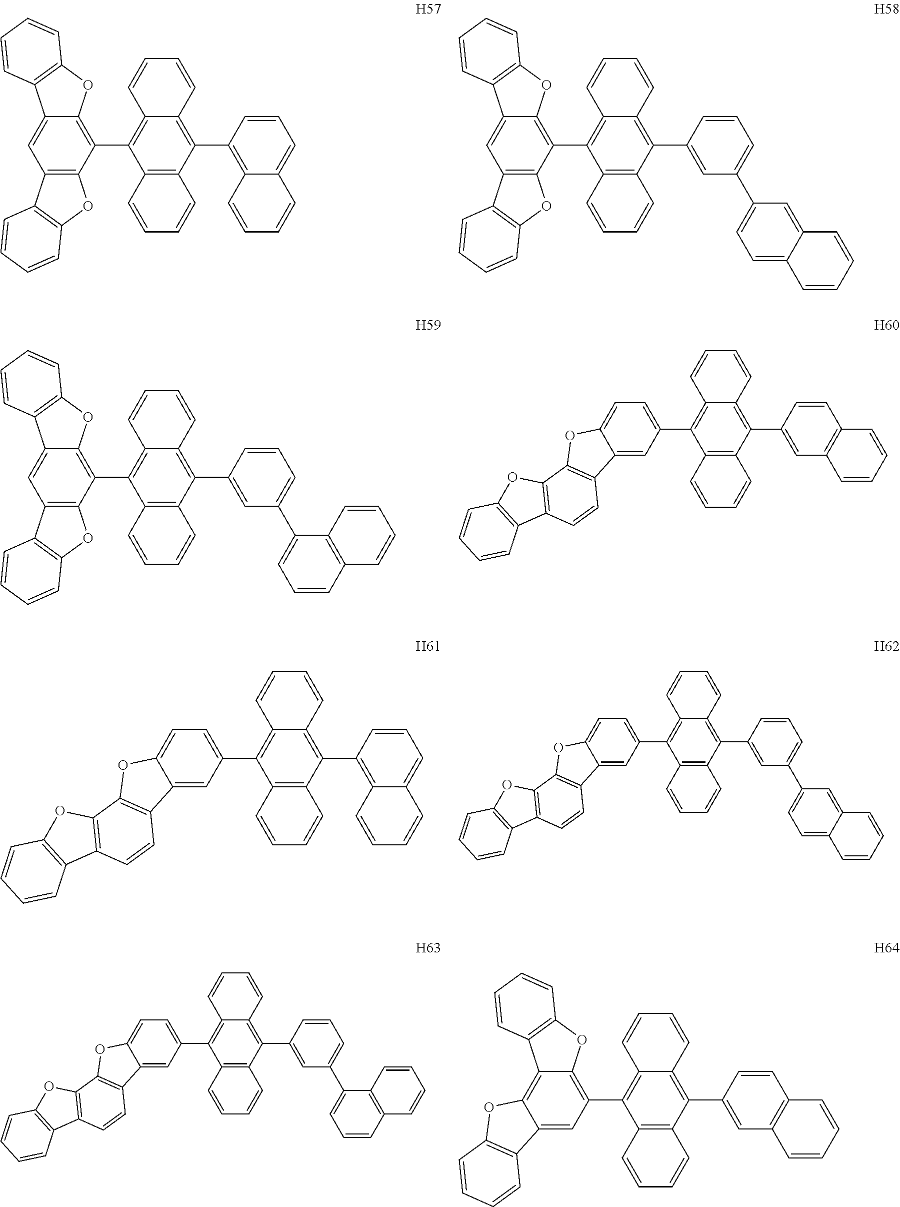

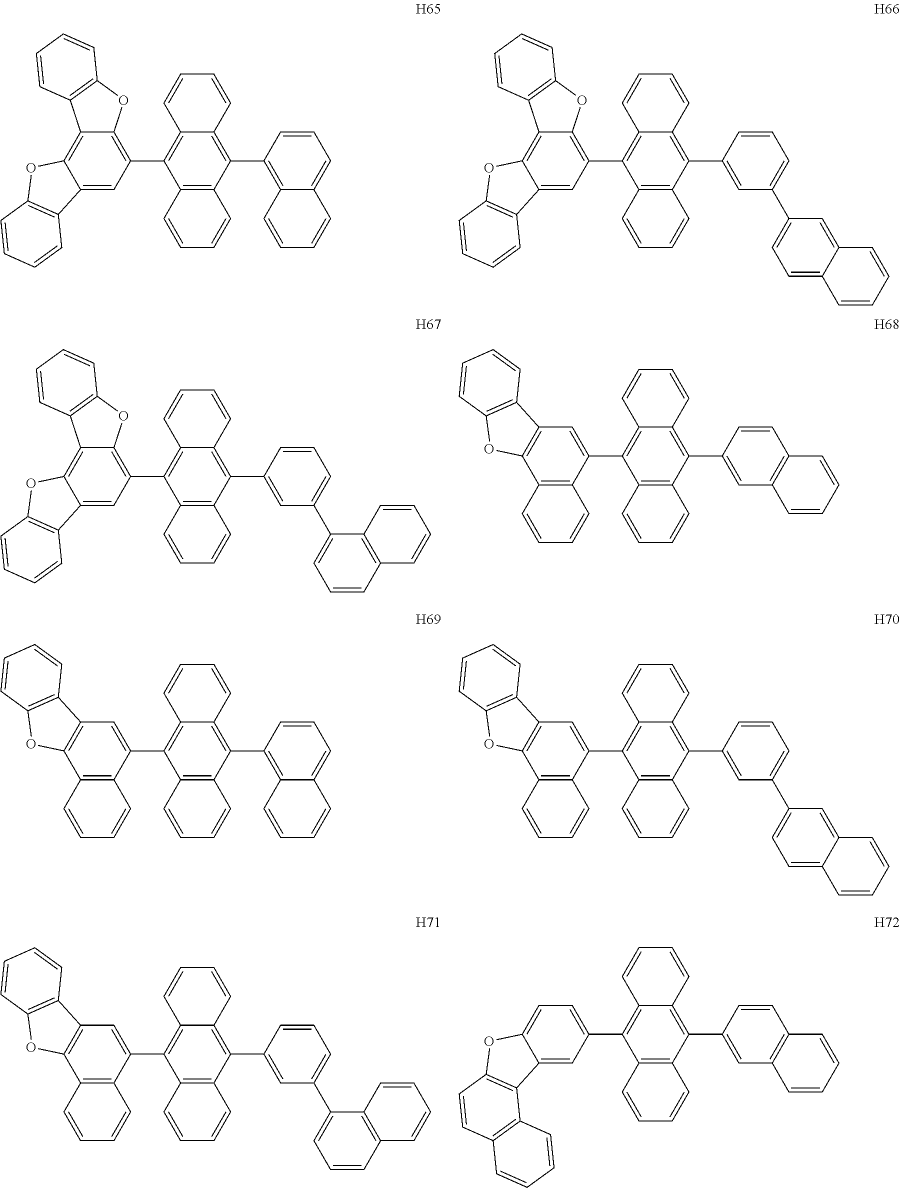

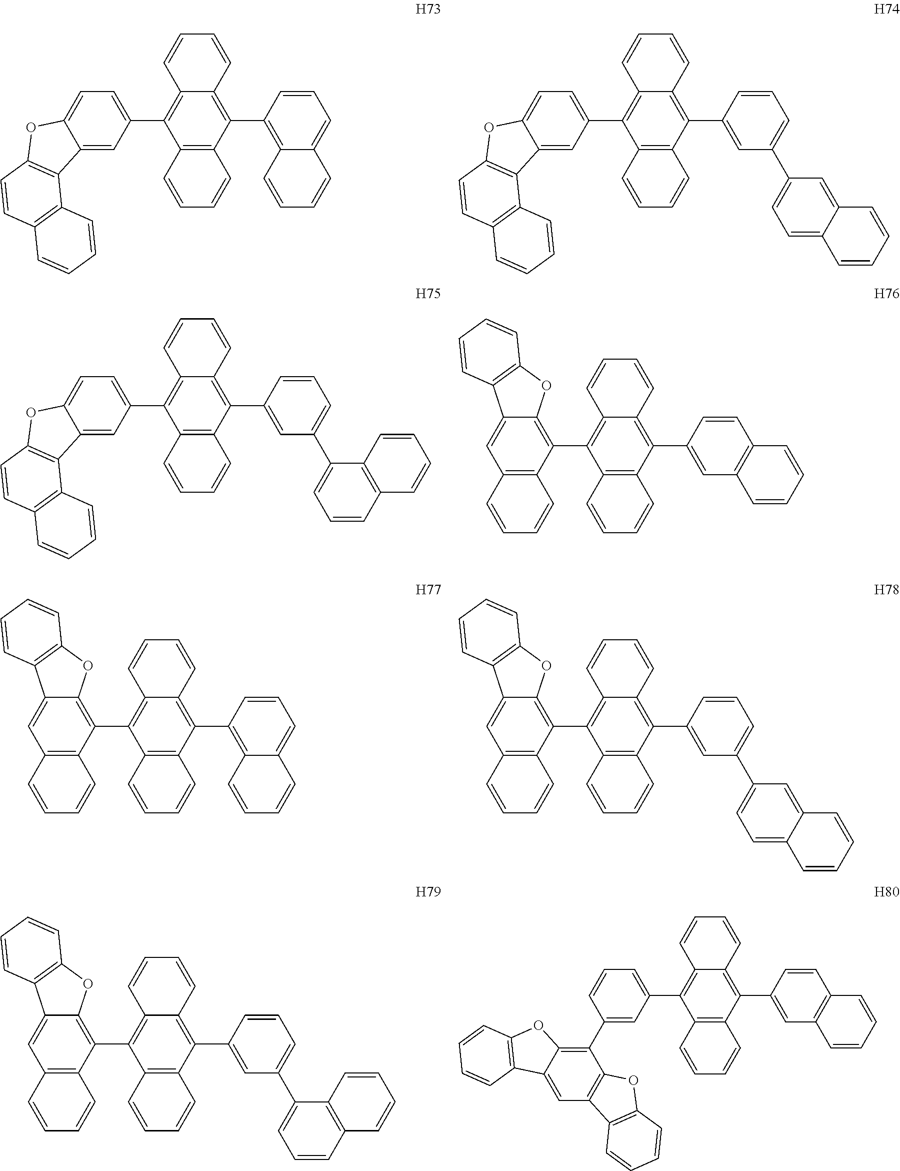

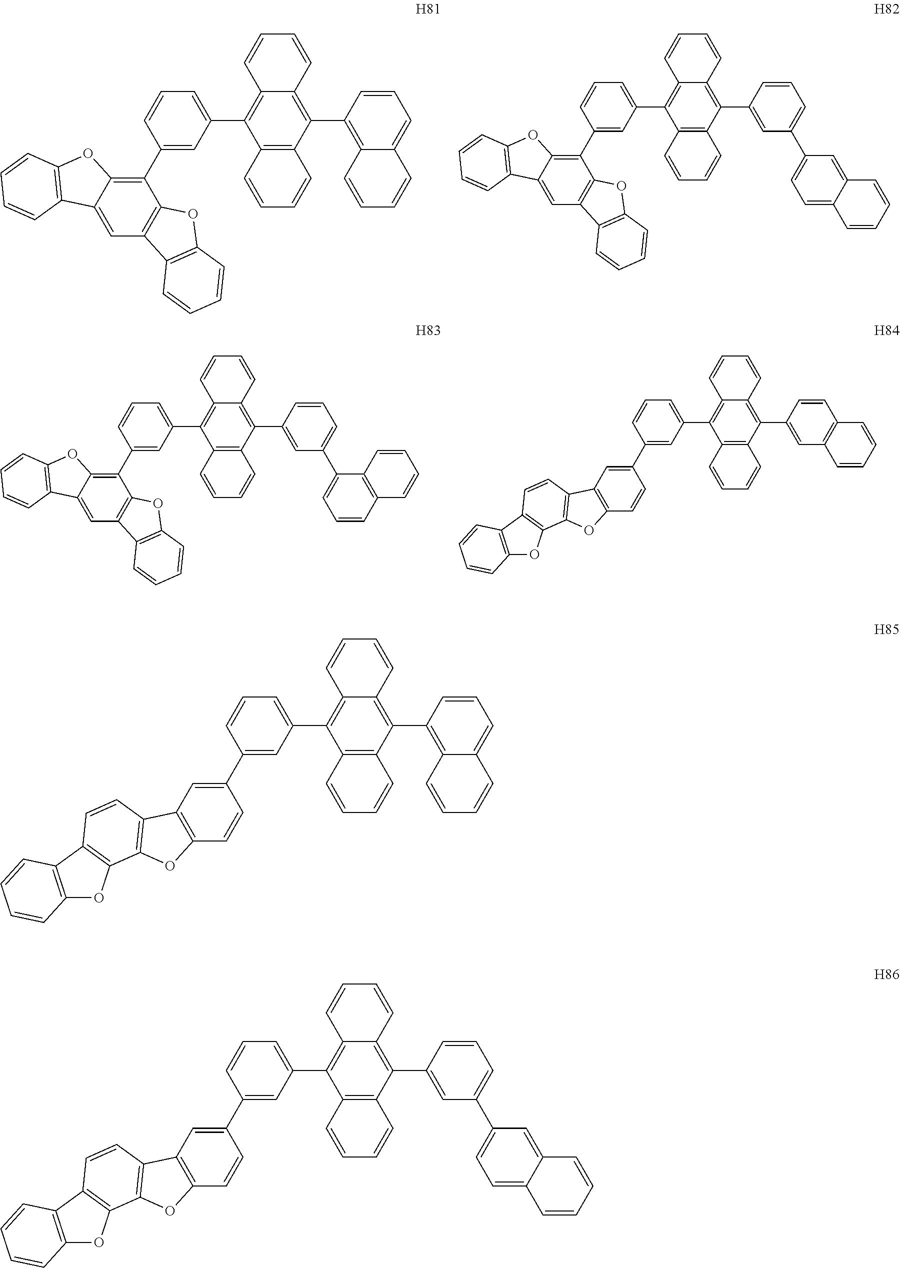

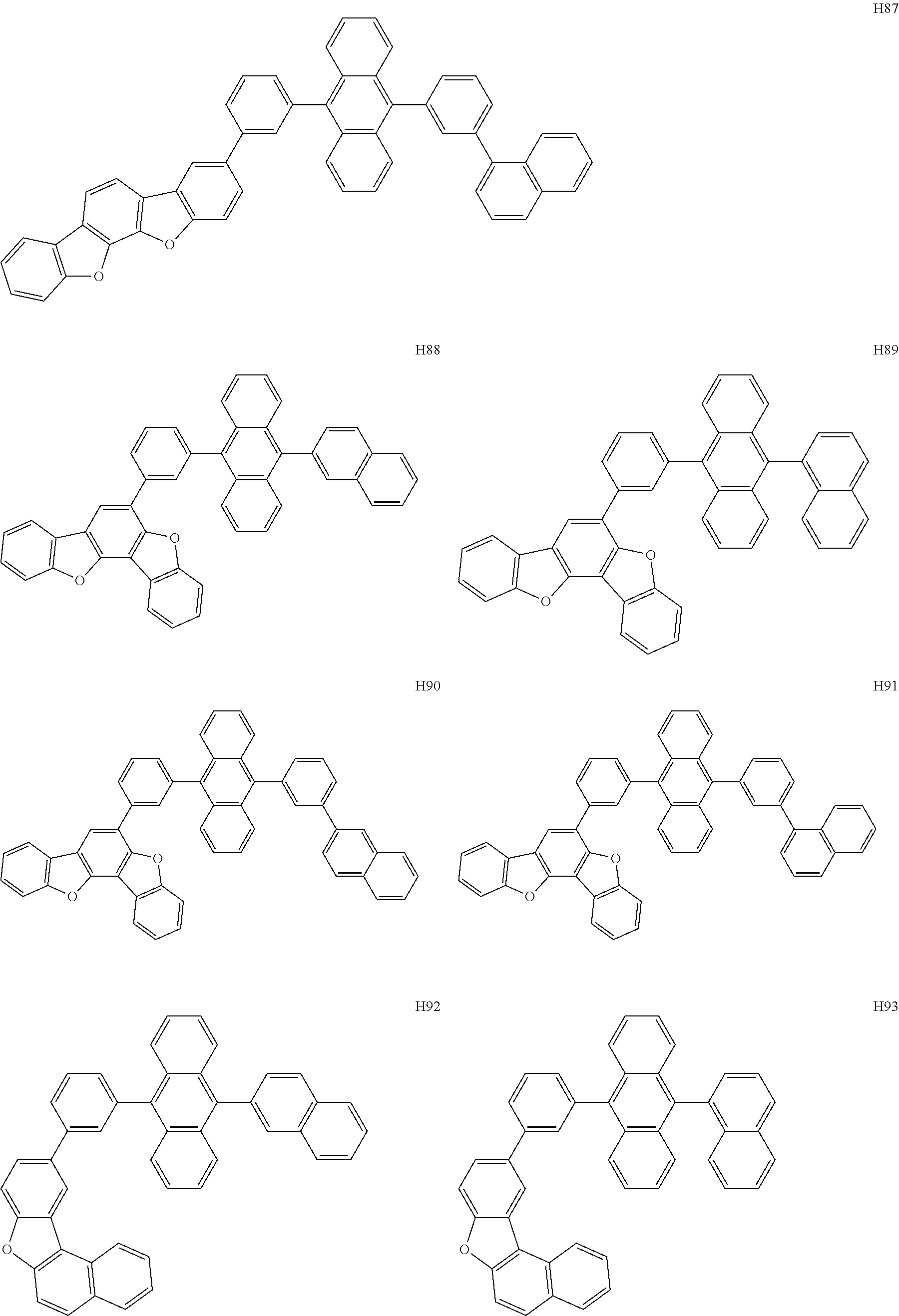

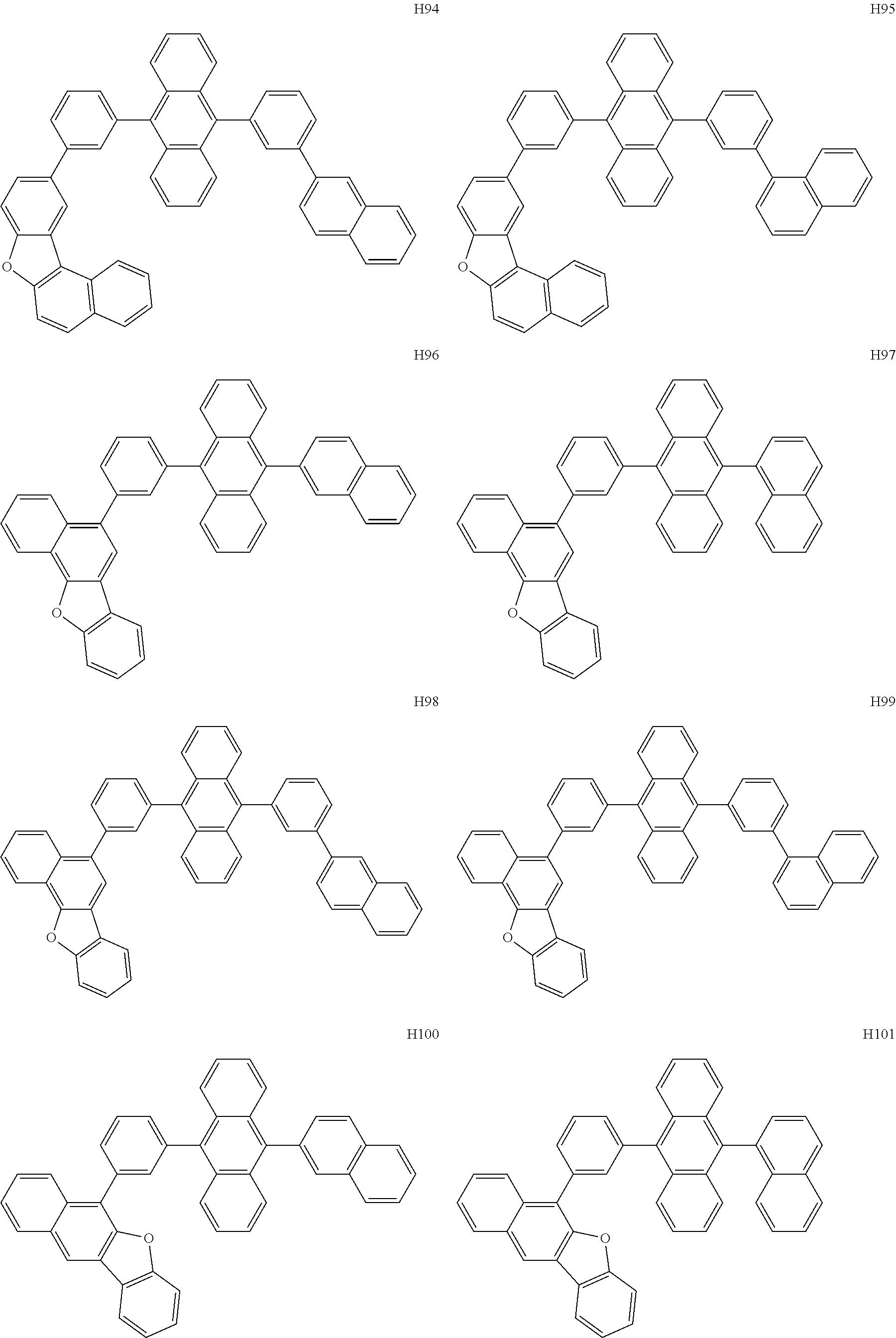

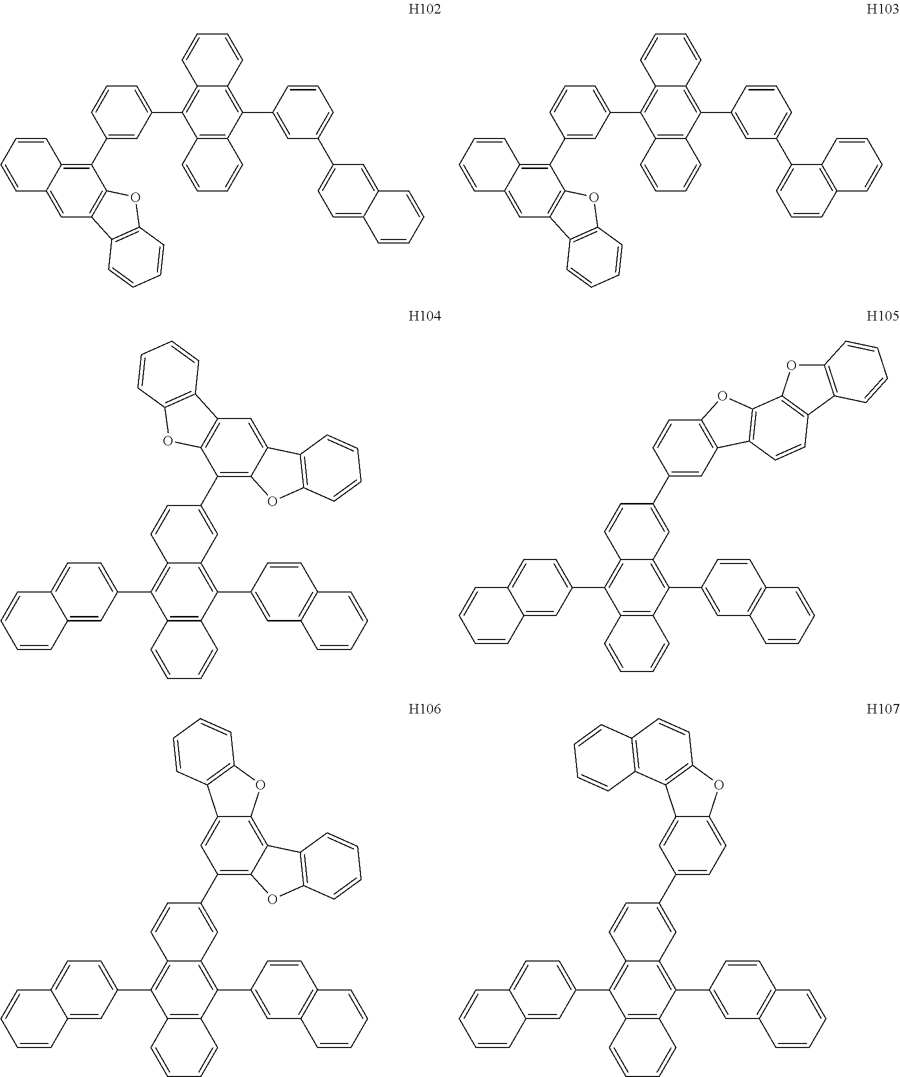

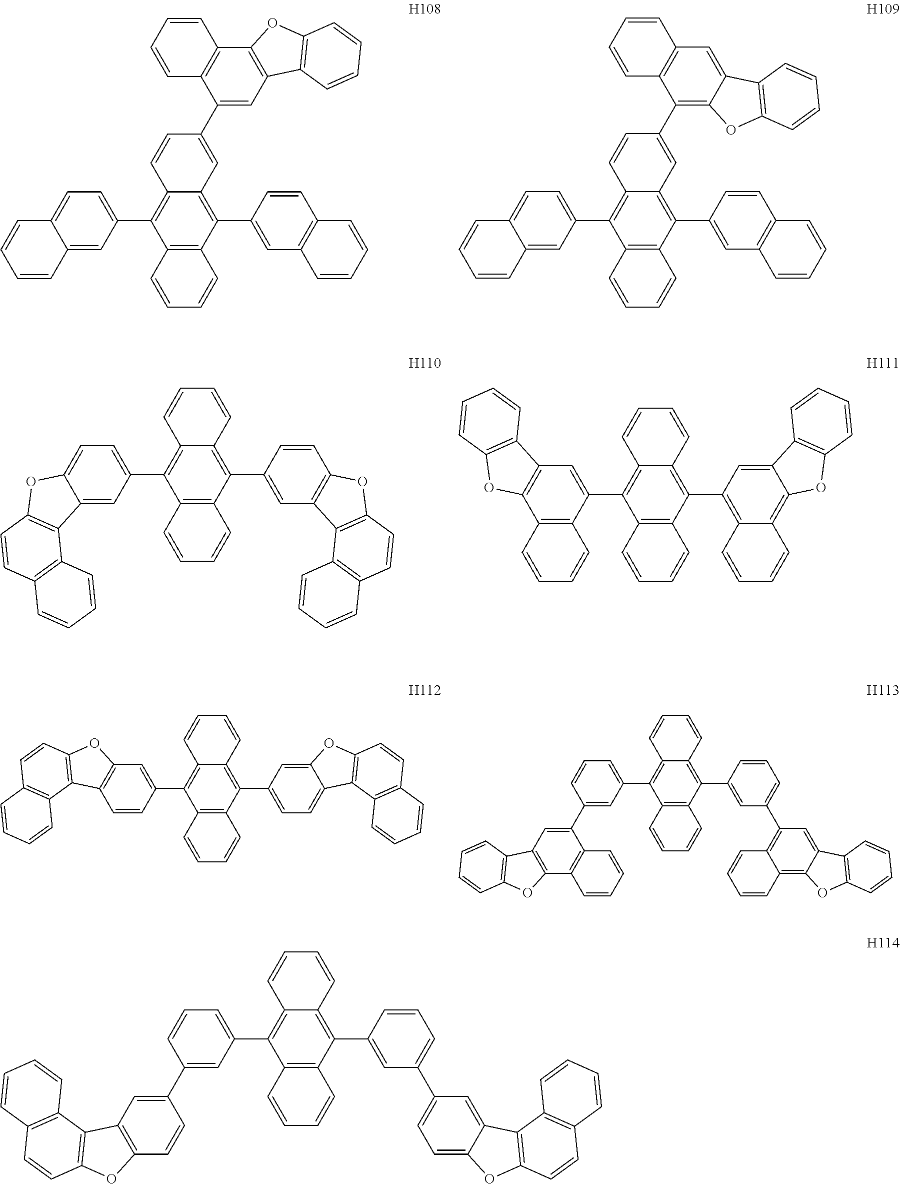

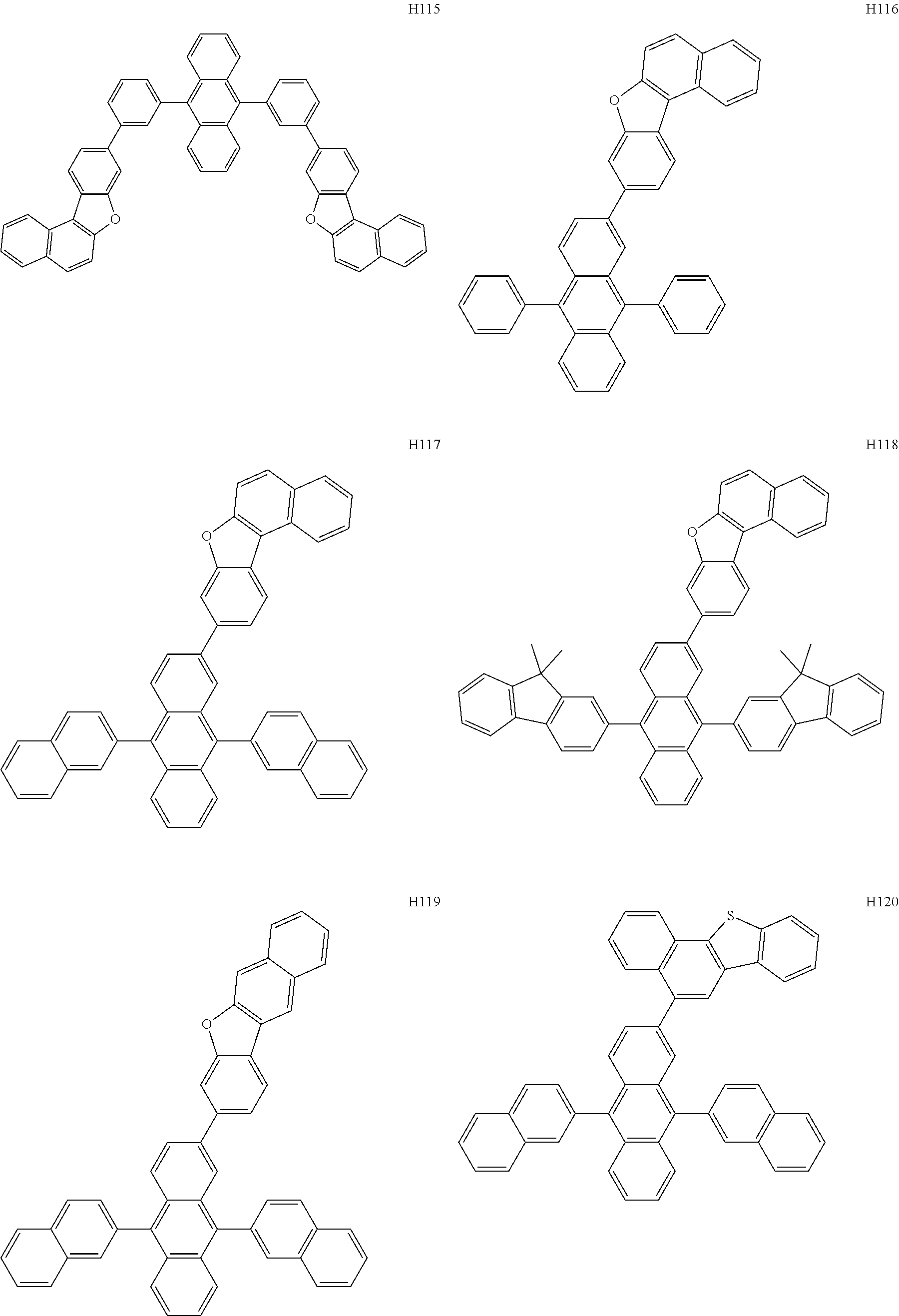

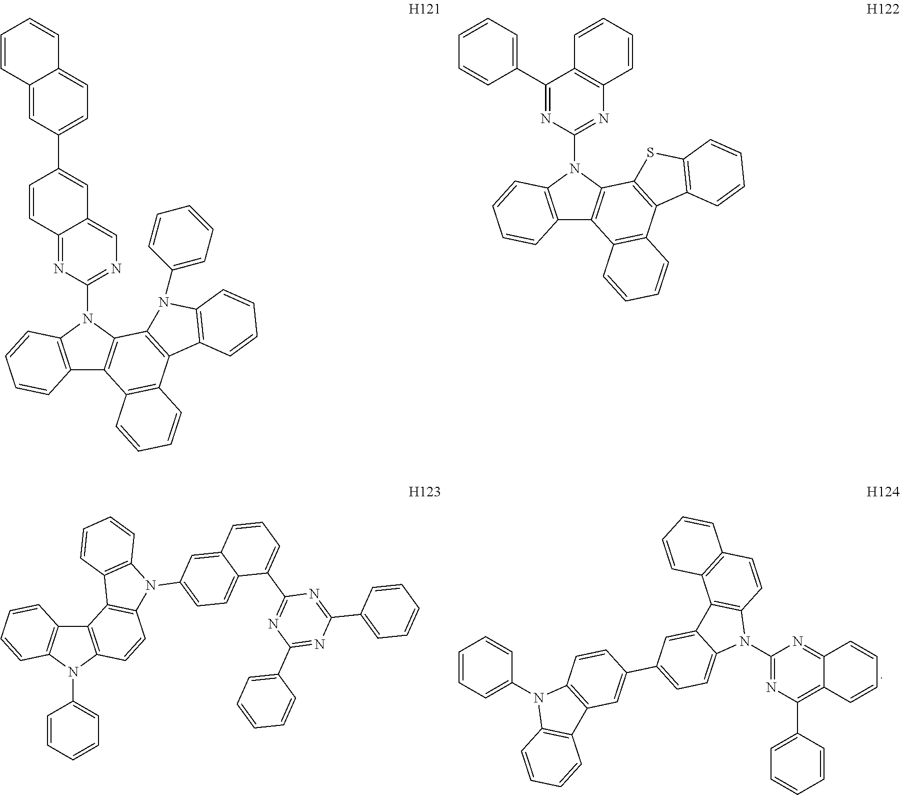 [SMILES: c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

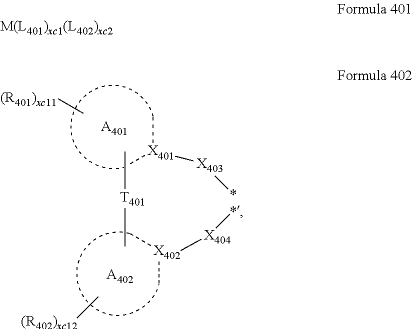

wherein, in Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein when xc1 is two or more, two or more of L₄₀₁(s) may be identical to or different from each other,

L₄₀₂ may be an organic ligand, and xc2 may be 0, 1, 2, 3, or 4, and when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C(Q₄₁₁)=*′,

X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each independently be the same as described herein with respect to Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each independently be the same as described herein with respect to Q₁,

xc11 and xc12 may each independently be an integer from 0 to 10, and

* and *′ in Formula 402 each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 402 is 2 or more, two ring A₄₀₁(s) in two or more of L₄₀₁(s) may be optionally linked to each other via T₄₀₂, which is a linking group, and/or two ring A₄₀₂(s) may be optionally linked to each other via T₄₀₃, which is a linking group (see Compounds PD1 to PD4 and PD7). T₄₀₂ and T₄₀₃ may each independently be the same as described herein with respect to T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or any combination thereof.

The phosphorescent dopant may include, for example, at least one of compounds PD1 to PD39, or any combination thereof:

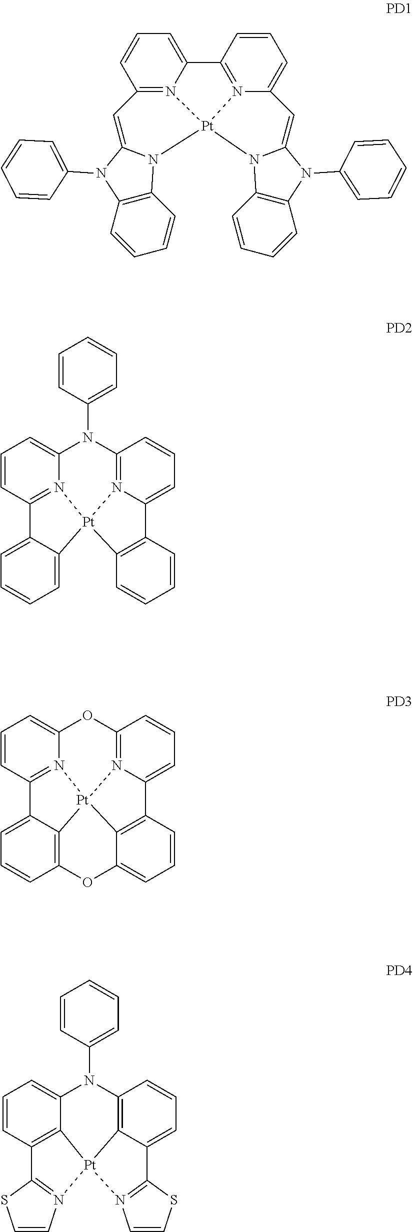

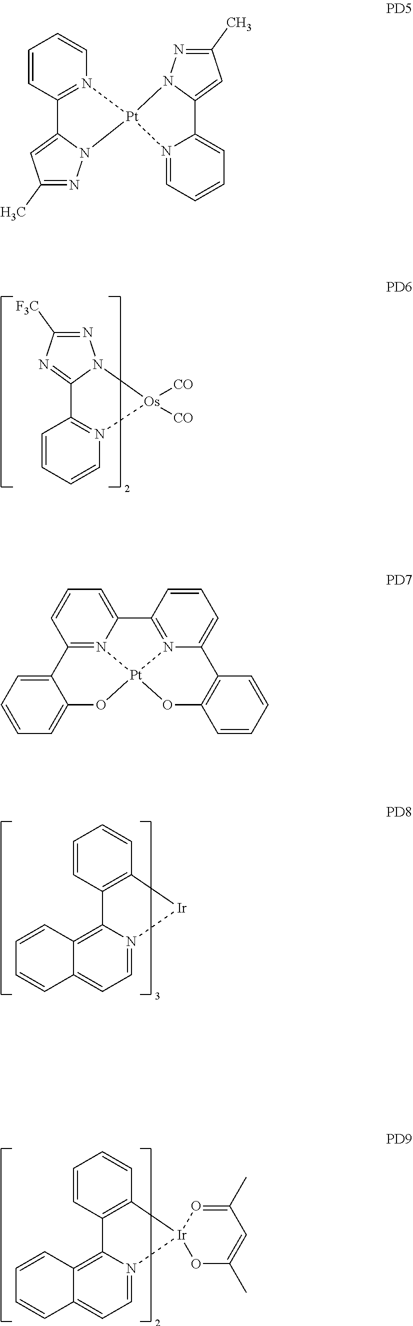

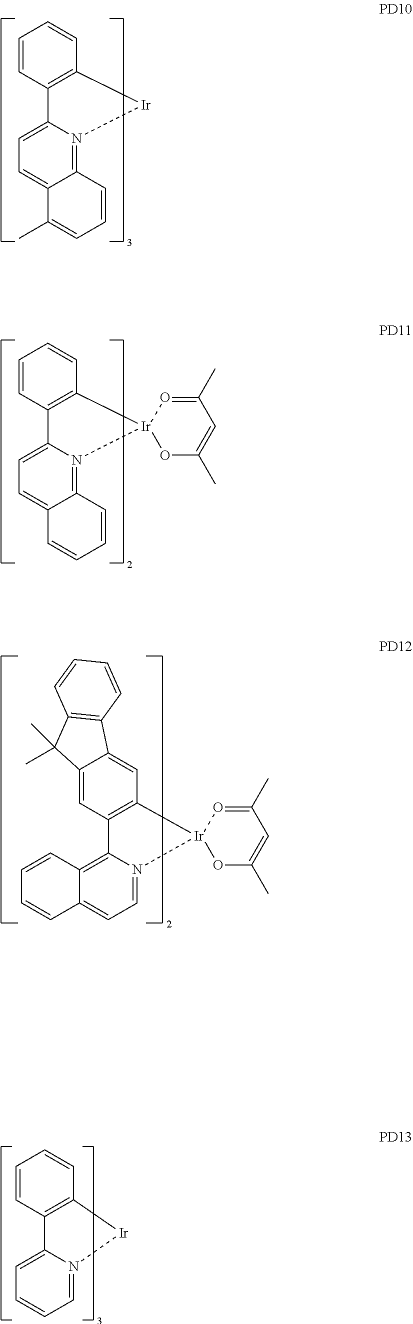

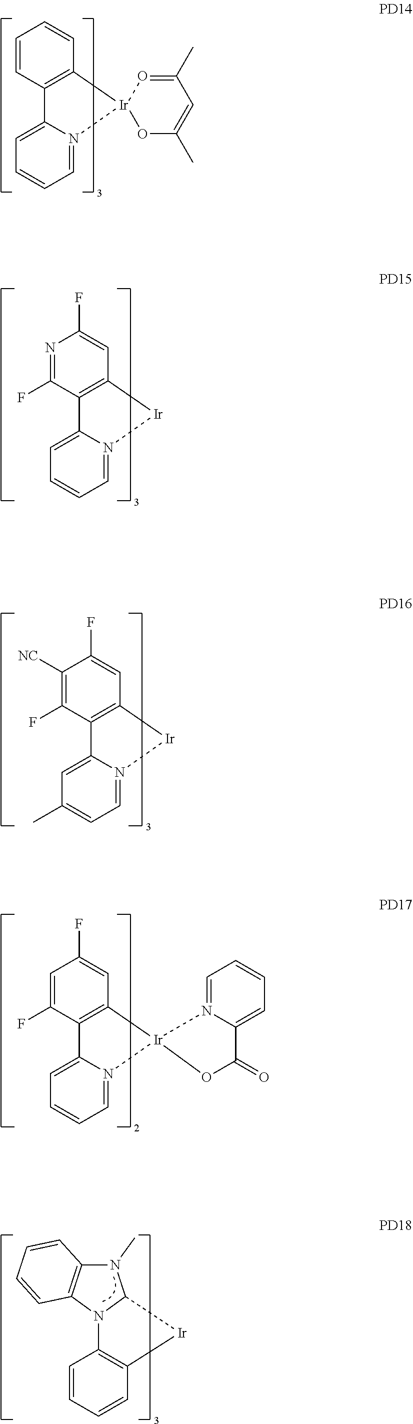

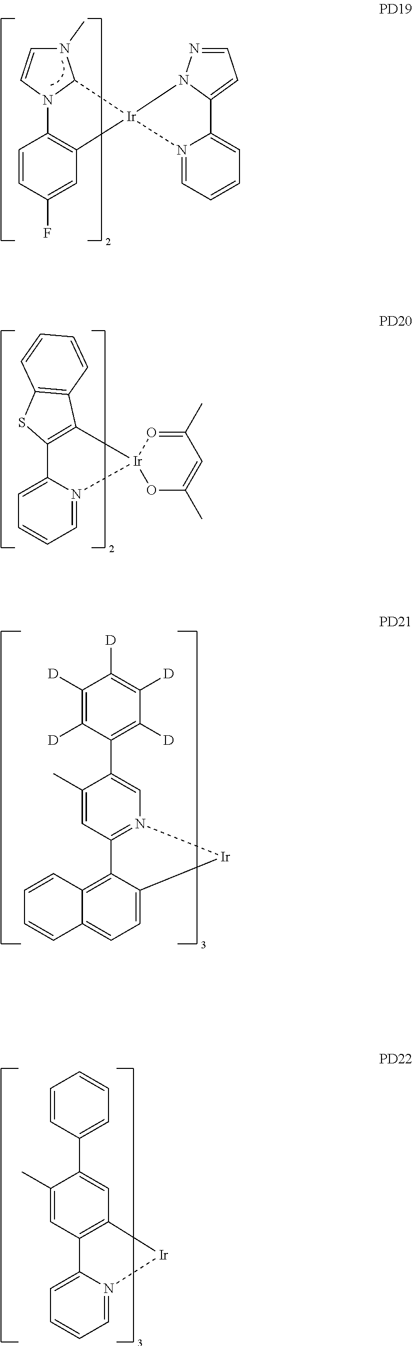

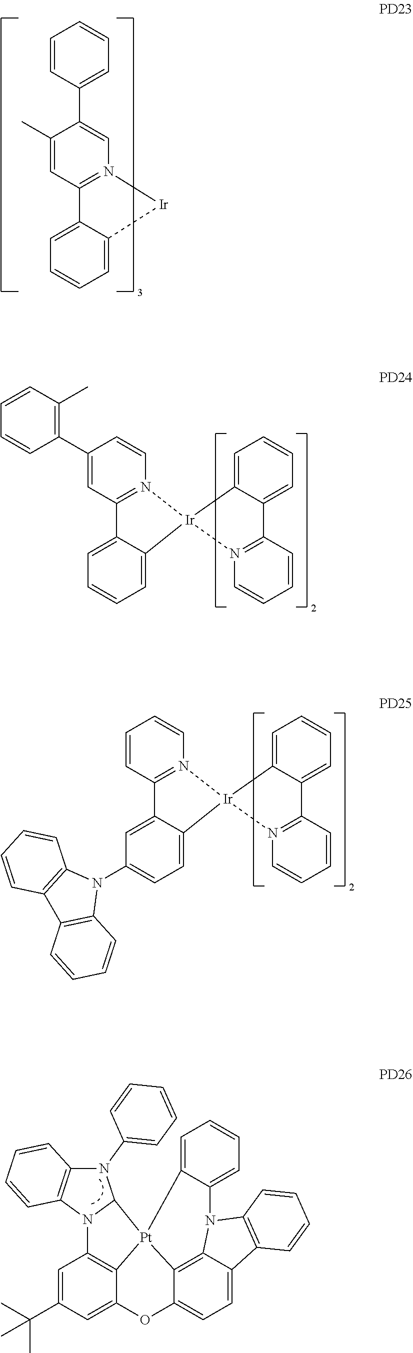

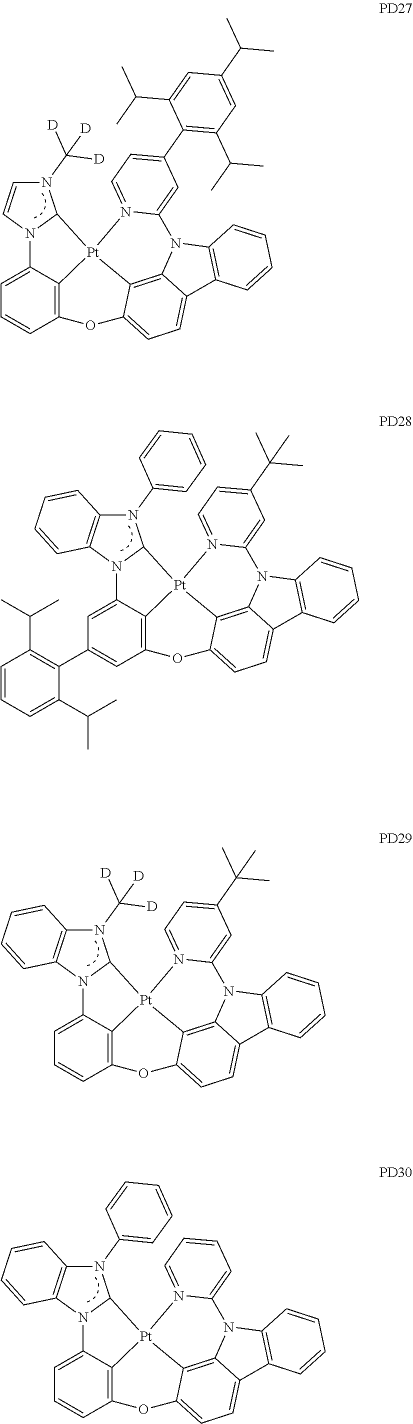

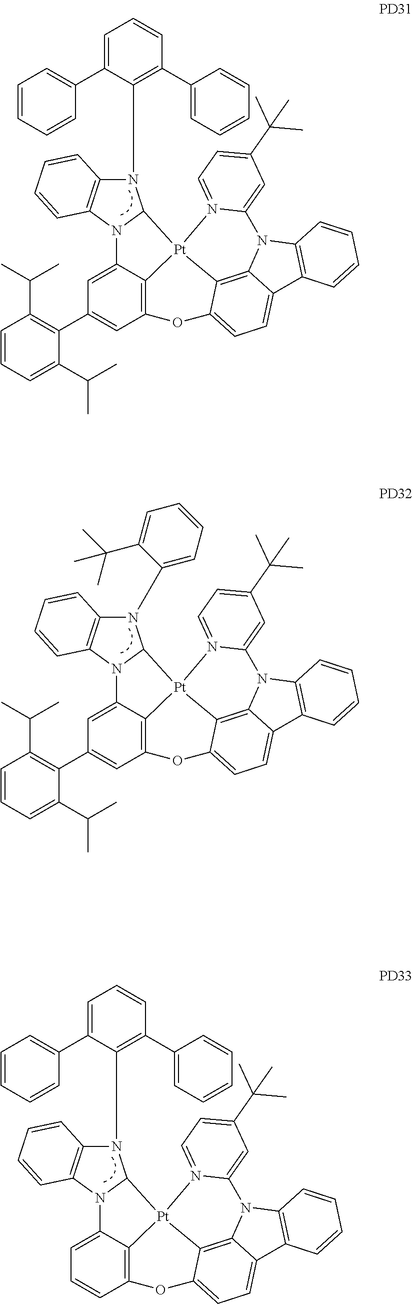

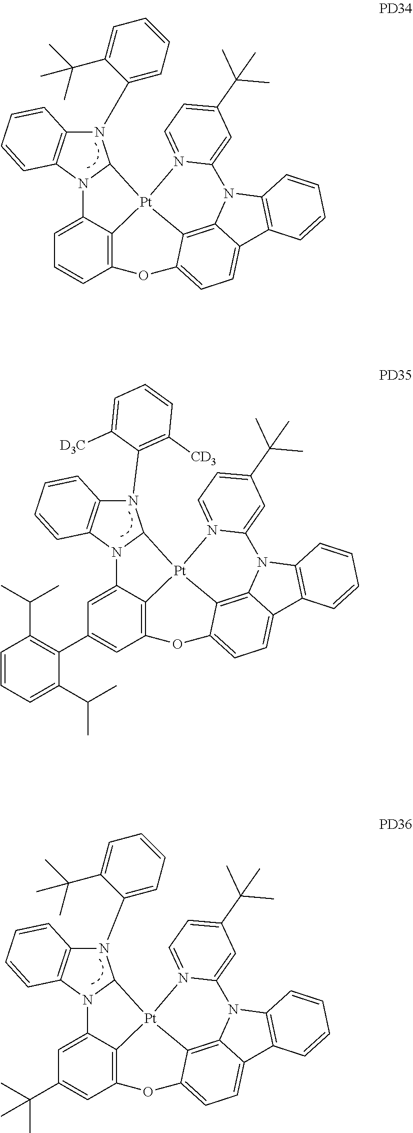

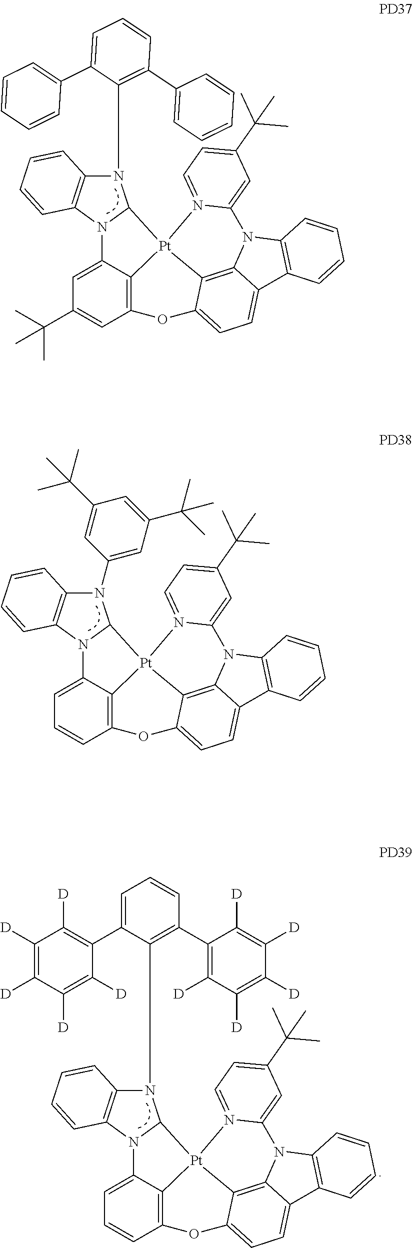

### Fluorescence Dopant

The fluorescence dopant may include an amine group-containing compound, a styryl group-containing compound, or any combination thereof.

For example, the fluorescence dopant may include a compound represented by Formula 501:

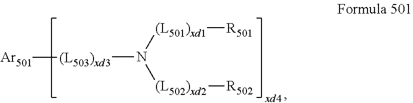

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In one or more embodiments, xd4 in Formula 501 may be 2.

For example, the fluorescence dopant may include: at least one of Compounds FD1 to FD36; DPVBi; DPAVBi; or any combination thereof:

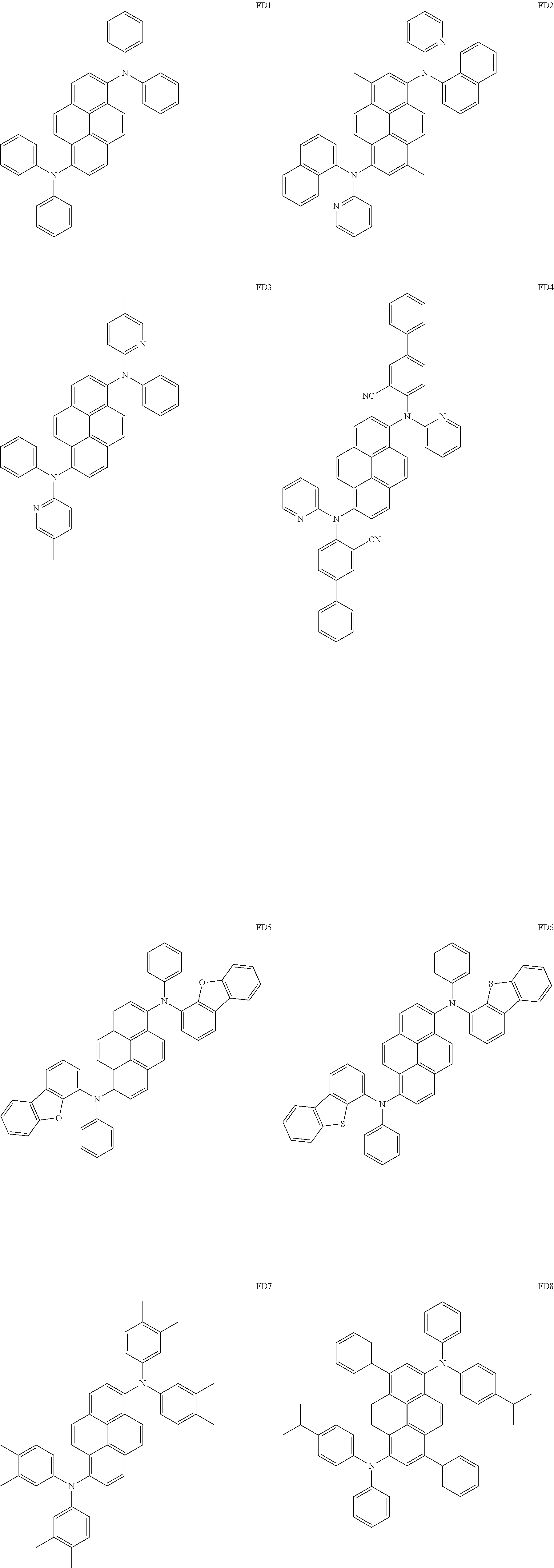

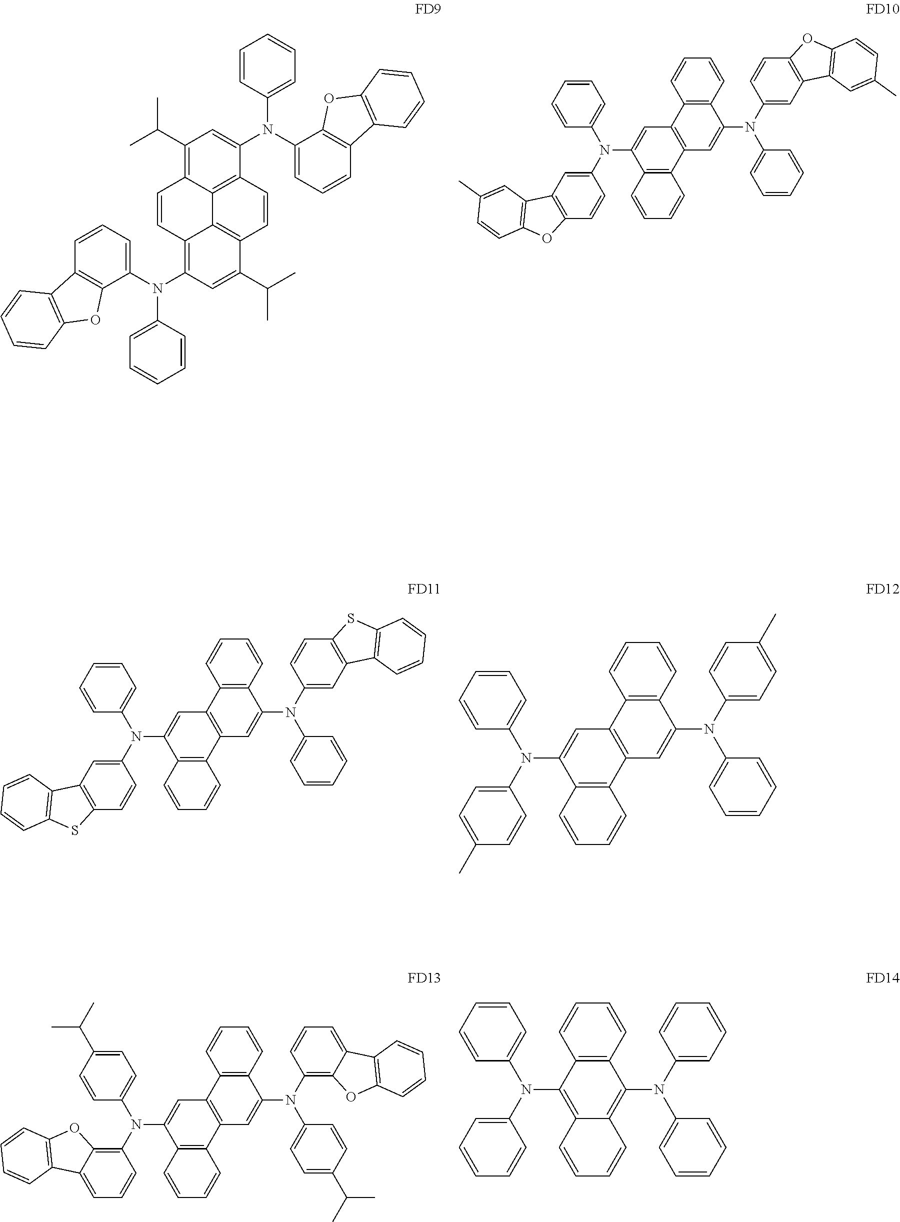

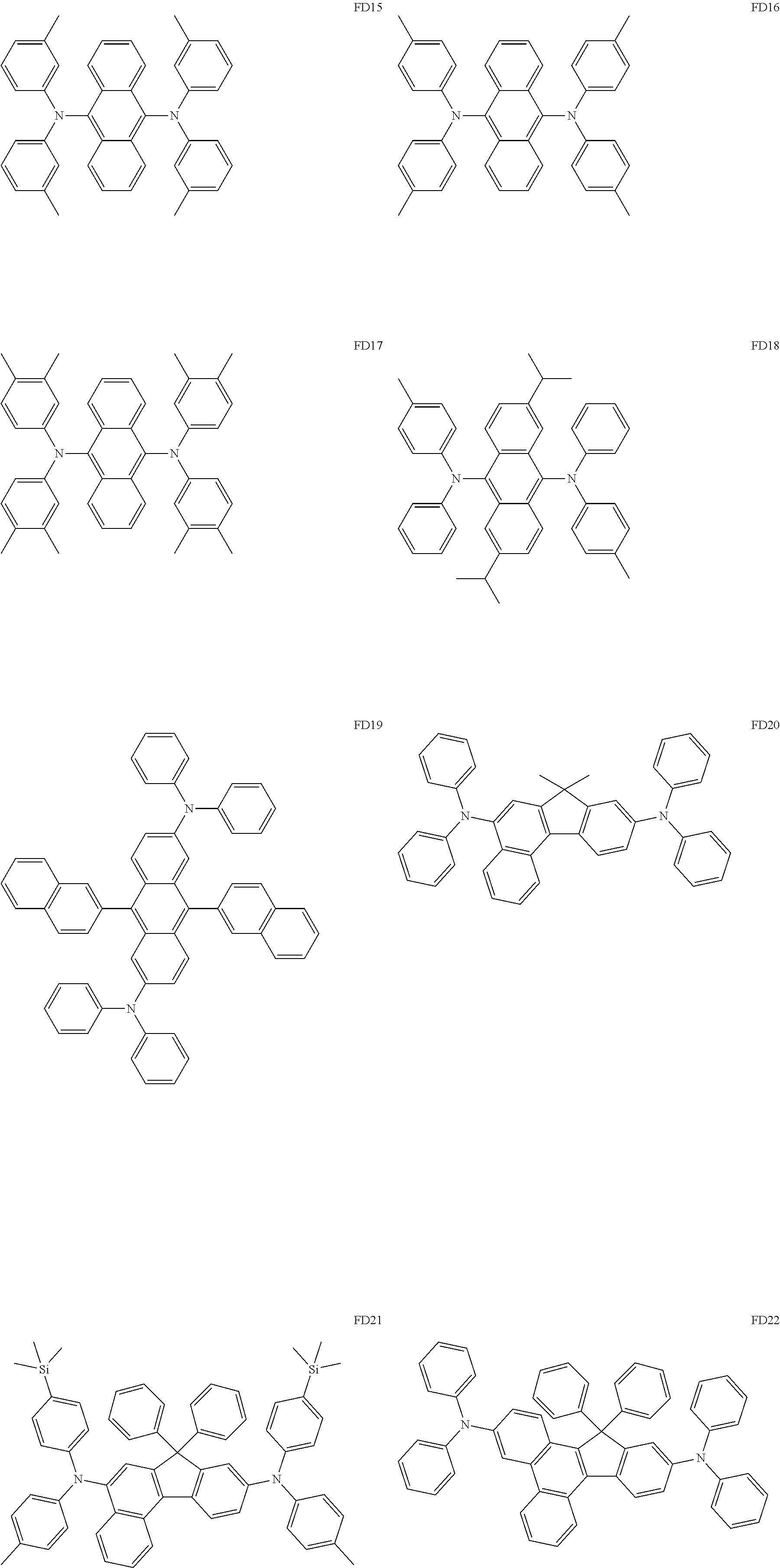

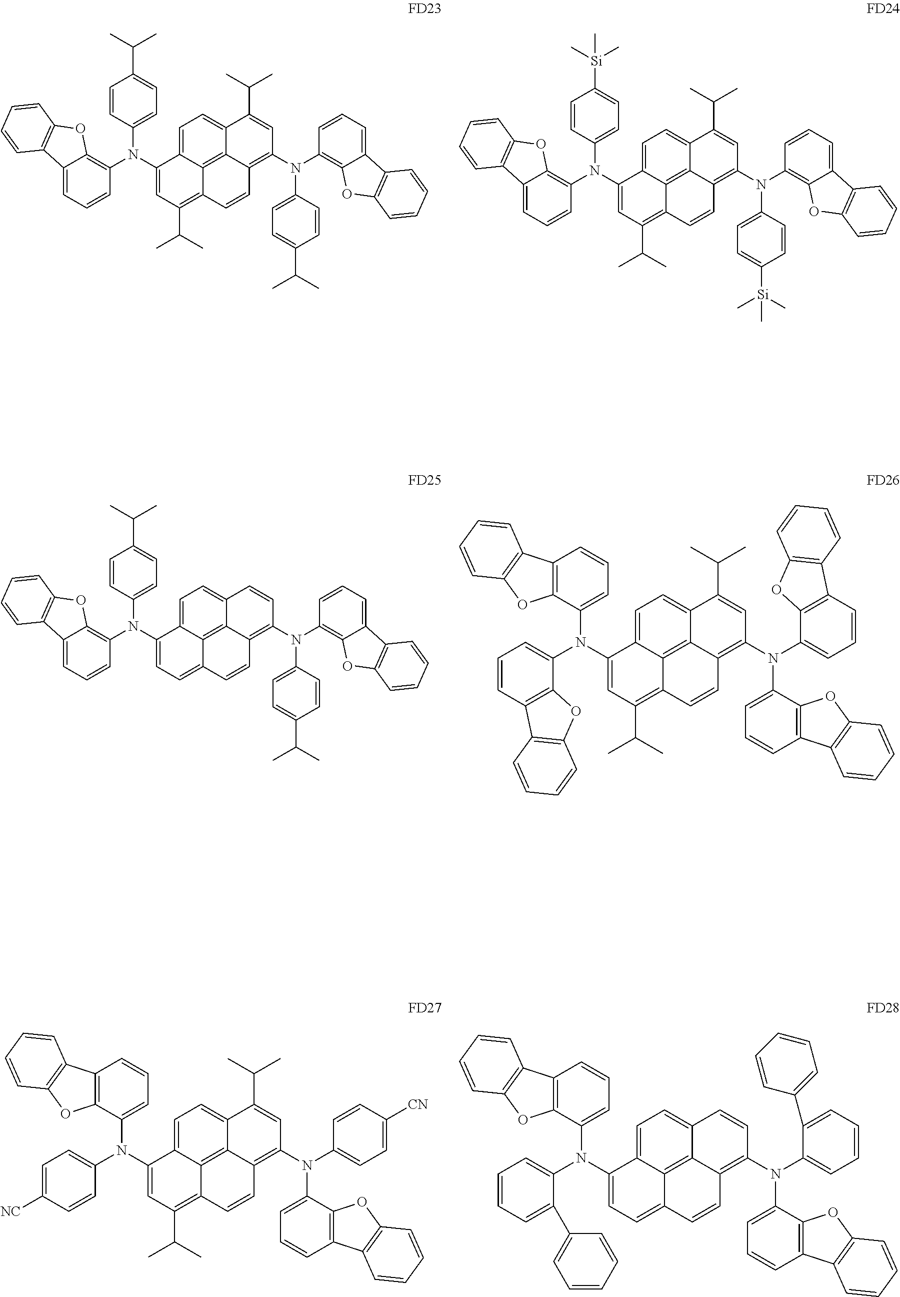

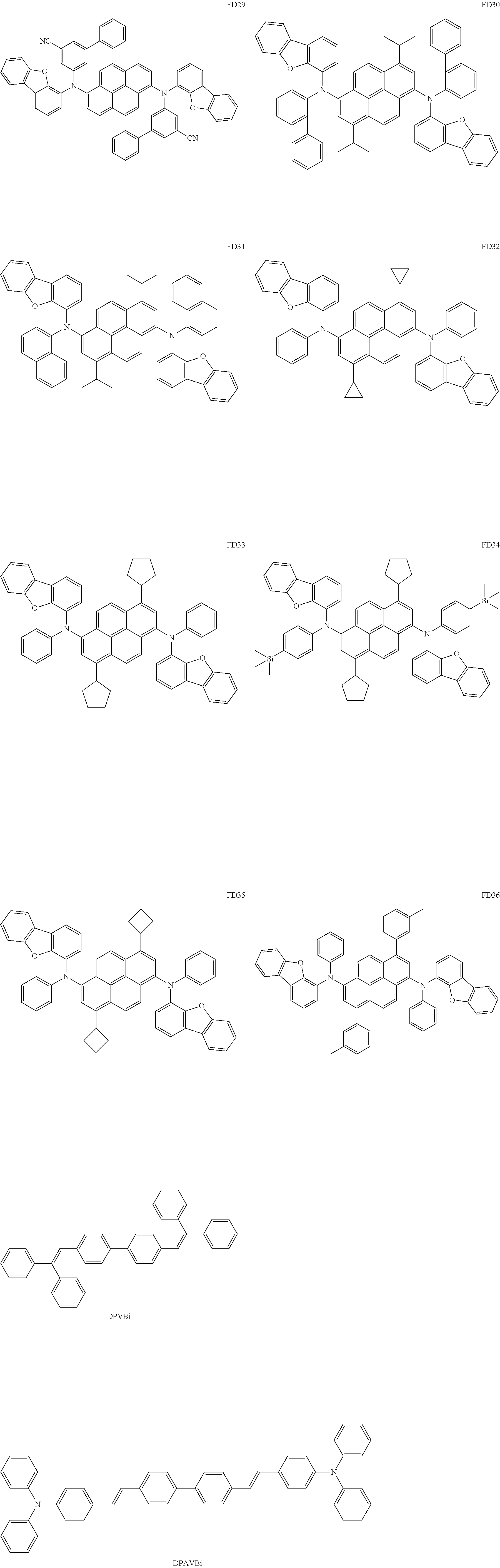

### Delayed Fluorescence Material

The emission layer may include a delayed fluorescence material.

In the present specification, the delayed fluorescence material may be selected from compounds capable of emitting delayed fluorescent light based on a delayed fluorescence emission mechanism.

The delayed fluorescence material included in the emission layer may act as a host or a dopant depending on the type or kind of other materials included in the emission layer.

In one or more embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may occur effectively, and thus, the luminescence efficiency of the light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, and/or a π electron-deficient nitrogen-containing C₁-C₆₀ cyclic group), and/or ii) a material including a C₈-C₆₀ polycyclic group in which two or more cyclic groups are condensed while sharing boron (B).

Examples of the delayed fluorescence material may include at least one of the following compounds DF1 to DF9:

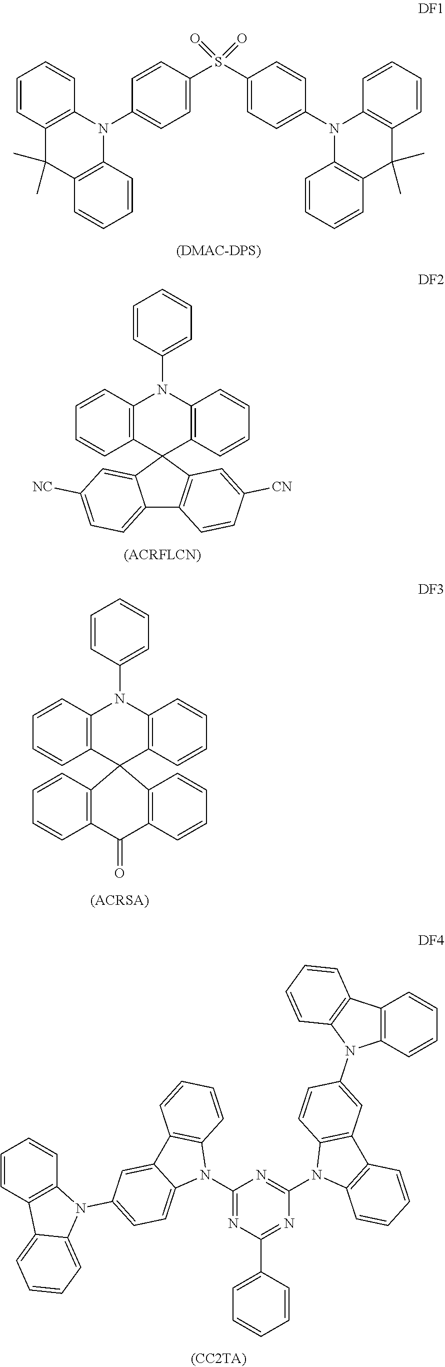

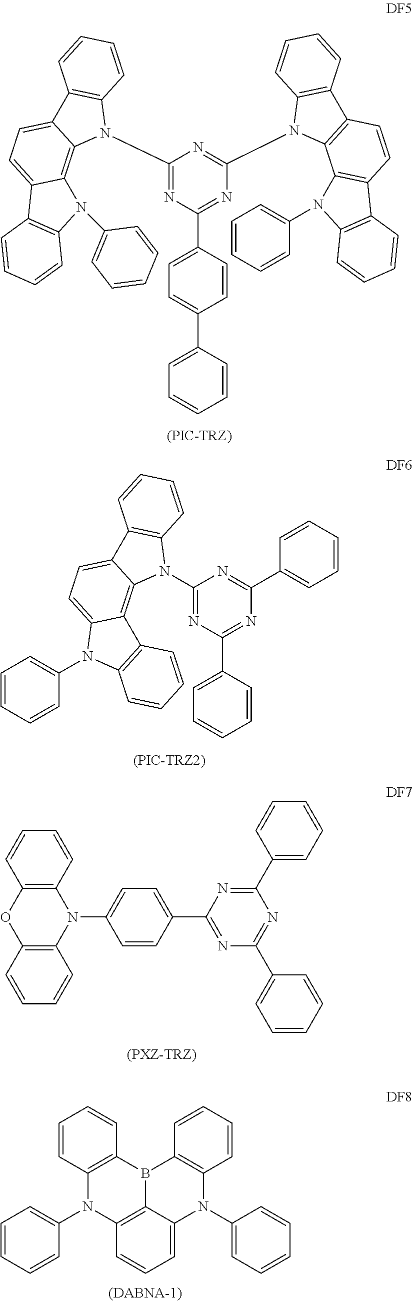

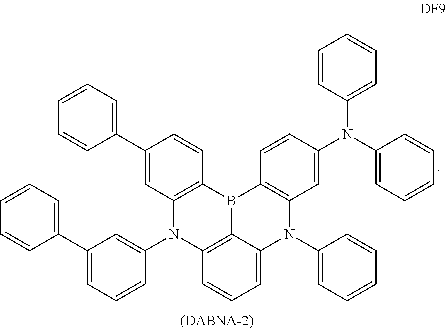

### Quantum Dot

The emission layer may include a quantum dot.

The term “quantum dots” as used herein refers to crystals of a semiconductor compound, and may include any suitable material capable of emitting light of one or more suitable emission wavelengths according to the size of the crystals.

A diameter of the quantum dot may be, for example, in a range of about 1 nm to about 10 nm.

The quantum dot may be synthesized by a wet chemical process, a metal organic (e.g., organometallic) chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts (e.g., serves) as a dispersant coordinated on the surface of the quantum dot crystal and controls the growth of the crystal so that the growth of quantum dot particles can be controlled or selected through a lower cost process, and is easier than vapor deposition methods, such as metal organic chemical vapor deposition (MOCVD) and/or molecular beam epitaxy (MBE),

The quantum dot may include one or more Group II-VI semiconductor compounds, Group III-V semiconductor compounds, Group III-VI semiconductor compounds, Group I-III-VI semiconductor compounds, Group IV-VI semiconductor compounds, Group IV elements or compounds, or any combination thereof.

Examples of the Group II-VI semiconductor compound may include (e.g., may be) a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or any combination thereof. In some embodiments, the Group III-V semiconductor compound may further include a Group II element. Examples of the Group III-V semiconductor compound further including a Group II element may include (e.g., may be) InZnP, InGaZnP, InAlZnP, etc.

Examples of the Group III-VI semiconductor compound may include (e.g., may be): a binary compound, such as GaS, GaSe, Ga₂Se₃, GaTe, InS, InSe, In₂S₃, In₂Se₃, and/or InTe; a ternary compound, such as InGaS₃, and/or InGaSe₃; and any combination thereof.

Examples of the Group I-III-VI semiconductor compound may include (e.g., may be): a ternary compound, such as AgInS, AgInS₂, CuInS, CuInS₂, CuGaO₂, AgGaO₂, and/or AgAlO₂; or any combination thereof.

Examples of the Group IV-VI semiconductor compound may include (e.g., may be): a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element compound, such as Si and/or Ge; a binary compound, such as SiC and/or SiGe; or any combination thereof.

Each element included in a multi-element compound such as the binary compound, the ternary compound, and/or the quaternary compound may be present at a substantially uniform concentration or non-uniform concentration in a particle.

In some embodiments, the quantum dot may have a single structure in which the concentration of each element in the quantum dot is substantially uniform, or a core-shell dual structure. In some embodiments, in a quantum dot with a core-shell structure, the material included in the core and the material included in the shell may be different from each other.

The shell of the quantum dot may act (e.g., serve) as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may include (e.g., may be) an oxide of metal, metalloid, or non-metal, a semiconductor compound, and any combination thereof. Examples of the oxide of metal, metalloid, or non-metal may include (e.g., may be) a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; and any combination thereof. Examples of the semiconductor compound may include (e.g., may be), as described herein, Group II-VI semiconductor compounds; Group III-V semiconductor compounds; Group III-VI semiconductor compounds; Group I-III-VI semiconductor compounds; Group IV-VI semiconductor compounds; and any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission wavelength spectrum of the quantum dot may be about 45 nm or less, for example, about 40 nm or less, for example, about 30 nm or less, and within these ranges, color purity or color reproducibility may be increased. In some embodiments, because the light emitted through the quantum dot is emitted in all directions, the wide viewing angle may be improved.

In addition, the quantum dot may be in the form of a spherical particle, a pyramidal particle, a multi-arm particle, a cubic nanoparticle, a nanotube particle, a nanowire particle, a nanofiber particle, or a nanoplate particle.

Because the energy band gap may be adjusted by controlling the size of the quantum dot, light having one or more suitable wavelength bands may be obtained from the quantum dot emission layer. Accordingly, by utilizing quantum dots of different sizes, a light-emitting device that emits light of one or more suitable wavelengths may be implemented. In one or more embodiments, the size of the quantum dot may be selected to emit red, green and/or blue light. In some embodiments, the size of the quantum dot may be configured to emit white light by combination of light of one or more suitable colors.

### Electron Transport Region in Interlayer 130

The electron transport region may have: i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, with the constituting layers of each structure being sequentially stacked from an emission layer in the respective stated order.

In an embodiment, the electron transport region (for example, the buffer layer, the hole blocking layer, the electron control layer, and/or the electron transport layer in the electron transport region) may include a metal-free compound including at least one π electron-deficient nitrogen-containing C₁-C₆₀ cyclic group.

For example, the electron transport region may include a compound represented by Formula 601 below:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each independently be the same as described herein with respect to Q₁,

xe21 may be 1, 2, 3, 4, or 5,

at least one selected from Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In other embodiments, Ar₆₀₁ in Formula 601 may be a substituted or unsubstituted anthracene group.

In other embodiments, the electron transport region may include a compound represented by Formula 601-1:

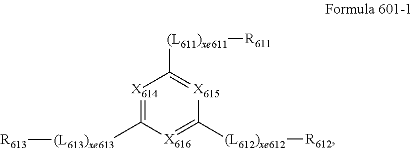

wherein, in Formula 601-1,

X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,

L₆₁₁ to L₆₁₃ may each independently be the same as described herein with respect to L₆₀₁,

xe611 to xe613 may each independently be the same as described herein with respect to xe1,

R₆₁₁ to R₆₁₃ may each independently be the same as described herein with respect to R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include at least one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

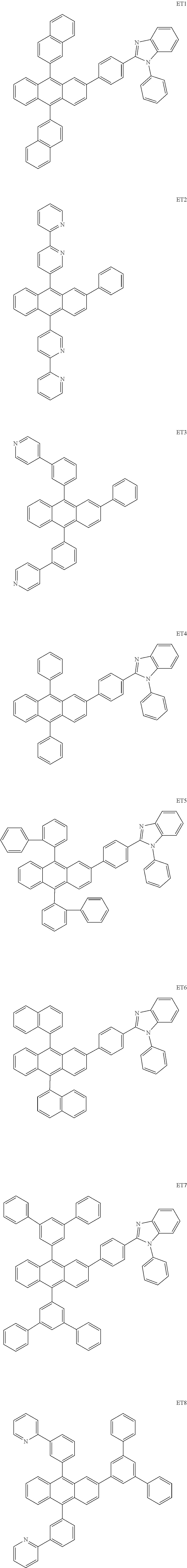

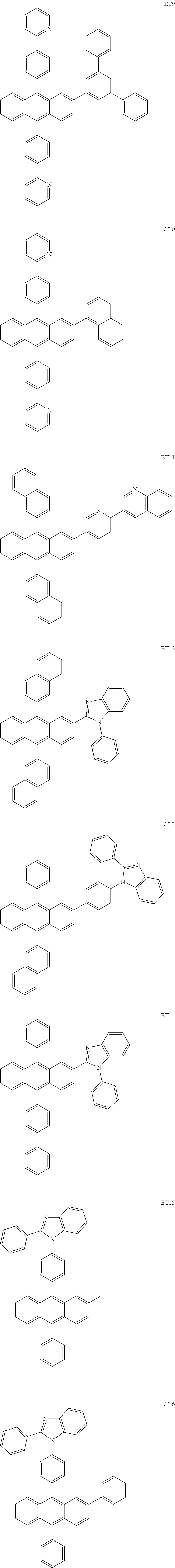

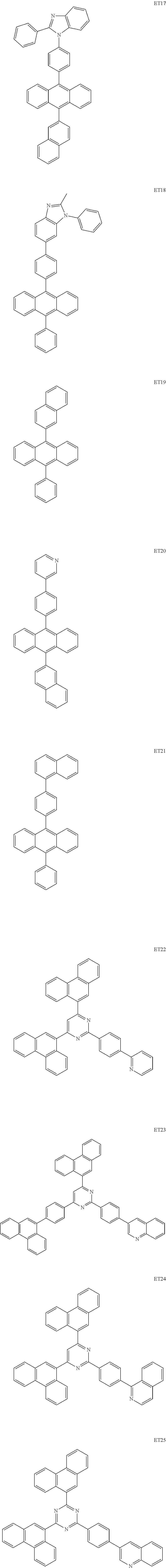

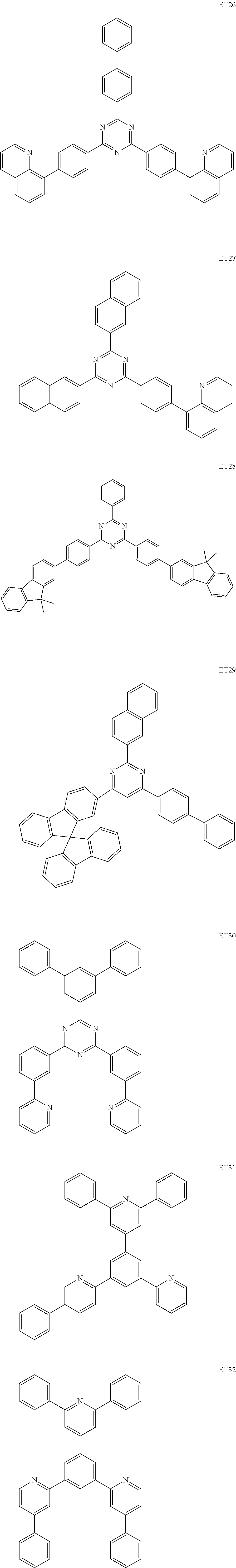

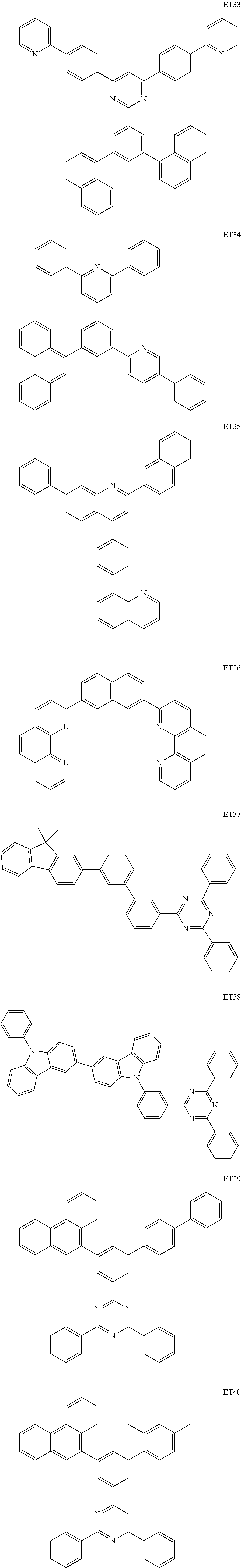

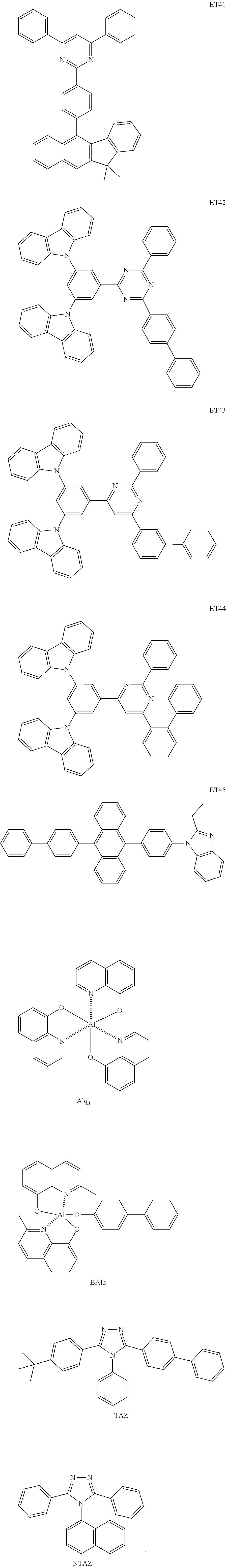

A thickness of the electron transport region may be from about 100 Å to about 5,000 Å, for example, from about 160 Å to about 4,000 Å. When the electron transport region includes a buffer layer, a hole blocking layer, an electron control layer, an electron transport layer, or any combination thereof, the thickness of the buffer layer, the hole blocking layer, or the electron control layer may each independently be from about 20 Å to about 1000 Å, for example, about 30 Å to about 300 Å, and the thickness of the electron transport layer may be from about 100 Å to about 1000 Å, for example, about 150 Å to about 500 Å. When the thicknesses of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of the alkali metal complex may be a Li ion, a Na ion, a K ion, a Rb ion, or a Cs ion, and the metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

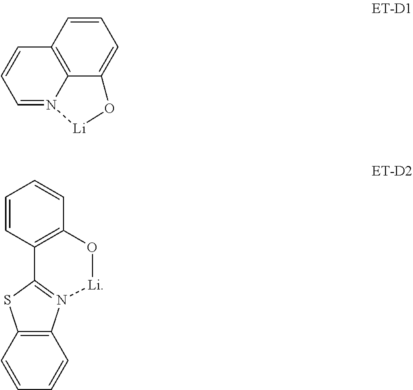

The electron transport region may include an electron injection layer that facilitates the injection of electrons from the second electrode 150. The electron injection layer may directly contact (e.g., physically contact) the second electrode 150.

The electron injection layer may have: i) a single-layered structure consisting of a single layer consisting of a single material, ii) a single-layered structure consisting of a single layer including (e.g., consisting of) a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be one or more oxides, halides (for example, fluorides, chlorides, bromides, and/or iodides), and/or tellurides of the alkali metal, the alkaline earth metal, and/or the rare earth metal, or any combination thereof.

The alkali metal-containing compound may include: one or more alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride may include (e.g., may be) LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, the electron injection layer may be a KI:Yb co-deposited layer, an RbI:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges described above, satisfactory electron injection characteristics may be obtained without a substantial increase in driving voltage.

### Second Electrode 150

The second electrode 150 may be located on the interlayer 130 having a structure as described above. The second electrode 150 may be a cathode, which is an electron injection electrode, and as the material for the second electrode 150, a metal, an alloy, an electrically conductive compound, or any combination thereof, each having a low-work function, may be utilized.

In one or more embodiments, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of (two or more) layers.

### Capping Layer

A first capping layer may be located outside the first electrode 110 (e.g., on the side of the first electrode 110 facing oppositely away from the second electrode 150), and/or a second capping layer may be located outside the second electrode 150 (e.g., on the side of the second electrode 150 facing oppositely away from the first electrode 110). In one or embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward (e.g., transmitted or provided to) the outside through the first electrode 110, which is a semi-transmissive electrode or a transmissive electrode, and the first capping layer, or light generated in an emission layer of the interlayer 130 of the light-emitting device 10 may be extracted toward (e.g., transmitted or provided to) the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. Optionally, the carbocyclic compound, the heterocyclic compound, and the amine group-containing compound may be substituted with a substituent including O, N, S, Se, Si, F, Cl, Br, I, or any combination thereof. In one or more embodiments, at least one of the first capping layer or the second capping layer may each independently include an amine group-containing compound.

For example, at least one of the first capping layer or the second capping layer may each independently include a compound represented by Formula 201, a compound represented by Formula 202, or any combination thereof.

In one or more embodiments, at least one of the first capping layer or the second capping layer may each independently include at least one of Compounds HT28 to HT33, at least one of Compounds CP1 to CP6, β-NPB, or any combination thereof:

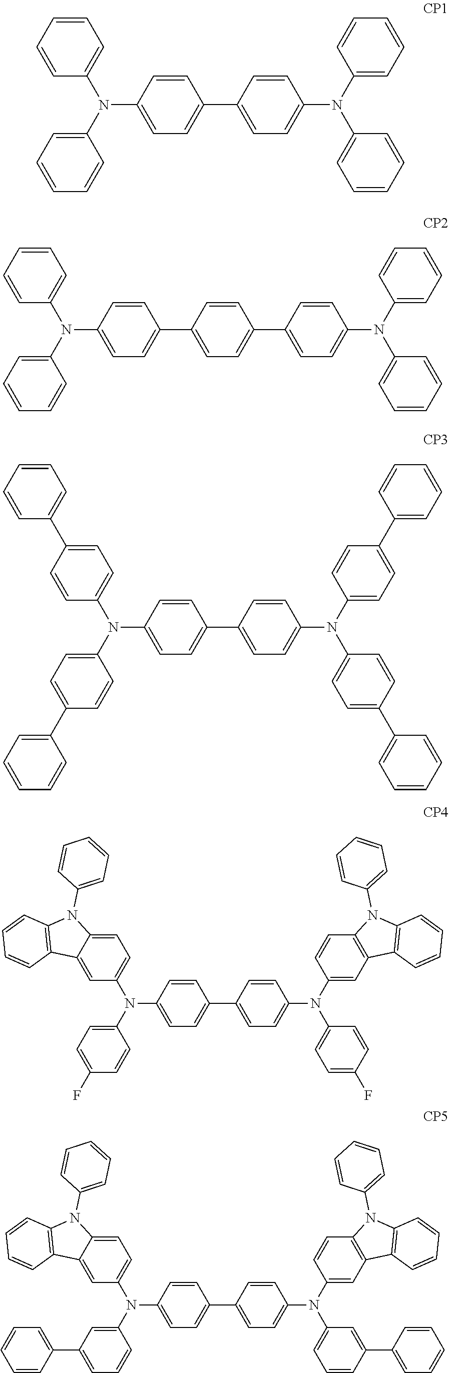 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

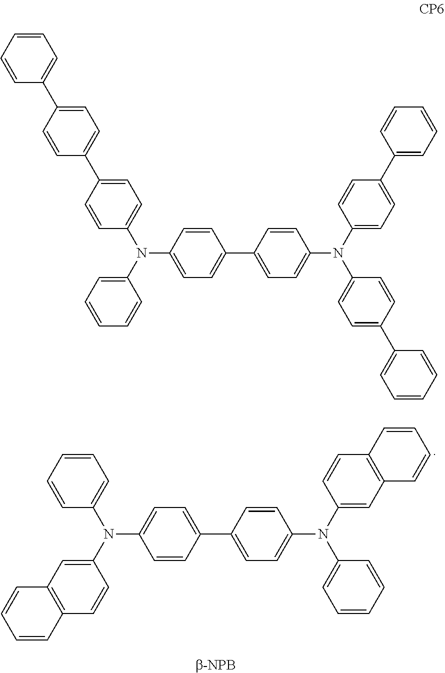

### Film

The condensed cyclic compound represented by Formula 1 may be included in one or more suitable films. Accordingly, another aspect provides a film including the condensed cyclic compound represented by Formula 1. The film may be, for example, an optical member (or a light control means) (for example, a color filter, a color conversion member, a capping layer, a light extraction efficiency enhancement layer, a selective light absorbing layer, a polarizing layer, a quantum dot-containing layer, and/or like), a light-blocking member (for example, a light reflective layer, a light absorbing layer, and/or the like), a protective member (for example, an insulating layer, a dielectric layer, and/or the like), and/or the like.

### Electronic Apparatus

The light-emitting device may be included in one or more suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be located in at least one traveling direction of light emitted from the light-emitting device. For example, the light emitted from the light-emitting device may be blue light or white light. For details on the light-emitting device, related description provided above may be referred to. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the plurality of subpixel areas, and the color conversion layer may include a plurality of color conversion areas respectively corresponding to the plurality of subpixel areas.

A pixel-defining film may be located among the plurality of subpixel areas to define each of the subpixel areas.

The color filter may further include a plurality of color filter areas and light-shielding patterns located among the plurality of color filter areas, and the color conversion layer may further include a plurality of color conversion areas and light-shielding patterns located among the plurality of color conversion areas.

The plurality of color filter areas (or the plurality of color conversion areas) may include a first area emitting a first color light, a second area emitting a second color light, and/or a third area emitting a third color light, wherein the first color light, the second color light, and/or the third color light may have different maximum emission wavelengths from one another. In one or more embodiments, the first color light may be red light, the second color light may be green light, and the third color light may be blue light. In one or more embodiments, the plurality of color filter areas (or the plurality of color conversion areas) may include quantum dots. For example, the first area may include a red quantum dot, the second area may include a green quantum dot, and the third area may not include (e.g., may exclude) any quantum dots. For details on the quantum dot, related descriptions provided herein may be referred to. The first area, the second area, and/or the third area may each further include a scatterer.

In one or more embodiments, the light-emitting device may be to emit a first light, the first area may be to absorb the first light to emit a first-first color light, the second area may be to absorb the first light to emit a second-first color light, and the third area may be to absorb the first light to emit a third-first color light. In this regard, the first-first color light, the second-first color light, and the third-first color light may have different maximum emission wavelengths. In particular, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein the source electrode or the drain electrode may be electrically connected to the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be located between the color conversion layer and/or color filter and the light-emitting device. The sealing portion allows light from the light-emitting device to be extracted to the outside, and concurrently (e.g., simultaneously) prevents or reduces penetration of ambient air and/or moisture into the light-emitting device. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin film encapsulation layer, the electronic apparatus may be flexible.

Various suitable functional layers may be additionally located on the sealing portion, in addition to the color filter and/or the color conversion layer, according to the usage of the electronic apparatus. Examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, etc.).

The authentication apparatus may further include, in addition to the light-emitting device as described above, a biometric information collector.

The electronic apparatus may be applied to one or more suitable displays, light sources, lighting apparatuses, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, one or more suitable measuring instruments, meters (for example, meters for a vehicle, an aircraft, and/or a vessel), projectors, and/or the like.

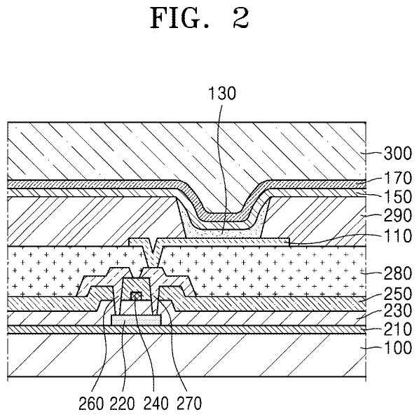

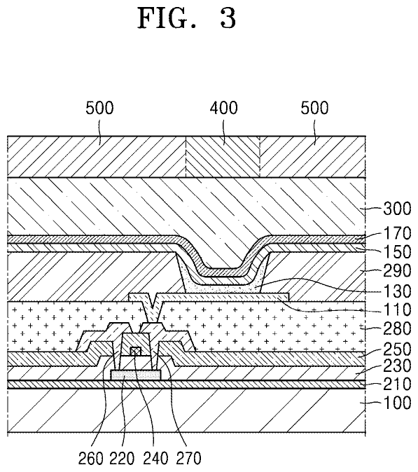

### Description of FIGS. 2 and 3

FIG. 2 is a cross-sectional view showing a light-emitting apparatus according to an embodiment of the present disclosure.

The light-emitting apparatus of FIG. 2 includes a substrate 100, a thin-film transistor (TFT), a light-emitting device, and an encapsulation portion (or an encapsulation layer) 300 that seals the light-emitting device.

The substrate 100 may be a flexible substrate, a glass substrate, or a metal substrate. A buffer layer 210 may be located on the substrate 100. The buffer layer 210 may prevent or reduce penetration of impurities through the substrate 100 and may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor such as silicon and/or polysilicon, an organic semiconductor, and/or an oxide semiconductor, and may include a source region, a drain region, and a channel region.

A gate insulating film 230 for insulating the activation layer 220 from the gate electrode 240 may be located on the activation layer 220, and the gate electrode 240 may be located on the gate insulating film 230.

An interlayer insulating film 250 may be located on the gate electrode 240. The interlayer insulating film 250 may be located between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220.

The TFT is electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be located on the passivation layer 280. The passivation layer 280 may be located to expose a portion of the drain electrode 270 and not fully covering the drain electrode 270, and the first electrode 110 may be located to be connected to the exposed portion of the drain electrode 270.

A pixel defining layer 290 including an insulating material may be located on the first electrode 110. The pixel defining layer 290 may expose a certain region of the first electrode 110, and an interlayer 130 may be formed in the exposed region of the first electrode 110. The pixel defining layer 290 may be a polyimide or polyacrylic organic film. In one embodiment, one or more layers of the interlayer 130 may extend beyond the upper portion of the pixel defining layer 290 in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or any combination of the inorganic film(s) and the organic film(s).

FIG. 3 shows a cross-sectional view showing a light-emitting apparatus according to an embodiment of the present disclosure.

The light-emitting apparatus of FIG. 3 is the same as the light-emitting apparatus of FIG. 2, except that a light-shielding pattern 500 and a functional region 400 are additionally located on the encapsulation portion 300. The functional region 400 may be i) a color filter area, ii) a color conversion area, or iii) a combination of the color filter area and the color conversion area. In an embodiment, the light-emitting device included in the light-emitting apparatus of FIG. 3 may be a tandem light-emitting device.

### Manufacturing Method

The layers included in the hole transport region, the emission layer, and the layers included in the electron transport region may be formed in a certain region by utilizing one or more suitable methods such as vacuum deposition, spin coating, casting, Langmuir-Blodgett (LB) deposition, ink-jet printing, laser-printing, laser-induced thermal imaging, and/or the like.

When layers constituting the hole transport region, an emission layer, and layers constituting the electron transport region are formed by vacuum deposition, the vacuum deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### DEFINITION OF TERMS

The term “C₃-C₆₀ carbocyclic group” as used herein refers to a cyclic group consisting of only carbon atoms as ring-forming atoms and having three to sixty carbon atoms, and the term “C₁-C₆₀ heterocyclic group” as used herein refers to a cyclic group that has, in addition to one to sixty carbon atoms, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The term “cyclic group” as used herein may include the C₃-C₆₀ carbocyclic group, and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

For example,

the C₃-C₆₀ carbocyclic group may be i) a group T1 or ii) a condensed cyclic group in which two or more groups T1 are condensed with each other (for example, the C₃-C₆₀ carbocyclic group may be a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a group T2, ii) a condensed cyclic group in which two or more groups T2 are condensed with each other, or iii) a condensed cyclic group in which at least one group T2 and at least one group T1 are condensed with each other (for example, the C₁-C₆₀ heterocyclic group may be a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) a group T1, ii) a condensed cyclic group in which two or more groups T1 are condensed with each other, iii) a group T3, iv) a condensed cyclic group in which two or more groups T3 are condensed with each other, or v) a condensed cyclic group in which at least one group T3 and at least one group T1 are condensed with each other (for example, the π electron-rich C₃-C₆₀ cyclic group may be the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) a group T4, ii) a condensed cyclic group in which two or more groups T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with one another (for example, the π electron-deficient nitrogen-containing C₁-C₆₀ cyclic group may be a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the group T3 may be a furan group, a thiophene group, a 1H-pyrrole group, a silole group, or a borole group, and

the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the π electron-rich C₃-C₆₀ cyclic group,” or “the π electron-deficient nitrogen-containing C₁-C₆₀ cyclic group” as used herein each refer to a group condensed to any cyclic group, a monovalent group, or a polyvalent group (for example, a divalent group, a trivalent group, a tetravalent group, etc.) according to the structure of a formula for which the corresponding term is used. For example, the “benzene group” may be a benzo group, a phenyl group, a phenylene group, and/or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group may include (e.g., may be) a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the divalent C₁-C₆₀ heterocyclic group may include (e.g., may be) a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof may include (e.g., may be) a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having substantially the same structure as the C₁-C₆₀ alkyl group.

The term “C₂-C₆₀ alkenyl group” as used herein refers to a monovalent hydrocarbon group having at least one carbon-carbon double bond in the middle and/or at a terminal end (e.g., the terminus) of the C₂-C₆₀ alkyl group, and examples thereof may include (e.g., may be) an ethenyl group, a propenyl group, and a butenyl group. The term “C₂-C₆₀ alkenylene group” as used herein refers to a divalent group having substantially the same structure as the C₂-C₆₀ alkenyl group.

The term “C₂-C₆₀ alkynyl group” as used herein refers to a monovalent hydrocarbon group having at least one carbon-carbon triple bond in the middle and/or at a terminal end (e.g., the terminus) of the C₂-C₆₀ alkyl group, and examples thereof may include an ethynyl group and a propynyl group. The term “C₂-C₆₀ alkynylene group” as used herein refers to a divalent group having substantially the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof may include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof may include (e.g., may be) a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having substantially the same structure as the C₃-C₁ cycloalkyl group.

The term “C₁-C₁₀ heterocycloalkyl group” as used herein refers to a monovalent saturated monocyclic group that includes, in addition to 1 to 10 carbon atoms, at least one heteroatom, as ring-forming atoms, and examples may include (e.g., may be) a 1,2,3,4-oxatriazolidinyl group, a tetrahydrofuranyl group, and a tetrahydrothiophenyl group. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has three to ten carbon atoms and at least one carbon-carbon double bond in the ring thereof and no aromaticity, and examples thereof may include (e.g., may be) a cyclopentenyl group, a cyclohexenyl group, and a cycloheptenyl group. The term “C₃-C₁₀ cycloalkenylene group” as used herein refers to a divalent group having substantially the same structure as the C₃-C₁₀ cycloalkenyl group.

The term “C₁-C₁₀ heterocycloalkenyl group” as used herein refers to a monovalent cyclic group that includes, in addition to 1 to 10 carbon atoms, at least one heteroatom, as a ring-forming atom, and having at least one carbon-carbon double bond in the cyclic structure thereof. Examples of the C₁-C₁₀ heterocycloalkenyl group may include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having substantially the same structure as the C₁-C₁₀ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Examples of the C₆-C₆₀ aryl group may include (e.g., may be) a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each independently include two or more rings, the two or more rings may be condensed with each other.

The term “C₁-C₆₀ heteroaryl group” as used herein refers to a monovalent group having a heterocyclic aromatic system that has, in addition to 1 to 60 carbon atoms, at least one heteroatom, as a ring-forming atom. The term “C₁-C₆₀ heteroarylene group” as used herein refers to a divalent group having a heterocyclic aromatic system that has, in addition to 1 to 60 carbon atoms, at least one heteroatom, as a ring-forming atom. Examples of the C₁-C₆₀ heteroaryl group may include (e.g., may be) a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a carbazolyl group, a dibenzofuranyl group, a dibenzothiophenyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group having two or more rings condensed to each other, only carbon atoms (for example, having 8 to 60 carbon atoms) as ring-forming atoms, and no aromaticity in its entire molecular structure (e.g., is not aromatic when considered as a whole). Examples of the monovalent non-aromatic condensed polycyclic group may include (e.g., may be) an indenyl group, a fluorenyl group, a spiro-bifluorenyl group, a benzofluorenyl group, an indenophenanthrenyl group, an adamantyl group, and an indeno anthracenyl group. The term “divalent non-aromatic condensed polycyclic group” as used herein refers to a divalent group having substantially the same structure as the monovalent non-aromatic condensed polycyclic group described above.

The term “monovalent non-aromatic condensed heteropolycyclic group” as used herein refers to a monovalent group having two or more rings condensed to each other, at least one heteroatom in addition to 1 to 60 carbon atoms, as ring-forming atoms, and non-aromaticity in its entire molecular structure (e.g., is not aromatic when considered as a whole). Examples of the monovalent non-aromatic condensed heteropolycyclic group may include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, an azaadamantyl group, and a benzothienodibenzothiophenyl group. The term “divalent non-aromatic condensed heteropolycyclic group” as used herein refers to a divalent group having substantially the same structure as the monovalent non-aromatic condensed heteropolycyclic group described above.

The term “C₆-C₆₀ aryloxy group” as used herein refers to a monovalent group represented by —OA₁₀₂ (wherein A₁₀₂ is a C₆-C₆₀ aryl group), and the term “C₆-C₆₀ arylthio group” as used herein refers to a monovalent group represented by —SA₁₀₃ (wherein A₁₀₃ is a C₆-C₆₀ aryl group).

The term “C₇-C₆₀ aryl alkyl group” as used herein refers to a monovalent group represented by -A₁₀₄A₁₀₅ (where A₁₀₄ may be a C₁-C₅₄ alkylene group, and A₁₀₅ may be a C₆-C₅₉ aryl group), and the term “C₂-C₆₀ heteroaryl alkyl group” as used herein refers to a monovalent group represented by -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as used herein refers to:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃ and Q₃₁ to Q₃₃ as used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ aryl alkyl group; or a C₂-C₆₀ heteroaryl alkyl group.

The term “heteroatom” as used herein refers to any atom other than a carbon atom. Examples of the heteroatom may include (e.g., may be) O, S, N, P, Si, B, Ge, Se, and any combinations thereof.

The term “third-row transition metal” as used herein includes hafnium (Hf), tantalum (Ta), tungsten (W), rhenium (Re), osmium (Os), iridium (Ir), platinum (Pt), gold (Au), and/or the like.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

Hereinafter, compounds according to embodiments and light-emitting devices according to embodiments will be described in more detail with reference to the following synthesis examples and examples. The wording “B was utilized instead of A” utilized in describing Synthesis Examples refers to that an identical molar equivalent of B was utilized in place of A.

### EXAMPLES

### Synthesis Example

### Synthesis Example 1: Synthesis of Compound 1

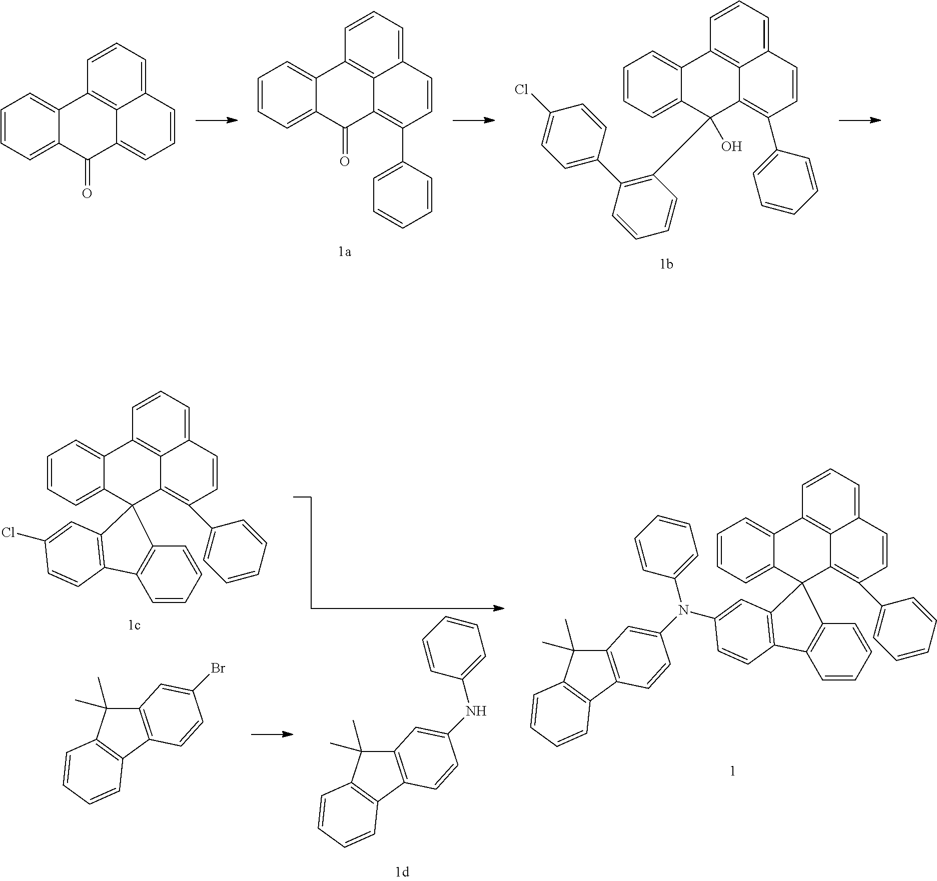

### 1-1. Synthesis of Intermediate 1a

7H-benzo[de]anthracen-7-one (1.0 eq) and anhydrous diethyl ether were cooled to 0° C. while being stirred. In a nitrogen atmosphere, phenyl magnesium bromide (1.5 eq) was slowly added dropwise thereto at 0° C., and stirred for 1 hour at 40° C. to obtain Intermediate 1a solution. After the mixture was cooled, ammonium chloride solution was slowly added dropwise thereto, and the mixture was washed three times utilizing ethyl acetate and then three times utilizing water, and the resulting organic layer was dried utilizing MgSO₄ and dried under reduced pressure. Intermediate 1a was obtained by column chromatography. (yield: 65%)

### 1-2. Synthesis of Intermediate 1b

2-bromo-4′-chloro-1,1′-biphenyl (1.0 eq) and tetrahydrofuran were cooled to −78° C. while stirring. n-butyl lithium (1.1 eq) was slowly added dropwise to the solution and stirred for 1 hour at −78° C. Intermediate 1a (1.1 eq) was slowly added dropwise thereto at −78° C. and stirred for 4 hours at room temperature. Once the mixture was washed three times utilizing ethyl acetate and water, the resulting organic layer was dried utilizing MgSO₄ and dried under reduced pressure. Intermediate 1b was obtained by column chromatography. (yield: 69%)

### 1-3. Synthesis of Intermediate 1c

Intermediate 1b (1.0 eq) was dissolved in acetic acid:hydrochloric acid in a volume ratio of 9:1 and stirred for 2 hours at 80° C. in a nitrogen atmosphere. Once the mixture was cooled and washed three times utilizing ethyl acetate and water, the resulting organic layer was dried utilizing MgSO₄ under reduced pressure. Intermediate 1c was obtained by column chromatography. (yield: 69%)

### 1-4. Synthesis of Intermediate 1d

2-bromo-9,9-dimethyl-9H-fluorene (1 eq.), aniline (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 1d was obtained by column chromatography. (yield: 85%)

### 1-5. Synthesis of Compound 1

Intermediate 1d (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 1 was obtained by column chromatography. (yield: 86%, HRMS (ESI): calcd.: 725.3083; found: 725.3085.)

### Synthesis Example 2: Synthesis of Compound 3

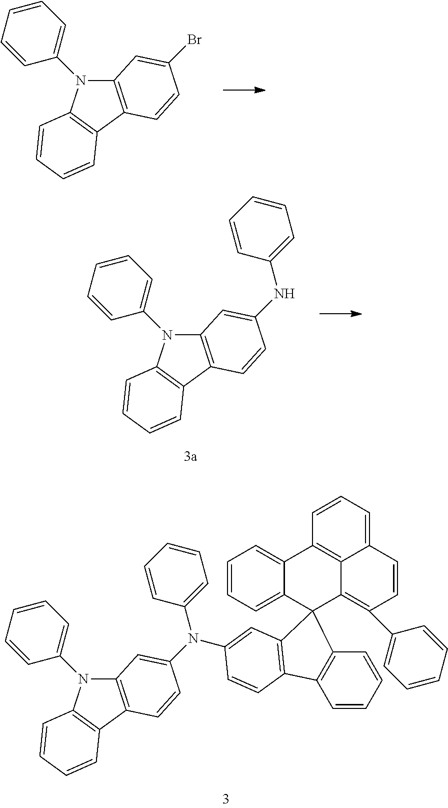 [SMILES: Brc1ccc2c3ccccc3n(-c3ccccc3)c2c1 ; c1ccc(Nc2ccc3c4ccccc4n(-c4ccccc4)c3c2)cc1 ; c1ccc(-c2ccc3cccc4c3c2C2(c3ccccc3-c3ccc(N(c5ccccc5)c5ccc6c7ccccc7n(-c7ccccc7)c6c5)cc32)c2ccccc2-4)cc1]

### 2-1. Synthesis of Intermediate 3a

2-bromo-9-phenyl-9H-carbazole (1 eq), aniline (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 3a was obtained by column chromatography. (yield: 85%)

### 2-2. Synthesis of Compound 3

Intermediate 3a (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 3 was obtained by column chromatography. (yield: 86%, HRMS (ESI): calcd.: 774.3035; found: 774.3037.)

### Synthesis Example 3: Synthesis of Compound 4

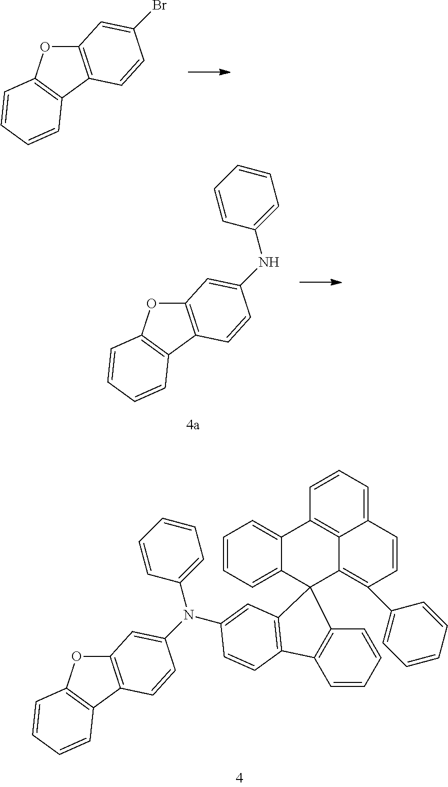 [SMILES: Brc1ccc2c(c1)oc1ccccc12 ; c1ccc(Nc2ccc3c(c2)oc2ccccc23)cc1 ; c1ccc(-c2ccc3cccc4c3c2C2(c3ccccc3-c3ccc(N(c5ccccc5)c5ccc6c(c5)oc5ccccc56)cc32)c2ccccc2-4)cc1]

### 3-1. Synthesis of Intermediate 4a

3-bromodibenzofuran (1 eq), aniline (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 4a was obtained by column chromatography. (yield: 85%)

### 3-2. Synthesis of Compound 4

Intermediate 4a (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 4 was obtained by column chromatography. (yield: 86%, HRMS (ESI): calcd.: 699.2562; found: 699.2564.)

### Synthesis Example 4: Synthesis of Compound 11

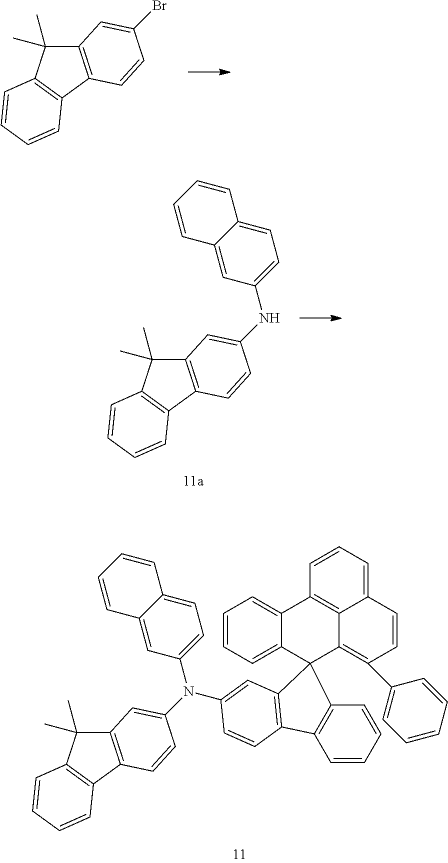 [SMILES: CC1(C)c2ccccc2-c2ccc(Br)cc21 ; CC1(C)c2ccccc2-c2ccc(Nc3ccc4ccccc4c3)cc21 ; CC1(C)c2ccccc2-c2ccc(N(c3ccc4c(c3)C3(c5ccccc5-4)c4ccccc4-c4cccc5ccc(-c6ccccc6)c3c45)c3ccc4ccccc4c3)cc21]

### 4-1. Synthesis of Intermediate 11a

2-bromo-9,9-dimethyl-9H-fluorene (1 eq), 2-aminonaphthalene(1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 11a was obtained by column chromatography. (yield: 85%)

### 4-2. Synthesis of Compound 11

Intermediate 11a (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 11 was obtained by column chromatography. (yield: 86%, HRMS (ESI): calcd.: 775.3239; found: 775.3241.)

### Synthesis Example 5: Synthesis of Compound 13

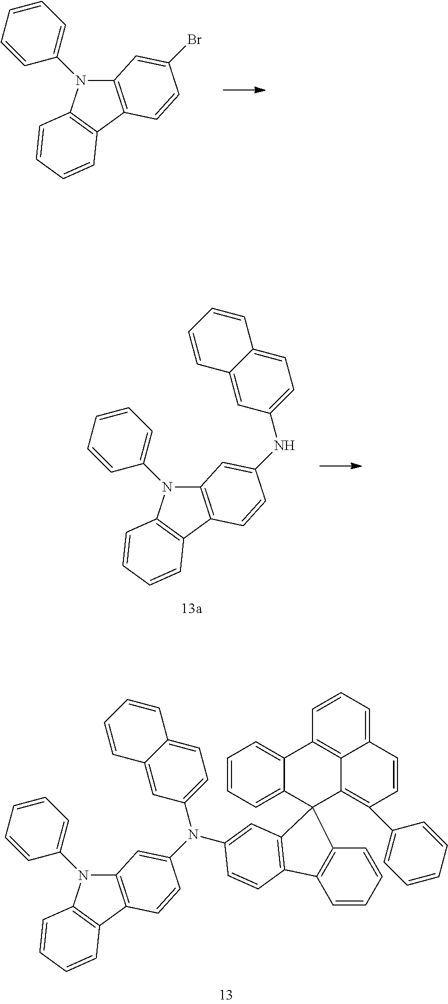 [SMILES: Brc1ccc2c3ccccc3n(-c3ccccc3)c2c1 ; c1ccc(-n2c3ccccc3c3ccc(Nc4ccc5ccccc5c4)cc32)cc1 ; c1ccc(-c2ccc3cccc4c3c2C2(c3ccccc3-c3ccc(N(c5ccc6ccccc6c5)c5ccc6c7ccccc7n(-c7ccccc7)c6c5)cc32)c2ccccc2-4)cc1]

### 5-1. Synthesis of Intermediate 13a

2-bromo-9-phenyl-9H-carbazole (1 eq), 2-aminonaphthalene (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 13a was obtained by column chromatography. (yield: 85%)

### 5-2. Synthesis of Compound 13

Intermediate 13a (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 13 was obtained by column chromatography. (yield: 86%, HRMS (ESI): calcd.: 785.2957; found: 785.2955.)

### Synthesis Example 6: Synthesis of Compound 16

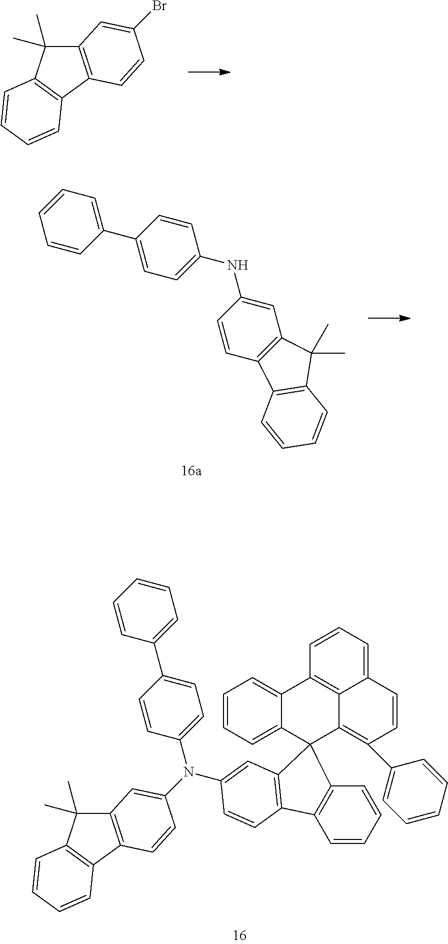 [SMILES: CC1(C)c2ccccc2-c2ccc(Br)cc21 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc4c(c3)C3(c5ccccc5-4)c4ccccc4-c4cccc5ccc(-c6ccccc6)c3c45)cc21]

### 6-1. Synthesis of Intermediate 16a

2-bromo-9,9-dimethyl-9H-fluorene (1 eq), [1,1′-biphenyl]-4-amine (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 16a was obtained by column chromatography. (yield: 85%)

### 6-2. Synthesis of Compound 16

Intermediate 16a (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 16 was obtained by column chromatography. (yield: 86%, HRMS (ESI): calcd.: 801.3396; found: 801.3398.)

### Synthesis Example 7: Synthesis of Compound 21

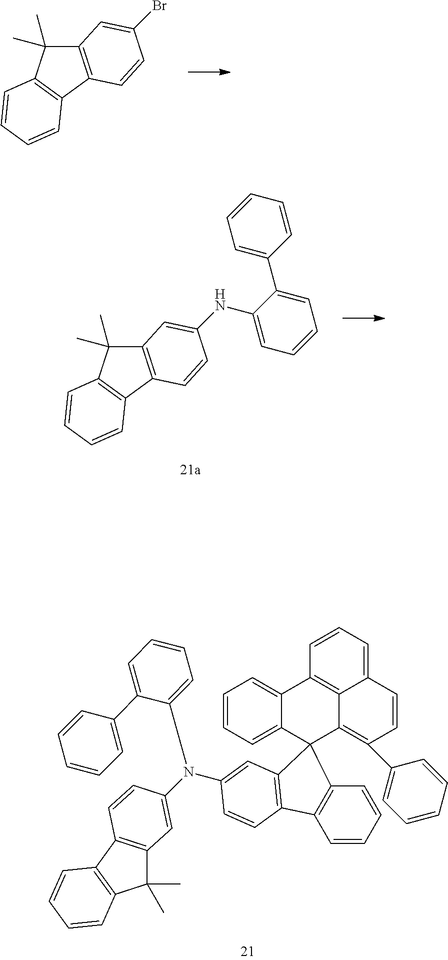 [SMILES: CC1(C)c2ccccc2-c2ccc(Br)cc21 ; CC1(C)c2ccccc2-c2ccc(Nc3ccccc3-c3ccccc3)cc21 ; CC1(C)c2ccccc2-c2ccc(N(c3ccc4c(c3)C3(c5ccccc5-4)c4ccccc4-c4cccc5ccc(-c6ccccc6)c3c45)c3ccccc3-c3ccccc3)cc21]

### 7-1. Synthesis of Intermediate 21a

2-bromo-9,9-dimethyl-9H-fluorene (1 eq), [1,1′-biphenyl]-2-amine (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 21a was obtained by column chromatography. (yield: 85%)

### 7-2. Synthesis of Compound 21

Intermediate 21a (1 eq), Intermediate 1c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 21 was obtained by column chromatography. (yield: 86%, HRMS (ESI): calcd.: 801.3396; found: 801.3398.)

### Synthesis Example 8: Synthesis of Compound 101

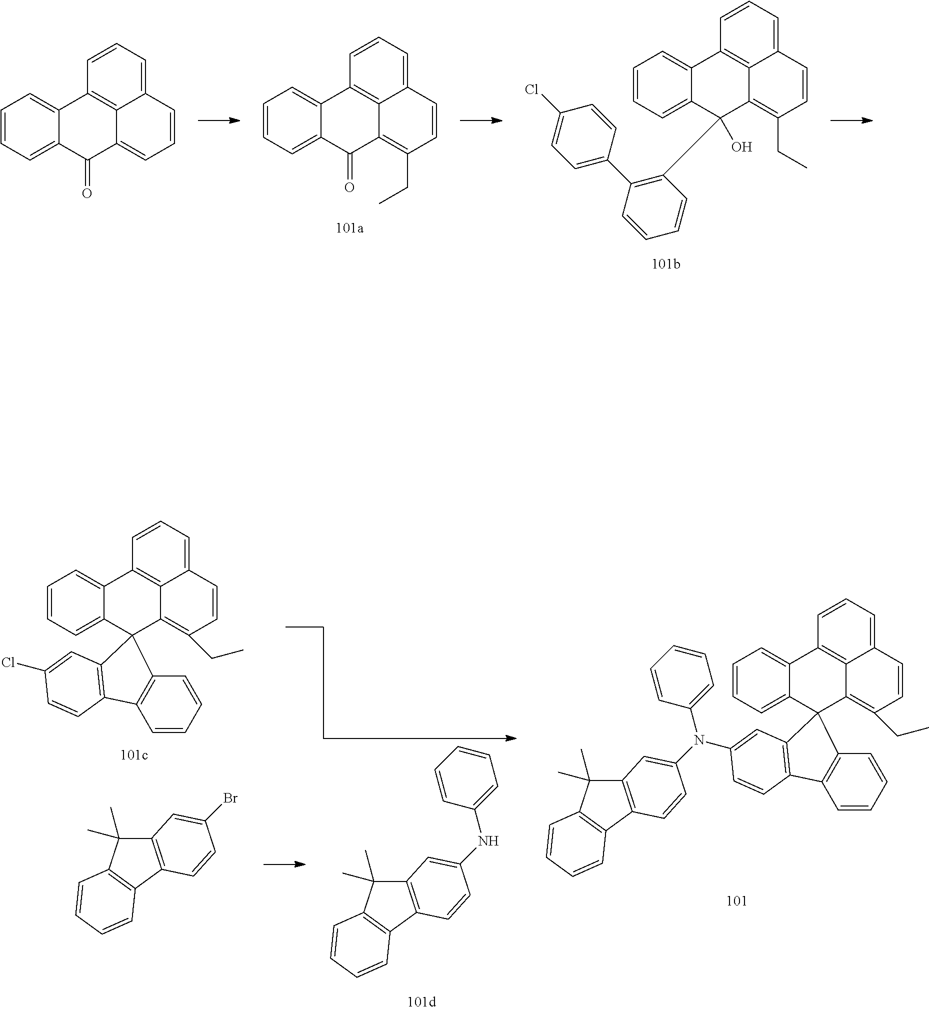

### 8-1. Synthesis of Intermediate 101a

7H-benzo[de]anthracen-7-one (1.0 eq) and anhydrous diethyl ether were cooled to 0° C. while being stirred. In a nitrogen atmosphere, ethyl magnesium bromide (1.5 eq) was slowly added dropwise thereto at 0° C., and stirred for 1 hour at 40° C. to obtain Intermediate 101a solution. After the mixture was cooled, ammonium chloride solution was slowly added dropwise thereto, and the mixture was washed three times utilizing ethyl acetate and water, and the resulting organic layer was dried utilizing MgSO₄ and dried under reduced pressure. Intermediate 101a was obtained by column chromatography. (yield: 65%)

### 8-2. Synthesis of Intermediate 101b

2-bromo-4′-chloro-1,1′-biphenyl (1.0 eq) and tetrahydrofuran were cooled to −78° C. while stirring. n-butyl lithium (1.1 eq) was slowly added dropwise to the solution and stirred for 1 hour at −78° C. Intermediate 101a (1.1 eq) was slowly added dropwise thereto at −78° C. and stirred for 4 hours at room temperature. Once the mixture was washed three times utilizing ethyl acetate and water, the resulting organic layer was dried utilizing MgSO₄ and dried under reduced pressure. Intermediate 101 b was obtained by column chromatography. (yield: 69%)

### 8-3. Synthesis of Intermediate 101c

Intermediate 101 b (1.0 eq) was dissolved in acetic acid:hydrochloric acid in a volume ratio of 9:1 and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 101c was obtained by column chromatography. (yield: 69%)

### 8-4. Synthesis of Intermediate 101d

2-bromo-9,9-dimethyl-9H-fluorene (1 eq), aniline (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Intermediate 101d was obtained by column chromatography. (yield: 85%)

### 8-5. Synthesis of Compound 101

Intermediate 101d (1 eq), Intermediate 101c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 101 was obtained by column chromatography. (yield: 86%, HRMS (ESI): calcd.: 677.3083; found: 677.3081.)

### Synthesis Example 9: Synthesis of Compound 121

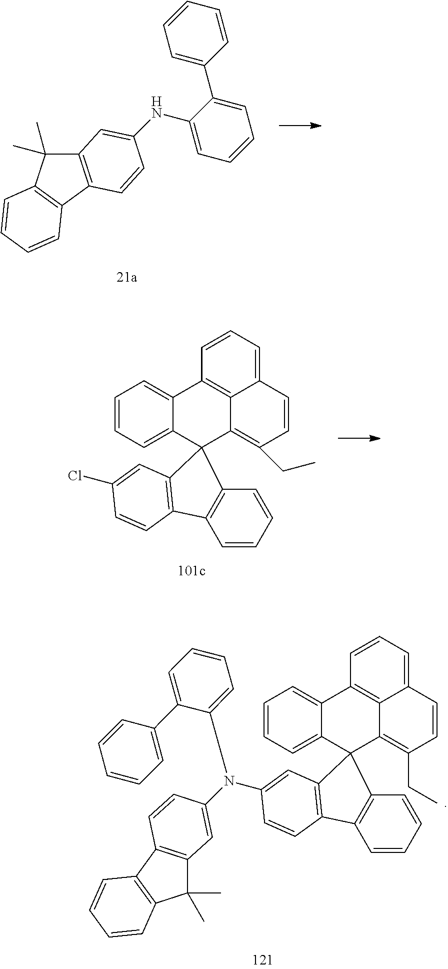 [SMILES: CC1(C)c2ccccc2-c2ccc(Nc3ccccc3-c3ccccc3)cc21 ; CCc1ccc2cccc3c2c1C1(c2ccccc2-c2ccc(Cl)cc21)c1ccccc1-3 ; CCc1ccc2cccc3c2c1C1(c2ccccc2-c2ccc(N(c4ccc5c(c4)C(C)(C)c4ccccc4-5)c4ccccc4-c4ccccc4)cc21)c1ccccc1-3]

Intermediate 21a (1 eq), Intermediate 101c (1.1 eq), tris(dibenzylideneacetone)dipalladium(0) (0.03 eq), tri-tert-butylphosphine (0.06 eq), and sodium tert-butoxide (2.0 eq) were dissolved in toluene and stirred for 2 hours at 80° C. in a nitrogen atmosphere. Once the mixture was cooled and washed three times utilizing ethyl acetate and water, the resulting organic layer was dried utilizing MgSO₄ and dried under reduced pressure. Compound 121 was obtained by column chromatography. (yield: 86%, HRMS (ESI): calcd.: 753.3396; found: 753.3396.)

### Evaluation Example 1

The HOMO energy and LUMO energy of the compounds utilized in Synthesis Examples 1 to 9 and Comparative Example were measured, and the results thereof are shown in Table 2.

Particularly, the HOMO energy and LUMO energy were measured utilizing the methods shown in Table 1.

TABLE 1

| | |
|---|---|
| HOMO | By utilizing cyclic voltammetry (CV) (electrolyte: |
| energy level | 0.1M Bu₄NPF₆/solvent: dimethylforamide (DMF)/ |
| evaluation | electrode: 3-electrode system (working electrode: |
| method | GC, reference electrode: Ag/AgCl, and auxiliary |
| | electrode: Pt)), the potential (V)-current (A) graph |
| | of each compound was obtained, and then, from |
| | the oxidation onset of the graph, the HOMO energy |
| | level of each compound was calculated. |
| LUMO | By utilizing cyclic voltammetry (CV) (electrolyte: |
| energy level | 0.1M Bu₄NPF₆/solvent: dimethylforamide (DMF)/ |
| evaluation | electrode: 3-electrode system (working electrode: |
| method | GC, reference electrode: Ag/AgCl, and auxiliary |
| | electrode: Pt)), the potential (V)-current (A) graph |
| | of each compound was obtained, and then, from |
| | the reduction onset of the graph, the LUMO energy |
| | level of each compound was calculated. |

TABLE 2

| | Compound | HOMO (eV) | LUMO (eV) |
|---|---|---|---|
| Synthesis | Compound 1 | −5.19 | −1.70 |
| Example 1 | | | |
| Synthesis | Compound 3 | −5.15 | −1.70 |
| Example 2 | | | |
| Synthesis | Compound 4 | −5.20 | −1.68 |
| Example 3 | | | |
| Synthesis | Compound 11 | −5.20 | −1.68 |
| Example 4 | | | |
| Synthesis | Compound 13 | −5.15 | −1.67 |
| Example 5 | | | |
| Synthesis | Compound 16 | −5.17 | −1.70 |
| Example 6 | | | |
| Synthesis | Compound 21 | −5.19 | −1.70 |
| Example 7 | | | |
| Synthesis | Compound 101 | −5.18 | −1.70 |
| Example 8 | | | |
| Synthesis | Compound 121 | −5.16 | −1.70 |
| Example 9 | | | |
| Comparative | NPB | −5.09 | −2.00 |
| Example 1 | | | |
| Comparative | Compound A | −5.18 | −1.71 |
| Example 2 | | | |
| Comparative | Compound B | −5.12 | −1.60 |
| Example 3 | | | |

### Comparative Example 1

As an anode, a glass substrate with 15 Ω/cm² (1,200 Å) ITO thereon, which was manufactured by Corning Inc. was cut to a size of 50 mm×50 mm×0.7 mm, and the glass substrate was sonicated by utilizing isopropyl alcohol and pure water for 5 minutes each, and then ultraviolet (UV) light was irradiated for 30 minutes thereto and ozone was exposed thereto for cleaning. Then, the resultant glass substrate was loaded onto a vacuum deposition apparatus.

On the glass substrate, first, 2-TNATA which is a suitable material in the art was vacuum-deposited to form a hole injection layer having a thickness of 600 Å, and 4,4′-bis[N-(1-naphthyl)-N-phenylamino]biphenyl (hereinafter, referred to as NPB) which is a hole transport material was vacuum-deposited as a hole transport compound to form a hole transport layer having a thickness of 300 Å.

9,10-di(naphthalen-2-yl)anthracene (hereinafter, referred to as ADN) which is a suitable blue fluorescence host in the art and 4,4′-bis[2-(4-(N,N-diphenylamino)phenyl)vinyl]biphenyl (hereinafter, referred to as DPAVBi) which is a suitable blue fluorescence dopant compound in the art were co-deposited on the hole transport layer at a weight ratio of 98:2 to form an emission layer having a thickness of 300 Å.

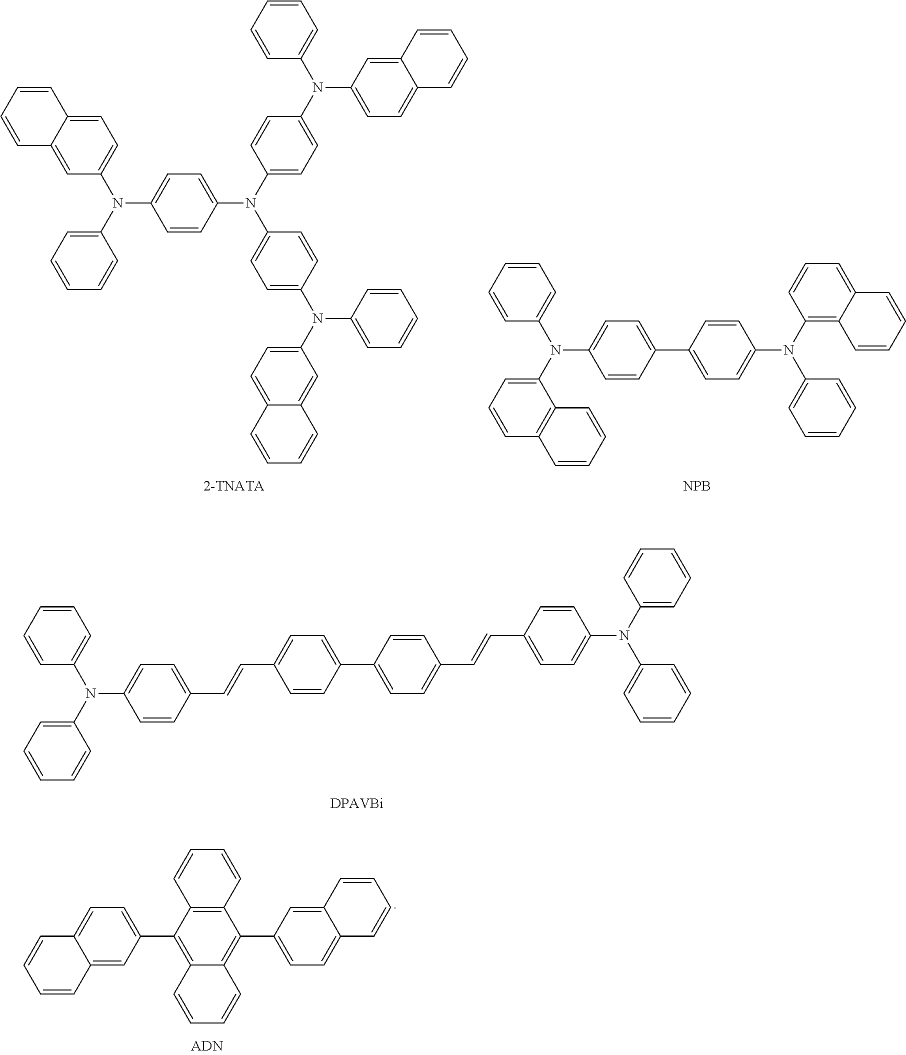

Then, Alq₃ was deposited on the emission layer to form an electron transport layer having a thickness of 300 Å, and then, LiF, which is a halogenated alkali metal, was deposited on the electron transport layer to form an electron injection layer having a thickness of 10 Å and Al was vacuum-deposited thereon to form an Al electrode having a thickness of 3,000 Å (cathode) to form an LiF/Al electrode, thereby completing the manufacture of an organic electroluminescent (EL) device.

### Comparative Example 2

An organic EL device was manufactured in substantially the same manner as in Comparative Example 1, except that Compound A was utilized instead of NPB in forming a hole transport layer.

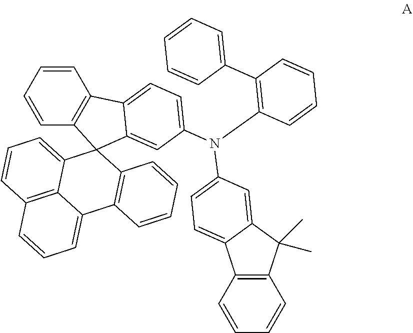 [SMILES: CC1(C)c2ccccc2-c2ccc(N(c3ccc4c(c3)C3(c5ccccc5-4)c4ccccc4-c4cccc5cccc3c45)c3ccccc3-c3ccccc3)cc21]

### Comparative Example 3

An organic EL device was manufactured in substantially the same manner as in Comparative Example 1, except that Compound B was utilized instead of NPB in forming a hole transport layer.

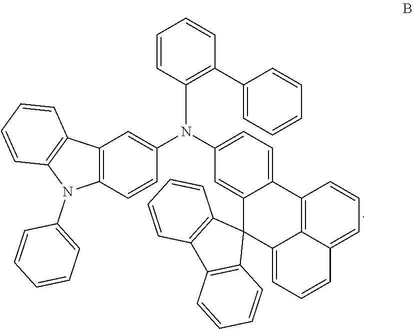 [SMILES: c1ccc(-c2ccccc2N(c2ccc3c(c2)C2(c4ccccc4-c4ccccc42)c2cccc4cccc-3c24)c2ccc3c(c2)c2ccccc2n3-c2ccccc2)cc1]

### Example 1

An organic EL device was manufactured in substantially the same manner as in Comparative Example 1, except that Compound 1 was utilized instead of NPB in forming a hole transport layer.

### Example 2

An organic EL device was manufactured in substantially the same manner as in Comparative Example 1, except that Compound 3 was utilized instead of NPB in forming a hole transport layer.

### Example 3

An organic EL device was manufactured in substantially the same manner as in Comparative Example 1, except that Compound 4 was utilized instead of NPB in forming a hole transport layer.

### Example 4

An organic EL device was manufactured in substantially the same manner as in Comparative Example 1, except that Compound 11 was utilized instead of NPB in forming a hole transport layer.

### Example 5

An organic EL device was manufactured in substantially the same manner as in Comparative Example 1, except that Compound 13 was utilized instead of NPB in forming a hole transport layer.

### Example 6

An organic EL device was manufactured in substantially the same manner as in Comparative Example 1, except that Compound 16 was utilized instead of NPB in forming a hole transport layer.

### Example 7

An organic EL device was manufactured in substantially the same manner as in Comparative Example 1, except that Compound 21 was utilized instead of NPB in forming a hole transport layer.

### Example 8

An organic EL device was manufactured in substantially the same manner as in Comparative Example 1, except that Compound 101 was utilized instead of NPB in forming a hole transport layer.

### Example 9

An organic EL device was manufactured in substantially the same manner as in Comparative Example 1, except that Compound 121 was utilized instead of NPB in forming a hole transport layer.

### Evaluation Example 2

To evaluate the characteristics of the organic electroluminescent device according to the Examples and Comparative Examples, the driving voltage, luminance (cd/m²), luminescence efficiency (Cd/A) at a current density of 50 mA/cm², and half lifespan at a current density of 100 mA/cm² were respectively measured utilizing Keithley MU236 and luminance meter PR650, and the results thereof are shown in Table 3. In Table 3, the half lifespan is a measure of the time (hr) taken for the luminance to reach 50% of the initial luminance.

TABLE 3

| | | | | | | | Half |
|---|---|---|---|---|---|---|---|
| | Hole | Driving | Current | | Luminescence | | lifespan |
| | transport | voltage | density | Luminance | efficiency | Emission | (hr @100 |
| | layer | (V) | (mA/cm²) | (cd/m²) | (cd/A) | color | mA/cm²) |
| Comparative | NPB | 7.01 | 50 | 2645 | 5.29 | Blue | 258 |
| Example 1 | | | | | | | |
| Comparative | Compound A | 4.22 | 50 | 3601 | 7.20 | Blue | 234 |
| Example 2 | | | | | | | |
| Comparative | Compound B | 4.62 | 50 | 3601 | 7.20 | Blue | 215 |
| Example 3 | | | | | | | |
| Example 1 | Compound 1 | 4.10 | 50 | 3750 | 7.5 | Blue | 359 |
| Example 2 | Compound 3 | 4.21 | 50 | 3790 | 7.58 | Blue | 375 |
| Example 3 | Compound 4 | 4.35 | 50 | 3650 | 7.3 | Blue | 342 |
| Example 4 | Compound 11 | 4.10 | 50 | 3720 | 7.44 | Blue | 355 |
| Example 5 | Compound 13 | 4.15 | 50 | 3780 | 7.56 | Blue | 376 |
| Example 6 | Compound 16 | 4.15 | 50 | 3730 | 7.46 | Blue | 378 |
| Example 7 | Compound 21 | 4.05 | 50 | 3760 | 7.52 | Blue | 362 |
| Example 8 | Compound 101 | 4.37 | 50 | 3690 | 7.38 | Blue | 362 |
| Example 9 | Compound 121 | 4.22 | 50 | 3705 | 7.41 | Blue | 370 |

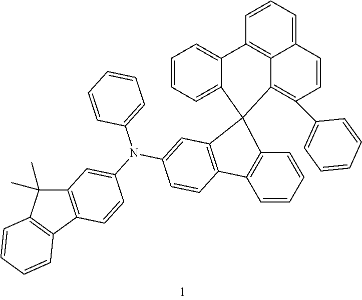 [SMILES: CC1(C)c2ccccc2-c2ccc(N(c3ccccc3)c3ccc4c(c3)C3(c5ccccc5-4)c4ccccc4-c4cccc5ccc(-c6ccccc6)c3c45)cc21]

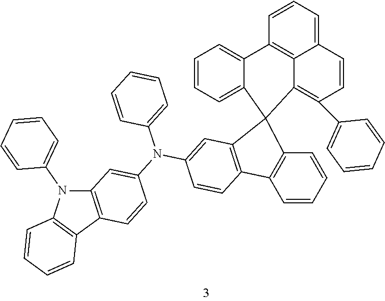 [SMILES: c1ccc(-c2ccc3cccc4c3c2C2(c3ccccc3-c3ccc(N(c5ccccc5)c5ccc6c7ccccc7n(-c7ccccc7)c6c5)cc32)c2ccccc2-4)cc1]

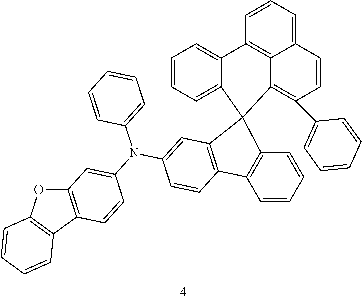 [SMILES: c1ccc(-c2ccc3cccc4c3c2C2(c3ccccc3-c3ccc(N(c5ccccc5)c5ccc6c(c5)oc5ccccc56)cc32)c2ccccc2-4)cc1]

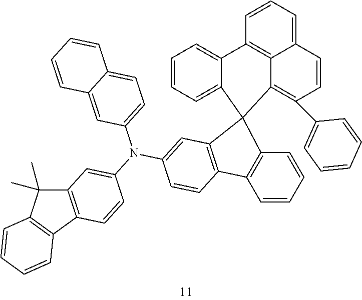 [SMILES: CC1(C)c2ccccc2-c2ccc(N(c3ccc4c(c3)C3(c5ccccc5-4)c4ccccc4-c4cccc5ccc(-c6ccccc6)c3c45)c3ccc4ccccc4c3)cc21]

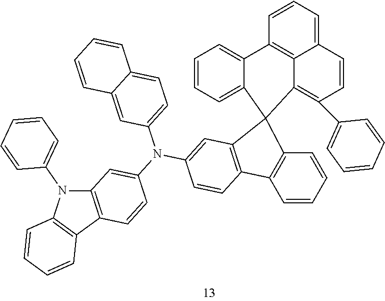 [SMILES: c1ccc(-c2ccc3cccc4c3c2C2(c3ccccc3-c3ccc(N(c5ccc6ccccc6c5)c5ccc6c7ccccc7n(-c7ccccc7)c6c5)cc32)c2ccccc2-4)cc1]

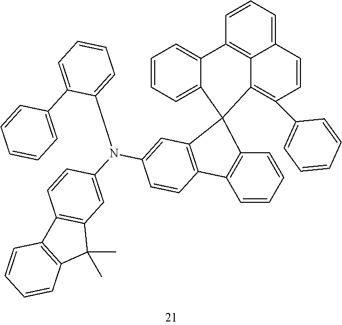 [SMILES: CC1(C)c2ccccc2-c2ccc(N(c3ccc4c(c3)C3(c5ccccc5-4)c4ccccc4-c4cccc5ccc(-c6ccccc6)c3c45)c3ccccc3-c3ccccc3)cc21]

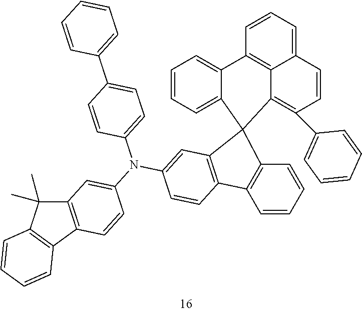 [SMILES: CC1(C)c2ccccc2-c2ccc(N(c3ccc(-c4ccccc4)cc3)c3ccc4c(c3)C3(c5ccccc5-4)c4ccccc4-c4cccc5ccc(-c6ccccc6)c3c45)cc21]

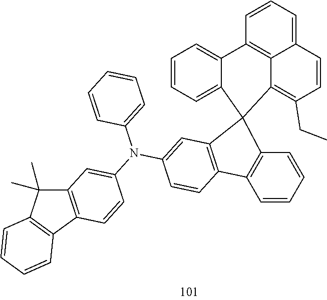 [SMILES: CCc1ccc2cccc3c2c1C1(c2ccccc2-c2ccc(N(c4ccccc4)c4ccc5c(c4)C(C)(C)c4ccccc4-5)cc21)c1ccccc1-3]

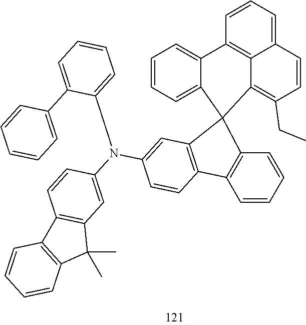 [SMILES: CCc1ccc2cccc3c2c1C1(c2ccccc2-c2ccc(N(c4ccc5c(c4)C(C)(C)c4ccccc4-5)c4ccccc4-c4ccccc4)cc21)c1ccccc1-3]

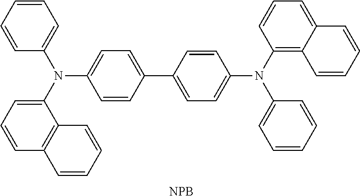 [SMILES: c1ccc(N(c2ccc(-c3ccc(N(c4ccccc4)c4cccc5ccccc45)cc3)cc2)c2cccc3ccccc23)cc1 ; "NPB"]

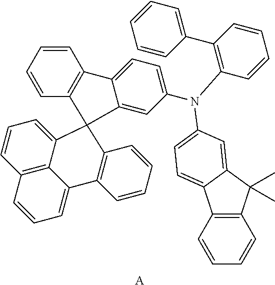

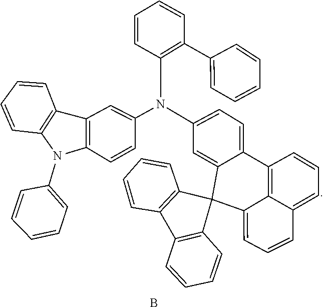

Referring to Table 3, it can be seen that the Examples utilizing the condensed cyclic compound represented by Formula 1 have excellent or suitable current-voltage-luminance (I-V-L) characteristics in which driving voltage and/or luminescence efficiency of each is improved as compared with those of the Comparative Examples, and particularly, when comparing the half lifespan, the lifespan improvement effect of each of the Examples are excellent or suitable compared to that of the Comparative Examples.

As described above, according to the one or more embodiments, a light-emitting device including a condensed cyclic compound represented by Formula 1 may have excellent or suitable characteristics in terms of luminescence efficiency and lifespan, without a significant increase in driving voltage. Accordingly, utilization of the light-emitting device may provide a high-quality electronic apparatus.

The use of “may” when describing embodiments of the present disclosure refers to “one or more embodiments of the present disclosure”.

As used herein, the terms “substantially”, “about”, and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic apparatus, the display device, and/or any other relevant devices or components according to embodiments of the present invention described herein may be implemented utilizing any suitable hardware, firmware (e.g. an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprises:
a first electrode;
a second electrode facing the first electrode;
an interlayer between the first electrode and the second electrode and comprising an emission layer; and
at least one condensed cyclic compound represented by Formula 1: 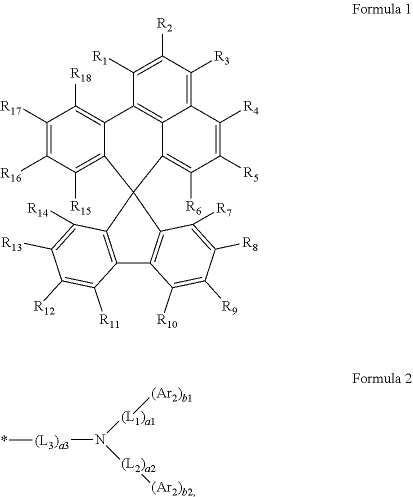
wherein, in Formulae 1 and 2,
at least one selected from the group consisting of R₇ to R₉, R₁₂ to R₁₄, and R₁₆ is a group represented by Formula 2, and remainder ones of these groups are each independently hydrogen or the same as R₁₀ₐ described below,
R₁ to R₆, R₁₀, R₁₁, R₁₅, R₁₇, and R₁₈ are each independently hydrogen or the same as R₁₀ₐ described below, and at least one selected from the group consisting of R₁ to R₆, R₁₀, R₁₁, R₁₅, R₁₇, and R₁₈ is not hydrogen,
L₁ to L₃ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
a1 to a3 are each independently an integer from 1 to 3,
Ar₁ and Ar₂ are each independently a C₅-C₆₀ carbocyclic group unsubstituted or substituted with at least one R₁₀ₐ or a C₁-C₆₀ heterocyclic group unsubstituted or substituted with at least one R₁₀ₐ,
b1 and b2 are each independently an integer from 1 to 10,
* indicates a binding site to an adjacent atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ aryl alkyl group, or a C₂-C₆₀ heteroaryl alkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein
the first electrode is an anode,
the second electrode is a cathode,
the interlayer further comprises a hole transport region between the emission layer and the first electrode,
the interlayer further comprises an electron transport region between the emission layer and the second electrode,
the hole transport region comprises a hole injection layer, a hole transport layer, an emission auxiliary layer, an electron blocking layer, or any combination thereof, and
the electron transport region comprises a buffer layer, a hole blocking layer, an electron control layer, an electron transport layer, an electron injection layer, or any combination thereof.

3. The light-emitting device of claim 2, wherein the hole transport region comprises the at least one condensed cyclic compound represented by Formula 1.

4. The light-emitting device of claim 2, wherein
the hole transport region comprises at least one selected from the group consisting of the hole injection layer, the hole transport layer, and the electron blocking layer, and
the at least one selected from the group consisting of the hole injection layer, the hole transport layer, and the electron blocking layer comprises the at least one condensed cyclic compound represented by Formula 1.

5. The light-emitting device of claim 1, wherein the emission layer is to emit blue light having a maximum emission wavelength of about 430 nm to about 490 nm.

6. An electronic apparatus comprising the light-emitting device of claim 1.

7. The electronic apparatus of claim 6, further comprising a thin-film transistor, wherein
the thin-film transistor comprises a source electrode and a drain electrode, and
the first electrode of the light-emitting device is electrically connected to the source electrode or the drain electrode of the thin-film transistor.

8. The electronic apparatus of claim 6, further comprising a color filter, a color conversion layer, a touch screen layer, a polarizing layer, or any combination thereof.

9. A condensed cyclic compound represented by Formula 1: 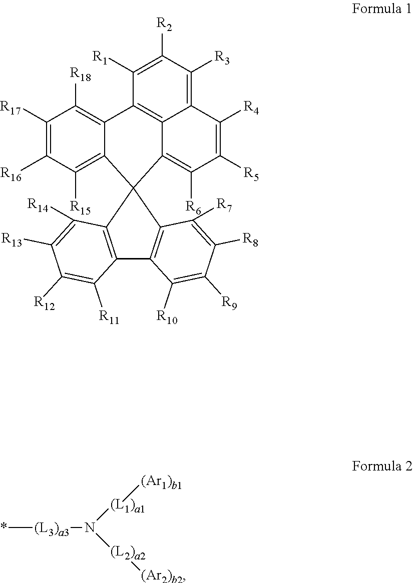
wherein, in Formulae 1 and 2,
at least one selected from the group consisting of R₇ to R₉, R₁₂ to R₁₄, and R₁₆ is a group represented by Formula 2, and remainder ones of these groups are each independently hydrogen or the same as R₁₀ₐ described below,
R₁ to R₆, R₁₀, R₁₁, R₁₅, R₁₇, and R₁₈ are each independently hydrogen or the same as R₁₀ₐ described below, and at least one selected from the group consisting of R₁ to R₆, R₁₀, R₁₁, R₁₅, R₁₇, and R₁₈ is not hydrogen,
L₁ to L₃ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
a1 to a3 are each independently an integer from 1 to 3,
Ar₁ and Ar₂ are each independently a C₅-C₆₀ carbocyclic group unsubstituted or substituted with at least one R₁₀ₐ or a C₁-C₆₀ heterocyclic group unsubstituted or substituted with at least one R₁₀ₐ,
b1 and b2 are each independently an integer from 1 to 10,
* indicates a binding site to an adjacent atom,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ aryl alkyl group, or a C₂-C₆₀ heteroaryl alkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

10. The condensed cyclic compound of claim 9, wherein
L₁ to L₃ in Formula 2 are each independently:
a single bond; or
a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-a fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

11. The condensed cyclic compound of claim 9, wherein L₁ to L₃ in Formula 2 are each independently:
a single bond; or
a group represented by one of Formulae 4-1 to 4-3: 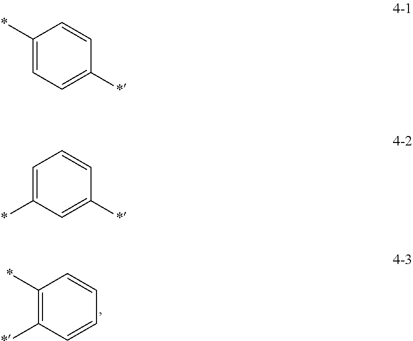
wherein * and *′ in Formulae 4-1 to 4-3 each indicate a binding site to an adjacent atom.

12. The condensed cyclic compound of claim 9, wherein a1 to a3 in Formula 2 are each independently 1 or 2.

13. The condensed cyclic compound of claim 9, wherein Ar₁ and Ar₂ in Formula 2 are each independently a cyclopentyl group, a cyclohexyl group, a cycloheptyl group, a cyclopentenyl group, a cyclohexenyl group, a phenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pyrrolyl group, an imidazolyl group, a pyrazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a carbazolyl group, a phenanthridinyl group, an acridinyl group, a phenanthrolinyl group, a phenazinyl group, a benzoxazolyl group, a benzimidazolyl group, a furanyl group, a benzofuranyl group, a thiophenyl group, a benzothiophenyl group, a thiazolyl group, an isothiazolyl group, a benzothiazolyl group, an isoxazolyl group, an oxazolyl group, a triazolyl group, a tetrazolyl group, an oxadiazolyl group, a triazinyl group, a benzoxazolyl group, a dibenzofuranyl group, a dibenzothiophenyl group, a benzocarbazolyl group, a dibenzocarbazolyl group, or a dibenzosilolyl group, each unsubstituted or substituted with deuterium, —F, —Cl, —Br, —I, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a bicyclo[2.2.2] octyl group, a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, an indenyl group, a fluorenyl group, a spiro-bifluorenyl group, a benzofluorenyl group, a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, —Si(Q₃₁)(Q₃₂)(Q₃₃), —B(Q₃₁)(Q₃₂), or any combination thereof.

14. The condensed cyclic compound of claim 9, wherein Ar₁ and Ar₂ in Formula 2 are each independently a group represented by one of Formulae 5-1 to 5-80: 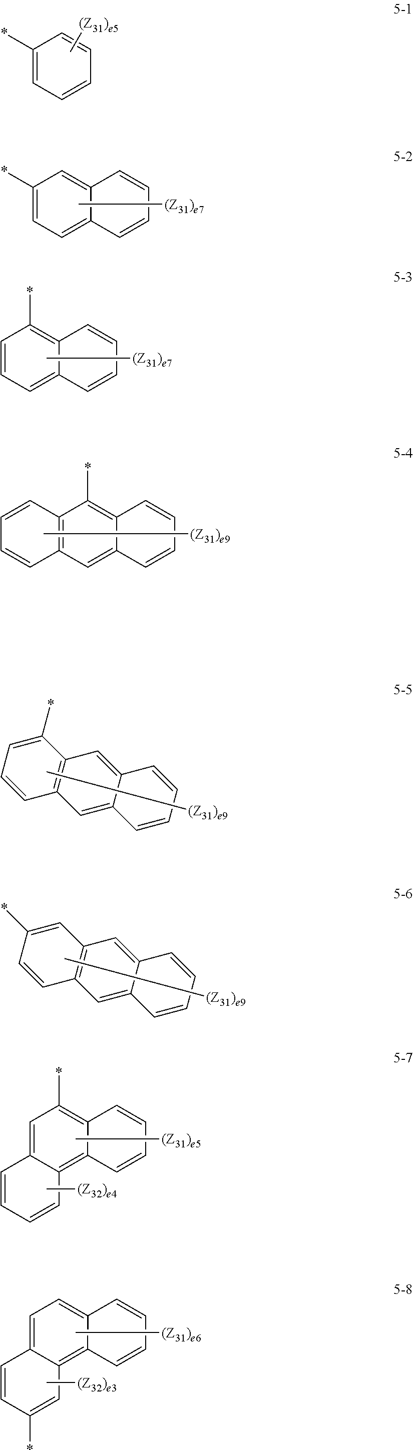 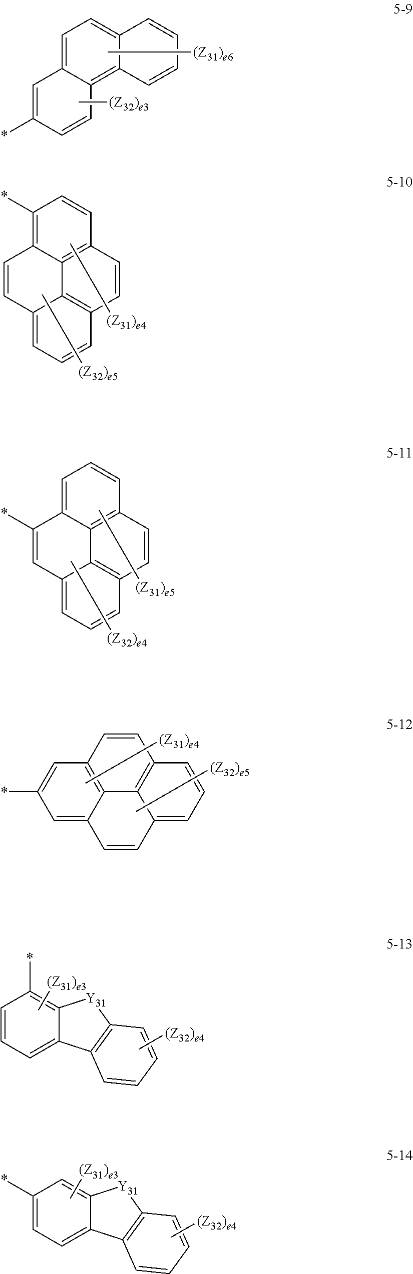 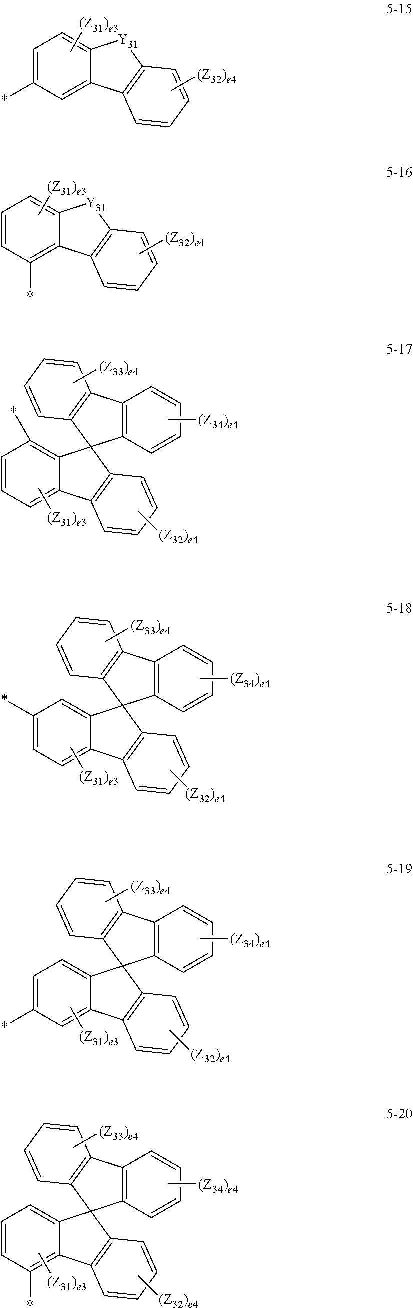 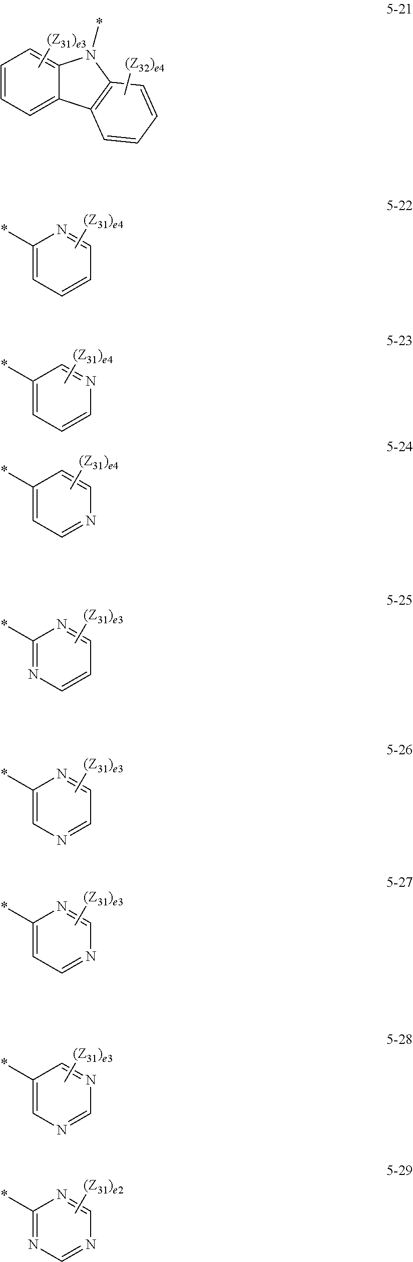 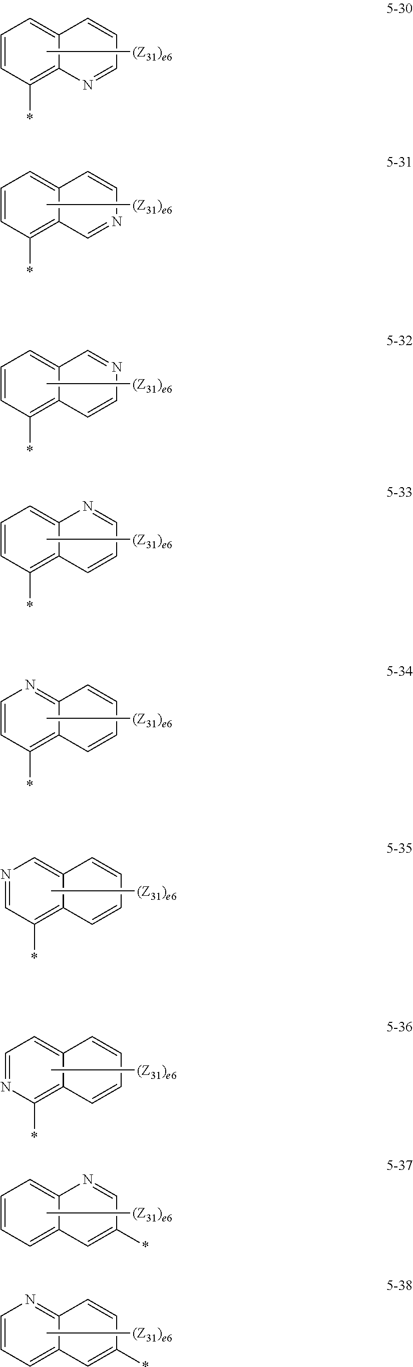 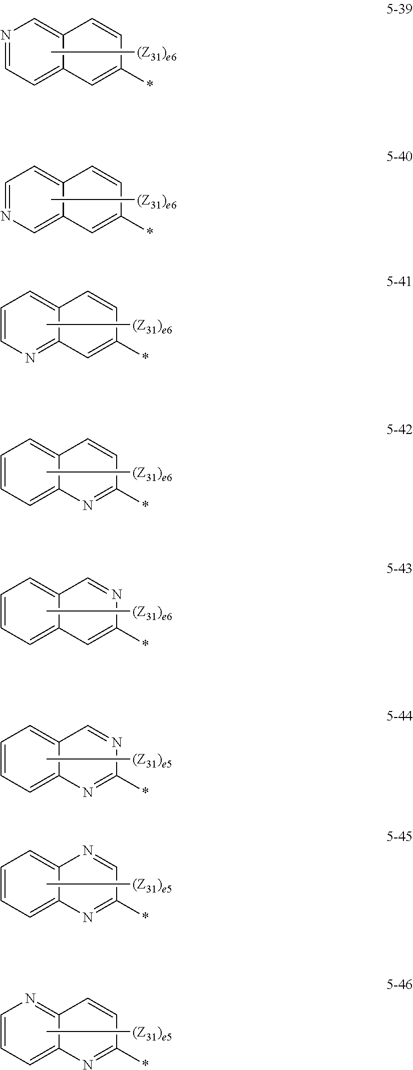 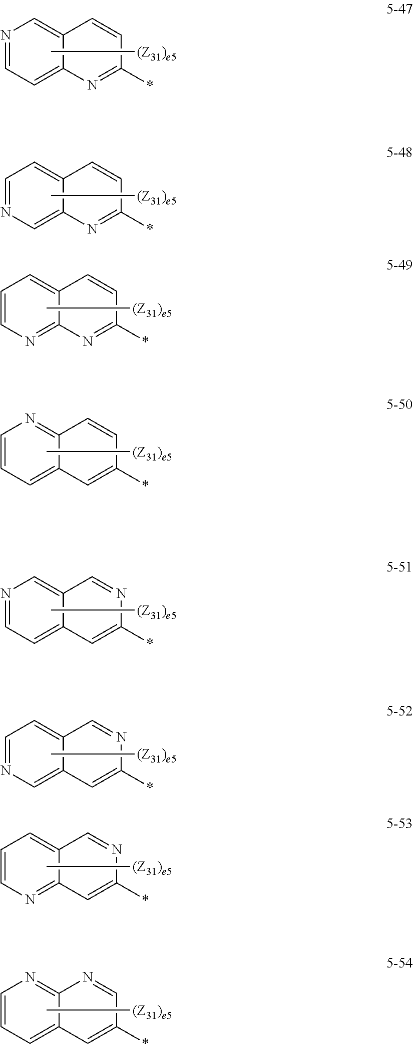 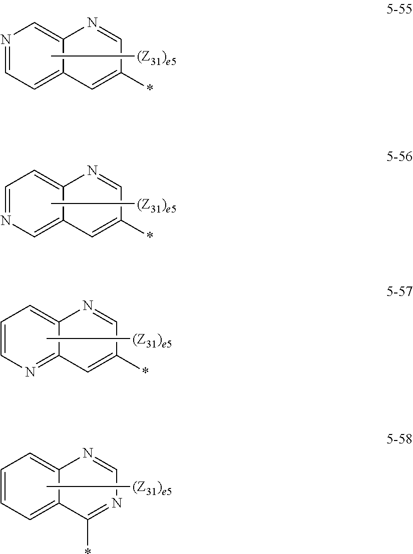 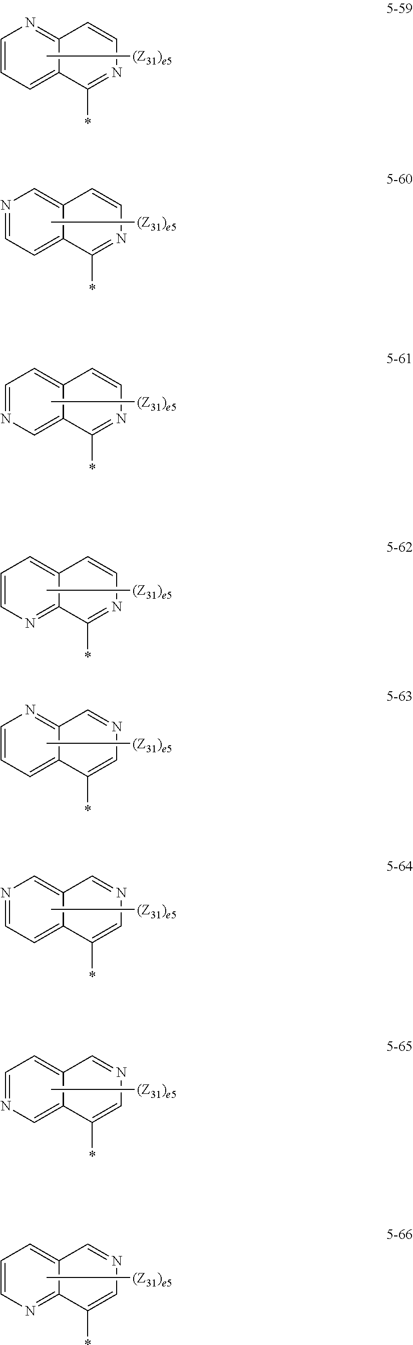 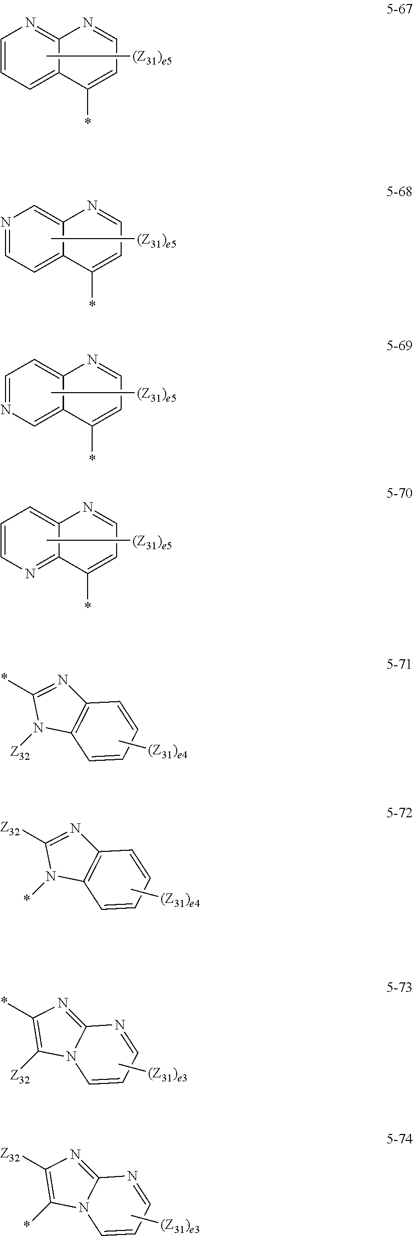 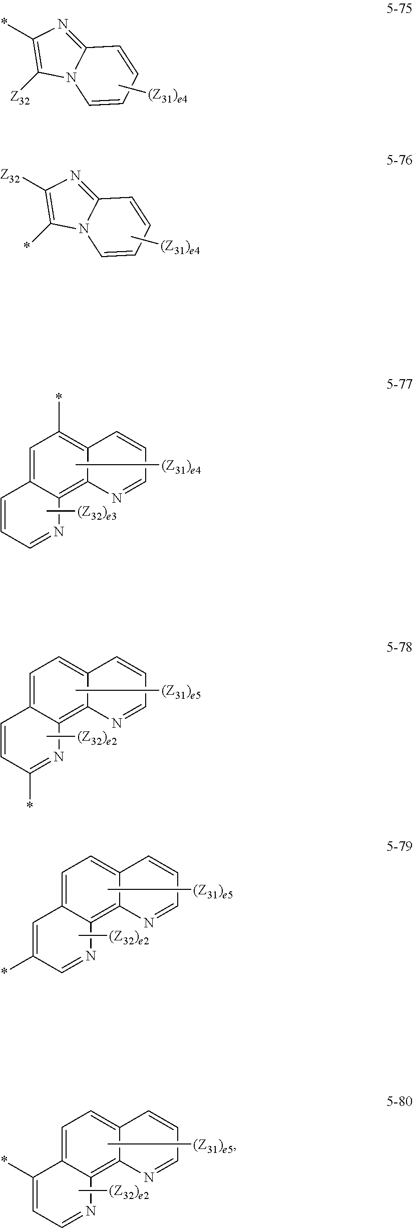
wherein, in Formulae 5-1 to 5-80,
Y₃₁ is O, S, C(Z₃₃)(Z₃₄), N(Z₃₅), or Si(Z₃₆)(Z₃₇),
Z₃₁ to Z₃₇ are each independently hydrogen, deuterium, —F, —Cl, —Br, —I, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, a dibenzosilolyl group, —Si(Q₃₁)(Q₃₂)(Q₃₃), or —B(Q₃₁)(Q₃₂),
e2 is an integer from 0 to 2,
e3 is an integer from 0 to 3,
e4 is an integer from 0 to 4,
e5 is an integer from 0 to 5,
e6 is an integer from 0 to 6,
e7 is an integer from 0 to 7,
e9 is an integer from 0 to 9, and
* indicates a binding site to an adjacent atom.

15. The condensed cyclic compound of claim 9, wherein b1 and b2 in Formula 2 are each independently 1 or 2.

16. The condensed cyclic compound of claim 9, wherein, in Formula 2, a group represented by *-(L₁)ₐ₁-(Ar₁)_{b1} and a group represented by *-(L₂)ₐ₂-(Ar₂)_{b2} are each independently a group represented by one of Formulae 7-1 to 7-10: 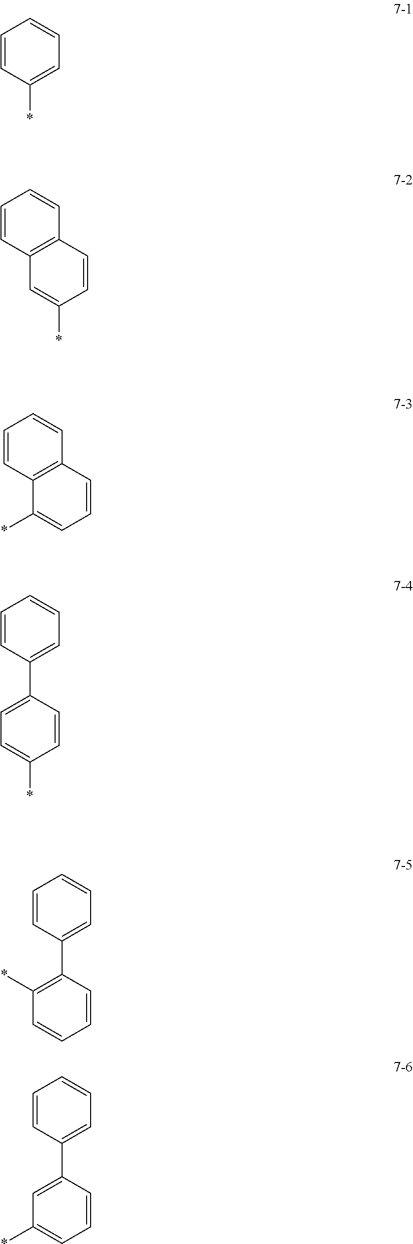 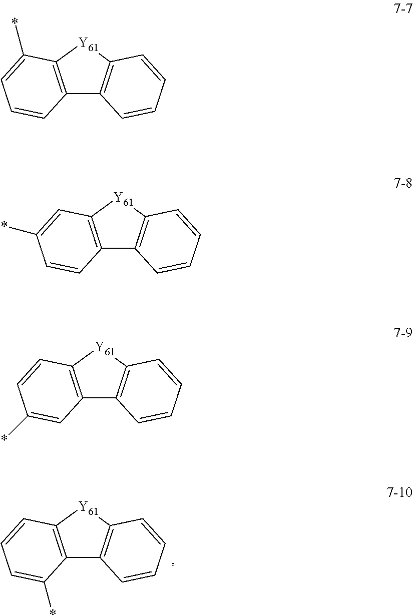
wherein, in Formulae 7-1 to 7-10,
Y₆₁ is O, S, N(R₆₁), or C(R₆₁)(R₆₂),
R₆₁ and R₆₂ are each independently: hydrogen; deuterium; —F; a cyano group; or a C₁-C₂₀ alkyl group or a phenyl group, each unsubstituted or substituted with deuterium, —F, a cyano group, a C₁-C₂₀ alkyl group, a phenyl group, or any combination thereof, and
* indicates a binding site to an adjacent atom.

17. The condensed cyclic compound of claim 9, wherein, in Formula 1,
one selected from the group consisting of R₇ to R₉, R₁₂ to R₁₄, and R₁₆ is a group represented by Formula 2, and remainder ones of these groups are each independently hydrogen, deuterium, —F, —Cl, —Br, —I, a hydroxyl group, a cyano group, a nitro group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, a dibenzosilolyl group, —Si(Q₃₁)(Q₃₂)(Q₃₃), or —B(Q₃₁)(Q₃₂), and
one selected from the group consisting of R₁ to R₆, R₁₀, R₁₁, R₁₅, R₁₇, and R₁₈ is a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrazinyl group, a pyrimidinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a triazinyl group, a dibenzofuranyl group, a dibenzothiophenyl group, a dibenzosilolyl group, —Si(Q₃₁)(Q₃₂)(Q₃₃), or —B(Q₃₁)(Q₃₂), and remainder ones of these groups are each hydrogen.

18. The condensed cyclic compound of claim 9, wherein, in Formula 1, one selected from the group consisting of R₇ to R₉, R₁₂ to R₁₄, and R₁₆ is a group represented by Formula 2, and remainder ones of these groups are each hydrogen.

19. The condensed cyclic compound of claim 9, wherein, in Formula 1, one selected from the group consisting of R₁ to R₆, R₁₀, R₁₁, R₁₅, R₁₇, and R₁₈ is not hydrogen, and remainder ones of these groups are each hydrogen.

20. The condensed cyclic compound of claim 9, wherein the condensed cyclic compound represented by Formula 1 is represented by any one of Formulae 1A-1 to 1A-12: 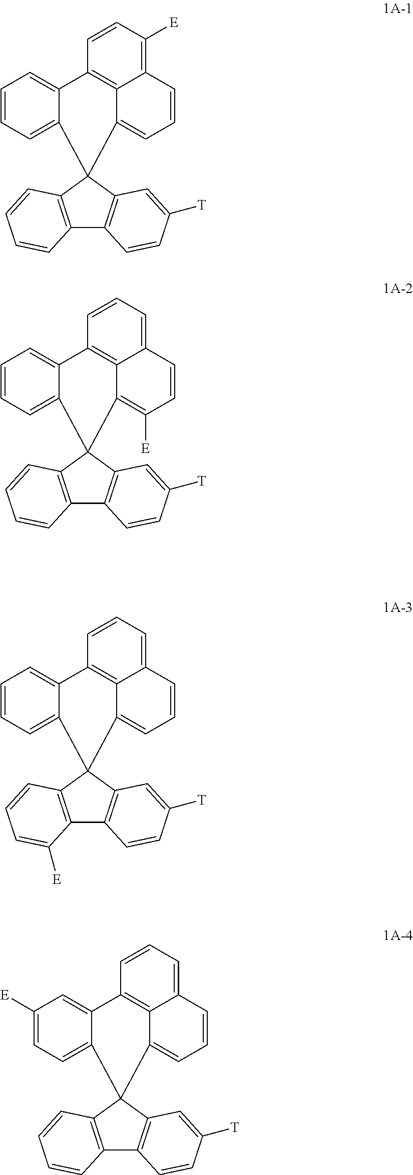 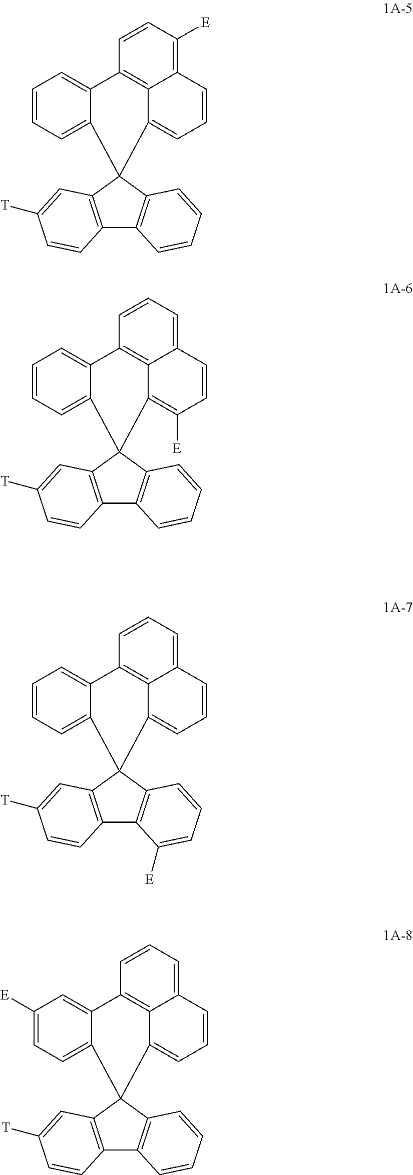 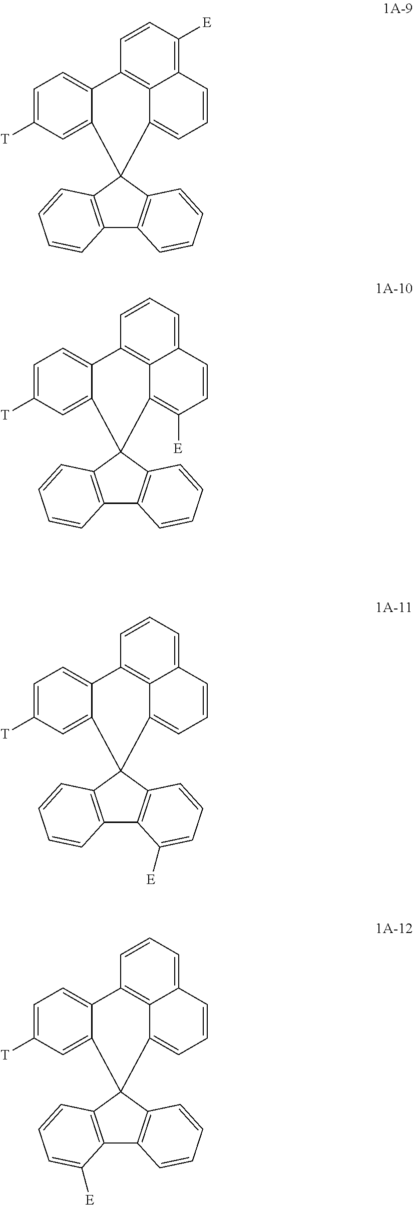
wherein, in Formulae 1A-1 to 1A-12,
T is a group represented by Formula 2, and
E is the same as R₁₀ₐ.
